SEQUENCE LISTING (610 sequences)

SEQ ID NO: 1 - protein, 1018 aa, synthetic construct
  REGION 1..1018: note = Description of Artificial Sequence: Synthetic polypeptide
  REGION 1..1018: note = Category: MG3 chimeric effectors
  REGION 1..1018: note = Description: MG3-6_1-4 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAICISFSRDFKYDKEIKKDIIKGFNPEIVKNAIDKIMPYPYANDKPFKGNTKPLETIYGLRTYGDKSYITQRVELNSIDKKATKIKSIIDETIKNDLLNKLKENPTEQEWKLMLQNYIHPKKQTKVKKVMISVSEGEITKDSNNRERMGEFVDFGTKGTQHQFKHSKRHKGQILYFNEKGVVEVMPVYSNIKTTDVKDKLQNMGCKLYNKGQMFYSGCLVDIPKPFKAGSKEYPAGRYQIKTIRSDKVAELEDACGNKISTNVKYLVPAEFKKVESK

SEQ ID NO: 2 - protein, 1031 aa, synthetic construct
  REGION 1..1031: note = Description of Artificial Sequence: Synthetic polypeptide
  REGION 1..1031: note = Category: MG3 chimeric effectors
  REGION 1..1031: note = Description: MG3-6_1-5 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAMCICFAPTSNAKKALSRKNILPEEIAKNPESDDARNFFAKYLAEVVPTKVAIKKPELEQTIYSKRVIGGRQTIVKKCNVRDLAYKGQNPKYDFDTLTKRIKDIINPVSKRVIEDFAKTEPTEAEWEDWCKYEAAIPSKNGSPTRLLRVLCKTKDDAERFKDLSKDGCGAYRKSKSHKGQFIWKDNKGNYLVAPVYIYSSKQKVYAELKNNPKCMGICDFFKTGCLVKISNEVVDEKKNRLWLKAGFYNLNSIAKEKRVYLTDVNGQEHKKIPLQHLMNAGMKRVETNTI

SEQ ID NO: 3 - protein, 1029 aa, synthetic construct
  REGION 1..1029: note = Description of Artificial Sequence: Synthetic polypeptide
  REGION 1..1029: note = Category: MG3 chimeric effectors
  REGION 1..1029: note = Description: MG3-6_1-6 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAMCLCFAPTGVDSRRAKLGEILPEKLRSEKAAREFFKSYLDKIMPVDVAPKKPRLEDGIYSKRIIGGKACMVKRNNLVDLAYKSGLKPVFDIPTLIKLVDKKEKGIINPQIRKMIGEFAATNPDESAWRKWCEEVRLPSKSGLGARVLRVLVYYGEADEYKDLSKDGCGAYRKGDGHKGQVVWESVDGKYYVEPVYVHASKAGVMAALNANPKKKRICGMFNSHCTVDVGDVYNDRGDFILPAGRYMVNTILTTGRCVLTNADGEKRNPININYLMRAGMRRVELSEL

SEQ ID NO: 4 - protein, 1029 aa, synthetic construct
  REGION 1..1029: note = Description of Artificial Sequence: Synthetic polypeptide
  REGION 1..1029: note = Category: MG3 chimeric effectors
  REGION 1..1029: note = Description: MG3-6_1-7 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAMCLCFAPTGVNSKRARVDMLLPPKIRSEKEAELFFRKYLDKLIPVDVAPKKPKLEDGIYSMRTVGGKKIMARRVNLVDLAYKSGLKPVYDVSVLIKLLDKKERGIINPQIRKLVADFARTNPSEDEWKKWCGECRLPSKNGLGTRVIRVLLNYGEPAEYKDLSKDGRGAFRRGDGHKGQIVWESTDGKYCVLPIYVHASKAKLLAELCANPKKKRICGIFTSHCMVKVGNTYNNKGELLLPEGVYMLNTIRTDGWIQLTSANGDKSKPININYLMKAGMKKVPVKDL

SEQ ID NO: 5 - protein, 1068 aa, synthetic construct
  REGION 1..1068: note = Description of Artificial Sequence: Synthetic polypeptide
  REGION 1..1068: note = Category: MG3 chimeric effectors
  REGION 1..1068: note = Description: MG3-6_2-4 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDALTLGLATALVPGIERKELRRALSLRQAKGDDATLLRSDPKLGEALRWRTEDRFEAAPLSGKLESAVRRALAEGRVVQHVPAKRQGMKVDSNFFGFVEFDETGRLRVRQKMRSPTTRRREIKTTVKNGKNLHTLSHLSLDPKSWLGAPDHPLRRKQLEHGLRTENDLANPKLGNIRGMLPIRENWGIALITKDGSPRLDVIPYINVHQWLEVLALENGGGSPVVLRKGHLVGFDAEKCPEEYCGAWMLLGVKDGRSGTTLELIRPWMVAPRKGGTKESSAKQAIKPASGYSEKEGKASGVFLQRSADVFLKLGLRPLDHDLTGIAAF

SEQ ID NO: 6 - protein, 1006 aa, synthetic construct
  REGION 1..1006: note = Description of Artificial Sequence: Synthetic polypeptide
  REGION 1..1006: note = Category: MG3 chimeric effectors
  REGION 1..1006: note = Description: MG3-6_2-7 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAVTQGLALLLFAPEDWPLLVKRNLPDSEQRHLKARYPFLDFSADKHISIQDLPEDTLHTISERLAECRVVRHIPAKMHGIIVDQTTWGTVAAGAITTLRQKTTEKNARCDENGKRFIKTTEKKRSLLLGGPDAPDGKLAKIKGAILVTENWGCALDPSPTVIPHFKVYPQLRALREKNGGRPIRILRKGSLIQVKAGTYQGIWSVASIKDNADGICLDINAADKVKLENRSDDSKINVRLDSLRKSGLKILKPKLTGACPTTSSP

SEQ ID NO: 7 - protein, 1109 aa, synthetic construct
  REGION 1..1109: note = Description of Artificial Sequence: Synthetic polypeptide
  REGION 1..1109: note = Category: MG3 chimeric effectors
  REGION 1..1109: note = Description: MG3-6_3-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVLTLQSPAIYRVLLTRVNLKHEHEVTGEAPEWRDYEGADQAEKVLYRRWQKNIATLAELMRQEIENNRVPVTRPIRLRKSRGAVHDATVMKALERDLWGEWDAQAIDRLVDPELHLALRKLFTSTKSKKIDVDATSQGLPERYLANQTVQLFDADAPSVMSPRGILRIGAGTHHARLLTWDDPKKGPQLGIQRVFAAEFGEILKDASSNDLFEAPIPFHTMSHRDLQPKVRAAVEQGLTRQIGWITQGDELEIDPADFVGEANAFGNFLREFPERSWSIAGLKKSNTIVIRPLLLSQEGVTAAISPHAAKIVENGIELSNSTLFTAPGTGIIRRTGLGRPRWDSGPAHLPESFNVHARMTQQSARD

SEQ ID NO: 8 - protein, 1122 aa, synthetic construct
  REGION 1..1122: note = Description of Artificial Sequence: Synthetic polypeptide
  REGION 1..1122: note = Category: MG3 chimeric effectors
  REGION 1..1122: note = Description: MG3-6_3-2 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVLTLLDPSVAKTLAMRLDLKREQQDSGRDTRWKEFKGLTPASQERFIKWCQASECLADMLRQQIEADRVPVVVPLRISPSNGAVHDDSVRPLTRQKIDSTWDRKSINRIVDPEIHVAMRRLLNNGTSLPEDKNRVLDLPDGNELGPHDEVELFSTSAASIKLRRGGSAEIGGSIHHARVYAWMGAKGQLEYGMMRVFGAEFPTLTKLSGSKDILRMPIHAGSMSYRDMQDRVRKPIESDIAVELGWITQGDELEILPEAHLETAGGLGDFLKSFPETQWTIDGFNDPSRLRVRPRLMSLEGRDTIDAMGHLSDTEKLKIKQALSKGLMVSASELLSHGAKIIRRDHLGRPRWRGNARPVSIELEQVANQLVNHRSVDGQ

SEQ ID NO: 9 - protein, 1125 aa, synthetic construct
  REGION 1..1125: note = Description of Artificial Sequence: Synthetic polypeptide
  REGION 1..1125: note = Category: MG3 chimeric effectors
  REGION 1..1125: note = Description: MG3-6_3-3 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNPSVAVTLEQRRMLKQENDYSSPRGQHDNGWRDFIGRGEASQSKFLHWKKTAVVLADLISEAIEQDTIPVVNPLRLRPQNGSVHKDTVEAVLERTVGDSWTDKQVSRIVDPNTYIAFLSLLGRKKELDADHQRLVSVSAGVKLLADERVQIFPEEAASILTPRGVVKIGDSIHHARLYGWKNQRGDIQVGMLRVFGAEFPWFMRESGVKDILRVPIPQGSQSYRDLAATTRKFIENGQATEFGWITQNDEIEISAEEYLATDKGDILSDFLGILPEIRWKVTGIEDNRRIRLRPLLLSSEAIPNMLNGRLLTQEEHDLIALVINKGVRVVVSTFLALPSTKIIRRNNLGIPRWRGNGHLPTSLDIQRAATQALEGRD

SEQ ID NO: 10 - protein, 1134 aa, synthetic construct
  REGION 1..1134: note = Description of Artificial Sequence: Synthetic polypeptide
  REGION 1..1134: note = Category: MG3 chimeric effectors
  REGION 1..1134: note = Description: MG3-6_3-4 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRAFYELELDKLDRDQLKPGEDWRNFTGLYEASQNKFSEWKKAATVLGDLLAEAIEDDAIAVVSPLRLRPQNGSVHDDTINAVKKLTLGSAWPADAVKRIVDPEIYLAMKDVLGKLKELPEDSARSLELSDGRYIEADDEVLFFPKKAASILTPRGAAEIGNSIHHARLYSWLTKKGELKFGMLRVYGAEFPWLMRESGSRDVLHMPIHPGSQSFRGMQDGVRKAVESGEAVEFGWITQDDELEFDPEDYIAHGGDDELNRLLRVMPERRWRVDGFYNAGTLRIRPALLSAEQLPSELQKKVADKTLSDVELILLRAVQRGLFVAISSFLPLESLKVIRRNNLGFPRWRGNGNLPTSFEVRSSALRALGVEG

SEQ ID NO: 11 - protein, 1123 aa, synthetic construct
  REGION 1..1123: note = Description of Artificial Sequence: Synthetic polypeptide
  REGION 1..1123: note = Category: MG3 chimeric effectors
  REGION 1..1123: note = Description: MG3-6_3-7 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVLTLLNRSVAVTLEQRRLIKQQREYSLEKSRRERDNVWRDFMGLGPAAQEKFAKWKKTAYVLADIIKEAISNDAIPVVSPLRLRPQNGSVHLDTVDAVLERTIGDAWTVDQVHRIVNPQIYLAFAGYLGNQKALDPDSSRVLALNDGRKLTAEDVIYVFPEKAASILTPRGVVKIGESVHHVRLYAWKNRKGKAEVGMLRVFGAEFPWLMRESGVKDVLRVPIHTGSQSYRDLSFTVRKNIEKGEAAEIGWLTQNEELEFNPESYLQEGGKDKLAKFLAFLPETRWRVDGFPMPDKLRIRPALLSREEIPEGVFRTEEQSLLEEALTKGLIIATKGLLSLPDVKVLRRNNLGIPRWRGGSYRPVSLDIQRAALAALDEQE

SEQ ID NO: 12 - protein, 1134 aa, synthetic construct
  REGION 1..1134: note = Description of Artificial Sequence: Synthetic polypeptide
  REGION 1..1134: note = Category: MG3 chimeric effectors
  REGION 1..1134: note = Description: MG3-6_3-8 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRAFYEQGLDKLDRDQLKPEEDWRNFIGLSLASQEKFLEWKKVTTVLGDLLAEAIEDDSIAVVSPLRLRPQNGRVHKDTIAAVKKQTLGSAWSADAVKRIVDPEIYLAMKDALGKSKVLPEDSARTLELSDGRYLEADDEVLFFPKNAASILTPRGVAEIGGSIHHARLYSWLTKKGELKIGMLRVYGAEFPWLMRESGSHDVLRMPIHPGSQSFRDMQDTTRKAVESSEAVEFAWITQNDELEFEPEDYIAHGGKDELRQFLEFMPECRWRVDGFKKNYQIRIRPAMLSREQLPSDIQRRLESKTLTENESLLLKALDTGLVVAIGGLLPLGTLKVIRRNNLGFPRWRGNGNLPTSFEVRSSALRALGVEG

SEQ ID NO: 13 - protein, 1039 aa, synthetic construct
  REGION 1..1039: note = Description of Artificial Sequence: Synthetic polypeptide
  REGION 1..1039: note = Category: MG3 chimeric effectors
  REGION 1..1039: note = Description: MG3-6_4-2 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAVAIALTDPAALKSISQAASDERRGGRVSFGAVALPWVDFIGDVQAAIEAINVSHRPSRKVNGALHEETFYGPRGMDGDGRPTGYVQRKPVERLSAKEIPNIPDPAVREAVQAKLDEVGGTPAQAFKDPANHPVRKRGIPVHKVRLRLNINPVQVGSGATERHVLTGSNHHMEIIEVRDAKGGKKWTGRLVHRLEAKRRALGRETIVDRAVQAGRQFQFSLSPGDMIELTGEDGERKLHVVRSISEGRIEYVDARDARKKADIRASGDWRKPAVGSLLRLHCRKVVVTPFGEIRYAND

SEQ ID NO: 14 - protein, 1051 aa, synthetic construct
  REGION 1..1051: note = Description of Artificial Sequence: Synthetic polypeptide
  REGION 1..1051: note = Category: MG3 chimeric effectors
  REGION 1..1051: note = Description: MG3-6_4-5 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAVVIALTGPGTVQALTRAALRAKELGRRLFVPLDPPWADRDSFLRDVRASVEAITVSYRVDRKVSGQLHEESNYSKPHMTVDNKGNLVEHRHIRKPLKDMSVEEVEAIVDDRVRKLVQEKLRQLGQEPKKAFADEANHPYFTTADGRLVPIHKARIRKTVATITVGPPQCPRHVAPGLNHHIEILAVRDPAGAVTHWEGELVSLFEAARRVKAGEPVVRRNHGPNKDFLFSLAKGEYVEMELQPGKRQLFRVTVISAKQIEFRLHHDARPTMLLRKTPGARVIRSPGSLFKAKARKVAVDPLGNVFPAND

SEQ ID NO: 15 - protein, 1061 aa, synthetic construct
  REGION 1..1061: note = Description of Artificial Sequence: Synthetic polypeptide
  REGION 1..1061: note = Category: MG3 chimeric effectors
  REGION 1..1061: note = Description: MG3-6_6-3 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAIVVAFTNRSTLKRLSDENKRIGTAEWMDADESGRATNDEIKRRLGGRIDLSEPWPTFRNDVEVSINNITVSHRVNRKVSGALHEETYYGPTDEPAPKNKEMMVLRKSVHQLSKKDLGLIRDETIRQIVNDEVQKRMDNGESQANAIASLEADPPFIISPKAKVPIRKVRLLMKKDPQIMHYFENKNGEEDRAALYGNNHHIAIYETSDKNGVKKQIGIVIPMMEAARRVKDGDPIVMKDYRPDHTFLYSLAKNDMIFNHEDEQIYRVQKINSDGTIMFRQNNVAMKGQSDPGVYFKSGSRLGASKIKISPIGEIFPAND

SEQ ID NO: 16 - protein, 990 aa, synthetic construct
  REGION 1..990: note = Description of Artificial Sequence: Synthetic polypeptide
  REGION 1..990: note = Category: MG3 chimeric effectors
  REGION 1..990: note = Description: MG3-6_14-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDACVIAACSPSLVIKTARINQETHWSITRGMNETQRRDAIMKALESVMPWETFANEVRAAHDFVVPTRFVPRKGKGELFEQTVYRYAGVNAQGKDIARKASSDKDIVMGNAVVSADEKSVIKVSEMLCLRLWHDPEAKKGQGAWYADPVYKADIPALKDGTYVPRIAKAHTGRKAWKPVPESAMAKPPLEIYFGDLVQIGDFIGRFSGYNINNANWSFTDRLTRLNLSCPTVGQLNNDLSPVVIRESPIK

SEQ ID NO: 17 - protein, 1096 aa, synthetic construct
  REGION 1..1096: note = Description of Artificial Sequence: Synthetic polypeptide
  REGION 1..1096: note = Category: MG3 chimeric effectors
  REGION 1..1096: note = Description: MG3-6_15-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAVIIACATQGIVNKVSRYSKSRELWDYEVDMETGEVLQKKNKNTKDVFPEPWLNFRYELEQKVRVRPLDIPETADITEMEEPFVSHMPNRKIHGPAHKETIRSGRLKEEGYTISKTALIDLKLTEDKEEIKGYYNKESDRLLYEALKKQLQRYGGKAKEAFKEPFHKPKADGTPGPIVNKVKIMEKSTMLIPVNGGKGLASNGNMVRIDVFRAEEKGKKKYYFIPVYVADTVKEELPNRAVLAHKPYEAWKIMKEENFIFSLYPNDLIFVDAGKEIPFKAALKGSTLDPEKKASRFLMYYKGADIATGSISGVNHDETYKARGVGIQSLREIKKCCIDVLGNISFASKEKRQTFR

SEQ ID NO: 18 - protein, 1110 aa, synthetic construct
  REGION 1..1110: note = Description of Artificial Sequence: Synthetic polypeptide
  REGION 1..1110: note = Category: MG3 chimeric effectors
  REGION 1..1110: note = Description: MG3-6_16-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDALTVALTRQSYIQRLNTLEASHEHMEKLVKEANTPYKEKKSLLEKWVALQPHFSVEEVTTQVDGILVSFRAGKRVTTPARRAVYHGGKRTIVQRGIQVPRGALTEDTIYGKLGDKFVVKYALDHPSMKPENIVDPTIRLLVENRITALGKKDAFKTPLYSAEGMEIKSVRCYTSLSEKGVVPIKYNEKGNAIGFAKKGNNHHVAIYKDQSGQYQEMVVSFWDAVERKLYGVPTVITNPKTVWDELLEKELPQDFLEKLPKDNWQYVLSMQENEMFVLGMEEDEFNDAIDTQDYNTLNKHLYRVQKLSHADYTFRFHTETKVDDKYDGVENGRNTSMSLKALVRIRSFNGLFTQFPHKVKIDIMGRITKA

SEQ ID NO: 19 - protein, 1174 aa, synthetic construct
  REGION 1..1174: note = Description of Artificial Sequence: Synthetic polypeptide
  REGION 1..1174: note = Category: MG3 chimeric effectors
  REGION 1..1174: note = Description: MG3-6_16-2 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDALVVACTKQSYIQRLNNLNTERDAMYQDIEAQSVEWKEKHSLLEKWIKLQPHPTVSEVTDKVDEILVSFKAGKRVATLGKRSVYKNGKKTVVQNNIIVPRGALCEESVYGQINLIEKNKPIKYLFENPSLIFKPYIKALVEERLKEYNGDTSKAISSLKNNPIYLRKDKSVVLEYGTCYKKEYVKKYSLNSIKAKDVDSIIDKHIREVVRQRLEDNNNNEKAAFASPLYADKQKQIPIKSVRCTTGINIAAPVNYNESNDPISFVKPGNNHHIAIYKDKDGKRQEHIVTFWHAVERKKYGMPVVITNPKEIWDLIIEKSLDLPESFLNCLPNSDWNYEISMQQNEMFVMGMSEDEFQDAIRNNDYKTLNKYLYRVQSVSESDYWLRLHIETMNDKTPEGNIIKKYYRIKSINTFFNFNPHKVKITLLGEIQSS

SEQ ID NO: 20 - protein, 1094 aa, synthetic construct
  REGION 1..1094: note = Description of Artificial Sequence: Synthetic polypeptide
  REGION 1..1094: note = Category: MG3 chimeric effectors
  REGION 1..1094: note = Description: MG3-6_18-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAYLNAVVGNVYHEKFTKNPLRFVRSGQEYSLNLSALFQNWNIYKGGRVIWQKGEDGSLETVRARMAKNDPMVTRYCTEGRGALYDLQPMKKSKGQLPLKSSDERLQHIDRYGGYNKLAGAYFTLAAYYKKGKRVKSIESVPLYLAAKLQRDPAALQQYLADQLGTDRVEILVPEIKLGTLFKWNGYPMTLSGRTGPQLLFRNAAELRTNAEQEQYIKKMSRYLEKCKGRKEPLPIRPAYDKLTPEENLQLYDAFTQWLTSGIYAKRLSLQGKFLLEKRDAFAALSPEAQVRQLMEILHLFQCNPVAANLSELGGAAHAGILLASKNIDGKVPVSIVHQSVTGYFTQEVCLNDL

SEQ ID NO: 21 - protein, 1101 aa, synthetic construct
  REGION 1..1101: note = Description of Artificial Sequence: Synthetic polypeptide
  REGION 1..1101: note = Category: MG3 chimeric effectors
  REGION 1..1101: note = Description: MG3-6_21-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVIACITPGMIQKITKYAQNHERFYATAKGYVDIETGEVLTRSEYEAMDDIRFPEPWPGFRSELEARVSEHPQEAIARLKLPHYENSEEIRPIFVSRMPNHKVTGAAHLETIRSKKGGAGSTVTKTALPDLKLDKNGEIAGYYRKEDDPLLYEALKARLKAFGGDGKKAFAEPFHKPKHNGEPGPIVKKVKIQESATLTVPVNHGIAANGSMVRLDVFHVDGDGYYFVPIYTSDTVKPELPNRAVVAGRRVQEWKVMDDSYFKFSLYPKDLIRIRSKKGIKLKAVNRNADLQEYSTNDCLCYFVKFNISTGALSVENHDRKFEQPGLGGKTLLSIEKYQVDVLGNYSPVALPEKRMKFR

SEQ ID NO: 22 - protein, 1172 aa, synthetic construct
  REGION 1..1172: note = Description of Artificial Sequence: Synthetic polypeptide
  REGION 1..1172: note = Category: MG3 chimeric effectors
  REGION 1..1172: note = Description: MG3-6_22-2 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAIAIACINRSIVNYLNNAAANQTEREDLRRAVCIPERNGQTKRQLRSPWHCFARDAENALRQIVVSFKQNLRVATKATNSYECFDTASGKKIRKHQSNREHYAIRKPLHKDSVYGEVILTSIASVNLKKALLKAERILDKRLKEKIFELRKLYNYSNKQIEEHLTKVCINCPEWKNYDFKKIAVRILSNDADATHIVAIRKPLDESFDEVKINTITDTGIQKILLNHLSRYADDPKKAFSPEGIEDMNANIASLNGGKQHLPIYKVRVSEKDNGGYFPIGQKGNRPKKYVTTAKDTNLFFAVYADSKGKRSYKTIDLRTAIECRKQGLSVAPSINEKGDKLLFTLSPNDLVYMPSEGEEANGFAIDNNLNKDQIYKMVSANNKQCFFIPHTVADFISRGEEYNSHNKIELTEDRRSIKEHCVPLKVNRLGK

SEQ ID NO: 23 - protein, 1112 aa, synthetic construct
  REGION 1..1112: note = Description of Artificial Sequence: Synthetic polypeptide
  REGION 1..1112: note = Category: MG3 chimeric effectors
  REGION 1..1112: note = Description: MG3-6_23-1 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAYLNIVVGNTYSTKFTNNPLNFIKAGAKRPQDNQFKYNMDKIFDYNVISRGERAWIAGSDGSICTVKKFMSRNTVLITRKAKEVHGALSNKATIWGKNVAKPGAYLPVKSTDLKAQDVTKYGGITSIANSGYTLAEYKVNGKTTRSLEALPVYLGRAEQLTEKTVVDYLSSSLQESSKKKIEDIQVRKLFIPQGSKVKIDGFCYYLGGKTGDSIYLNNAVPLYLSSTSEEYLRKLLKAVENNNYNERDKNGQIILTAPKNVQLLSSIFDKLRSKPFSNNKWNIYFSIVNGKETKVEQLFSKLSIDKQAEVISQIVIWINSSRQNVNLSLIGGSAHSGTQALSKTVSRLNECMLISQSITGIYEHSVDLLTI

SEQ ID NO: 24 - protein, 1090 aa, synthetic construct
  REGION 1..1090: note = Description of Artificial Sequence: Synthetic polypeptide
  REGION 1..1090: note = Category: MG3 chimeric effectors
  REGION 1..1090: note = Description: MG3-6_SaCas9 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 25 - protein, 1123 aa, synthetic construct
  REGION 1..1123: note = Description of Artificial Sequence: Synthetic polypeptide
  REGION 1..1123: note = Category: MG3 chimeric effectors
  REGION 1..1123: note = Description: MG3-6_SpCas9 chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - protein, 1105 aa, synthetic construct
  REGION 1..1105: note = Description of Artificial Sequence: Synthetic polypeptide
  REGION 1..1105: note = Category: MG3 chimeric effectors
  REGION 1..1105: note = Description: MG3-6_15-1-WP chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRTFYEQGLDKLDRNQLKPEEDWRDFTGLAPASQEKFLEWRKAATILGDLLAEAIEDDSIAVVSPLRLRPQNGSVHLEKIHGPAHKETIRSGRLKEEGYTISKTALIDLKLTEDKEEIKGYYNKESDRLLYEALKKQLQRYGGKAKEAFKEPFHKPKADGTPGPIVNKVKIMEKSTMLIPVNGGKGLASNGNMVRIDVFRAEEKGKKKYYFIPVYVADTVKEELPNRAVLAHKPYEAWKIMKEENFIFSLYPNDLIFVDAGKEIPFKAALKGSTLDPEKKASRFLMYYKGADIATGSISGVNHDETYKARGVGIQSLREIKKCCIDVLGNISFASKEKRQTFR

SEQ ID NO: 27 - protein, 1074 aa, synthetic construct
  REGION 1..1074: note = Description of Artificial Sequence: Synthetic polypeptide
  REGION 1..1074: note = Category: MG3 chimeric effectors
  REGION 1..1074: note = Description: MG3-6_15-1-P chimera
MSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRTFYEQGLDKLDRNQLKPEEDWRDFTGLAPASQEKFLEWRKAATILGDLLAEAIEDDSIAVVSPLRLRPQNGSVHLETISAVKKQTLGSDWPADAVKRIVDPEIYLAMKDALGKLKELPEDSARSLELPDGRFVEADDEVLFFPENAASILTPRGVAEINMVRIDVFRAEEKGKKKYYFIPVYVADTVKEELPNRAVLAHKPYEAWKIMKEENFIFSLYPNDLIFVDAGKEIPFKAALKGSTLDPEKKASRFLMYYKGADIATGSISGVNHDETYKARGVGIQSLREIKKCCIDVLGNISFASKEKRQTFR

SEQ ID NO: 28 - DNA, 121 bp, synthetic construct
  misc_feature 1..121: note = Description of Artificial Sequence: Synthetic polynucleotide
  misc_feature 1..121: note = Category: MG1 sgRNA
  misc_feature 1..121: note = Description: MG1-4 sgRNA
  misc_difference 1..22: note = any nucleotide
  source 1..121: mol_type = other DNA
nnnnnnnnnnnnnnnnnnnnnngttttgatttactcgaaagagtccaatcataattgaccggagaataattgattcctctacaatgtacgaataaattcattctctaaaccttaaaaattt

SEQ ID NO: 29 - DNA, 99 bp, synthetic construct
  misc_feature 1..99: note = Description of Artificial Sequence: Synthetic oligonucleotide
  misc_feature 1..99: note = Category: MG1 sgRNA
  misc_feature 1..99: note = Description: MG1-5 sgRNA
  misc_difference 1..22: note = any nucleotide
  source 1..99: mol_type = other DNA
nnnnnnnnnnnnnnnnnnnnnngttttgacttgaaaaagtcttaactgattttgccgaattttaagctctacgtagtaccttggaattcggcatatttt

SEQ ID NO: 30 - DNA, 99 bp, synthetic construct
  misc_feature 1..99: note = Description of Artificial Sequence: Synthetic oligonucleotide
  misc_feature 1..99: note = Category: MG1 sgRNA
  misc_feature 1..99: note = Description: MG1-6 sgRNA
  misc_difference 1..22: note = any nucleotide
  source 1..99: mol_type = other DNA
nnnnnnnnnnnnnnnnnnnnnngttttgacttgaaaaagtcttaactgattttgccgaatttcaagctctgcattgcaccttggcattcggcatatttt

SEQ ID NO: 31 - DNA, 99 bp, synthetic construct
  misc_feature 1..99: note = Description of Artificial Sequence: Synthetic oligonucleotide
  misc_feature 1..99: note = Category: MG1 sgRNA
  misc_feature 1..99: note = Description: MG1-7 sgRNA
  misc_difference 1..22: note = any nucleotide
  source 1..99: mol_type = other DNA
nnnnnnnnnnnnnnnnnnnnnngttttgactttgaaaaaagtcttaactgattttgccgaattttaagctctgcatggcaccttgaaattcggcatttt

SEQ ID NO: 32 - DNA, 100 bp, synthetic construct
  misc_feature 1..100: note = Description of Artificial Sequence: Synthetic polynucleotide
  misc_feature 1..100: note = Category: MG2 sgRNA
  misc_feature 1..100: note = Description: MG2-7 sgRNA
  misc_difference 1..22: note = any nucleotide
  source 1..100: mol_type = other DNA
nnnnnnnnnnnnnnnnnnnnnngctttgccttggaaacaagacaaagttaattaaggcagttcggacctctactttgtacgtcaggatagaaagcctttt

SEQ ID NO: 33 - DNA, 115 bp, synthetic construct
  misc_feature 1..115: note = Description of Artificial Sequence: Synthetic polynucleotide
  misc_feature 1..115: note = Category: MG3 sgRNA
  misc_feature 1..115: note = Description: MG3-3 sgRNA
  misc_difference 1..22: note = any nucleotide
  source 1..115: mol_type = other DNA
nnnnnnnnnnnnnnnnnnnnnngttgggaatcttgaaaaagattcccaataaggcacatttttagtgctgacttctcaccgtccagggttcattgaacaatgggcggtatgtttt

SEQ ID NO: 34 - DNA, 126 bp, synthetic construct
  misc_feature 1..126: note = Description of Artificial Sequence: Synthetic polynucleotide
  misc_feature 1..126: note = Category: MG3 sgRNA
  misc_feature 1..126: note = Description: MG3-4 sgRNA
  misc_difference 1..22: note = any nucleotide
  source 1..126: mol_type = other DNA
nnnnnnnnnnnnnnnnnnnnnngttgagaatctttcattagaaataacgaaagattcttaataaggcgtccttccgatgctgacttctcaccgtccgttttccaataggagcgggcggtatgtttt

SEQ ID NO: 35 - DNA, 110 bp, synthetic construct
  misc_feature 1..110: note = Description of Artificial Sequence: Synthetic polynucleotide
  misc_feature 1..110: note = Category: MG3 sgRNA
  misc_feature 1..110: note = Description: MG3-6 sgRNA
  misc_difference 1..22: note = any nucleotide
  source 1..110: mol_type = other DNA
nnnnnnnnnnnnnnnnnnnnnngttgagaatcgaaagattcttaataaggcatccttccgatgctgacttctcaccgtccgttttccaataggagcgggcggtatgtttt

SEQ ID NO: 36 - DNA, 110 bp, synthetic construct
  misc_feature 1..110: note = Description of Artificial Sequence: Synthetic polynucleotide
  misc_feature 1..110: note = Category: MG3 sgRNA
  misc_feature 1..110: note = Description: MG3-7 sgRNA
  misc_difference 1..22: note = any nucleotide
  source 1..110: mol_type = other DNA
nnnnnnnnnnnnnnnnnnnnnngttgggaaccgaaaggttcccaataaggcgcatcttggcgctgacttctcaccgtcctcttgctgcttagcagagggcggtatgtttt

SEQ ID NO: 37 - DNA, 110 bp, synthetic construct
  misc_feature 1..110: note = Description of Artificial Sequence: Synthetic polynucleotide
  misc_feature 1..110: note = Category: MG3 sgRNA
  misc_feature 1..110: note = Description: MG3-8 sgRNA
  misc_difference 1..22: note = any nucleotide
  source 1..110: mol_type = other DNA
nnnnnnnnnnnnnnnnnnnnnngttgagaatcgaaagattcttaataaggcatccttccgatgctgacttctcaccgtccggctcctcttaggaacgggcggtatgtttt

SEQ ID NO: 38 - DNA, 124 bp, synthetic construct
  misc_feature 1..124: note = Description of Artificial Sequence: Synthetic polynucleotide
  misc_feature 1..124: note = Category: MG4 sgRNA
  misc_feature 1..124: note = Description: MG4-5 sgRNA
  misc_difference 1..22: note = any nucleotide
  source 1..124: mol_type = other DNA
nnnnnnnnnnnnnnnnnnnnnngctgtggcttgcgggggaaaccccttgtcacagtaagggactttcgttcgcgaaaggcaacctcgccagcatcgctgggcgaggaccagggcaaggcgattt

SEQ ID NO: 39 - DNA, 118 bp, synthetic construct
  misc_feature 1..118: note = Description of Artificial Sequence: Synthetic polynucleotide
  misc_feature 1..118: note = Category: MG14 sgRNA
  misc_feature 1..118: note = Description: MG14-1 sgRNA
  source 1..118: mol_type = other DNA
  misc_difference 1..22: note = any nucleotide
nnnnnnnnnnnnnnnnnnnnnngtcttgagcgaaagctccagacaaggggagccacttaagtggcttacccgtaaagtaacccccgttcaatcttcggattgggcggggcgaactttt

SEQ ID NO: 40 - DNA, 118 bp, synthetic construct
  misc_feature 1..118: note = Description of Artificial Sequence: Synthetic polynucleotide
  misc_feature 1..118: note = Category: MG15 sgRNA
  misc_feature 1..118: note = Description: MG15-1 sgRNA
  misc_difference 1..22: note = any nucleotide
  source 1..118: mol_type = other DNA
nnnnnnnnnnnnnnnnnnnnnngttgtaattccctagaaataggttattacaataaggtccaacaggagtgttggtaccgtaaagctctaacggcacccacgggtgccgttatctttt

SEQ ID NO: 41 - DNA, 162 bp, synthetic construct
  misc_feature 1..162: note = Description of Artificial Sequence: Synthetic polynucleotide
  misc_feature 1..162: note = Category: MG16 sgRNA
  misc_feature 1..162: note = Description: MG16-2 sgRNA
  misc_difference 1..22: note = any nucleotide
  source 1..162: mol_type = other DNA
nnnnnnnnnnnnnnnnnnnnnngttgtgaattgctttaaattgaaaaatttaagcaattcacaataaggattattccgttgtgaaaacatttaaagcggggtcaacagcctcgctttctttttgagtctatgagacattaggtcaataagtctatgagtttt

SEQ ID NO: 42 - DNA, 101 bp, synthetic construct
  misc_feature 1..101: note = Description of Artificial Sequence: Synthetic polynucleotide
  misc_feature 1..101: note = Category: MG18 sgRNA
  misc_feature 1..101: note = Description: MG18-1 sgRNA
  source 1..101: mol_type = other DNA
  misc_difference 1..22: note = any nucleotide
nnnnnnnnnnnnnnnnnnnnnngtttgagagtagtgaaaactacgagttcaaatacaattttttcaaattgccctatagggccctcacagtgtgagatttt

SEQ ID NO: 43 - DNA, 117 bp, synthetic construct
  misc_feature 1..117: note = Description of Artificial Sequence: Synthetic polynucleotide
  misc_feature 1..117: note = Category: MG21 sgRNA
  misc_feature 1..117: note = Description: MG21-1 sgRNA
  misc_difference 1..22: note = any nucleotide
  source 1..117: mol_type = other DNA
nnnnnnnnnnnnnnnnnnnnnngttgtagttccccttttgaaaaaaagtgtgttactgcaataaggtaaaacaccacgaagctctgccctaactgccttagcagttagggcatcttt

SEQ ID NO: 44 - DNA, 134 bp, synthetic construct
  misc_feature 1..134: note = Description of Artificial Sequence: Synthetic polynucleotide
  misc_feature 1..134: note = Category: MG22 sgRNA
  misc_feature 1..134: note = Description: MG22-1 sgRNA
  misc_difference 1..22: note = any nucleotide
  source 1..134: mol_type = other DNA
nnnnnnnnnnnnnnnnnnnnnngttgtgaattgctttcaaattagaaataattgaaagcaattcgcaataaggattattccgttgtgaaaacatttcgagtggcttcgtgaaattcacgaagtcacttcgtttt

SEQ ID NO: 45 - DNA, 113 bp, synthetic construct
  misc_feature 1..113: note = Description of Artificial Sequence: Synthetic polynucleotide
  misc_feature 1..113: note = Category: MG23 sgRNA
  misc_feature 1..113: note = Description: MG23-1 sgRNA
  misc_difference 1..22: note = any nucleotide
  source 1..113: mol_type = other DNA
nnnnnnnnnnnnnnnnnnnnnngtttgagaacctgaaaaggtgagtgcaaataaggtttaaccgaaattgtttacctgcattgtgcagtataagaaagaccgcgaggtctttt

SEQ ID NO: 46-66 - skipped (21 entries)

SEQ ID NO: 67 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 45 for targeting albumin intron 1
  source 1..110: mol_type = other DNA
tgccagttcccgatcgttacaggttgagaatcgaaagattcttaataaggcatccttccgatgctgacttctcaccgtccgttttccaataggagcgggcggtatgtttt

SEQ ID NO: 68 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 78 for targeting albumin intron 1
  source 1..110: mol_type = other DNA
aaataccaggcttccattactagttgagaatcgaaagattcttaataaggcatccttccgatgctgacttctcaccgtccgttttccaataggagcgggcggtatgtttt

SEQ ID NO: 69 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 24 for targeting albumin intron 1
  source 1..110: mol_type = other DNA
atttacaaacatgacagaaacagttgagaatcgaaagattcttaataaggcatccttccgatgctgacttctcaccgtccgttttccaataggagcgggcggtatgtttt

SEQ ID NO: 70 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 34 for targeting albumin intron 1
  source 1..110: mol_type = other DNA
cttaggtcagtgaagagaagaagttgagaatcgaaagattcttaataaggcatccttccgatgctgacttctcaccgtccgttttccaataggagcgggcggtatgtttt

SEQ ID NO: 71 - DNA, 110 bp, synthetic construct
  misc_feature 1..110: note = Description of Artificial Sequence: Synthetic polynucleotide
  misc_feature 1..110: note = Category: MG3 Hepa1-6 targeting guides(included in sgRNA)
  misc_feature 1..110: note = Description: MG3-6_3-4 guide sequence 44 for targeting albumin intron 1
  source 1..110: mol_type = other DNA
atgccagttcccgatcgttacagttgagaatcgaaagattcttaataaggcatccttccgatgctgacttctcaccgtccgttttccaataggagcgggcggtatgtttt

SEQ ID NO: 72 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 87 for targeting albumin intron 1
  source 1..110: mol_type = other DNA
cttctcggcgaaacacacccctgttgagaatcgaaagattcttaataaggcatccttccgatgctgacttctcaccgtccgttttccaataggagcgggcggtatgtttt

SEQ ID NO: 73 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 81 for targeting albumin intron 1
  source 1..110: mol_type = other DNA
ctagaaaaatacaagcagagatgttgagaatcgaaagattcttaataaggcatccttccgatgctgacttctcaccgtccgttttccaataggagcgggcggtatgtttt

SEQ ID NO: 74 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 72 for targeting albumin intron 1
  source 1..110: mol_type = other DNA
aataataatctagaaatcagcagttgagaatcgaaagattcttaataaggcatccttccgatgctgacttctcaccgtccgttttccaataggagcgggcggtatgtttt

SEQ ID NO: 75 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 16 for targeting albumin intron 1
  source 1..110: mol_type = other DNA
ctgcctgctcgaccatgctatagttgagaatcgaaagattcttaataaggcatccttccgatgctgacttctcaccgtccgttttccaataggagcgggcggtatgtttt

SEQ ID NO: 76 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 59 for targeting albumin intron 1
  source 1..110: mol_type = other DNA
aggcaggccctatgagaccgtagttgagaatcgaaagattcttaataaggcatccttccgatgctgacttctcaccgtccgttttccaataggagcgggcggtatgtttt

SEQ ID NO: 77 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 30 for targeting albumin intron 1
  source 1..110: mol_type = other DNA
ttttaaaaataataatgttggtgttgagaatcgaaagattcttaataaggcatccttccgatgctgacttctcaccgtccgttttccaataggagcgggcggtatgtttt

SEQ ID NO: 78 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 20 for targeting albumin intron 1
  source 1..110: mol_type = other DNA
tcgaccatgctatactaaaaatgttgagaatcgaaagattcttaataaggcatccttccgatgctgacttctcaccgtccgttttccaataggagcgggcggtatgtttt

SEQ ID NO: 79 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 84 for targeting albumin intron 1
  source 1..110: mol_type = other DNA
atacaagcagagatgaaaaaacgttgagaatcgaaagattcttaataaggcatccttccgatgctgacttctcaccgtccgttttccaataggagcgggcggtatgtttt

SEQ ID NO: 80 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 33 for targeting albumin intron 1
  source 1..110: mol_type = other DNA
gcttaggtcagtgaagagaagagttgagaatcgaaagattcttaataaggcatccttccgatgctgacttctcaccgtccgttttccaataggagcgggcggtatgtttt

SEQ ID NO: 81 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 64 for targeting albumin intron 1
  source 1..110: mol_type = other DNA
aaagaaatttaaagctaagcttgttgagaatcgaaagattcttaataaggcatccttccgatgctgacttctcaccgtccgttttccaataggagcgggcggtatgtttt

SEQ ID NO: 82 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 53 for targeting albumin intron 1
  source 1..110: mol_type = other DNA
ctaagacaatggtaaataagaagttgagaatcgaaagattcttaataaggcatccttccgatgctgacttctcaccgtccgttttccaataggagcgggcggtatgtttt

SEQ ID NO: 83 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 73 for targeting albumin intron 1
  source 1..110: mol_type = other DNA
taatctagaaatcagcactaaagttgagaatcgaaagattcttaataaggcatccttccgatgctgacttctcaccgtccgttttccaataggagcgggcggtatgtttt

SEQ ID NO: 84 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 74 for targeting albumin intron 1
  source 1..110: mol_type = other DNA
aatctagaaatcagcactaaaggttgagaatcgaaagattcttaataaggcatccttccgatgctgacttctcaccgtccgttttccaataggagcgggcggtatgtttt

SEQ ID NO: 85 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 13 for targeting albumin intron 1
  source 1..110: mol_type = other DNA
ctttaaatttcttttaattaaagttgagaatcgaaagattcttaataaggcatccttccgatgctgacttctcaccgtccgttttccaataggagcgggcggtatgtttt

SEQ ID NO: 86 - DNA, 110 bp, synthetic construct
  misc_feature 1..110: note = Description of Artificial Sequence: Synthetic polynucleotide
  misc_feature 1..110: note = Category: MG3 Hepa1-6 targeting guides (included in sgRNA)
  misc_feature 1..110: note = Description: MG3-6_3-4 guide sequence 19 for targeting albumin intron 1
  source 1..110: mol_type = other DNA
ctcgaccatgctatactaaaaagttgagaatcgaaagattcttaataaggcatccttccgatgctgacttctcaccgtccgttttccaataggagcgggcggtatgtttt

SEQ ID NO: 87 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MG3-6(718-840) for MG3-6(1-840)_MG15-1(818-1082)
  source 1..50: mol_type = other DNA
gtcacccgcgaaagtcgtcgcgccggcggcatcgatgaacgcatcctgtt

SEQ ID NO: 88 - DNA, 54 bp, synthetic construct
  misc_feature 1..54: note = Description of Artificial Sequence: Synthetic oligonucleotide
  misc_feature 1..54: note = Category: primer
  misc_feature 1..54: note = Description: Amplify MG3-6(718-840) for MG3-6(1-840)_MG15-1(818-1082)
  source 1..54: mol_type = other DNA
tagtctctttgtgggcaggaccgtggatcttctctaagtgaacagatccatttt

SEQ ID NO: 89 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify MG15-1(818-1082) for MG3-6(1-840)_MG15-1(818-1082)
  source 1..48: mol_type = other DNA
cgcccgcaaaatggatctgttcacttagagaagatccacggtcctgcc

SEQ ID NO: 90 - DNA, 50 bp, synthetic construct
  misc_feature 1..50: note = Description of Artificial Sequence: Synthetic oligonucleotide
  misc_feature 1..50: note = Category: primer
  misc_feature 1..50: note = Description: Amplify MG15-1(818-1082) for MG3-6(1-840)_MG15-1(818-1082)
  source 1..50: mol_type = other DNA
gttatcagtggtggtggtggtggtgctcgagacggaacgtttggcgcttc

SEQ ID NO: 91 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify MG3-6(718-922) for MG3-6(1-922)_MG15-1(931-1082)
  source 1..48: mol_type = other DNA
ggcgcgaaagacgtcgatgcgaaccatgttgatttccgctacgccacg

SEQ ID NO: 92 - DNA, 48 bp, synthetic construct
  misc_feature 1..48: note = Description of Artificial Sequence: Synthetic oligonucleotide
  misc_feature 1..48: note = Category: primer
  misc_feature 1..48: note = Description: Amplify MG15-1(931-1082) for MG3-6(1-922)_MG15-1(931-1082)
  source 1..48: mol_type = other DNA
tcctgaccccgcgtggcgtagcggaaatcaacatggttcgcatcgacg

SEQ ID NO: 93 - DNA, 38 bp, synthetic construct
  misc_feature 1..38: note = Description of Artificial Sequence: Synthetic oligonucleotide
  misc_feature 1..38: note = Category: primer
  misc_feature 1..38: note = Description: LA065 primer used for PCR in PAM enrichment assay
  misc_difference 14..18: note = Any nucleotide
  source 1..38: mol_type = other DNA
gctcttccgatctnnnnnattgacggcggcatcggact

SEQ ID NO: 94 - DNA, 35 bp, synthetic construct
  misc_feature 1..35: note = Description of Artificial Sequence: Synthetic oligonucleotide
  misc_feature 1..35: note = Category: primer
  misc_feature 1..35: note = Description: LA125 primer used for PCR in PAM enrichment assay
  misc_difference 14..18: note = Any nucleotide
  source 1..35: mol_type = other DNA
gctcttccgatctnnnnnggtgttggcgggtgtcg

SEQ ID NO: 95 - DNA, 20 bp, synthetic construct
  misc_feature 1..20: note = Description of Artificial Sequence: Synthetic oligonucleotide
  misc_feature 1..20: note = Category: primer
  misc_feature 1..20: note = Description: LA003 PAM enrichment adapter (heteroduplex of LA003/LA011)
  source 1..20: mol_type = other DNA
attgacggcggcatcggact

SEQ ID NO: 96 - DNA, 21 bp, synthetic construct
  misc_feature 1..21: note = Description of Artificial Sequence: Synthetic oligonucleotide
  misc_feature 1..21: note = Category: primer
  misc_feature 1..21: note = Description: LA011 PAM enrichment adapter (heteroduplex of LA003/LA011)
  source 1..21: mol_type = other DNA
agtccgatgccgccgtcaatt

SEQ ID NO: 97 - DNA, 20 bp, synthetic construct
  misc_feature 1..20: note = Description of Artificial Sequence: Synthetic oligonucleotide
  misc_feature 1..20: note = Category: primer
  misc_feature 1..20: note = Description: 57F primer used for Sanger sequencing of edited Hepa1-6 cells
  source 1..20: mol_type = other DNA
tctggcaaaatgaagtgggt

SEQ ID NO: 98 - DNA, 20 bp, synthetic construct
  misc_feature 1..20: note = Description of Artificial Sequence: Synthetic oligonucleotide
  misc_feature 1..20: note = Category: primer
  misc_feature 1..20: note = Description: 1072R primer used for Sanger sequencing of edited Hepa1-6 cells
  source 1..20: mol_type = other DNA
tgccacattgctcagcacag

SEQ ID NO: 99 - DNA, 20 bp, synthetic construct
  misc_feature 1..20: note = Description of Artificial Sequence: Synthetic oligonucleotide
  misc_feature 1..20: note = Category: primer
  misc_feature 1..20: note = Description: 132F primer used for Sanger sequencing of edited Hepa1-6 cells
  source 1..20: mol_type = other DNA
cgccgagaagcacgtaagag

SEQ ID NO: 100 - DNA, 21 bp, synthetic construct
  misc_feature 1..21: note = Description of Artificial Sequence: Synthetic oligonucleotide
  misc_feature 1..21: note = Category: primer
  misc_feature 1..21: note = Description: 282F primer used for Sanger sequencing of edited Hepa1-6 cells
  source 1..21: mol_type = other DNA
ttgcatctgagaacccttagg

SEQ ID NO: 101 - DNA, 23 bp, synthetic construct
  misc_feature 1..23: note = Description of Artificial Sequence: Synthetic oligonucleotide
  misc_feature 1..23: note = Category: primer
  misc_feature 1..23: note = Description: 446R primer used for Sanger sequencing of edited Hepa1-6 cells
  source 1..23: mol_type = other DNA
ccgtaataaattcaactgtatcc

SEQ ID NO: 102 - DNA, 20 bp, synthetic construct
  misc_feature 1..20: note = Description of Artificial Sequence: Synthetic oligonucleotide
  misc_feature 1..20: note = Category: primer
  misc_feature 1..20: note = Description: 460F primer used for Sanger sequencing of edited Hepa1-6 cells
  source 1..20: mol_type = other DNA
gcctgctcgaccatgctata

SEQ ID NO: 103 - DNA, 8755 bp, synthetic construct
  misc_feature 1..8755: note = Description of Artificial Sequence: Synthetic polynucleotide
  misc_feature 1..8755: note = Category: plasmid
  misc_feature 1..8755: note = Description: pMGX3-6 plasmid
  source 1..8755: mol_type = other DNA
ctcgagcaccaccaccaccaccactgataacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggaattgtgagcggataacaattcccctctagaaataattttgtttaactttaagaaggagatataccatgtcaaccgatatgaagaattatcgcattggcgtggacgtcggggatcgcagcgtaggcttggcagcgattgagttcgacgacgacgggctgccgattcagaaactggcattagttacctttcgtcatgacggtggcctcgacccaaccaagaacaaaacgcctatgtcgcgcaaagagacccgcgggatcgcgcgccgcaccatgcgtatgaatcgcgaacgcaaacgccgccttcgcaatttggacaacgtactggagaacttaggctatagcgtacctgagggacctgaaccggaaacttacgaagcctggacgagccgcgcactcttggcaagcattaaattagcatccgccgacgaattaaacgaacacctggttcgtgccgttcgccacatggcgcgccatcgcggctgggcaaacccctggtggagcctggatcaactcgaaaaggcatcacaagaacccagcgaaaccttcgaaatcatcttagcacgcgcacgcgaattattcggcgagaaagtaccagcgaaccccacccttgggatgctgggagcgctggcggcaaacaacgaagtgctgctccgcccacgtgacgagaagaaacgcaaaacgggatacgtacgtggaacgccgttgatgttcgcacaagtgcgccaaggcgatcaattagccgaattacgccgcatctgtgaagtacaaggcatcgaagatcaatatgaagcgctgcgtctgggtgtctttgatcacaaacatccgtacgtcccaaaagagcgcgttggcaaagatccgttgaacccgagcaccaaccgtaccatccgcgcaagccttgaattccaagaatttcgtatcttagacagtgttgccaatctccgcgttcgtatcggcagtcgcgccaaacgcgaactcaccgaagcggaatacgacgcagcggtagaatttctgatggattacgcggacaaagagcagcctagttgggcggacgtggcggaaaagatcggggtgccgggcaatcgtctggtagcccccgtcttagaagacgtacaacagaaaacagccccttacgatcgttcttctgcggcgttcgaaaaggctatggggaagaaaactgaagcccgccaatggtgggagtcgacggacgacgatcaactgcgcagccttctgatcgcgttcctggtggacgccactaatgacaccgaggaagctgccgcggaagccggattaagcgaattgtacaaatcctggcccgcggaagagcgcgaggctttaagcaatatcgacttcgaaaagggtcgcgtggcgtactcacaagaaacactgtccaagttaagtgaatatatgcacgaatatcgcgtcgggctgcatgaagcacgtaaagcagtatttggggttgacgacacgtggcgcccacctctggacaaattagaagagcctaccggacaacctgcagtagaccgtgtcctcaccatcttacgccgcttcgtattggactgcgaacgccaatggggtcgccctcgtgccatcaccgtagagcacactcgcactggtcttatgggcccgactcaacgccaaaagatcttgaacgaacaaaagaagaatcgcgcagacaacgaacgcatccgtgacgaactgcgcgaaagcggagttgacaacccaagtcgtgctgaggtgcgccgtcacttaatcgttcaagaacaagaatgccaatgtctgtattgtggtaccatgatcacaactaccaccagcgaactcgaccacatcgtaccacgcgcaggcggtggcagcagccgccgcgaaaacttggccgccgtatgtcgcgcctgcaacgcaaagaagaaacgcgagttgttttacgcctgggcgggaccggtcaaatcccaagaaacaatcgaacgcgtgcgtcaactgaaagcattcaaagactcaaagaaagctaagatgtttaagaaccaaatccgccgcctgaaccaaacagaagcggacgaacctatcgacgaacgctctctcgcgagcaccagctacgctgccgttgccgtccgcgaacgcttagagcaacatttcaacgaaggactggccctcgatgacaaatcacgtgtcgttttagacgtgtatgcgggcgcggtcacccgcgaaagtcgtcgcgccggcggcatcgatgaacgcatcctgttgcgtggcgaacgtgacaagaatcgcttcgacgttcgtcaccatgccgtggacgcggcagtaatgacgttgttgaatcgctctgtggcattaacgctcgaacaacgttcgcaacttcgtcgtaccttctacgaacaaggcttggacaagttagatcgcaaccaattaaaacccgaggaagactggcgcgatttcacagggttagccccagccagccaagagaaatttctggaatggcgcaaagcggcgaccatcttaggggatcttctggccgaagcaattgaagacgatagcatcgccgtggtatctccgttacgccttcgcccgcaaaatggatctgttcacttagagaccattagcgcggttaagaaacagacgctgggaagcgactggccggcagacgccgtcaaacgcatcgtcgaccctgaaatctaccttgcaatgaaagacgcgctggggaaattaaaagaattgccggaagactccgctcgtagcctggaattacctgacgggcgtttcgtggaagcggacgacgaagtgctcttctttcccgaaaatgcggccagcatcctgaccccgcgtggcgtagcggaaatcggcggaagcatccaccacgcgcgtctgtatgggtggttaaccaagaaaggtgaattaaaagttggcatgctgcgcgtatacggcgccgaatttccatggcttatgcgcgaatcgggcagccgtaacgtgctctctatgcctatccatcgcggcagccaatcatttcgcgacatgcaagatactacccgcaaggcggtagagtccggggaagcagtagaattcgcatggatcacgcaaaatgacgaattagaattcgaccctgacgattatatcgcgcacggcggtaaagacgaactccgccagttcttaggcttcatgcctgagtgtcgttggcgcgtcgacggcttcaagaagaattaccaaattcgcatccgtccggccatgttgtcgcgcgagcaacttcccagtgatatccaacgccgcctggagagcaagaccttgacgaagaatgagagccttctgttaaaggccttagacacgggcttagtggtggccatcggtggcctgctgccgttggagaccctgaaagtgatccgtcgtaacaatctgggttttccgcgctggcgtggcaacggtaacttgcccaccagtttcgaggtgcgctccagcgctctgcgcgcgcttggagtagagggg

SEQ ID NO: 104 - DNA, 8599 bp, synthetic construct
  misc_feature 1..8599: note = Description of Artificial Sequence: Synthetic polynucleotide
  misc_feature 1..8599: note = Category: plasmid
  misc_feature 1..8599: note = Description: pMGX15-1 plasmid
  source 1..8599: mol_type = other DNA
ctcgagcaccaccaccaccaccactgataacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggaattgtgagcggataacaattcccctctagaaataattttgtttaactttaagaaggagatataccatgaactatgtgctcggcctggacgtcggtattgcttcagtgggatgggccgtgcttgagttgaatgaagaggacaaccccattcgcattgagggtcttggtgcgcgcatcttcgataaagcggaagtgcccaaaaccggggccagtctcgccgcacctcgtcgcatgagtcgtggcatccgtcgcgtgattcgtcgtcgccgctttcgcttacaacgcgtccgctcatacctgaagaagcacaacattctccctgccgagaaggttgatcacttatacgacgttccatctgccatcgacatctacgagctccgtaagcgcgcactgactgagaaggtcacagccgaggagtgggcgcgcctgttaatcttcttcgcgaaacaccgtggtttcaagtccaaccgcaagaaggcttcgggagacgccgacgagggcgagatgctcaaggcgatcgcagcaaacgccgaaatcctgaagaactatcgtacagtgggagagatgctgtgcgacaacgagaagtttcgtaaacgtaagcgtaatcgcgatggcgcatacaactttactgtctctcgtgcgatgctgatggaagaaattcacacgctgttcgagattcaacgcaacttgggccagaagttcgcggacgagaagctggaagaggagtacgtgatcctcttcgccgctcaacgtaaattcgatgaaggacctggagaaaattcgccgtacgcggggaaccagattgagaagatgatcggttcctgcacgttagagggcaagaaagagaagcgtgctccaaaggcctcatacgcatttatggcatttaatttatggcaaaagatcaatcacttgaaagtcaatcgtcgtggtagtgaacgcttcctgaccgaagaggagcgccgccgcattgcggacctggcgtggaagaaggagaagcttacctacggttcactgcgtaaggtgctctcgttggaccccgaggatcgcttcgttggcctgcgttacgacctcaagaaaggcaaggccgagaacttagaagaacccgtttctgaggcagaaaagcgttccttttcatgggtcaaggattatcactccattcgtaaggcccttgacaaggtggagaagaatcgcattttgaagctctctcacgagcaactggacacaattgccaccgcgtttagtgtttacaagaacgaagagaccattgaaaagtacctggaagagggcggcattggtaaggccgacggtgaggcgctgcttgagcgtttgagcggatttagcaagtttggtcacctgagcctgaaggcgtgttataagctgctgccgttcctggagaagggcgaggtgtactcacgcgcttgtgagttggcggggtacgactttagtaaatcgtccttggagaacattgacgacattccgaacccggtcgtaaagcgctcgatttctcagacccttaaggtgattaacgcgatcattctgcgctacggcaacccagttgaggttcacattgagttagcacgtgagctcgcccgttcttttcgcgaccgcaagaagatggagaagggtatgcaagagaaccaagcgaagaacgagaaaattcgtcagcgcctgattagtgagttcggggtgctgcgcccaaaaggcatggacattgttaagttcaaattatgtgaggaacaaaacggggagtgtgcctactcgcaaaagcactttgacatggaaaagttacttaaggacccgagttacgccgaggttgaccacattatcccgtactcacgctcattcgacgacagttataacaacaaggtgctggttctgacgaaggagaatcgtgagaaacgtaatcgtatcccgatggagtacctcgccgacgcgcctgagcgcaagaaccgtttcattcactgggtcaaatccacaatccgcaatagtcgcaaacgtgagaatcttctgcgtaccgattacacggcgactaccgagaatgagtggaagcgccgtaacctgcaggacactcaatacatttcgaaatatttatataactacctgcgccatcacctggtccttgcgaaaggttacacagagcgtaagcgtcgcatcattccggttaacggtgctgtaaccgcatactttcgcaaacgcctgggtattaacaagattcgtgagaatggcgacctgcatcacgcggtggacgccgttattattgcgtgtgccacacagggaatcgtcaacaaagtttcccgctatagtaaatcgcgcgaactctgggactacgaggtcgacatggagacgggtgaggtgctgcagaagaagaacaagaataccaaggatgtctttccggagccgtggcttaactttcgttacgagttggagcagaaggttcgtgtgcgccccttggacattcccgagacggcagacatcacggagatggaagagccgttcgttagtcatatgcctaaccgtaagatccacggtcctgcccacaaagagactatccgctctggccgccttaaagaggaaggttacacaatcagcaagaccgctctcatcgacttaaagcttactgaggacaaggaagagattaagggctattataacaaggagtcggaccgccttttgtacgaggctttaaagaagcagctgcagcgctacggtggcaaagctaaggaagcgttcaaggagccgtttcacaagcctaaagcagatggtaccccgggcccaatcgttaacaaggtaaagatcatggagaagagcactatgttaatcccggtgaacggcgggaagggtctcgcgtcaaacggcaacatggttcgcatcgacgtctttcgcgccgaggagaaaggtaagaagaaatattacttcatcccggtttatgttgctgacactgttaaggaagagcttccgaaccgtgcggtcttggcccacaaaccatacgaagcctggaaaattatgaaggaagaaaacttcatcttctccctctacccgaacgaccttatcttcgtcgacgccggtaaggaaatcccgttcaaagccgcactgaagggtagcacccttgacccggaaaagaaagcatcgcgtttcctgatgtactacaaaggggccgacatcgctaccggtagtattagtggcgtgaaccacgacgaaacctacaaagcacgcggcgtaggtattcagtcactgcgcgagattaagaaatgttgcattgacgtgctgggcaacatcagcttcgcttccaaagagaagcgccaaacgttccgt

SEQ ID NO: 105 - DNA, 7579 bp, synthetic construct
  misc_feature 1..7579: note = Description of Artificial Sequence: Synthetic polynucleotide
  misc_feature 1..7579: note = Category: plasmid
  misc_feature 1..7579: note = Description: pAL3 plasmid
  source 1..7579: mol_type = other DNA
ctcgagcaccaccaccaccaccactgataacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggaattgtgagcggataacaattcccctctagaaataattttgtttaactttaagaaggagatataccatgtcaaccgatatgaagaattatcgcattggcgtggacgtcggggatcgcagcgtaggcttggcagcgattgagttcgacgacgacgggctgccgattcagaaactggcattagttacctttcgtcatgacggtggcctcgacccaaccaagaacaaaacgcctatgtcgcgcaaagagacccgcgggatcgcgcgccgcaccatgcgtatgaatcgcgaacgcaaacgccgccttcgcaatttggacaacgtactggagaacttaggctatagcgtacctgagggacctgaaccggaaacttacgaagcctggacgagccgcgcactcttggcaagcattaaattagcatccgccgacgaattaaacgaacacctggttcgtgccgttcgccacatggcgcgccatcgcggctgggcaaacccctggtggagcctggatcaactcgaaaaggcatcacaagaacccagcgaaaccttcgaaatcatcttagcacgcgcacgcgaattattcggcgagaaagtaccagcgaaccccacccttgggatgctgggagcgctggcggcaaacaacgaagtgctgctccgcccacgtgacgagaagaaacgcaaaacgggatacgtacgtggaacgccgttgatgttcgcacaagtgcgccaaggcgatcaattagccgaattacgccgcatctgtgaagtacaaggcatcgaagatcaatatgaagcgctgcgtctgggtgtctttgatcacaaacatccgtacgtcccaaaagagcgcgttggcaaagatccgttgaacccgagcaccaaccgtaccatccgcgcaagccttgaattccaagaatttcgtatcttagacagtgttgccaatctccgcgttcgtatcggcagtcgcgccaaacgcgaactcaccgaagcggaatacgacgcagcggtagaatttctgatggattacgcggacaaagagcagcctagttgggcggacgtggcggaaaagatcggggtgccgggcaatcgtctggtagcccccgtcttagaagacgtacaacagaaaacagccccttacgatcgttcttctgcggcgttcgaaaaggctatggggaagaaaactgaagcccgccaatggtgggagtcgacggacgacgatcaactgcgcagccttctgatcgcgttcctggtggacgccactaatgacaccgaggaagctgccgcggaagccggattaagcgaattgtacaaatcctggcccgcggaagagcgcgaggctttaagcaatatcgacttcgaaaagggtcgcgtggcgtactcacaagaaacactgtccaagttaagtgaatatatgcacgaatatcgcgtcgggctgcatgaagcacgtaaagcagtatttggggttgacgacacgtggcgcccacctctggacaaattagaagagcctaccggacaacctgcagtagaccgtgtcctcaccatcttacgccgcttcgtattggactgcgaacgccaatggggtcgccctcgtgccatcaccgtagagcacactcgcactggtcttatgggcccgactcaacgccaaaagatcttgaacgaacaaaagaagaatcgcgcagacaacgaacgcatccgtgacgaactgcgcgaaagcggagttgacaacccaagtcgtgctgaggtgcgccgtcacttaatcgttcaagaacaagaatgccaatgtctgtattgtggtaccatgatcacaactaccaccagcgaactcgaccacatcgtaccacgcgcaggcggtggcagcagccgccgcgaaaacttggccgccgtatgtcgcgcctgcaacgcaaagaagaaacgcgagttgttttacgcctgggcgggaccggtcaaatcccaagaaacaatcgaacgcgtgcgtcaactgaaagcattcaaagactcaaagaaagctaagatgtttaagaaccaaatccgccgcctgaaccaaacagaagcggacgaacctatcgacgaacgctctctcgcgagcaccagctacgctgccgttgccgtccgcgaacgcttagagcaacatttcaacgaaggactggccctcgatgacaaatcacgtgtcgttttagacgtgtatgcgggcgcggtcacccgcgaaagtcgtcgcgccggcggcatcgatgaacgcatcctgttgcgtggcgaacgtgacaagaatcgcttcgacgttcgtcaccatgccgtggacgcg

SEQ ID NO: 106 - DNA, 2356 bp, synthetic construct
  misc_feature 1..2356: note = Description of Artificial Sequence: Synthetic polynucleotide
  misc_feature 1..2356: note = Category: plasmid
  misc_feature 1..2356: note = Description: PAM library U67/U40 plasmid
  misc_difference 2277..2284: note = Any nucleotide
  source 1..2356: mol_type = other DNA
aatttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaattcattaaaaattgaattgacattaacctataaaaataggcgtcgaggccctttcgtcttccgtgagccaccacgtcgcaagcctcgacnnnnnnnntggagatatcttgaaccttgcatccccggaagagagtcaatcccggaagagagtcaattcagggtggtgaat

SEQ ID NO: 107 - DNA, 2801 bp, synthetic construct
  misc_feature 1..2801: note = Description of Artificial Sequence: Synthetic polynucleotide
  misc_feature 1..2801: note = Category: plasmid
  misc_feature 1..2801: note = Description: pMG010 plasmid
  source 1..2801: mol_type = other DNA
gcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccttgaagactaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcaagtcccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgt

SEQ ID NO: 108 - DNA, 3773 bp, synthetic construct
  misc_feature 1..3773: note = Description of Artificial Sequence: Synthetic polynucleotide
  misc_feature 1..3773: note = Category: MG3-6/3-4 containing 5'UTR, NLS, CDS, NLS, 3'UTR, polyA tail
  misc_feature 1..3773: note = Description: MG3-6/3-4 mRNA
  source 1..3773: mol_type = other DNA
aaaagccagctccagcaggcgctgctcactcctccccatcctctccctctgtccctctgtccctctgaccctgcactgtcccagcaccatggcccccaagaagaagcggaaagttggcggcggaggcagctccacggatatgaagaactacaggatcggggtcgatgtgggagaccggtccgtgggactcgccgccatcgaatttgatgatgatggcttgcccatccaaaagctagctctcgtgactttccgacacgatggcgggctagatcctacaaagaataagactcccatgagccggaaggaaacaagaggtattgcaagacggacgatgaggatgaaccgggagagaaagcggcgtctgcgaaacctggataatgtccttgaaaatctgggatactcagtcccagaaggcccagagccagagacctatgaggcatggacctcacgggccctgctggcttccatcaagctggctagcgctgatgagctgaatgagcatttggtgcgggctgtgcgacatatggctaggcacagaggatgggcgaatccttggtggtccctcgaccagctggagaaagccagccaggagccttctgagacatttgagattatacttgctagggctcgagagctgtttggtgaaaaggttcctgccaatcctactctgggtatgctcggcgccttggcagccaataatgaggttctcctgcgaccgagagatgaaaagaagaggaagacaggctatgtgcgagggactcctctgatgtttgcgcaggtccgtcagggtgaccagcttgctgagctgcgacggatttgcgaggtgcagggaattgaggaccagtacgaggccctcagacttggggtgttcgaccataagcacccctatgtgcccaaggaaagagtggggaaggaccccctcaatccatctacaaatagaacgatacgggctagtttggagtttcaggagttcaggatactggattctgtagctaaccttagggtacggattgggtctcgtgcgaagcgggagctgactgaggcagagtatgatgccgctgttgagttcctaatggactatgctgataaggaacaaccatcctgggctgatgttgctgagaaaataggcgtccccgggaaccggttggtcgctcctgtgctcgaggatgtccagcaaaagaccgctccatatgaccgctccagcgctgctttcgagaaagcaatgggcaagaagaccgaggctcggcagtggtgggaatctacagatgatgaccagcttagatctctgcttattgcatttcttgtagatgcaacaaacgatacagaagaggcggcggctgaggcaggcctttcagagctatataagtcttggcctgcagaggaacgagaagcactaagtaacattgatttcgagaaaggtagggttgcctattctcaggagaccttgtcaaaactaagcgagtacatgcatgagtaccgtgtgggactccacgaggctaggaaggccgtgttcggcgtagatgatacctggcgaccgccgttggataagctggaagaacccactggccagccggctgtggatcgcgtgctaactatactgaggcggtttgtcctggattgtgagaggcagtggggtcgtccccgagcaataacagtggaacataccaggaccggcttgatgggacctacccagaggcagaaaattctgaatgagcagaagaagaaccgagctgataatgagcggattcgcgatgagctacgggagtctggcgtggataatccttctcgggccgaagtacggcggcatttgattgtacaggagcaggagtgtcagtgcttgtactgcggcactatgattacgaccacaacatcagagctggatcatattgtgcccagggctggggggggatcttcccgaagggagaatctggcagctgtttgcagagcttgtaatgccaagaagaagagagaactcttctatgcatgggctggcccagttaagtctcaggagaccattgagagagttagacagcttaaggcctttaaggattctaagaaggccaaaatgttcaagaatcagattaggcgattgaatcagaccgaggccgatgagcccattgatgagcggtccctggcatcaacaagttatgccgctgtggctgtgcgagagagactagaacagcactttaatgagggcctcgcgctggacgataagagccgggtggtgcttgatgtctacgctggggccgtgacacgggagtccaggcgggcaggtggtattgacgagaggattctgctgcggggagagcgagataagaaccgatttgatgtccgacatcacgcggtcgatgccgcagtcatgactttgcttaataggagcgtcgcactcacccttgagcagagaagccagctacgcagggctttctatgagctggagctagataaacttgaccgagatcagcttaagcccggcgaagattggcggaactttacgggcctctacgaggcaagccagaataagttctcagagtggaagaaggccgctacagtactcggggatctactcgctgaagccattgaggatgatgccattgctgtcgtgagccctctgcgattgaggccgcagaatggcagcgtccacgatgacacaattaatgcggttaagaagctgactttggggtccgcatggccagccgatgccgtgaagaggatagttgatccagagatatacttggcaatgaaggacgtcctaggaaaactaaaggagctacccgaggatagcgcaaggtccttggagctctctgatggcaggtacatagaggctgatgatgaggttctgttcttccctaagaaggcagcttccatactgactccaagaggcgctgcagagatagggaattctatacatcatgcccgcctttattcttggctgaccaagaagggagaattgaagttcggaatgctccgagtgtatggggcggagttcccgtggctcatgcgggaatccggttccagagatgttctccatatgcctattcatccaggaagtcagtcattccgcggcatgcaggatggggttcgaaaggcagtagagagcggcgaagctgtggagtttggatggattacccaggatgatgagttggagtttgatcctgaagactacattgctcacggcggcgatgatgagcttaataggctacttagagtaatgcctgagagaaggtggcgggtcgatggcttctataatgccggaacgcttaggattagaccagcacttctctctgcggagcagctgccttcagagctccagaagaaggtggcagataagactctgagtgacgttgagcttattctgctgcgcgccgtacagcgtgggttgtttgttgctataagtagctttctccctctggagagcctgaaggttataaggcggaataacttgggcttcccaagatggcggggaaacggaaatttgccaacatctttcgaggtcaggagcagtgctctcagggctctcggggttgaaggatctggcggaaaaagacctgccgccacaaagaaagccggacaggccaagaaaaagaagtgaccacacccccattcccccactccagatagaacttcagttatatctcacgtgtctggagttggatcccttgaagactaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 109 - protein, 1279 aa, synthetic construct
  REGION 1..1279: note = Description of Artificial Sequence: Synthetic polypeptide
  REGION 1..1279: note = Category: MG29 chimeric effectors
  REGION 1..1279: note = Description: MG29-1_29-5 chimera
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPYKRDKIKLNFEAPTLLNGWDLNKERANRSVLLLKNGNYYLAIMHPNHTDIFKKYMEMDNSDNYEKINYKLISDANRMLPRVFFSKKGIKTYDPPKSILELYKKGEHIKGPSFKLESLHRLIDYFKSVVSKYKADPGDQYGWEVFDFKFSPTSQYEDIGQFYKELEKQAYRVWFTPISSTYIEEAAKHGKLFLFQIYNKDFSPYAKGRPNLHTLYWKSLFEKENLQDVITKLNGEAEIFFRHHSIKKADTVIHKAGETIKNKNENNPKQESTFKHDIIKDRRYTVDKILFHVPITINFKNDKVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 110 - protein, 1291 aa, synthetic construct
  REGION 1..1291: note = Description of Artificial Sequence: Synthetic polypeptide
  REGION 1..1291: note = Category: MG29 chimeric effectors
  REGION 1..1291: note = Description: MG29-1_57-1 chimera
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPQDDVKENKLKLNFGNSSLLGGWSDGQEKTKAATLLRCENDLYLCILKKRDVFDTSKDNPIYSSPSNASRLIIRNLKFQTLAGKGFLSEYGISYGEMSKDDPKKAIQCLQEIIKERYVKKYPLLEKFVTIKYADKSEFDAEINETLKECYVCEFVPINWNLIVEKQNNDELFLFKILCKDYKPNSIGKRDLQTIYWEDVLSDASNHQLCAGAEIFMRKPIANESPLTHRIGSKLVNKRDKDGNTIPDKFYREIYLYANGKTKVISKEAKKYIDEEKATIKDVNHEIIKDKRFYGDSQEPQYMFHCPIKLNYKTKVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 111 - DNA, 39 bp, synthetic construct
  misc_feature 1..39: note = Description of Artificial Sequence: Synthetic oligonucleotide
  misc_feature 1..39: note = Category: MG29 sgRNA
  misc_feature 1..39: note = Description: MG29-1 sgRNA
  misc_difference 20..39: note = Any nucleotide
  source 1..39: mol_type = other DNA
aatttctactgttgtagatnnnnnnnnnnnnnnnnnnnn

SEQ ID NO: 112 - DNA, 39 bp, synthetic construct
  misc_feature 1..39: note = Description of Artificial Sequence: Synthetic oligonucleotide
  misc_feature 1..39: note = Category: MG29 sgRNA
  misc_feature 1..39: note = Description: MG29-5 sgRNA
  misc_difference 20..39: note = Any nucleotide
  source 1..39: mol_type = other DNA
aatttctacagttgtagatnnnnnnnnnnnnnnnnnnnn

SEQ ID NO: 113 - DNA, 39 bp, synthetic construct
  misc_feature 1..39: note = Description of Artificial Sequence: Synthetic oligonucleotide
  misc_feature 1..39: note = Category: MG57 sgRNA
  misc_feature 1..39: note = Description: MG57-1 sgRNA
  source 1..39: mol_type = other DNA
  misc_difference 20..39: note = any nucleotide
aatttctactatcgtagatnnnnnnnnnnnnnnnnnnnn

SEQ ID NO: 114-117 - skipped (4 entries)

SEQ ID NO: 118 - DNA, 35 bp, synthetic construct
  misc_feature 1..35: note = Description of Artificial Sequence: Synthetic oligonucleotide
  misc_feature 1..35: note = Category: primer
  misc_feature 1..35: note = Description: LA179 primer used for PCR in PAM enrichment assay
  source 1..35: mol_type = other DNA
  misc_difference 14..18: note = any nucleotide
gctcttccgatctnnnnngcagctggcacgacagg

SEQ ID NO: 119 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC A1
  source 1..110: mol_type = other RNA
gccgtgtaccagctgagagactgttgagaatcgaaagattcttaataaggcatccttccgatgctgacttctcaccgtccgttttccaataggagcgggcggtatgtttt

SEQ ID NO: 120 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC B1
  source 1..110: mol_type = other RNA
attcaccgattttgattctcaagttgagaatcgaaagattcttaataaggcatccttccgatgctgacttctcaccgtccgttttccaataggagcgggcggtatgtttt

SEQ ID NO: 121 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC C1
  source 1..110: mol_type = other RNA
gattctgatgtgtatatcacaggttgagaatcgaaagattcttaataaggcatccttccgatgctgacttctcaccgtccgttttccaataggagcgggcggtatgtttt

SEQ ID NO: 122 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC D1
  source 1..110: mol_type = other RNA
aacagtgctgtggcctggagcagttgagaatcgaaagattcttaataaggcatccttccgatgctgacttctcaccgtccgttttccaataggagcgggcggtatgtttt

SEQ ID NO: 123 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC E1
  source 1..110: mol_type = other RNA
ggctggggaagaaggtgtcttcgttgagaatcgaaagattcttaataaggcatccttccgatgctgacttctcaccgtccgttttccaataggagcgggcggtatgtttt

SEQ ID NO: 124 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC F1
  source 1..110: mol_type = other RNA
gttttgtctgtgatatacacatgttgagaatcgaaagattcttaataaggcatccttccgatgctgacttctcaccgtccgttttccaataggagcgggcggtatgtttt

SEQ ID NO: 125 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC G1
  source 1..110: mol_type = other RNA
ttactttgtgacacatttgtttgttgagaatcgaaagattcttaataaggcatccttccgatgctgacttctcaccgtccgttttccaataggagcgggcggtatgtttt

SEQ ID NO: 126 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC H1
  source 1..110: mol_type = other RNA
ttgtgacacatttgtttgagaagttgagaatcgaaagattcttaataaggcatccttccgatgctgacttctcaccgtccgttttccaataggagcgggcggtatgtttt

SEQ ID NO: 127 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC A2
  source 1..110: mol_type = other RNA
tgtgacacatttgtttgagaatgttgagaatcgaaagattcttaataaggcatccttccgatgctgacttctcaccgtccgttttccaataggagcgggcggtatgtttt

SEQ ID NO: 128 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC B2
  source 1..110: mol_type = other RNA
atttgtttgagaatcaaaatcggttgagaatcgaaagattcttaataaggcatccttccgatgctgacttctcaccgtccgttttccaataggagcgggcggtatgtttt

SEQ ID NO: 129 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC C2
  source 1..110: mol_type = other RNA
ttcctgtgatgtcaagctggtcgttgagaatcgaaagattcttaataaggcatccttccgatgctgacttctcaccgtccgttttccaataggagcgggcggtatgtttt

SEQ ID NO: 130 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC D2
  source 1..110: mol_type = other RNA
tcctgtgatgtcaagctggtcggttgagaatcgaaagattcttaataaggcatccttccgatgctgacttctcaccgtccgttttccaataggagcgggcggtatgtttt

SEQ ID NO: 131 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC E2
  source 1..110: mol_type = other RNA
gtcaagctggtcgagaaaagctgttgagaatcgaaagattcttaataaggcatccttccgatgctgacttctcaccgtccgttttccaataggagcgggcggtatgtttt

SEQ ID NO: 132 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC F2
  source 1..110: mol_type = other RNA
agcttgacatcacaggaactttgttgagaatcgaaagattcttaataaggcatccttccgatgctgacttctcaccgtccgttttccaataggagcgggcggtatgtttt

SEQ ID NO: 133 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC G2
  source 1..110: mol_type = other RNA
gacatcacaggaactttctaaagttgagaatcgaaagattcttaataaggcatccttccgatgctgacttctcaccgtccgttttccaataggagcgggcggtatgtttt

SEQ ID NO: 134 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC H2
  source 1..110: mol_type = other RNA
ttacagatacgaacctaaacttgttgagaatcgaaagattcttaataaggcatccttccgatgctgacttctcaccgtccgttttccaataggagcgggcggtatgtttt

SEQ ID NO: 135 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC A3
  source 1..110: mol_type = other RNA
aaaacctgtcagtgattgggttgttgagaatcgaaagattcttaataaggcatccttccgatgctgacttctcaccgtccgttttccaataggagcgggcggtatgtttt

SEQ ID NO: 136 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC B3
  source 1..110: mol_type = other RNA
gattgggttccgaatcctcctcgttgagaatcgaaagattcttaataaggcatccttccgatgctgacttctcaccgtccgttttccaataggagcgggcggtatgtttt

SEQ ID NO: 137 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC C3
  source 1..110: mol_type = other RNA
ggaacccaatcactgacaggttgttgagaatcgaaagattcttaataaggcatccttccgatgctgacttctcaccgtccgttttccaataggagcgggcggtatgtttt

SEQ ID NO: 138 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRAC
  misc_feature 1..110: note = Description: MG3-6/3-4 TRAC D3
  source 1..110: mol_type = other RNA
ttgaaagtttaggttcgtatctgttgagaatcgaaagattcttaataaggcatccttccgatgctgacttctcaccgtccgttttccaataggagcgggcggtatgtttt

SEQ ID NO: 139 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC A1
  source 1..22: mol_type = other DNA
gccgtgtaccagctgagagact

SEQ ID NO: 140 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC B1
  source 1..22: mol_type = other DNA
attcaccgattttgattctcaa

SEQ ID NO: 141 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC C1
  source 1..22: mol_type = other DNA
gattctgatgtgtatatcacag

SEQ ID NO: 142 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC D1
  source 1..22: mol_type = other DNA
aacagtgctgtggcctggagca

SEQ ID NO: 143 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC E1
  source 1..22: mol_type = other DNA
ggctggggaagaaggtgtcttc

SEQ ID NO: 144 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC F1
  source 1..22: mol_type = other DNA
gttttgtctgtgatatacacat

SEQ ID NO: 145 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC G1
  source 1..22: mol_type = other DNA
ttactttgtgacacatttgttt

SEQ ID NO: 146 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC H1
  source 1..22: mol_type = other DNA
ttgtgacacatttgtttgagaa

SEQ ID NO: 147 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC A2
  source 1..22: mol_type = other DNA
tgtgacacatttgtttgagaat

SEQ ID NO: 148 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC B2
  source 1..22: mol_type = other DNA
atttgtttgagaatcaaaatcg

SEQ ID NO: 149 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC C2
  source 1..22: mol_type = other DNA
ttcctgtgatgtcaagctggtc

SEQ ID NO: 150 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC D2
  source 1..22: mol_type = other DNA
tcctgtgatgtcaagctggtcg

SEQ ID NO: 151 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC E2
  source 1..22: mol_type = other DNA
gtcaagctggtcgagaaaagct

SEQ ID NO: 152 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC F2
  source 1..22: mol_type = other DNA
agcttgacatcacaggaacttt

SEQ ID NO: 153 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC G2
  source 1..22: mol_type = other DNA
gacatcacaggaactttctaaa

SEQ ID NO: 154 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC H2
  source 1..22: mol_type = other DNA
ttacagatacgaacctaaactt

SEQ ID NO: 155 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC A3
  source 1..22: mol_type = other DNA
aaaacctgtcagtgattgggtt

SEQ ID NO: 156 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC B3
  source 1..22: mol_type = other DNA
gattgggttccgaatcctcctc

SEQ ID NO: 157 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC C3
  source 1..22: mol_type = other DNA
ggaacccaatcactgacaggtt

SEQ ID NO: 158 - DNA, 22 bp, synthetic construct
  misc_feature 1..22: note = Description of Artificial Sequence: Synthetic oligonucleotide
  misc_feature 1..22: note = Category: DNA sequence of TRAC target site
  misc_feature 1..22: note = Description: MG3-6/3-4 TRAC D3
  source 1..22: mol_type = other DNA
ttgaaagtttaggttcgtatct

SEQ ID NO: 159 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M A1
  source 1..110: mol_type = other RNA
tcacgctggatagcctccaggcgttgagaatcgaaagattcttaataaggcatccttccgatgctgacttctcaccgtccgttttccaataggagcgggcggtatgtttt

SEQ ID NO: 160 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M B1
  source 1..110: mol_type = other RNA
ggtttactcacgtcatccagcagttgagaatcgaaagattcttaataaggcatccttccgatgctgacttctcaccgtccgttttccaataggagcgggcggtatgtttt

SEQ ID NO: 161 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M C1
  source 1..110: mol_type = other RNA
actcacgtcatccagcagagaagttgagaatcgaaagattcttaataaggcatccttccgatgctgacttctcaccgtccgttttccaataggagcgggcggtatgtttt

SEQ ID NO: 162 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M D1
  source 1..110: mol_type = other RNA
tcatccagcagagaatggaaaggttgagaatcgaaagattcttaataaggcatccttccgatgctgacttctcaccgtccgttttccaataggagcgggcggtatgtttt

SEQ ID NO: 163 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M E1
  source 1..110: mol_type = other RNA
agagaatggaaagtcaaatttcgttgagaatcgaaagattcttaataaggcatccttccgatgctgacttctcaccgtccgttttccaataggagcgggcggtatgtttt

SEQ ID NO: 164 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M F1
  source 1..110: mol_type = other RNA
cgacattgaagttgacttactggttgagaatcgaaagattcttaataaggcatccttccgatgctgacttctcaccgtccgttttccaataggagcgggcggtatgtttt

SEQ ID NO: 165 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M G1
  source 1..110: mol_type = other RNA
ttgacttactgaagaatggagagttgagaatcgaaagattcttaataaggcatccttccgatgctgacttctcaccgtccgttttccaataggagcgggcggtatgtttt

SEQ ID NO: 166 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M H1
  source 1..110: mol_type = other RNA
ttactgaagaatggagagagaagttgagaatcgaaagattcttaataaggcatccttccgatgctgacttctcaccgtccgttttccaataggagcgggcggtatgtttt

SEQ ID NO: 167 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M A2
  source 1..110: mol_type = other RNA
tactgaagaatggagagagaatgttgagaatcgaaagattcttaataaggcatccttccgatgctgacttctcaccgtccgttttccaataggagcgggcggtatgtttt

SEQ ID NO: 168 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M B2
  source 1..110: mol_type = other RNA
actgaagaatggagagagaattgttgagaatcgaaagattcttaataaggcatccttccgatgctgacttctcaccgtccgttttccaataggagcgggcggtatgtttt

SEQ ID NO: 169 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M C2
  source 1..110: mol_type = other RNA
tctttctatctcttgtactacagttgagaatcgaaagattcttaataaggcatccttccgatgctgacttctcaccgtccgttttccaataggagcgggcggtatgtttt

SEQ ID NO: 170 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M D2
  source 1..110: mol_type = other RNA
tactacactgaattcacccccagttgagaatcgaaagattcttaataaggcatccttccgatgctgacttctcaccgtccgttttccaataggagcgggcggtatgtttt

SEQ ID NO: 171 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M E2
  source 1..110: mol_type = other RNA
actacactgaattcacccccacgttgagaatcgaaagattcttaataaggcatccttccgatgctgacttctcaccgtccgttttccaataggagcgggcggtatgtttt

SEQ ID NO: 172 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M F2
  source 1..110: mol_type = other RNA
ctacactgaattcacccccactgttgagaatcgaaagattcttaataaggcatccttccgatgctgacttctcaccgtccgttttccaataggagcgggcggtatgtttt

SEQ ID NO: 173 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M G2
  source 1..110: mol_type = other RNA
atactcatctttttcagtgggggttgagaatcgaaagattcttaataaggcatccttccgatgctgacttctcaccgtccgttttccaataggagcgggcggtatgtttt

SEQ ID NO: 174 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M H2
  source 1..110: mol_type = other RNA
gaattcagtgtagtacaagagagttgagaatcgaaagattcttaataaggcatccttccgatgctgacttctcaccgtccgttttccaataggagcgggcggtatgtttt

SEQ ID NO: 175 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M A3
  source 1..110: mol_type = other RNA
gagatagaaagaccagtccttggttgagaatcgaaagattcttaataaggcatccttccgatgctgacttctcaccgtccgttttccaataggagcgggcggtatgtttt

SEQ ID NO: 176 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M B3
  source 1..110: mol_type = other RNA
cagtccttgctgaaagacaagtgttgagaatcgaaagattcttaataaggcatccttccgatgctgacttctcaccgtccgttttccaataggagcgggcggtatgtttt

SEQ ID NO: 177 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M C3
  source 1..110: mol_type = other RNA
agtcaacttcaatgtcggatgggttgagaatcgaaagattcttaataaggcatccttccgatgctgacttctcaccgtccgttttccaataggagcgggcggtatgtttt

SEQ ID NO: 178 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M D3
  source 1..110: mol_type = other RNA
aaacccagacacatagcaattcgttgagaatcgaaagattcttaataaggcatccttccgatgctgacttctcaccgtccgttttccaataggagcgggcggtatgtttt

SEQ ID NO: 179 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M E3
  source 1..110: mol_type = other RNA
aacccagacacatagcaattcagttgagaatcgaaagattcttaataaggcatccttccgatgctgacttctcaccgtccgttttccaataggagcgggcggtatgtttt

SEQ ID NO: 180 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M F3
  source 1..110: mol_type = other RNA
ctgctggatgacgtgagtaaacgttgagaatcgaaagattcttaataaggcatccttccgatgctgacttctcaccgtccgttttccaataggagcgggcggtatgtttt

SEQ ID NO: 181 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M G3
  source 1..110: mol_type = other RNA
acctgaatctttggagtacctggttgagaatcgaaagattcttaataaggcatccttccgatgctgacttctcaccgtccgttttccaataggagcgggcggtatgtttt

SEQ ID NO: 182 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M H3
  source 1..110: mol_type = other RNA
tgctgcttacatgtctcgatctgttgagaatcgaaagattcttaataaggcatccttccgatgctgacttctcaccgtccgttttccaataggagcgggcggtatgtttt

SEQ ID NO: 183 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M A4
  source 1..110: mol_type = other RNA
gctgcttacatgtctcgatctagttgagaatcgaaagattcttaataaggcatccttccgatgctgacttctcaccgtccgttttccaataggagcgggcggtatgtttt

SEQ ID NO: 184 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting B2M
  misc_feature 1..110: note = Description: MG3-6/3-4 B2M B4
  source 1..110: mol_type = other RNA
ctgcttacatgtctcgatctatgttgagaatcgaaagattcttaataaggcatccttccgatgctgacttctcaccgtccgttttccaataggagcgggcggtatgtttt

SEQ ID NO: 185 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M A1
  source 1..22: mol_type = other DNA
tcacgctggatagcctccaggc

SEQ ID NO: 186 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M B1
  source 1..22: mol_type = other DNA
ggtttactcacgtcatccagca

SEQ ID NO: 187 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M C1
  source 1..22: mol_type = other DNA
actcacgtcatccagcagagaa

SEQ ID NO: 188 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M D1
  source 1..22: mol_type = other DNA
tcatccagcagagaatggaaag

SEQ ID NO: 189 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M E1
  source 1..22: mol_type = other DNA
agagaatggaaagtcaaatttc

SEQ ID NO: 190 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M F1
  source 1..22: mol_type = other DNA
cgacattgaagttgacttactg

SEQ ID NO: 191 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M G1
  source 1..22: mol_type = other DNA
ttgacttactgaagaatggaga

SEQ ID NO: 192 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M H1
  source 1..22: mol_type = other DNA
ttactgaagaatggagagagaa

SEQ ID NO: 193 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M A2
  source 1..22: mol_type = other DNA
tactgaagaatggagagagaat

SEQ ID NO: 194 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M B2
  source 1..22: mol_type = other DNA
actgaagaatggagagagaatt

SEQ ID NO: 195 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M C2
  source 1..22: mol_type = other DNA
tctttctatctcttgtactaca

SEQ ID NO: 196 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M D2
  source 1..22: mol_type = other DNA
tactacactgaattcaccccca

SEQ ID NO: 197 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M E2
  source 1..22: mol_type = other DNA
actacactgaattcacccccac

SEQ ID NO: 198 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M F2
  source 1..22: mol_type = other DNA
ctacactgaattcacccccact

SEQ ID NO: 199 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M G2
  source 1..22: mol_type = other DNA
atactcatctttttcagtgggg

SEQ ID NO: 200 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M H2
  source 1..22: mol_type = other DNA
gaattcagtgtagtacaagaga

SEQ ID NO: 201 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M A3
  source 1..22: mol_type = other DNA
gagatagaaagaccagtccttg

SEQ ID NO: 202 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M B3
  source 1..22: mol_type = other DNA
cagtccttgctgaaagacaagt

SEQ ID NO: 203 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M C3
  source 1..22: mol_type = other DNA
agtcaacttcaatgtcggatgg

SEQ ID NO: 204 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M D3
  source 1..22: mol_type = other DNA
aaacccagacacatagcaattc

SEQ ID NO: 205 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M E3
  source 1..22: mol_type = other DNA
aacccagacacatagcaattca

SEQ ID NO: 206 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M F3
  source 1..22: mol_type = other DNA
ctgctggatgacgtgagtaaac

SEQ ID NO: 207 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M G3
  source 1..22: mol_type = other DNA
acctgaatctttggagtacctg

SEQ ID NO: 208 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M H3
  source 1..22: mol_type = other DNA
tgctgcttacatgtctcgatct

SEQ ID NO: 209 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M A4
  source 1..22: mol_type = other DNA
gctgcttacatgtctcgatcta

SEQ ID NO: 210 - DNA, 22 bp, synthetic construct
  misc_feature 1..22: note = Description of Artificial Sequence: Synthetic oligonucleotide
  misc_feature 1..22: note = Category: DNA sequence of B2M target site
  misc_feature 1..22: note = Description: MG3-6/3-4 B2M B4
  source 1..22: mol_type = other DNA
ctgcttacatgtctcgatctat

SEQ ID NO: 211 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 A1
  source 1..110: mol_type = other RNA
cagaagcagagatctcccacacgttgagaatcgaaagattcttaataaggcatccttccgatgctgacttctcaccgtccgttttccaataggagcgggcggtatgtttt

SEQ ID NO: 212 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 B1
  source 1..110: mol_type = other RNA
ccacgtggagctgagctggtgggttgagaatcgaaagattcttaataaggcatccttccgatgctgacttctcaccgtccgttttccaataggagcgggcggtatgtttt

SEQ ID NO: 213 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 C1
  source 1..110: mol_type = other RNA
agtccagttctacgggctctcggttgagaatcgaaagattcttaataaggcatccttccgatgctgacttctcaccgtccgttttccaataggagcgggcggtatgtttt

SEQ ID NO: 214 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 D1
  source 1..110: mol_type = other RNA
gattaggtgagaccagctaccagttgagaatcgaaagattcttaataaggcatccttccgatgctgacttctcaccgtccgttttccaataggagcgggcggtatgtttt

SEQ ID NO: 215 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 E1
  source 1..110: mol_type = other RNA
attaggtgagaccagctaccaggttgagaatcgaaagattcttaataaggcatccttccgatgctgacttctcaccgtccgttttccaataggagcgggcggtatgtttt

SEQ ID NO: 216 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 F1
  source 1..110: mol_type = other RNA
ttaggtgagaccagctaccagggttgagaatcgaaagattcttaataaggcatccttccgatgctgacttctcaccgtccgttttccaataggagcgggcggtatgtttt

SEQ ID NO: 217 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 G1
  source 1..110: mol_type = other RNA
tgagaccagctaccagggaaaagttgagaatcgaaagattcttaataaggcatccttccgatgctgacttctcaccgtccgttttccaataggagcgggcggtatgtttt

SEQ ID NO: 218 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 H1
  source 1..110: mol_type = other RNA
caggtagcagacaagactagatgttgagaatcgaaagattcttaataaggcatccttccgatgctgacttctcaccgtccgttttccaataggagcgggcggtatgtttt

SEQ ID NO: 219 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 A2
  source 1..110: mol_type = other RNA
aggtagcagacaagactagatcgttgagaatcgaaagattcttaataaggcatccttccgatgctgacttctcaccgtccgttttccaataggagcgggcggtatgtttt

SEQ ID NO: 220 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 B2
  source 1..110: mol_type = other RNA
agcagacaagactagatccaaagttgagaatcgaaagattcttaataaggcatccttccgatgctgacttctcaccgtccgttttccaataggagcgggcggtatgtttt

SEQ ID NO: 221 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 C2
  source 1..110: mol_type = other RNA
ggaaccagcgcacaccatgaaggttgagaatcgaaagattcttaataaggcatccttccgatgctgacttctcaccgtccgttttccaataggagcgggcggtatgtttt

SEQ ID NO: 222 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 D2
  source 1..110: mol_type = other RNA
gtggctgacatctgcatggcaggttgagaatcgaaagattcttaataaggcatccttccgatgctgacttctcaccgtccgttttccaataggagcgggcggtatgtttt

SEQ ID NO: 223 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 E2
  source 1..110: mol_type = other RNA
ggcctgggagtctgtgccaactgttgagaatcgaaagattcttaataaggcatccttccgatgctgacttctcaccgtccgttttccaataggagcgggcggtatgtttt

SEQ ID NO: 224 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 F2
  source 1..110: mol_type = other RNA
ctgactttacttttaattgcctgttgagaatcgaaagattcttaataaggcatccttccgatgctgacttctcaccgtccgttttccaataggagcgggcggtatgtttt

SEQ ID NO: 225 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 G2
  source 1..110: mol_type = other RNA
tgactttacttttaattgcctagttgagaatcgaaagattcttaataaggcatccttccgatgctgacttctcaccgtccgttttccaataggagcgggcggtatgtttt

SEQ ID NO: 226 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 H2
  source 1..110: mol_type = other RNA
gactttacttttaattgcctatgttgagaatcgaaagattcttaataaggcatccttccgatgctgacttctcaccgtccgttttccaataggagcgggcggtatgtttt

SEQ ID NO: 227 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 A3
  source 1..110: mol_type = other RNA
gggaaggagaagctggagtcacgttgagaatcgaaagattcttaataaggcatccttccgatgctgacttctcaccgtccgttttccaataggagcgggcggtatgtttt

SEQ ID NO: 228 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 B3
  source 1..110: mol_type = other RNA
ggaaggagaagctggagtcaccgttgagaatcgaaagattcttaataaggcatccttccgatgctgacttctcaccgtccgttttccaataggagcgggcggtatgtttt

SEQ ID NO: 229 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 C3
  source 1..110: mol_type = other RNA
aactcctggctcttaataacccgttgagaatcgaaagattcttaataaggcatccttccgatgctgacttctcaccgtccgttttccaataggagcgggcggtatgtttt

SEQ ID NO: 230 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 D3
  source 1..110: mol_type = other RNA
aactttctcttctgcaggtcaagttgagaatcgaaagattcttaataaggcatccttccgatgctgacttctcaccgtccgttttccaataggagcgggcggtatgtttt

SEQ ID NO: 231 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 E3
  source 1..110: mol_type = other RNA
actccacttccagggctgccttgttgagaatcgaaagattcttaataaggcatccttccgatgctgacttctcaccgtccgttttccaataggagcgggcggtatgtttt

SEQ ID NO: 232 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 F3
  source 1..110: mol_type = other RNA
ctccacttccagggctgccttcgttgagaatcgaaagattcttaataaggcatccttccgatgctgacttctcaccgtccgttttccaataggagcgggcggtatgtttt

SEQ ID NO: 233 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 G3
  source 1..110: mol_type = other RNA
tcctttctcttgacctgcagaagttgagaatcgaaagattcttaataaggcatccttccgatgctgacttctcaccgtccgttttccaataggagcgggcggtatgtttt

SEQ ID NO: 234 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 H3
  source 1..110: mol_type = other RNA
agccaggagttgtgaggattgagttgagaatcgaaagattcttaataaggcatccttccgatgctgacttctcaccgtccgttttccaataggagcgggcggtatgtttt

SEQ ID NO: 235 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 A4
  source 1..110: mol_type = other RNA
agtagtagggcccattgaccacgttgagaatcgaaagattcttaataaggcatccttccgatgctgacttctcaccgtccgttttccaataggagcgggcggtatgtttt

SEQ ID NO: 236 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 B4
  source 1..110: mol_type = other RNA
tgcaagttatcttctgaggcacgttgagaatcgaaagattcttaataaggcatccttccgatgctgacttctcaccgtccgttttccaataggagcgggcggtatgtttt

SEQ ID NO: 237 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 C4
  source 1..110: mol_type = other RNA
agttatcttctgaggcacctgagttgagaatcgaaagattcttaataaggcatccttccgatgctgacttctcaccgtccgttttccaataggagcgggcggtatgtttt

SEQ ID NO: 238 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 D4
  source 1..110: mol_type = other RNA
gttatcttctgaggcacctgaagttgagaatcgaaagattcttaataaggcatccttccgatgctgacttctcaccgtccgttttccaataggagcgggcggtatgtttt

SEQ ID NO: 239 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 E4
  source 1..110: mol_type = other RNA
tcaagaaccatgagagagggaggttgagaatcgaaagattcttaataaggcatccttccgatgctgacttctcaccgtccgttttccaataggagcgggcggtatgtttt

SEQ ID NO: 240 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 F4
  source 1..110: mol_type = other RNA
caagaaccatgagagagggagagttgagaatcgaaagattcttaataaggcatccttccgatgctgacttctcaccgtccgttttccaataggagcgggcggtatgtttt

SEQ ID NO: 241 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 G4
  source 1..110: mol_type = other RNA
ttacccgaggtaaagccacagtgttgagaatcgaaagattcttaataaggcatccttccgatgctgacttctcaccgtccgttttccaataggagcgggcggtatgtttt

SEQ ID NO: 242 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 H4
  source 1..110: mol_type = other RNA
ccgaggtaaagccacagtctgagttgagaatcgaaagattcttaataaggcatccttccgatgctgacttctcaccgtccgttttccaataggagcgggcggtatgtttt

SEQ ID NO: 243 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 A5
  source 1..110: mol_type = other RNA
cagtctgaaagaaagcagggaggttgagaatcgaaagattcttaataaggcatccttccgatgctgacttctcaccgtccgttttccaataggagcgggcggtatgtttt

SEQ ID NO: 244 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 B5
  source 1..110: mol_type = other RNA
agtctgaaagaaagcagggagagttgagaatcgaaagattcttaataaggcatccttccgatgctgacttctcaccgtccgttttccaataggagcgggcggtatgtttt

SEQ ID NO: 245 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 C5
  source 1..110: mol_type = other RNA
gtctgaaagaaagcagggagaggttgagaatcgaaagattcttaataaggcatccttccgatgctgacttctcaccgtccgttttccaataggagcgggcggtatgtttt

SEQ ID NO: 246 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 D5
  source 1..110: mol_type = other RNA
gaaagaaagcagggagaggaaagttgagaatcgaaagattcttaataaggcatccttccgatgctgacttctcaccgtccgttttccaataggagcgggcggtatgtttt

SEQ ID NO: 247 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 E5
  source 1..110: mol_type = other RNA
gagaccttattttcataggcaagttgagaatcgaaagattcttaataaggcatccttccgatgctgacttctcaccgtccgttttccaataggagcgggcggtatgtttt

SEQ ID NO: 248 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 F5
  source 1..110: mol_type = other RNA
gatgagagttacacaggccacagttgagaatcgaaagattcttaataaggcatccttccgatgctgacttctcaccgtccgttttccaataggagcgggcggtatgtttt

SEQ ID NO: 249 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 G5
  source 1..110: mol_type = other RNA
agctgcttggctctgttgggctgttgagaatcgaaagattcttaataaggcatccttccgatgctgacttctcaccgtccgttttccaataggagcgggcggtatgtttt

SEQ ID NO: 250 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 H5
  source 1..110: mol_type = other RNA
tgttgggctgagaatctgggaggttgagaatcgaaagattcttaataaggcatccttccgatgctgacttctcaccgtccgttttccaataggagcgggcggtatgtttt

SEQ ID NO: 251 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC1
  misc_feature 1..110: note = Description: MG3-6/3-4 TRBC1 A6
  source 1..110: mol_type = other RNA
ggaacaccttgttcaggtcctcgttgagaatcgaaagattcttaataaggcatccttccgatgctgacttctcaccgtccgttttccaataggagcgggcggtatgtttt

SEQ ID NO: 252 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 A1
  source 1..22: mol_type = other DNA
cagaagcagagatctcccacac

SEQ ID NO: 253 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 B1
  source 1..22: mol_type = other DNA
ccacgtggagctgagctggtgg

SEQ ID NO: 254 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 C1
  source 1..22: mol_type = other DNA
agtccagttctacgggctctcg

SEQ ID NO: 255 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 D1
  source 1..22: mol_type = other DNA
gattaggtgagaccagctacca

SEQ ID NO: 256 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 E1
  source 1..22: mol_type = other DNA
attaggtgagaccagctaccag

SEQ ID NO: 257 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 F1
  source 1..22: mol_type = other DNA
ttaggtgagaccagctaccagg

SEQ ID NO: 258 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 G1
  source 1..22: mol_type = other DNA
tgagaccagctaccagggaaaa

SEQ ID NO: 259 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 H1
  source 1..22: mol_type = other DNA
caggtagcagacaagactagat

SEQ ID NO: 260 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 A2
  source 1..22: mol_type = other DNA
aggtagcagacaagactagatc

SEQ ID NO: 261 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 B2
  source 1..22: mol_type = other DNA
agcagacaagactagatccaaa

SEQ ID NO: 262 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 C2
  source 1..22: mol_type = other DNA
ggaaccagcgcacaccatgaag

SEQ ID NO: 263 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 D2
  source 1..22: mol_type = other DNA
gtggctgacatctgcatggcag

SEQ ID NO: 264 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 E2
  source 1..22: mol_type = other DNA
ggcctgggagtctgtgccaact

SEQ ID NO: 265 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 F2
  source 1..22: mol_type = other DNA
ctgactttacttttaattgcct

SEQ ID NO: 266 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 G2
  source 1..22: mol_type = other DNA
tgactttacttttaattgccta

SEQ ID NO: 267 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 H2
  source 1..22: mol_type = other DNA
gactttacttttaattgcctat

SEQ ID NO: 268 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 A3
  source 1..22: mol_type = other DNA
gggaaggagaagctggagtcac

SEQ ID NO: 269 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 B3
  source 1..22: mol_type = other DNA
ggaaggagaagctggagtcacc

SEQ ID NO: 270 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 C3
  source 1..22: mol_type = other DNA
aactcctggctcttaataaccc

SEQ ID NO: 271 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 D3
  source 1..22: mol_type = other DNA
aactttctcttctgcaggtcaa

SEQ ID NO: 272 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 E3
  source 1..22: mol_type = other DNA
actccacttccagggctgcctt

SEQ ID NO: 273 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 F3
  source 1..22: mol_type = other DNA
ctccacttccagggctgccttc

SEQ ID NO: 274 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 G3
  source 1..22: mol_type = other DNA
tcctttctcttgacctgcagaa

SEQ ID NO: 275 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 H3
  source 1..22: mol_type = other DNA
agccaggagttgtgaggattga

SEQ ID NO: 276 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 A4
  source 1..22: mol_type = other DNA
agtagtagggcccattgaccac

SEQ ID NO: 277 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 B4
  source 1..22: mol_type = other DNA
tgcaagttatcttctgaggcac

SEQ ID NO: 278 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 C4
  source 1..22: mol_type = other DNA
agttatcttctgaggcacctga

SEQ ID NO: 279 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 D4
  source 1..22: mol_type = other DNA
gttatcttctgaggcacctgaa

SEQ ID NO: 280 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 E4
  source 1..22: mol_type = other DNA
tcaagaaccatgagagagggag

SEQ ID NO: 281 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 F4
  source 1..22: mol_type = other DNA
caagaaccatgagagagggaga

SEQ ID NO: 282 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 G4
  source 1..22: mol_type = other DNA
ttacccgaggtaaagccacagt

SEQ ID NO: 283 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 H4
  source 1..22: mol_type = other DNA
ccgaggtaaagccacagtctga

SEQ ID NO: 284 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 A5
  source 1..22: mol_type = other DNA
cagtctgaaagaaagcagggag

SEQ ID NO: 285 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 B5
  source 1..22: mol_type = other DNA
agtctgaaagaaagcagggaga

SEQ ID NO: 286 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 C5
  source 1..22: mol_type = other DNA
gtctgaaagaaagcagggagag

SEQ ID NO: 287 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 D5
  source 1..22: mol_type = other DNA
gaaagaaagcagggagaggaaa

SEQ ID NO: 288 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 E5
  source 1..22: mol_type = other DNA
gagaccttattttcataggcaa

SEQ ID NO: 289 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 F5
  source 1..22: mol_type = other DNA
gatgagagttacacaggccaca

SEQ ID NO: 290 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 G5
  source 1..22: mol_type = other DNA
agctgcttggctctgttgggct

SEQ ID NO: 291 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 H5
  source 1..22: mol_type = other DNA
tgttgggctgagaatctgggag

SEQ ID NO: 292 - DNA, 22 bp, synthetic construct
  misc_feature 1..22: note = Description of Artificial Sequence: Synthetic oligonucleotide
  misc_feature 1..22: note = Category: DNA sequence of TRBC1 target site
  misc_feature 1..22: note = Description: MG3-6/3-4 TRBC1 A6
  source 1..22: mol_type = other DNA
ggaacaccttgttcaggtcctc

SEQ ID NO: 293 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 A1
  source 1..110: mol_type = other RNA
acctcttccctttccagaggacgttgagaatcgaaagattcttaataaggcatccttccgatgctgacttctcaccgtccgttttccaataggagcgggcggtatgtttt

SEQ ID NO: 294 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 B1
  source 1..110: mol_type = other RNA
cctcttccctttccagaggaccgttgagaatcgaaagattcttaataaggcatccttccgatgctgacttctcaccgtccgttttccaataggagcgggcggtatgtttt

SEQ ID NO: 295 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 C1
  source 1..110: mol_type = other RNA
ctcttccctttccagaggacctgttgagaatcgaaagattcttaataaggcatccttccgatgctgacttctcaccgtccgttttccaataggagcgggcggtatgtttt

SEQ ID NO: 296 - RNA, 110 nt, synthetic construct; identical to SEQ ID NO: 211
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 D1
  source 1..110: mol_type = other RNA

SEQ ID NO: 297 - RNA, 110 nt, synthetic construct; identical to SEQ ID NO: 212
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 E1
  source 1..110: mol_type = other RNA

SEQ ID NO: 298 - RNA, 110 nt, synthetic construct; identical to SEQ ID NO: 213
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 F1
  source 1..110: mol_type = other RNA

SEQ ID NO: 299 - RNA, 110 nt, synthetic construct; identical to SEQ ID NO: 214
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 G1
  source 1..110: mol_type = other RNA

SEQ ID NO: 300 - RNA, 110 nt, synthetic construct; identical to SEQ ID NO: 215
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 H1
  source 1..110: mol_type = other RNA

SEQ ID NO: 301 - RNA, 110 nt, synthetic construct; identical to SEQ ID NO: 216
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 A2
  source 1..110: mol_type = other RNA

SEQ ID NO: 302 - RNA, 110 nt, synthetic construct; identical to SEQ ID NO: 217
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 B2
  source 1..110: mol_type = other RNA

SEQ ID NO: 303 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 C2
  source 1..110: mol_type = other RNA
tagcggacaagactagatccaggttgagaatcgaaagattcttaataaggcatccttccgatgctgacttctcaccgtccgttttccaataggagcgggcggtatgtttt

SEQ ID NO: 304 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 D2
  source 1..110: mol_type = other RNA
cccccaccaagaagcatagagggttgagaatcgaaagattcttaataaggcatccttccgatgctgacttctcaccgtccgttttccaataggagcgggcggtatgtttt

SEQ ID NO: 305 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 E2
  source 1..110: mol_type = other RNA
tctgctctcgaaccagggcatggttgagaatcgaaagattcttaataaggcatccttccgatgctgacttctcaccgtccgttttccaataggagcgggcggtatgtttt

SEQ ID NO: 306 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 F2
  source 1..110: mol_type = other RNA
ggaacatcacacatgggcataagttgagaatcgaaagattcttaataaggcatccttccgatgctgacttctcaccgtccgttttccaataggagcgggcggtatgtttt

SEQ ID NO: 307 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 G2
  source 1..110: mol_type = other RNA
cctaatatatcctatcacctcagttgagaatcgaaagattcttaataaggcatccttccgatgctgacttctcaccgtccgttttccaataggagcgggcggtatgtttt

SEQ ID NO: 308 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 H2
  source 1..110: mol_type = other RNA
accataatgaagccagactggggttgagaatcgaaagattcttaataaggcatccttccgatgctgacttctcaccgtccgttttccaataggagcgggcggtatgtttt

SEQ ID NO: 309 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 A3
  source 1..110: mol_type = other RNA
ccataatgaagccagactgggggttgagaatcgaaagattcttaataaggcatccttccgatgctgacttctcaccgtccgttttccaataggagcgggcggtatgtttt

SEQ ID NO: 310 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 B3
  source 1..110: mol_type = other RNA
cataatgaagccagactggggagttgagaatcgaaagattcttaataaggcatccttccgatgctgacttctcaccgtccgttttccaataggagcgggcggtatgtttt

SEQ ID NO: 311 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 C3
  source 1..110: mol_type = other RNA
gccagactggggagaaaatgcagttgagaatcgaaagattcttaataaggcatccttccgatgctgacttctcaccgtccgttttccaataggagcgggcggtatgtttt

SEQ ID NO: 312 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 D3
  source 1..110: mol_type = other RNA
ggagaaaatgcagggaatatcagttgagaatcgaaagattcttaataaggcatccttccgatgctgacttctcaccgtccgttttccaataggagcgggcggtatgtttt

SEQ ID NO: 313 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 E3
  source 1..110: mol_type = other RNA
ggagacaaccagcgagccctacgttgagaatcgaaagattcttaataaggcatccttccgatgctgacttctcaccgtccgttttccaataggagcgggcggtatgtttt

SEQ ID NO: 314 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 F3
  source 1..110: mol_type = other RNA
tactcctgctgtgccatagcccgttgagaatcgaaagattcttaataaggcatccttccgatgctgacttctcaccgtccgttttccaataggagcgggcggtatgtttt

SEQ ID NO: 315 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 G3
  source 1..110: mol_type = other RNA
ctgtgccatagcccctgaaaccgttgagaatcgaaagattcttaataaggcatccttccgatgctgacttctcaccgtccgttttccaataggagcgggcggtatgtttt

SEQ ID NO: 316 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 H3
  source 1..110: mol_type = other RNA
tgtgccatagcccctgaaacccgttgagaatcgaaagattcttaataaggcatccttccgatgctgacttctcaccgtccgttttccaataggagcgggcggtatgtttt

SEQ ID NO: 317 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 A4
  source 1..110: mol_type = other RNA
gtgccatagcccctgaaaccctgttgagaatcgaaagattcttaataaggcatccttccgatgctgacttctcaccgtccgttttccaataggagcgggcggtatgtttt

SEQ ID NO: 318 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 B4
  source 1..110: mol_type = other RNA
tgttctctcttccacaggtcaagttgagaatcgaaagattcttaataaggcatccttccgatgctgacttctcaccgtccgttttccaataggagcgggcggtatgtttt

SEQ ID NO: 319 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 C4
  source 1..110: mol_type = other RNA
gaaaggattccagaggctagctgttgagaatcgaaagattcttaataaggcatccttccgatgctgacttctcaccgtccgttttccaataggagcgggcggtatgtttt

SEQ ID NO: 320 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 D4
  source 1..110: mol_type = other RNA
ggatggttttggagctagcctcgttgagaatcgaaagattcttaataaggcatccttccgatgctgacttctcaccgtccgttttccaataggagcgggcggtatgtttt

SEQ ID NO: 321 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 E4
  source 1..110: mol_type = other RNA
ccctggttcgagagcagagacggttgagaatcgaaagattcttaataaggcatccttccgatgctgacttctcaccgtccgttttccaataggagcgggcggtatgtttt

SEQ ID NO: 322 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 F4
  source 1..110: mol_type = other RNA
agcagagacggcgaaagatagagttgagaatcgaaagattcttaataaggcatccttccgatgctgacttctcaccgtccgttttccaataggagcgggcggtatgtttt

SEQ ID NO: 323 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 G4
  source 1..110: mol_type = other RNA
gcagagacggcgaaagatagaggttgagaatcgaaagattcttaataaggcatccttccgatgctgacttctcaccgtccgttttccaataggagcgggcggtatgtttt

SEQ ID NO: 324 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 H4
  source 1..110: mol_type = other RNA
cagagacggcgaaagatagagagttgagaatcgaaagattcttaataaggcatccttccgatgctgacttctcaccgtccgttttccaataggagcgggcggtatgtttt

SEQ ID NO: 325 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 A5
  source 1..110: mol_type = other RNA
ttaccggaggtgaagccacagtgttgagaatcgaaagattcttaataaggcatccttccgatgctgacttctcaccgtccgttttccaataggagcgggcggtatgtttt

SEQ ID NO: 326 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 B5
  source 1..110: mol_type = other RNA
cggaggtgaagccacagtctgagttgagaatcgaaagattcttaataaggcatccttccgatgctgacttctcaccgtccgttttccaataggagcgggcggtatgtttt

SEQ ID NO: 327 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 C5
  source 1..110: mol_type = other RNA
ggaggtgaagccacagtctgaagttgagaatcgaaagattcttaataaggcatccttccgatgctgacttctcaccgtccgttttccaataggagcgggcggtatgtttt

SEQ ID NO: 328 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 D5
  source 1..110: mol_type = other RNA
acagtctgaaagaaaacagggggttgagaatcgaaagattcttaataaggcatccttccgatgctgacttctcaccgtccgttttccaataggagcgggcggtatgtttt

SEQ ID NO: 329 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 E5
  source 1..110: mol_type = other RNA
cagtctgaaagaaaacaggggagttgagaatcgaaagattcttaataaggcatccttccgatgctgacttctcaccgtccgttttccaataggagcgggcggtatgtttt

SEQ ID NO: 330 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 F5
  source 1..110: mol_type = other RNA
agtctgaaagaaaacaggggaagttgagaatcgaaagattcttaataaggcatccttccgatgctgacttctcaccgtccgttttccaataggagcgggcggtatgtttt

SEQ ID NO: 331 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 G5
  source 1..110: mol_type = other RNA
gtctgaaagaaaacaggggaaggttgagaatcgaaagattcttaataaggcatccttccgatgctgacttctcaccgtccgttttccaataggagcgggcggtatgtttt

SEQ ID NO: 332 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 H5
  source 1..110: mol_type = other RNA
acaggggaagaaaaatggatgagttgagaatcgaaagattcttaataaggcatccttccgatgctgacttctcaccgtccgttttccaataggagcgggcggtatgtttt

SEQ ID NO: 333 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 A6
  source 1..110: mol_type = other RNA
gcgaagtggtcactatgatcttgttgagaatcgaaagattcttaataaggcatccttccgatgctgacttctcaccgtccgttttccaataggagcgggcggtatgtttt

SEQ ID NO: 334 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 B6
  source 1..110: mol_type = other RNA
ttaggaaaccaggaccccagaagttgagaatcgaaagattcttaataaggcatccttccgatgctgacttctcaccgtccgttttccaataggagcgggcggtatgtttt

SEQ ID NO: 335 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 C6
  source 1..110: mol_type = other RNA
tatggctggtcctcagggagacgttgagaatcgaaagattcttaataaggcatccttccgatgctgacttctcaccgtccgttttccaataggagcgggcggtatgtttt

SEQ ID NO: 336 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 D6
  source 1..110: mol_type = other RNA
ctaaggtgtcaggatctgaagggttgagaatcgaaagattcttaataaggcatccttccgatgctgacttctcaccgtccgttttccaataggagcgggcggtatgtttt

SEQ ID NO: 337 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting TRBC2
  misc_feature 1..110: note = Description: MG3-6/3-4 TRBC2 E6
  source 1..110: mol_type = other RNA
ggaacacgtttttcaggtcctcgttgagaatcgaaagattcttaataaggcatccttccgatgctgacttctcaccgtccgttttccaataggagcgggcggtatgtttt

SEQ ID NO: 338 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 A1
  source 1..22: mol_type = other DNA
acctcttccctttccagaggac

SEQ ID NO: 339 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 B1
  source 1..22: mol_type = other DNA
cctcttccctttccagaggacc

SEQ ID NO: 340 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 C1
  source 1..22: mol_type = other DNA
ctcttccctttccagaggacct

SEQ ID NO: 341 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 D1
  source 1..22: mol_type = other DNA
cagaagcagagatctcccacac

SEQ ID NO: 342 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 E1
  source 1..22: mol_type = other DNA
ccacgtggagctgagctggtgg

SEQ ID NO: 343 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 F1
  source 1..22: mol_type = other DNA
agtccagttctacgggctctcg

SEQ ID NO: 344 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 G1
  source 1..22: mol_type = other DNA
gattaggtgagaccagctacca

SEQ ID NO: 345 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 H1
  source 1..22: mol_type = other DNA
attaggtgagaccagctaccag

SEQ ID NO: 346 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 A2
  source 1..22: mol_type = other DNA
ttaggtgagaccagctaccagg

SEQ ID NO: 347 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 B2
  source 1..22: mol_type = other DNA
tgagaccagctaccagggaaaa

SEQ ID NO: 348 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 C2
  source 1..22: mol_type = other DNA
tagcggacaagactagatccag

SEQ ID NO: 349 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 D2
  source 1..22: mol_type = other DNA
cccccaccaagaagcatagagg

SEQ ID NO: 350 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 E2
  source 1..22: mol_type = other DNA
tctgctctcgaaccagggcatg

SEQ ID NO: 351 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 F2
  source 1..22: mol_type = other DNA
ggaacatcacacatgggcataa

SEQ ID NO: 352 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 G2
  source 1..22: mol_type = other DNA
cctaatatatcctatcacctca

SEQ ID NO: 353 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 H2
  source 1..22: mol_type = other DNA
accataatgaagccagactggg

SEQ ID NO: 354 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 A3
  source 1..22: mol_type = other DNA
ccataatgaagccagactgggg

SEQ ID NO: 355 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 B3
  source 1..22: mol_type = other DNA
cataatgaagccagactgggga

SEQ ID NO: 356 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 C3
  source 1..22: mol_type = other DNA
gccagactggggagaaaatgca

SEQ ID NO: 357 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 D3
  source 1..22: mol_type = other DNA
ggagaaaatgcagggaatatca

SEQ ID NO: 358 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 E3
  source 1..22: mol_type = other DNA
ggagacaaccagcgagccctac

SEQ ID NO: 359 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 F3
  source 1..22: mol_type = other DNA
tactcctgctgtgccatagccc

SEQ ID NO: 360 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 G3
  source 1..22: mol_type = other DNA
ctgtgccatagcccctgaaacc

SEQ ID NO: 361 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 H3
  source 1..22: mol_type = other DNA
tgtgccatagcccctgaaaccc

SEQ ID NO: 362 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 A4
  source 1..22: mol_type = other DNA
gtgccatagcccctgaaaccct

SEQ ID NO: 363 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 B4
  source 1..22: mol_type = other DNA
tgttctctcttccacaggtcaa

SEQ ID NO: 364 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 C4
  source 1..22: mol_type = other DNA
gaaaggattccagaggctagct

SEQ ID NO: 365 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 D4
  source 1..22: mol_type = other DNA
ggatggttttggagctagcctc

SEQ ID NO: 366 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 E4
  source 1..22: mol_type = other DNA
ccctggttcgagagcagagacg

SEQ ID NO: 367 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 F4
  source 1..22: mol_type = other DNA
agcagagacggcgaaagataga

SEQ ID NO: 368 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 G4
  source 1..22: mol_type = other DNA
gcagagacggcgaaagatagag

SEQ ID NO: 369 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 H4
  source 1..22: mol_type = other DNA
cagagacggcgaaagatagaga

SEQ ID NO: 370 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 A5
  source 1..22: mol_type = other DNA
ttaccggaggtgaagccacagt

SEQ ID NO: 371 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 B5
  source 1..22: mol_type = other DNA
cggaggtgaagccacagtctga

SEQ ID NO: 372 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 C5
  source 1..22: mol_type = other DNA
ggaggtgaagccacagtctgaa

SEQ ID NO: 373 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 D5
  source 1..22: mol_type = other DNA
acagtctgaaagaaaacagggg

SEQ ID NO: 374 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 E5
  source 1..22: mol_type = other DNA
cagtctgaaagaaaacagggga

SEQ ID NO: 375 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 F5
  source 1..22: mol_type = other DNA
agtctgaaagaaaacaggggaa

SEQ ID NO: 376 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 G5
  source 1..22: mol_type = other DNA
gtctgaaagaaaacaggggaag

SEQ ID NO: 377 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 H5
  source 1..22: mol_type = other DNA
acaggggaagaaaaatggatga

SEQ ID NO: 378 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 A6
  source 1..22: mol_type = other DNA
gcgaagtggtcactatgatctt

SEQ ID NO: 379 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 B6
  source 1..22: mol_type = other DNA
ttaggaaaccaggaccccagaa

SEQ ID NO: 380 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 C6
  source 1..22: mol_type = other DNA
tatggctggtcctcagggagac

SEQ ID NO: 381 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 D6
  source 1..22: mol_type = other DNA
ctaaggtgtcaggatctgaagg

SEQ ID NO: 382 - DNA, 22 bp, synthetic construct
  misc_feature 1..22: note = Description of Artificial Sequence: Synthetic oligonucleotide
  misc_feature 1..22: note = Category: DNA sequence of TRBC2 target site
  misc_feature 1..22: note = Description: MG3-6/3-4 TRBC2 E6
  source 1..22: mol_type = other DNA
ggaacacgtttttcaggtcctc

SEQ ID NO: 383 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A1
  source 1..110: mol_type = other RNA
ttgttcctctagttatttcctcgttgagaatcgaaagattcttaataaggcatccttccgatgctgacttctcaccgtccgttttccaataggagcgggcggtatgtttt

SEQ ID NO: 384 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B1
  source 1..110: mol_type = other RNA
atttgattctctatctccagaggttgagaatcgaaagattcttaataaggcatccttccgatgctgacttctcaccgtccgttttccaataggagcgggcggtatgtttt

SEQ ID NO: 385 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C1
  source 1..110: mol_type = other RNA
tttgattctctatctccagagcgttgagaatcgaaagattcttaataaggcatccttccgatgctgacttctcaccgtccgttttccaataggagcgggcggtatgtttt

SEQ ID NO: 386 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D1
  source 1..110: mol_type = other RNA
aagatttgctatgttagacgatgttgagaatcgaaagattcttaataaggcatccttccgatgctgacttctcaccgtccgttttccaataggagcgggcggtatgtttt

SEQ ID NO: 387 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E1
  source 1..110: mol_type = other RNA
agatttgctatgttagacgatggttgagaatcgaaagattcttaataaggcatccttccgatgctgacttctcaccgtccgttttccaataggagcgggcggtatgtttt

SEQ ID NO: 388 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F1
  source 1..110: mol_type = other RNA
gatttgctatgttagacgatgtgttgagaatcgaaagattcttaataaggcatccttccgatgctgacttctcaccgtccgttttccaataggagcgggcggtatgtttt

SEQ ID NO: 389 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G1
  source 1..110: mol_type = other RNA
actttgtccataagacgaaggggttgagaatcgaaagattcttaataaggcatccttccgatgctgacttctcaccgtccgttttccaataggagcgggcggtatgtttt

SEQ ID NO: 390 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H1
  source 1..110: mol_type = other RNA
agggccaaattaatgacatattgttgagaatcgaaagattcttaataaggcatccttccgatgctgacttctcaccgtccgttttccaataggagcgggcggtatgtttt

SEQ ID NO: 391 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A2
  source 1..110: mol_type = other RNA
gggccaaattaatgacatatttgttgagaatcgaaagattcttaataaggcatccttccgatgctgacttctcaccgtccgttttccaataggagcgggcggtatgtttt

SEQ ID NO: 392 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B2
  source 1..110: mol_type = other RNA
tatgatctatcgctgcaaaccagttgagaatcgaaagattcttaataaggcatccttccgatgctgacttctcaccgtccgttttccaataggagcgggcggtatgtttt

SEQ ID NO: 393 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C2
  source 1..110: mol_type = other RNA
atgatctatcgctgcaaaccaggttgagaatcgaaagattcttaataaggcatccttccgatgctgacttctcaccgtccgttttccaataggagcgggcggtatgtttt

SEQ ID NO: 394 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D2
  source 1..110: mol_type = other RNA
caaaccagtgaaatcaaagaaggttgagaatcgaaagattcttaataaggcatccttccgatgctgacttctcaccgtccgttttccaataggagcgggcggtatgtttt

SEQ ID NO: 395 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E2
  source 1..110: mol_type = other RNA
aaaccagtgaaatcaaagaagagttgagaatcgaaagattcttaataaggcatccttccgatgctgacttctcaccgtccgttttccaataggagcgggcggtatgtttt

SEQ ID NO: 396 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F2
  source 1..110: mol_type = other RNA
acaagtcaaaaatgaagaggtagttgagaatcgaaagattcttaataaggcatccttccgatgctgacttctcaccgtccgttttccaataggagcgggcggtatgtttt

SEQ ID NO: 397 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G2
  source 1..110: mol_type = other RNA
gaatatgtcacttgaactcaacgttgagaatcgaaagattcttaataaggcatccttccgatgctgacttctcaccgtccgttttccaataggagcgggcggtatgtttt

SEQ ID NO: 398 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H2
  source 1..110: mol_type = other RNA
tcacttgaactcaactcaaaacgttgagaatcgaaagattcttaataaggcatccttccgatgctgacttctcaccgtccgttttccaataggagcgggcggtatgtttt

SEQ ID NO: 399 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A3
  source 1..110: mol_type = other RNA
tcaaaacttgaaagcctcctaggttgagaatcgaaagattcttaataaggcatccttccgatgctgacttctcaccgtccgttttccaataggagcgggcggtatgtttt

SEQ ID NO: 400 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B3
  source 1..110: mol_type = other RNA
caaaacttgaaagcctcctagagttgagaatcgaaagattcttaataaggcatccttccgatgctgacttctcaccgtccgttttccaataggagcgggcggtatgtttt

SEQ ID NO: 401 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C3
  source 1..110: mol_type = other RNA
aaaacttgaaagcctcctagaagttgagaatcgaaagattcttaataaggcatccttccgatgctgacttctcaccgtccgttttccaataggagcgggcggtatgtttt

SEQ ID NO: 402 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D3
  source 1..110: mol_type = other RNA
aaacttgaaagcctcctagaaggttgagaatcgaaagattcttaataaggcatccttccgatgctgacttctcaccgtccgttttccaataggagcgggcggtatgtttt

SEQ ID NO: 403 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E3
  source 1..110: mol_type = other RNA
aacttgaaagcctcctagaagagttgagaatcgaaagattcttaataaggcatccttccgatgctgacttctcaccgtccgttttccaataggagcgggcggtatgtttt

SEQ ID NO: 404 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F3
  source 1..110: mol_type = other RNA
gttctggagtttcaggttgattgttgagaatcgaaagattcttaataaggcatccttccgatgctgacttctcaccgtccgttttccaataggagcgggcggtatgtttt

SEQ ID NO: 405 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G3
  source 1..110: mol_type = other RNA
cactggtttgcagcgatagatcgttgagaatcgaaagattcttaataaggcatccttccgatgctgacttctcaccgtccgttttccaataggagcgggcggtatgtttt

SEQ ID NO: 406 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H3
  source 1..110: mol_type = other RNA
actggtttgcagcgatagatcagttgagaatcgaaagattcttaataaggcatccttccgatgctgacttctcaccgtccgttttccaataggagcgggcggtatgtttt

SEQ ID NO: 407 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A4
  source 1..110: mol_type = other RNA
cgatagatcataaaaagactgagttgagaatcgaaagattcttaataaggcatccttccgatgctgacttctcaccgtccgttttccaataggagcgggcggtatgtttt

SEQ ID NO: 408 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B4
  source 1..110: mol_type = other RNA
cccaactgaaggaggccattgggttgagaatcgaaagattcttaataaggcatccttccgatgctgacttctcaccgtccgttttccaataggagcgggcggtatgtttt

SEQ ID NO: 409 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C4
  source 1..110: mol_type = other RNA
ccaactgaaggaggccattggcgttgagaatcgaaagattcttaataaggcatccttccgatgctgacttctcaccgtccgttttccaataggagcgggcggtatgtttt

SEQ ID NO: 410 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D4
  source 1..110: mol_type = other RNA
cttgattttggctctggagatagttgagaatcgaaagattcttaataaggcatccttccgatgctgacttctcaccgtccgttttccaataggagcgggcggtatgtttt

SEQ ID NO: 411 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E4
  source 1..110: mol_type = other RNA
ttttggctctggagatagagaagttgagaatcgaaagattcttaataaggcatccttccgatgctgacttctcaccgtccgttttccaataggagcgggcggtatgtttt

SEQ ID NO: 412 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F4
  source 1..110: mol_type = other RNA
tctggagatagagaatcaaatggttgagaatcgaaagattcttaataaggcatccttccgatgctgacttctcaccgtccgttttccaataggagcgggcggtatgtttt

SEQ ID NO: 413 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G4
  source 1..110: mol_type = other RNA
gaattgtcttgatcaattctgggttgagaatcgaaagattcttaataaggcatccttccgatgctgacttctcaccgtccgttttccaataggagcgggcggtatgtttt

SEQ ID NO: 414 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H4
  source 1..110: mol_type = other RNA
aattgtcttgatcaattctggagttgagaatcgaaagattcttaataaggcatccttccgatgctgacttctcaccgtccgttttccaataggagcgggcggtatgtttt

SEQ ID NO: 415 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A5
  source 1..110: mol_type = other RNA
ggaggaaataactagaggaacagttgagaatcgaaagattcttaataaggcatccttccgatgctgacttctcaccgtccgttttccaataggagcgggcggtatgtttt

SEQ ID NO: 416 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B5
  source 1..110: mol_type = other RNA
gaggaaataactagaggaacaagttgagaatcgaaagattcttaataaggcatccttccgatgctgacttctcaccgtccgttttccaataggagcgggcggtatgtttt

SEQ ID NO: 417 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C5
  source 1..110: mol_type = other RNA
actctctatatccagacttttggttgagaatcgaaagattcttaataaggcatccttccgatgctgacttctcaccgtccgttttccaataggagcgggcggtatgtttt

SEQ ID NO: 418 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D5
  source 1..110: mol_type = other RNA
ctctctatatccagacttttgtgttgagaatcgaaagattcttaataaggcatccttccgatgctgacttctcaccgtccgttttccaataggagcgggcggtatgtttt

SEQ ID NO: 419 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E5
  source 1..110: mol_type = other RNA
tctctatatccagacttttgtagttgagaatcgaaagattcttaataaggcatccttccgatgctgacttctcaccgtccgttttccaataggagcgggcggtatgtttt

SEQ ID NO: 420 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F5
  source 1..110: mol_type = other RNA
aacaattaaaccaacagcataggttgagaatcgaaagattcttaataaggcatccttccgatgctgacttctcaccgtccgttttccaataggagcgggcggtatgtttt

SEQ ID NO: 421 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G5
  source 1..110: mol_type = other RNA
attaaaccaacagcatagtcaagttgagaatcgaaagattcttaataaggcatccttccgatgctgacttctcaccgtccgttttccaataggagcgggcggtatgtttt

SEQ ID NO: 422 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H5
  source 1..110: mol_type = other RNA
aaccaacagcatagtcaaataagttgagaatcgaaagattcttaataaggcatccttccgatgctgacttctcaccgtccgttttccaataggagcgggcggtatgtttt

SEQ ID NO: 423 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A6
  source 1..110: mol_type = other RNA
accaacagcatagtcaaataaagttgagaatcgaaagattcttaataaggcatccttccgatgctgacttctcaccgtccgttttccaataggagcgggcggtatgtttt

SEQ ID NO: 424 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B6
  source 1..110: mol_type = other RNA
gatgctattatcttgtttttctgttgagaatcgaaagattcttaataaggcatccttccgatgctgacttctcaccgtccgttttccaataggagcgggcggtatgtttt

SEQ ID NO: 425 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C6
  source 1..110: mol_type = other RNA
aggactagtattcaagaacccagttgagaatcgaaagattcttaataaggcatccttccgatgctgacttctcaccgtccgttttccaataggagcgggcggtatgtttt

SEQ ID NO: 426 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D6
  source 1..110: mol_type = other RNA
ggactagtattcaagaacccacgttgagaatcgaaagattcttaataaggcatccttccgatgctgacttctcaccgtccgttttccaataggagcgggcggtatgtttt

SEQ ID NO: 427 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E6
  source 1..110: mol_type = other RNA
aagaactactccctttcttcaggttgagaatcgaaagattcttaataaggcatccttccgatgctgacttctcaccgtccgttttccaataggagcgggcggtatgtttt

SEQ ID NO: 428 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F6
  source 1..110: mol_type = other RNA
actactccctttcttcagttgagttgagaatcgaaagattcttaataaggcatccttccgatgctgacttctcaccgtccgttttccaataggagcgggcggtatgtttt

SEQ ID NO: 429 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G6
  source 1..110: mol_type = other RNA
ctactccctttcttcagttgaagttgagaatcgaaagattcttaataaggcatccttccgatgctgacttctcaccgtccgttttccaataggagcgggcggtatgtttt

SEQ ID NO: 430 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H6
  source 1..110: mol_type = other RNA
cctttcttcagttgaatgaaatgttgagaatcgaaagattcttaataaggcatccttccgatgctgacttctcaccgtccgttttccaataggagcgggcggtatgtttt

SEQ ID NO: 431 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A7
  source 1..110: mol_type = other RNA
ggtgctcttggcttggaagatagttgagaatcgaaagattcttaataaggcatccttccgatgctgacttctcaccgtccgttttccaataggagcgggcggtatgtttt

SEQ ID NO: 432 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B7
  source 1..110: mol_type = other RNA
gtgctcttggcttggaagataggttgagaatcgaaagattcttaataaggcatccttccgatgctgacttctcaccgtccgttttccaataggagcgggcggtatgtttt

SEQ ID NO: 433 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C7
  source 1..110: mol_type = other RNA
atagagaaatttctgtgggttcgttgagaatcgaaagattcttaataaggcatccttccgatgctgacttctcaccgtccgttttccaataggagcgggcggtatgtttt

SEQ ID NO: 434 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D7
  source 1..110: mol_type = other RNA
gaatactagtccttctgagctggttgagaatcgaaagattcttaataaggcatccttccgatgctgacttctcaccgtccgttttccaataggagcgggcggtatgtttt

SEQ ID NO: 435 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E7
  source 1..110: mol_type = other RNA
ttattgattctaggcattcctggttgagaatcgaaagattcttaataaggcatccttccgatgctgacttctcaccgtccgttttccaataggagcgggcggtatgtttt

SEQ ID NO: 436 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F7
  source 1..110: mol_type = other RNA
gtctactgtgatgttatatcaggttgagaatcgaaagattcttaataaggcatccttccgatgctgacttctcaccgtccgttttccaataggagcgggcggtatgtttt

SEQ ID NO: 437 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G7
  source 1..110: mol_type = other RNA
ctgatataacatcacagtagacgttgagaatcgaaagattcttaataaggcatccttccgatgctgacttctcaccgtccgttttccaataggagcgggcggtatgtttt

SEQ ID NO: 438 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H7
  source 1..110: mol_type = other RNA
tgatataacatcacagtagacagttgagaatcgaaagattcttaataaggcatccttccgatgctgacttctcaccgtccgttttccaataggagcgggcggtatgtttt

SEQ ID NO: 439 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A8
  source 1..110: mol_type = other RNA
gatataacatcacagtagacatgttgagaatcgaaagattcttaataaggcatccttccgatgctgacttctcaccgtccgttttccaataggagcgggcggtatgtttt

SEQ ID NO: 440 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B8
  source 1..110: mol_type = other RNA
cacttgtatgttcacctctgttgttgagaatcgaaagattcttaataaggcatccttccgatgctgacttctcaccgtccgttttccaataggagcgggcggtatgtttt

SEQ ID NO: 441 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C8
  source 1..110: mol_type = other RNA
tataaatggtggtacattcagcgttgagaatcgaaagattcttaataaggcatccttccgatgctgacttctcaccgtccgttttccaataggagcgggcggtatgtttt

SEQ ID NO: 442 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D8
  source 1..110: mol_type = other RNA
tggtacattcagcaggaatgccgttgagaatcgaaagattcttaataaggcatccttccgatgctgacttctcaccgtccgttttccaataggagcgggcggtatgtttt

SEQ ID NO: 443 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E8
  source 1..110: mol_type = other RNA
gtccatggacattaattcaacagttgagaatcgaaagattcttaataaggcatccttccgatgctgacttctcaccgtccgttttccaataggagcgggcggtatgtttt

SEQ ID NO: 444 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F8
  source 1..110: mol_type = other RNA
ttcaacatcgaatagatggatcgttgagaatcgaaagattcttaataaggcatccttccgatgctgacttctcaccgtccgttttccaataggagcgggcggtatgtttt

SEQ ID NO: 445 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G8
  source 1..110: mol_type = other RNA
atagatggatcacaaaacttcagttgagaatcgaaagattcttaataaggcatccttccgatgctgacttctcaccgtccgttttccaataggagcgggcggtatgtttt

SEQ ID NO: 446 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H8
  source 1..110: mol_type = other RNA
ttcaatgaaacgtgggagaactgttgagaatcgaaagattcttaataaggcatccttccgatgctgacttctcaccgtccgttttccaataggagcgggcggtatgtttt

SEQ ID NO: 447 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A9
  source 1..110: mol_type = other RNA
agtccccttaccatcaagcctcgttgagaatcgaaagattcttaataaggcatccttccgatgctgacttctcaccgtccgttttccaataggagcgggcggtatgtttt

SEQ ID NO: 448 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B9
  source 1..110: mol_type = other RNA
tttgtgatccatctattcgatggttgagaatcgaaagattcttaataaggcatccttccgatgctgacttctcaccgtccgttttccaataggagcgggcggtatgtttt

SEQ ID NO: 449 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C9
  source 1..110: mol_type = other RNA
tgaattaatgtccatggactacgttgagaatcgaaagattcttaataaggcatccttccgatgctgacttctcaccgtccgttttccaataggagcgggcggtatgtttt

SEQ ID NO: 450 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D9
  source 1..110: mol_type = other RNA
tttacgaattgagttggaagacgttgagaatcgaaagattcttaataaggcatccttccgatgctgacttctcaccgtccgttttccaataggagcgggcggtatgtttt

SEQ ID NO: 451 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E9
  source 1..110: mol_type = other RNA
ggcaatgtccccaatgcaatccgttgagaatcgaaagattcttaataaggcatccttccgatgctgacttctcaccgtccgttttccaataggagcgggcggtatgtttt

SEQ ID NO: 452 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F9
  source 1..110: mol_type = other RNA
gcaatgtccccaatgcaatcccgttgagaatcgaaagattcttaataaggcatccttccgatgctgacttctcaccgtccgttttccaataggagcgggcggtatgtttt

SEQ ID NO: 453 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G9
  source 1..110: mol_type = other RNA
gttttctacttgggatcacaaagttgagaatcgaaagattcttaataaggcatccttccgatgctgacttctcaccgtccgttttccaataggagcgggcggtatgtttt

SEQ ID NO: 454 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H9
  source 1..110: mol_type = other RNA
ccttttgctttgtgatcccaaggttgagaatcgaaagattcttaataaggcatccttccgatgctgacttctcaccgtccgttttccaataggagcgggcggtatgtttt

SEQ ID NO: 455 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A10
  source 1..110: mol_type = other RNA
cttttgctttgtgatcccaagtgttgagaatcgaaagattcttaataaggcatccttccgatgctgacttctcaccgtccgttttccaataggagcgggcggtatgtttt

SEQ ID NO: 456 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B10
  source 1..110: mol_type = other RNA
ttgtgatcccaagtagaaaacagttgagaatcgaaagattcttaataaggcatccttccgatgctgacttctcaccgtccgttttccaataggagcgggcggtatgtttt

SEQ ID NO: 457 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C10
  source 1..110: mol_type = other RNA
agttggtttcgtgatttcccaagttgagaatcgaaagattcttaataaggcatccttccgatgctgacttctcaccgtccgttttccaataggagcgggcggtatgtttt

SEQ ID NO: 458 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D10
  source 1..110: mol_type = other RNA
gttggtttcgtgatttcccaaggttgagaatcgaaagattcttaataaggcatccttccgatgctgacttctcaccgtccgttttccaataggagcgggcggtatgtttt

SEQ ID NO: 459 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E10
  source 1..110: mol_type = other RNA
gtttcgtgatttcccaagtaaagttgagaatcgaaagattcttaataaggcatccttccgatgctgacttctcaccgtccgttttccaataggagcgggcggtatgtttt

SEQ ID NO: 460 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F10
  source 1..110: mol_type = other RNA
ttccagtcttccaactcaattcgttgagaatcgaaagattcttaataaggcatccttccgatgctgacttctcaccgtccgttttccaataggagcgggcggtatgtttt

SEQ ID NO: 461 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G10
  source 1..110: mol_type = other RNA
agtatatcttctctaggcccaagttgagaatcgaaagattcttaataaggcatccttccgatgctgacttctcaccgtccgttttccaataggagcgggcggtatgtttt

SEQ ID NO: 462 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H10
  source 1..110: mol_type = other RNA
gtatatcttctctaggcccaacgttgagaatcgaaagattcttaataaggcatccttccgatgctgacttctcaccgtccgttttccaataggagcgggcggtatgtttt

SEQ ID NO: 463 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A11
  source 1..110: mol_type = other RNA
tctaggcccaaccaaaattctcgttgagaatcgaaagattcttaataaggcatccttccgatgctgacttctcaccgtccgttttccaataggagcgggcggtatgtttt

SEQ ID NO: 464 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B11
  source 1..110: mol_type = other RNA
ctaggcccaaccaaaattctccgttgagaatcgaaagattcttaataaggcatccttccgatgctgacttctcaccgtccgttttccaataggagcgggcggtatgtttt

SEQ ID NO: 465 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C11
  source 1..110: mol_type = other RNA
gcccaaccaaaattctcctgaagttgagaatcgaaagattcttaataaggcatccttccgatgctgacttctcaccgtccgttttccaataggagcgggcggtatgtttt

SEQ ID NO: 466 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D11
  source 1..110: mol_type = other RNA
tggtggtggcatgatgagtgtggttgagaatcgaaagattcttaataaggcatccttccgatgctgacttctcaccgtccgttttccaataggagcgggcggtatgtttt

SEQ ID NO: 467 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E11
  source 1..110: mol_type = other RNA
ggtggtggcatgatgagtgtgggttgagaatcgaaagattcttaataaggcatccttccgatgctgacttctcaccgtccgttttccaataggagcgggcggtatgtttt

SEQ ID NO: 468 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F11
  source 1..110: mol_type = other RNA
tgatgagtgtggagaaaacaacgttgagaatcgaaagattcttaataaggcatccttccgatgctgacttctcaccgtccgttttccaataggagcgggcggtatgtttt

SEQ ID NO: 469 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G11
  source 1..110: mol_type = other RNA
tgtggagaaaacaacctaaatggttgagaatcgaaagattcttaataaggcatccttccgatgctgacttctcaccgtccgttttccaataggagcgggcggtatgtttt

SEQ ID NO: 470 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 H11
  source 1..110: mol_type = other RNA
ggtaaatataacaaaccaagaggttgagaatcgaaagattcttaataaggcatccttccgatgctgacttctcaccgtccgttttccaataggagcgggcggtatgtttt

SEQ ID NO: 471 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 A12
  source 1..110: mol_type = other RNA
gaagaggattatcttggaagtcgttgagaatcgaaagattcttaataaggcatccttccgatgctgacttctcaccgtccgttttccaataggagcgggcggtatgtttt

SEQ ID NO: 472 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 B12
  source 1..110: mol_type = other RNA
aagaggattatcttggaagtctgttgagaatcgaaagattcttaataaggcatccttccgatgctgacttctcaccgtccgttttccaataggagcgggcggtatgtttt

SEQ ID NO: 473 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 C12
  source 1..110: mol_type = other RNA
tcaaaatggaaggttatactctgttgagaatcgaaagattcttaataaggcatccttccgatgctgacttctcaccgtccgttttccaataggagcgggcggtatgtttt

SEQ ID NO: 474 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 D12
  source 1..110: mol_type = other RNA
caaaatggaaggttatactctagttgagaatcgaaagattcttaataaggcatccttccgatgctgacttctcaccgtccgttttccaataggagcgggcggtatgtttt

SEQ ID NO: 475 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 E12
  source 1..110: mol_type = other RNA
atgttgatccatccaacagattgttgagaatcgaaagattcttaataaggcatccttccgatgctgacttctcaccgtccgttttccaataggagcgggcggtatgtttt

SEQ ID NO: 476 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 F12
  source 1..110: mol_type = other RNA
catccaacagattcagaaagctgttgagaatcgaaagattcttaataaggcatccttccgatgctgacttctcaccgtccgttttccaataggagcgggcggtatgtttt

SEQ ID NO: 477 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting ANGPTL3
  misc_feature 1..110: note = Description: MG3-6/3-4 ANGPTL3 G12
  source 1..110: mol_type = other RNA
gcctcagttcattcaaagctttgttgagaatcgaaagattcttaataaggcatccttccgatgctgacttctcaccgtccgttttccaataggagcgggcggtatgtttt

SEQ ID NO: 478 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A1
  source 1..22: mol_type = other DNA
ttgttcctctagttatttcctc

SEQ ID NO: 479 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B1
  source 1..22: mol_type = other DNA
atttgattctctatctccagag

SEQ ID NO: 480 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C1
  source 1..22: mol_type = other DNA
tttgattctctatctccagagc

SEQ ID NO: 481 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D1
  source 1..22: mol_type = other DNA
aagatttgctatgttagacgat

SEQ ID NO: 482 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E1
  source 1..22: mol_type = other DNA
agatttgctatgttagacgatg

SEQ ID NO: 483 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F1
  source 1..22: mol_type = other DNA
gatttgctatgttagacgatgt

SEQ ID NO: 484 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G1
  source 1..22: mol_type = other DNA
actttgtccataagacgaaggg

SEQ ID NO: 485 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H1
  source 1..22: mol_type = other DNA
agggccaaattaatgacatatt

SEQ ID NO: 486 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A2
  source 1..22: mol_type = other DNA
gggccaaattaatgacatattt

SEQ ID NO: 487 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B2
  source 1..22: mol_type = other DNA
tatgatctatcgctgcaaacca

SEQ ID NO: 488 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C2
  source 1..22: mol_type = other DNA
atgatctatcgctgcaaaccag

SEQ ID NO: 489 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D2
  source 1..22: mol_type = other DNA
caaaccagtgaaatcaaagaag

SEQ ID NO: 490 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E2
  source 1..22: mol_type = other DNA
aaaccagtgaaatcaaagaaga

SEQ ID NO: 491 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F2
  source 1..22: mol_type = other DNA
acaagtcaaaaatgaagaggta

SEQ ID NO: 492 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G2
  source 1..22: mol_type = other DNA
gaatatgtcacttgaactcaac

SEQ ID NO: 493 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H2
  source 1..22: mol_type = other DNA
tcacttgaactcaactcaaaac

SEQ ID NO: 494 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A3
  source 1..22: mol_type = other DNA
tcaaaacttgaaagcctcctag

SEQ ID NO: 495 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B3
  source 1..22: mol_type = other DNA
caaaacttgaaagcctcctaga

SEQ ID NO: 496 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C3
  source 1..22: mol_type = other DNA
aaaacttgaaagcctcctagaa

SEQ ID NO: 497 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D3
  source 1..22: mol_type = other DNA
aaacttgaaagcctcctagaag

SEQ ID NO: 498 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E3
  source 1..22: mol_type = other DNA
aacttgaaagcctcctagaaga

SEQ ID NO: 499 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F3
  source 1..22: mol_type = other DNA
gttctggagtttcaggttgatt

SEQ ID NO: 500 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G3
  source 1..22: mol_type = other DNA
cactggtttgcagcgatagatc

SEQ ID NO: 501 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H3
  source 1..22: mol_type = other DNA
actggtttgcagcgatagatca

SEQ ID NO: 502 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A4
  source 1..22: mol_type = other DNA
cgatagatcataaaaagactga

SEQ ID NO: 503 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B4
  source 1..22: mol_type = other DNA
cccaactgaaggaggccattgg

SEQ ID NO: 504 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C4
  source 1..22: mol_type = other DNA
ccaactgaaggaggccattggc

SEQ ID NO: 505 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D4
  source 1..22: mol_type = other DNA
cttgattttggctctggagata

SEQ ID NO: 506 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E4
  source 1..22: mol_type = other DNA
ttttggctctggagatagagaa

SEQ ID NO: 507 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F4
  source 1..22: mol_type = other DNA
tctggagatagagaatcaaatg

SEQ ID NO: 508 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G4
  source 1..22: mol_type = other DNA
gaattgtcttgatcaattctgg

SEQ ID NO: 509 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H4
  source 1..22: mol_type = other DNA
aattgtcttgatcaattctgga

SEQ ID NO: 510 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A5
  source 1..22: mol_type = other DNA
ggaggaaataactagaggaaca

SEQ ID NO: 511 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B5
  source 1..22: mol_type = other DNA
gaggaaataactagaggaacaa

SEQ ID NO: 512 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C5
  source 1..22: mol_type = other DNA
actctctatatccagacttttg

SEQ ID NO: 513 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D5
  source 1..22: mol_type = other DNA
ctctctatatccagacttttgt

SEQ ID NO: 514 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E5
  source 1..22: mol_type = other DNA
tctctatatccagacttttgta

SEQ ID NO: 515 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F5
  source 1..22: mol_type = other DNA
aacaattaaaccaacagcatag

SEQ ID NO: 516 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G5
  source 1..22: mol_type = other DNA
attaaaccaacagcatagtcaa

SEQ ID NO: 517 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H5
  source 1..22: mol_type = other DNA
aaccaacagcatagtcaaataa

SEQ ID NO: 518 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A6
  source 1..22: mol_type = other DNA
accaacagcatagtcaaataaa

SEQ ID NO: 519 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B6
  source 1..22: mol_type = other DNA
gatgctattatcttgtttttct

SEQ ID NO: 520 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C6
  source 1..22: mol_type = other DNA
aggactagtattcaagaaccca

SEQ ID NO: 521 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D6
  source 1..22: mol_type = other DNA
ggactagtattcaagaacccac

SEQ ID NO: 522 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E6
  source 1..22: mol_type = other DNA
aagaactactccctttcttcag

SEQ ID NO: 523 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F6
  source 1..22: mol_type = other DNA
actactccctttcttcagttga

SEQ ID NO: 524 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G6
  source 1..22: mol_type = other DNA
ctactccctttcttcagttgaa

SEQ ID NO: 525 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H6
  source 1..22: mol_type = other DNA
cctttcttcagttgaatgaaat

SEQ ID NO: 526 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A7
  source 1..22: mol_type = other DNA
ggtgctcttggcttggaagata

SEQ ID NO: 527 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B7
  source 1..22: mol_type = other DNA
gtgctcttggcttggaagatag

SEQ ID NO: 528 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C7
  source 1..22: mol_type = other DNA
atagagaaatttctgtgggttc

SEQ ID NO: 529 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D7
  source 1..22: mol_type = other DNA
gaatactagtccttctgagctg

SEQ ID NO: 530 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E7
  source 1..22: mol_type = other DNA
ttattgattctaggcattcctg

SEQ ID NO: 531 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F7
  source 1..22: mol_type = other DNA
gtctactgtgatgttatatcag

SEQ ID NO: 532 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G7
  source 1..22: mol_type = other DNA
ctgatataacatcacagtagac

SEQ ID NO: 533 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H7
  source 1..22: mol_type = other DNA
tgatataacatcacagtagaca

SEQ ID NO: 534 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A8
  source 1..22: mol_type = other DNA
gatataacatcacagtagacat

SEQ ID NO: 535 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B8
  source 1..22: mol_type = other DNA
cacttgtatgttcacctctgtt

SEQ ID NO: 536 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C8
  source 1..22: mol_type = other DNA
tataaatggtggtacattcagc

SEQ ID NO: 537 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D8
  source 1..22: mol_type = other DNA
tggtacattcagcaggaatgcc

SEQ ID NO: 538 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E8
  source 1..22: mol_type = other DNA
gtccatggacattaattcaaca

SEQ ID NO: 539 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F8
  source 1..22: mol_type = other DNA
ttcaacatcgaatagatggatc

SEQ ID NO: 540 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G8
  source 1..22: mol_type = other DNA
atagatggatcacaaaacttca

SEQ ID NO: 541 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H8
  source 1..22: mol_type = other DNA
ttcaatgaaacgtgggagaact

SEQ ID NO: 542 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A9
  source 1..22: mol_type = other DNA
agtccccttaccatcaagcctc

SEQ ID NO: 543 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B9
  source 1..22: mol_type = other DNA
tttgtgatccatctattcgatg

SEQ ID NO: 544 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C9
  source 1..22: mol_type = other DNA
tgaattaatgtccatggactac

SEQ ID NO: 545 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D9
  source 1..22: mol_type = other DNA
tttacgaattgagttggaagac

SEQ ID NO: 546 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E9
  source 1..22: mol_type = other DNA
ggcaatgtccccaatgcaatcc

SEQ ID NO: 547 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F9
  source 1..22: mol_type = other DNA
gcaatgtccccaatgcaatccc

SEQ ID NO: 548 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G9
  source 1..22: mol_type = other DNA
gttttctacttgggatcacaaa

SEQ ID NO: 549 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H9
  source 1..22: mol_type = other DNA
ccttttgctttgtgatcccaag

SEQ ID NO: 550 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A10
  source 1..22: mol_type = other DNA
cttttgctttgtgatcccaagt

SEQ ID NO: 551 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B10
  source 1..22: mol_type = other DNA
ttgtgatcccaagtagaaaaca

SEQ ID NO: 552 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C10
  source 1..22: mol_type = other DNA
agttggtttcgtgatttcccaa

SEQ ID NO: 553 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D10
  source 1..22: mol_type = other DNA
gttggtttcgtgatttcccaag

SEQ ID NO: 554 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E10
  source 1..22: mol_type = other DNA
gtttcgtgatttcccaagtaaa

SEQ ID NO: 555 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F10
  source 1..22: mol_type = other DNA
ttccagtcttccaactcaattc

SEQ ID NO: 556 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G10
  source 1..22: mol_type = other DNA
agtatatcttctctaggcccaa

SEQ ID NO: 557 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H10
  source 1..22: mol_type = other DNA
gtatatcttctctaggcccaac

SEQ ID NO: 558 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A11
  source 1..22: mol_type = other DNA
tctaggcccaaccaaaattctc

SEQ ID NO: 559 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B11
  source 1..22: mol_type = other DNA
ctaggcccaaccaaaattctcc

SEQ ID NO: 560 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C11
  source 1..22: mol_type = other DNA
gcccaaccaaaattctcctgaa

SEQ ID NO: 561 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D11
  source 1..22: mol_type = other DNA
tggtggtggcatgatgagtgtg

SEQ ID NO: 562 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E11
  source 1..22: mol_type = other DNA
ggtggtggcatgatgagtgtgg

SEQ ID NO: 563 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F11
  source 1..22: mol_type = other DNA
tgatgagtgtggagaaaacaac

SEQ ID NO: 564 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G11
  source 1..22: mol_type = other DNA
tgtggagaaaacaacctaaatg

SEQ ID NO: 565 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 H11
  source 1..22: mol_type = other DNA
ggtaaatataacaaaccaagag

SEQ ID NO: 566 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 A12
  source 1..22: mol_type = other DNA
gaagaggattatcttggaagtc

SEQ ID NO: 567 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 B12
  source 1..22: mol_type = other DNA
aagaggattatcttggaagtct

SEQ ID NO: 568 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 C12
  source 1..22: mol_type = other DNA
tcaaaatggaaggttatactct

SEQ ID NO: 569 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 D12
  source 1..22: mol_type = other DNA
caaaatggaaggttatactcta

SEQ ID NO: 570 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 E12
  source 1..22: mol_type = other DNA
atgttgatccatccaacagatt

SEQ ID NO: 571 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 F12
  source 1..22: mol_type = other DNA
catccaacagattcagaaagct

SEQ ID NO: 572 - DNA, 22 bp, synthetic construct
  misc_feature 1..22: note = Description of Artificial Sequence: Synthetic oligonucleotide
  misc_feature 1..22: note = Category: DNA sequence of ANGPTL3 target site
  misc_feature 1..22: note = Description: MG3-6/3-4 ANGPTL3 G12
  source 1..22: mol_type = other DNA
gcctcagttcattcaaagcttt

SEQ ID NO: 573 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 A1
  source 1..110: mol_type = other RNA
acccctccacggtaccgggcgggttgagaatcgaaagattcttaataaggcatccttccgatgctgacttctcaccgtccgttttccaataggagcgggcggtatgtttt

SEQ ID NO: 574 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 B1
  source 1..110: mol_type = other RNA
accagcatacagagtgaccaccgttgagaatcgaaagattcttaataaggcatccttccgatgctgacttctcaccgtccgttttccaataggagcgggcggtatgtttt

SEQ ID NO: 575 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 C1
  source 1..110: mol_type = other RNA
ccagcatacagagtgaccaccggttgagaatcgaaagattcttaataaggcatccttccgatgctgacttctcaccgtccgttttccaataggagcgggcggtatgtttt

SEQ ID NO: 576 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 D1
  source 1..110: mol_type = other RNA
cagggtcatggtcaccgacttcgttgagaatcgaaagattcttaataaggcatccttccgatgctgacttctcaccgtccgttttccaataggagcgggcggtatgtttt

SEQ ID NO: 577 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 E1
  source 1..110: mol_type = other RNA
cctcccaggcctggagtttattgttgagaatcgaaagattcttaataaggcatccttccgatgctgacttctcaccgtccgttttccaataggagcgggcggtatgtttt

SEQ ID NO: 578 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 F1
  source 1..110: mol_type = other RNA
ctcccaggcctggagtttattcgttgagaatcgaaagattcttaataaggcatccttccgatgctgacttctcaccgtccgttttccaataggagcgggcggtatgtttt

SEQ ID NO: 579 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 G1
  source 1..110: mol_type = other RNA
caggctggaccagctggcttttgttgagaatcgaaagattcttaataaggcatccttccgatgctgacttctcaccgtccgttttccaataggagcgggcggtatgtttt

SEQ ID NO: 580 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 H1
  source 1..110: mol_type = other RNA
ggtggccccaactgtgatgaccgttgagaatcgaaagattcttaataaggcatccttccgatgctgacttctcaccgtccgttttccaataggagcgggcggtatgtttt

SEQ ID NO: 581 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 A2
  source 1..110: mol_type = other RNA
gccccgccgcttcccactcctggttgagaatcgaaagattcttaataaggcatccttccgatgctgacttctcaccgtccgttttccaataggagcgggcggtatgtttt

SEQ ID NO: 582 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 B2
  source 1..110: mol_type = other RNA
agtgtgctgaccatacagtcctgttgagaatcgaaagattcttaataaggcatccttccgatgctgacttctcaccgtccgttttccaataggagcgggcggtatgtttt

SEQ ID NO: 583 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 C2
  source 1..110: mol_type = other RNA
cctgcaaaacagctgccaacctgttgagaatcgaaagattcttaataaggcatccttccgatgctgacttctcaccgtccgttttccaataggagcgggcggtatgtttt

SEQ ID NO: 584 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 D2
  source 1..110: mol_type = other RNA
ctgcaaaacagctgccaacctggttgagaatcgaaagattcttaataaggcatccttccgatgctgacttctcaccgtccgttttccaataggagcgggcggtatgtttt

SEQ ID NO: 585 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 E2
  source 1..110: mol_type = other RNA
aatggcgtagacaccctcacccgttgagaatcgaaagattcttaataaggcatccttccgatgctgacttctcaccgtccgttttccaataggagcgggcggtatgtttt

SEQ ID NO: 586 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 F2
  source 1..110: mol_type = other RNA
tcctgctgccatgccccaggtcgttgagaatcgaaagattcttaataaggcatccttccgatgctgacttctcaccgtccgttttccaataggagcgggcggtatgtttt

SEQ ID NO: 587 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting PCSK9
  misc_feature 1..110: note = Description: MG3-6/3-4 PCSK9 G2
  source 1..110: mol_type = other RNA
tggaatgcaaagtcaaggagcagttgagaatcgaaagattcttaataaggcatccttccgatgctgacttctcaccgtccgttttccaataggagcgggcggtatgtttt

SEQ ID NO: 588 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 A1
  source 1..22: mol_type = other DNA
acccctccacggtaccgggcgg

SEQ ID NO: 589 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 B1
  source 1..22: mol_type = other DNA
accagcatacagagtgaccacc

SEQ ID NO: 590 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 C1
  source 1..22: mol_type = other DNA
ccagcatacagagtgaccaccg

SEQ ID NO: 591 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 D1
  source 1..22: mol_type = other DNA
cagggtcatggtcaccgacttc

SEQ ID NO: 592 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 E1
  source 1..22: mol_type = other DNA
cctcccaggcctggagtttatt

SEQ ID NO: 593 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 F1
  source 1..22: mol_type = other DNA
ctcccaggcctggagtttattc

SEQ ID NO: 594 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 G1
  source 1..22: mol_type = other DNA
caggctggaccagctggctttt

SEQ ID NO: 595 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 H1
  source 1..22: mol_type = other DNA
ggtggccccaactgtgatgacc

SEQ ID NO: 596 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 A2
  source 1..22: mol_type = other DNA
gccccgccgcttcccactcctg

SEQ ID NO: 597 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 B2
  source 1..22: mol_type = other DNA
agtgtgctgaccatacagtcct

SEQ ID NO: 598 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 C2
  source 1..22: mol_type = other DNA
cctgcaaaacagctgccaacct

SEQ ID NO: 599 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 D2
  source 1..22: mol_type = other DNA
ctgcaaaacagctgccaacctg

SEQ ID NO: 600 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 E2
  source 1..22: mol_type = other DNA
aatggcgtagacaccctcaccc

SEQ ID NO: 601 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 F2
  source 1..22: mol_type = other DNA
tcctgctgccatgccccaggtc

SEQ ID NO: 602 - DNA, 22 bp, synthetic construct
  misc_feature 1..22: note = Description of Artificial Sequence: Synthetic oligonucleotide
  misc_feature 1..22: note = Category: DNA sequence of PSCK9 target site
  misc_feature 1..22: note = Description: MG3-6/3-4 PCSK9 G2
  source 1..22: mol_type = other DNA
tggaatgcaaagtcaaggagca

SEQ ID NO: 603 - DNA, 3705 bp, synthetic construct
  misc_feature 1..3705: note = Description of Artificial Sequence: Synthetic polynucleotide
  misc_feature 1..3705: note = Category: MG3-6/3-4 coding sequence
  misc_feature 1..3705: note = Description: DNA sequence of MG3-6/3-4 coding sequence
  source 1..3705: mol_type = other DNA
atgcatgcgcggccgcaagctttaatacgactcactataaggaaaagccagctccagcaggcgctgctcactcctccccatcctctccctctgtccctctgtccctctgaccctgcactgtcccagcaccatggcccccaagaagaagcggaaagttggcggcggaggcagcagcaccgacatgaagaactaccggatcggcgtggacgtgggcgatagatctgttggactggccgccatcgagttcgacgatgatggactgcccatccagaagctggccctggtcacctttagacacgatggcggactggaccccaccaagaacaagacccctatgagccggaaagagacacggggaatcgccagacggaccatgcggatgaacagagagcggaagcggcggctgagaaacctggacaacgtgctggaaaacctgggctactctgtgcctgagggccctgagcctgagacatatgaggcctggacaagcagagccctgctggcctctatcaaactggcctctgccgacgagctgaacgaacaccttgtcagagccgtgcggcacatggccagacatagaggatgggccaatccttggtggtccctggaccagctggaaaaggccagccaagagcctagcgagacattcgagatcatcctggccagagccagagagctgttcggcgagaaggtgcccgctaatcctacactgggaatgctgggagccctggccgctaacaatgaggtgctgctgaggcccagggacgagaagaagagaaagaccggatacgtgcggggcacccctctgatgtttgctcaagttcgacagggcgatcagctggccgagctgcggagaatttgtgaagtgcagggcatcgaggaccagtacgaggctctgagactgggcgtgttcgaccacaagcacccctacgtgcccaaagaaagagtgggcaaagaccctctgaaccccagcaccaacagaaccatcagagccagcctggaatttcaagagttccgcatcctggacagcgtggccaatctgagagtgcggatcggcagcagagccaagagggaactgacagaggccgagtatgatgccgccgtggaattcctgatggactacgccgacaaagagcagcctagctgggccgatgtggccgagaaaattggcgtgcccggcaacagactggtggcccctgttctggaagatgtgcagcagaaaacagccccttacgacagaagcagcgccgcctttgagaaggccatgggcaagaaaaccgaggccagacagtggtgggagtccaccgatgatgaccagctgagaagcctgctgattgccttcctggtggacgccaccaacgacacagaagaagccgctgctgaagccggcctgagcgagctgtataagtcttggcctgccgaggaaagagaggccctgtccaacatcgacttcgagaagggcagagtggcctacagccaagaaaccctgagcaagctgagcgagtacatgcacgagtacagagtgggactgcacgaggctagaaaggccgtgttcggagtggatgatacctggcggcctcctctggataagctggaagaacctacaggacagcctgccgtggacagagtgctgaccatcctgagaagattcgtgctggactgcgagcggcaatggggcagacctagagccatcaccgtggaacacacacggacaggcctgatgggcccaacacagagacagaagatcctgaacgagcagaagaagaaccgggccgacaacgagagaatccgggatgagctgagagaatctggcgtggacaacccctccagagccgaagttcggagacacctgatcgtgcaagagcaagagtgccagtgcctgtactgcggcaccatgatcaccaccaccacaagcgagctggaccacatcgttcctagagccggtggcggcagcagcagaagggaaaatctggccgctgtgtgcagagcctgcaacgccaagaagaaacgcgagctgttctacgcctgggctggcccagtgaagtcccaagagacaatcgagagagtcagacagctgaaggcctttaaggacagcaagaaagccaagatgttcaagaaccagatccgccggctgaaccagaccgaggccgatgagcctatcgacgaaagaagcctggccagcacatcttacgccgctgtggccgttagagagcggctggaacagcacttcaacgaaggcctggcactggacgacaagtccagagtggtgctggatgtgtatgccggcgctgtgaccagagagtctcgtagagctggcggcatcgacgagcggattctgctgagaggcgagcgggacaagaacagattcgatgtgcggcatcacgccgtggacgctgctgttatgaccctgctgaacagatccgtggctctgaccctggaacagagatcacagctgaggcgggccttctacgagctggaactggacaaactggaccgggaccagctcaagcctggcgaggattggagaaacttcaccggcctgtacgaggcctctcagaacaagttcagcgagtggaagaaagccgccacagtgctgggagatctgctggctgaagccatcgaggatgacgccattgccgtggtgtctccactgagactgaggccccagaatggcagcgtgcacgacgataccatcaacgccgtgaagaagctgacactgggctctgcctggcctgcagacgctgtgaagagaatcgtggaccccgagatctacctggctatgaaggacgtgctgggcaagctgaaagagctgcccgaggattctgccagatctctggaactgtccgacggccggtacatcgaagccgatgacgaggtgctgttcttcccaaagaaggccgctagcatcctgacacctagaggcgccgctgagatcggcaactctatccaccatgccagactgtatagctggctgaccaagaagggcgagctgaagtttggcatgctgagagtgtacggcgccgagtttccctggctgatgagagagtctggaagccgcgacgtgctgcatatgcctattcaccctggcagccagagcttcagaggcatgcaggatggcgtgcggaaagccgtggaaagcggagaggctgtggaattcggctggatcacccaggacgatgagctggaattcgaccccgaggactacattgcccacggcggagatgacgaactgaacagactgctgcgagtgatgcccgagagaaggtggcgagtggacggcttctataacgccggcacactgagaatcagacccgctctgctgtctgctgagcagctgccttctgagctgcagaaaaaggtggccgacaagaccctgagcgacgtggaactgatcctgctgagggctgttcagcggggactgttcgtggccatcagcagctttctgcccctggaaagcctgaaagtgatccggcggaacaatctgggcttccccaggtggcgcggaaacggaaatctgcccaccagctttgaagtgcggagcagcgctctgagagccctgggagttgaaggatctggcggaaaaagacctgccgccacaaagaaagccggacaggccaagaaaaagaagtgaccacacccccattcccccactccagatagaacttcagttatatctcacgtgtctggagttggatccatgcatgc

SEQ ID NO: 604 - protein, 1166 aa, synthetic construct
  REGION 1..1166: note = Description of Artificial Sequence: Synthetic polypeptide
  REGION 1..1166: note = Category: MG3-6/3-4 cassette coding sequence
  REGION 1..1166: note = Description: Protein sequence of MG3-6/3-4 cassette coding sequence (includes NLS)
MAPKKKRKVGGGGSSTDMKNYRIGVDVGDRSVGLAAIEFDDDGLPIQKLALVTFRHDGGLDPTKNKTPMSRKETRGIARRTMRMNRERKRRLRNLDNVLENLGYSVPEGPEPETYEAWTSRALLASIKLASADELNEHLVRAVRHMARHRGWANPWWSLDQLEKASQEPSETFEIILARARELFGEKVPANPTLGMLGALAANNEVLLRPRDEKKRKTGYVRGTPLMFAQVRQGDQLAELRRICEVQGIEDQYEALRLGVFDHKHPYVPKERVGKDPLNPSTNRTIRASLEFQEFRILDSVANLRVRIGSRAKRELTEAEYDAAVEFLMDYADKEQPSWADVAEKIGVPGNRLVAPVLEDVQQKTAPYDRSSAAFEKAMGKKTEARQWWESTDDDQLRSLLIAFLVDATNDTEEAAAEAGLSELYKSWPAEEREALSNIDFEKGRVAYSQETLSKLSEYMHEYRVGLHEARKAVFGVDDTWRPPLDKLEEPTGQPAVDRVLTILRRFVLDCERQWGRPRAITVEHTRTGLMGPTQRQKILNEQKKNRADNERIRDELRESGVDNPSRAEVRRHLIVQEQECQCLYCGTMITTTTSELDHIVPRAGGGSSRRENLAAVCRACNAKKKRELFYAWAGPVKSQETIERVRQLKAFKDSKKAKMFKNQIRRLNQTEADEPIDERSLASTSYAAVAVRERLEQHFNEGLALDDKSRVVLDVYAGAVTRESRRAGGIDERILLRGERDKNRFDVRHHAVDAAVMTLLNRSVALTLEQRSQLRRAFYELELDKLDRDQLKPGEDWRNFTGLYEASQNKFSEWKKAATVLGDLLAEAIEDDAIAVVSPLRLRPQNGSVHDDTINAVKKLTLGSAWPADAVKRIVDPEIYLAMKDVLGKLKELPEDSARSLELSDGRYIEADDEVLFFPKKAASILTPRGAAEIGNSIHHARLYSWLTKKGELKFGMLRVYGAEFPWLMRESGSRDVLHMPIHPGSQSFRGMQDGVRKAVESGEAVEFGWITQDDELEFDPEDYIAHGGDDELNRLLRVMPERRWRVDGFYNAGTLRIRPALLSAEQLPSELQKKVADKTLSDVELILLRAVQRGLFVAISSFLPLESLKVIRRNNLGFPRWRGNGNLPTSFEVRSSALRALGVEGSGGKRPAATKKAGQAKKKK

SEQ ID NO: 605 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting mouse HAO-1
  misc_feature 1..110: note = Description: mH364-7-1
  source 1..110: mol_type = other RNA
gagctggccactgtgcgaggtagttgagaatcgaaagattcttaataaggcatccttccgatgctgacttctcaccgtccgttttccaataggagcgggcggtatgtttt

SEQ ID NO: 606 - RNA, 110 nt, synthetic construct
  misc_feature 1..110: note = Description of Artificial Sequence: Synthetic polynucleotide
  misc_feature 1..110: note = Category: MG3-6/3-4 sgRNA targeting mouse HAO-1
  misc_feature 1..110: note = Description: mH364-20-1
  source 1..110: mol_type = other RNA
ttcagcaagtccactgttgtctgttgagaatcgaaagattcttaataaggcatccttccgatgctgacttctcaccgtccgttttccaataggagcgggcggtatgtttt

SEQ ID NO: 607 - RNA, 110 nt, synthetic construct; identical to SEQ ID NO: 605
  misc_feature 1..110: note = Description of Artificial Sequence: Synthetic polynucleotide
  misc_feature 1..110: note = Category: MG3-6/3-4 sgRNA targeting mouse HAO-1
  misc_feature 1..110: note = Description: mH364-7-35
  source 1..110: mol_type = other RNA

SEQ ID NO: 608 - RNA, 110 nt, synthetic construct; identical to SEQ ID NO: 606
  misc_feature 1..110: note = Description of Artificial Sequence: Synthetic polynucleotide
  misc_feature 1..110: note = Category: MG3-6/3-4 sgRNA targeting mouse HAO-1
  misc_feature 1..110: note = Description: mH364-20-35
  source 1..110: mol_type = other RNA

SEQ ID NO: 609 - RNA, 110 nt, synthetic construct; identical to SEQ ID NO: 605
  misc_feature 1..110: note = Description of Artificial Sequence: Synthetic polynucleotide
  misc_feature 1..110: note = Category: MG3-6/3-4 sgRNA targeting mouse HAO-1
  misc_feature 1..110: note = Description: mH364-7-42
  source 1..110: mol_type = other RNA

SEQ ID NO: 610 - RNA, 110 nt, synthetic construct; identical to SEQ ID NO: 605
  misc_feature 1..110: note = Description of Artificial Sequence: Synthetic polynucleotide
  misc_feature 1..110: note = Category: MG3-6/3-4 sgRNA targeting mouse HAO-1
  misc_feature 1..110: note = Description: mH364-7-45
  source 1..110: mol_type = other RNA

(19) United States
(12) Patent Application Publication
(10) Pub. No.: US 2023/0416710 A1
(43) Pub. Date: Dec. 28, 2023

(54) ENGINEERED AND CHIMERIC NUCLEASES

(71) Applicant: Metagenomi, Inc., Emeryville, CA (US)

(72) Inventors: Brian C. THOMAS, Emeryville, CA (US); Christopher BROWN, Emeryville, CA (US); Cristina BUTTERFIELD, Emeryville, CA (US); Jyun-Liang LIN, Emeryville, CA (US); Alan BROOKS, Emeryville, CA (US); Morayma M. TEMOCHE-DIAZ, Emeryville, CA (US); Greg COST, Emeryville, CA (US); Rebecca LAMOTHE, Emeryville, CA (US)

(21) Appl. No.: 18/056,629

(22) Filed: Nov. 17, 2022

Related U.S. Application Data
(63) Continuation of application No. PCT/US22/13396, filed on Jan. 21, 2022.
(60) Provisional application No. 63/140,620, filed on Jan. 22, 2021.
(60) Provisional application No. 63/237,484, filed on Aug. 26, 2021.

(51) Int. Cl. C12N 9/22

(52) U.S. Cl. CPC C12N 9/22

(57) ABSTRACT

Disclosed herein are engineered nucleases and nuclease systems, including chimeric nucleases and chimeric nuclease systems. Engineered and chimeric nucleases disclosed herein include nucleic acid guided nuclease. Additionally disclosed herein are methods of generating engineered nucleases and methods of using the same.

### CROSS-REFERENCE

This application is a continuation of International Application No. PCT/PCT/US2022/013396 entitled “NOVEL ENGINEERED AND CHIMERIC NUCLEASES”, filed Jan. 21, 2022, which claims the benefit of U.S. Provisional Application No. 63/237,484, entitled “NOVEL ENGINEERED AND CHIMERIC NUCLEASES”, filed on Aug. 26, 2021, and U.S. Provisional Application No. 63/140,620 entitled “NOVEL ENGINEERED AND CHIMERIC NUCLEASES” filed on Jan. 22, 2021, each of which is incorporated by reference herein in its entirety.

This application is related to International Application No. PCT/US2021/031136 entitled “ENZYMES WITH RUVC DOMAINS”, filed on May 6, 2021, and PCT/US2020/018432, filed on Feb. 14, 2020, entitled “ENZYMES WITH RUVC DOMAINS”, each of which is incorporated by reference herein in its entirety.

### SEQUENCE LISTING

The instant application contains a Sequence Listing which has been submitted electronically in XML format and is hereby incorporated by reference in its entirety. Said XML copy, created on Nov. 17, 2022, is named 55921-717_301_SL.txt and is 1,351,136 bytes in size.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides for a fusion endonuclease comprising: (a) an N-terminal sequence comprising at least part of a RuvC domain, a REC domain, or an HNH domain of an endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696 or a variant thereof; and (b) a C-terminal sequence comprising WED, TOPO, or CTD domains of an endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 697-721 or variants thereof, wherein said N-terminal sequence and said C-terminal sequence do not naturally occur together in a same reading frame. In some embodiments, the endonuclease is a Class II, type II Cas endonuclease. In some embodiments, the endonuclease is a Class II, type V Cas endonuclease. In some embodiments, said N-terminal sequence and said C-terminal sequence are derived from different organisms. In some embodiments, said N-terminal sequence further comprises RuvC-I, BH, or RuvC-II domains. In some embodiments, said C-terminal sequence further comprises a PAM-interacting domain. In some embodiments, said fusion endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27 or 108. In some embodiments, said fusion endonuclease is configured to bind to a PAM that is not nnRGGnT (SEQ ID NO: 53). In some embodiments, said fusion endonuclease is configured to bind to a PAM that comprises any one of SEQ ID NOs:46-52 or 54-66.

In some aspects, the present disclosure provides for an endonuclease comprising an engineered amino acid sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27 or 108, or a variant thereof.

In some aspects, the present disclosure provides for an endonuclease comprising an engineered amino acid sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 109-110, or a variant thereof.

In some aspects, the present disclosure provides for a nucleic acid comprising a sequence encoding any of the endonucleases, fusion endonucleases, or Cas enzymes described herein. In some aspects, the sequence is codon-optimized for expression in a host cell. In some embodiments, the host cell is prokaryotic, eukaryotic, mammal, or human.

In some aspects, the present disclosure provides for a vector comprising any of the nucleic acid sequences described herein.

In some aspects, the present disclosure provides for a host cell comprising any of the vectors, systems, or nucleic acids described herein. In some embodiments, the host cell is prokaryotic, eukaryotic, mammal, or human.

In some aspects, the present disclosure provides for an engineered nuclease system, comprising: (a) any of the nucleases, Cas enzymes, or fusion endonucleases described herein; and (b) an engineered guide ribonucleic structure configured to form a complex with said endonuclease comprising: a guide ribonucleic acid configured to hybridize to a target deoxyribonucleic acid sequence; wherein said guide ribonucleic acid sequence is configured to bind to said endonuclease. In some embodiments, said guide ribonucleic acid further comprises a tracr ribonucleic acid sequence configured to bind said endonuclease. In some embodiments, said endonuclease is derived from an uncultivated microorganism. In some embodiments, said endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, said endonuclease has less than 86% identity to a SpyCas9 endonuclease. In some embodiments, said system further comprises a source of Mg′. In some embodiments, said endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 8-12, 26-27, or 108, or a variant thereof. In some embodiments, said guide ribonucleic acid sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to non-degenerate nucleotides of any one of SEQ ID NOs: 33, 34, 44, 45, 78, 84, or 87.

In some aspects, the present disclosure provides for an engineered nuclease comprising: (a) a class II, type II Cas enzyme RuvC or HNH domain having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a RuvC or HNH domain of any one of SEQ ID NOs: 1-27, 108, or 109-110, or variants thereof and (b) a class II, type II Cas enzyme PAM-interacting (PI) domain having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a PAM-interacting (PI) domain any one of SEQ ID NOs: 1-27, 108, or 109-110, or variants thereof. In some embodiments, (a) and (b) do not naturally occur together. In some embodiments, said class II, type II Cas enzyme is derived from an uncultivated microorganism. In some embodiments, said endonuclease has less than 86% identity to a SpyCas9 endonuclease. In some embodiments, said engineered nuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27 or a variant thereof.

In some aspects, the present disclosure provides for an engineered nuclease system, comprising: (a) any of the endonucleases described herein; and (b) an engineered guide ribonucleic structure configured to form a complex with said endonuclease comprising: a guide ribonucleic acid sequence configured to hybridize to a target deoxyribonucleic acid sequence and configured to bind to said endonuclease. In some embodiments, said guide ribonucleic acid further comprises a tracr ribonucleic acid sequence configured to bind said endonuclease. In some embodiments, said guide ribonucleic acid sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of any one of SEQ ID NOs: 28-32 or 33-44, or a variant thereof. In some embodiments, the system further comprises a PAM sequence compatible with said nuclease adjacent to said target nucleic acid site. In some embodiments, said PAM sequence is located 3′ of said target deoxyribonucleic acid sequence. In some embodiments, said PAM sequence is located 5′ of said target deoxyribonucleic acid sequence. In some embodiments, said PAM sequence comprises any one of SEQ ID NOs:46-66.

In some aspects, the present disclosure provides for a method of targeting the albumin gene, comprising introducing any of the systems described herein to a cell, wherein said guide ribonucleic acid sequence is configured to hybridize to a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity any one of SEQ ID NOs: 67-86. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for a method of targeting the HAO1 gene or locus, comprising introducing any of the systems described herein to a cell, wherein said guide ribonucleic acid sequence is configured to hybridize to a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 611-633. In some embodiments, said guide ribonucleic acid sequence is configured to hybridize to a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 615, 618, 620, 624, or 626. In some embodiments, said guide ribonucleic acid comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs:645-684. In some embodiments, said guide ribonucleic acid comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 645-649, 652-656, 660-671, 674-675, or 681-684, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 645-649, 652-656, 660-671, 674-675, or 681-684. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some embodiments, the present disclosure provides for a method of disrupting an HAO-1 locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said HAO-1 locus, wherein said engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 611-626 or 627-633. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO:10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722. In some embodiments, said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 618, 620, 624, or 626, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a targeting sequence of any one of SEQ ID NOs: 618, 620, 624, or 626. In some embodiments, said engineered guide RNA comprises the nucleotide sequence of any one of the guide RNAs from Table 9 or Table 12. In some embodiments, the cell is a mammalian cell. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for a method of disrupting a TRAC locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said TRAC locus, wherein said engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 139-158; or wherein said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 119-138. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises any of the fusion endonucleases described herein. In some embodiments, said class 2, type II Cas endonuclease comprises the fusion endonuclease having at least 55% identity to SEQ ID NO:10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722. In some embodiments, said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 121, 132, 136, 130, 134, 135, or 137, or a sequence having at least 80% identity to a targeting sequence of any one of SEQ ID NOs: 121, 132, 136, 130, 134, 135, or 137. In some embodiments, said engineered guide RNA comprises a nucleotide sequence of any one of the guide RNAs from Table 7A. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some embodiments, the present disclosure provides for a method of disrupting a B2M locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said B2M locus, wherein said engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 185-210; or wherein said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 159-184. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises any of the fusion endonucleases described herein. In some embodiments, said class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 159, 165, 168, 174, or 184, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 159, 165, 168, 174, or 184. In some embodiments, said engineered guide RNA comprises a nucleotide sequence of any one of the guide RNAs from Table 7B. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for a method of disrupting a TRBC1 locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said TRBC1 locus, wherein said engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 252-292; or wherein the engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 211-251. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises any of the fusion endonucleases described herein. In some embodiments, said class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO:10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, said engineered guide RNA is comprises a sequence having at least 80% identity to any one of SEQ ID NOs: 211, 212, 215, 241, or 242, or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 211, 212, 215, 241, or 242. In some embodiments, said engineered guide RNA comprises a nucleotide sequence of any one of the guide RNAs from Table 7C. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for a method of disrupting a TRBC2 locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said TRBC2 locus, wherein said engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 338-382; or wherein said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 293-337. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, the class 2, type II Cas endonuclease any of the fusion endonucleases described herein. In some embodiments, said class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO:10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 296, 306, or 332, or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID Nos: 296, 306, or 332. In some embodiments, said engineered guide RNA comprises a nucleotide sequence of any one of the guide RNAs from Table 7C. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for a method of disrupting an ANGPTL3 locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said ANGPTL3 locus, wherein said engineered guide RNA is configured to hybridize to or comprises a targeting sequence having at least 80% identity to SEQ ID NOs: 478-572; or wherein said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 383-477. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises any of the fusion endonucleases described herein. In some embodiments, said class 2, type II Cas endonuclease comprises a fusion endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a non-degenerate nucleotides of SEQ ID NO: 722. In some embodiments, said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 419, 425, 431, 439, 447, 453, 461, 467, 471, or 473, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 419, 425, 431, 439, 447, 453, 461, 467, 471, or 473. In some embodiments, said engineered guide RNA comprises a nucleotide sequence of any one of the guide RNAs from Table 7D. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for a method of disrupting a PCSK9 locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said PCSK9 locus, wherein said engineered guide RNA is configured to hybridize to or comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NOs: 588-602; or wherein said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 573-587. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises any of the fusion endonucleases described herein. In some embodiments, said class 2, type II Cas endonuclease comprises a fusion endonuclease comprising a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the non-degenerate nucleotides of SEQ ID NO: 722. In some embodiments, said engineered guide comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 574, 578, 581, or 585. In some embodiments, said engineered guide RNA comprises a nucleotide sequence of any one of the guide RNAs from Table 7E. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some embodiments, the present disclosure provides for a method of disrupting an albumin locus in a cell, comprising introducing to said cell: (a) any of the endonucleases described herein; and (b) an engineered guide RNA, wherein said engineered guide RNA is configured to form a complex with said endonuclease and said engineered guide RNA comprises a targeting sequence configured to hybridize to a region of said albumin locus, wherein said engineered guide RNA comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 67-86 or 646-695, or wherein said engineered guide RNA comprises a targeting sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a targeting sequence of any one of SEQ ID NOs: 67-86 or 646-695. In some embodiments, the endonuclease is a class 2, type II Cas endonuclease. In some embodiments, said class 2, type II Cas endonuclease comprises any of the fusion or engineered endonucleases described herein. In some embodiments the endonuclease comprises any of the fusion or engineered endonucleases described herein. In some embodiments, said class 2, type II Cas endonuclease comprises any of the type II Cas endonucleases described herein. In some embodiments, said class 2, type II Cas endonuclease comprises a fusion endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 10 or a variant thereof. In some embodiments, said engineered guide RNA comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of SEQ ID NO: 722. In some embodiments, said engineered guide RNA is complementary to or comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any one of SEQ ID NOs: 67, 68, 70, 71, 72, 76, 79, 80, 647, 648, 649, 653, 654, 655, 656, 673, 680, 681, or 682. In some embodiments, said engineered guide RNA comprises a nucleotide sequence of any one of the guide RNAs from Table 6. In some embodiments, introducing to said cell further comprises contacting said cell with a nucleic acid or vector encoding said fusion protein or said guide polynucleotide. or comprises contacting said cell with a lipid nanoparticle (LNP) comprising said vector or nucleic acid. In some embodiments, introducing to said cell further comprises contacting said cell with a ribonucleoprotein complex (RNP) comprising said fusion protein or said guide polynucleotide or comprises contacting said cell with a lipid nanoparticle (LNP) comprising said RNP.

In some aspects, the present disclosure provides for an endonuclease comprising an engineered amino acid sequence having at least 55% sequence identity to any one of SEQ ID NOs: 1-27, 108, or 109-110.

In some aspects, the present disclosure provides an engineered nuclease system, comprising the endonuclease described herein, and an engineered guide ribonucleic structure configured to form a complex with the endonuclease comprising: a guide ribonucleic acid sequence configured to hybridize to a target deoxyribonucleic acid sequence; and a tracr ribonucleic acid sequence configured to bind to said endonuclease. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 86% identity to a SpyCas9 endonuclease. In some embodiments, the system further comprises a source of MG′.

In some aspects, the present disclosure provides for an engineered nuclease comprising: (a) a class II, type II Cas enzyme RuvC and HNH domain having at least 55% sequence identity to a RuvC and HNH domain of any one of SEQ ID NOs: 1-27, 108, or 109-110; and (b) a class II, type II Cas enzyme PAM-interacting (PI) domain having at least 55% sequence identity to a PAM-interacting (PI) domain any one of SEQ ID NOs: 1-27, 108, or 109-110. In some embodiments, (a) and (b) do not naturally occur together. In some embodiments, the class II, type II Cas enzyme is derived from an uncultivated microorganism. In some embodiments, the endonuclease has less than 86% identity to a SpyCas9 endonuclease. In some embodiments, the engineered nuclease comprises a sequence having at least 55% sequence identity to any one of SEQ ID NOs: 1-27.

In some aspects, the present disclosure provides for an engineered nuclease system, comprising: an endonuclease according to any of the aspects or embodiments described herein; and an engineered guide ribonucleic structure configured to form a complex with the endonuclease comprising: a guide ribonucleic acid sequence configured to hybridize to a target deoxyribonucleic acid sequence; and a tracr ribonucleic acid sequence configured to bind to the endonuclease. In some embodiments, the guide ribonucleic acid sequence comprises a sequence having at least 80% sequence identity to non-degenerate nucleotides of any one of SEQ ID NOs: 28-32 or 33-44, or a variant thereof. In some embodiments, the system further comprises a PAM sequence compatible with the nuclease adjacent to the target nucleic acid site. In some embodiments, the PAM sequence is located 3′ of the target deoxyribonucleic acid sequence. In some embodiments, the PAM sequence comprises any one of SEQ ID NOs:46-66.

In some embodiments, the present disclosure provides for an engineered single-molecule heterologous guide polynucleotide compatible with a class II, type II enzyme according to any of the aspects or embodiments described herein, wherein the heterologous guide polynucleotide comprises chemical modifications according to any one of SEQ ID NOs: 645-684.

In some aspects, the present disclosure provides for a method of targeting the albumin gene, comprising introducing a system according to any one of the aspects or embodiments described herein to a cell, wherein the guide ribonucleic acid sequence is configured to hybridize to a sequence comprising any one of SEQ ID NOs: 67-86.

In some aspects, the present disclosure provides for a method of targeting the HAO1 gene, comprising introducing a system according to any one of the aspects or embodiments described herein to a cell, wherein the guide ribonucleic acid sequence is configured to hybridize to any one of SEQ ID NOs: 611-633. In some embodiments, the guide ribonucleic acid sequence is configured to hybridize to any one of SEQ ID NOs: 615, 618, 620, 624, or 626. In some embodiments, the guide ribonucleic acid comprises a sequence according to any one of SEQ ID NOs:645-684. In some embodiments, the guide ribonucleic acid comprises a sequence according to any one of SEQ ID NOs: 645-649, 652-656, 660-671, 674-675, or 681-684.

In some aspects, the present disclosure provides cells comprising the endonucleases described herein. In some aspects, the present disclosure provides cells comprising any nucleic acid molecule described herein. In some aspects, the present disclosure provides cells comprising any engineered nuclease system described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A-1B depicts the natural PAM specificities of various effectors described herein. FIG. 1A shows a phylogenetic tree of the various effectors described herein. FIG. 1B is a table of the PAM specificities of natural RNA guided CRISPR-associated endonucleases.

FIG. 2 demonstrates the concept of domain swapping between RNA guided CRISPR-associated nucleases.

FIGS. 3A and 3B depict the alignment of multiple sequences to guide the determination of an optimal breakpoint. FIG. 3A shows SaCas9 and SpCas9 aligned to several proteins described herein and the terminal conserved residue (an alanine residue) of these sequences are identified as the proposed C-terminus of the swapped section. FIG. 3B depicts the C-terminal domain of a SaCas9 protein to be swapped spans of the RuvC-III, WED, TOPO, and CTD domains. The PAM Interaction domain is composed of the TOPO domain and the CTD domain. Active site residues (D10, E477, and H701 of RuvC domain and D556, D557, and N580 of the NHN domain) are not included in the swapped C-terminal domain.

FIG. 4 depicts the screening of chimeras with an in vitro PAM enrichment assay when recombining MG3-6 with various C-terminal domains from closely and distantly related nucleases. sgRNAs from N-terminal parental domains were used for RNA guided nuclease activities.

FIG. 5A-5B depicts PAM sequences (FIG. 5A) and Seq Logo depictions of PAM sequences (FIG. 5B) of functional chimeras described herein. Given the breakpoint swapping of predicted C-terminal domains of RuvC-III, WED, TOPO and CTD, chimeras were functional if recombined with closely related nucleases. The engineered chimeras tended to preserve PAM specificities from the natural protein's PAM interacting domains, even if the natural protein was not functional in the same experiment.

FIG. 6 shows the screening of chimeras with an in vitro PAM enrichment assay with chimeras recombining MG3-6 with various c-terminal domains from closely and distantly related nucleases. sgRNAs from C-terminal parental domains were used for RNA guided nuclease activities. Numbers in parentheses indicate sgRNA species. Using sgRNAs from C-terminal parental domains did not rescue activities.

FIG. 7 shows predicted structures of MG3-6 and MG15-1. The WED and PI domains of MG3-6 were swapped with those of MG15-1 counterparts to generate chimera 1 (C1). Alternatively, the PI domain of MG3-6 was swapped with MG15-1's counterpart to generate chimera 2 (C2).

FIG. 8A-8B depicts an in vitro PAM enrichment assay and Sanger sequencing results for PAM specificities. C1: MG3-6+MG15-1 (WP) and C2: MG3-6+MG15-1 (P). The engineered chimeras tend to preserve PAM specificities from the natural proteins' PAM interacting domains. PAM enrichment assay was performed in triplicate. (FIG. 8A) shows an agarose gel depiction of the assay indicating that sequences were cleaved in the presence of the active enzymes and (FIG. 8B) shows SeqLogo depictions of PAM sequences determined by the assay.

FIG. 9A-9B depicts the activity of a chimera described herein in mammalian cells. mRNA codifying for the chimera was co-transfected with 20 different sgRNAs (see e.g. SEQ ID Nos: 67-86) into Hepa 1-6 cells. Editing was assessed by Sanger sequencing and Inference of CRISPR edits (ICE). FIG. 9A shows the editing efficiency of the tested guides. Two biological replicates are shown. FIG. 9B shows the indel profiles created by representative guides.

FIG. 10 depicts the results of a guide screen in Hepa1-6 cells; guides were delivered as mRNA and gRNA using lipofectamine Messenger Max.

FIG. 11A depicts the structural portion of the MG3-6/3-4 guide. FIG. 11B depicts the structural portion of the MG3-6 guide.

FIG. 12 depicts the activity of chemically modified MG3-6/3-4 guides in Hepa1-6 cells when delivered as mRNA and gRNA using lipofectamine Messenger Max.

FIG. 13 depicts the stability of chemically modified MG3-6/3-4 guides over 9 hours at 37° C.

FIG. 14 depicts the stability of chemically modified MG3-6/3-4 guides over 21 hours at 37° C.

FIG. 15A-15B depicts the in vitro screening of Type V-A chimeras. FIG. 15A depicts the agarose gel of amplified cleavage products for each cleavage reaction. Positive enrichment is observed with the MG29-1+MG29-5 chimera, domain swap from the same family (numbers in parentheses indicate sgRNA species). FIG. 15B depicts Seqlogo depictions of PAMs for parent enzymes and the chimeras derived therefrom.

FIG. 16 depicts the gene-editing outcomes at the DNA level for TRAC in HEK293T cells.

FIG. 17 depicts the gene-editing outcomes at the DNA level for B2M in HEK293T cells.

FIG. 18 depicts the gene-editing outcomes at the DNA and phenotypic levels for TRAC in T cells.

FIG. 19 depicts the gene-editing outcomes at the DNA level for B2M in T cells.

FIG. 20 depicts the gene-editing outcomes at the phenotypic level for TRBC1 and TRBC2 in T cells.

FIG. 21 depicts the gene-editing outcomes at the DNA level for ANGPTL3 in Hep3B cells.

FIG. 22 depicts the gene-editing outcomes at the DNA level for PCSK9 in Hep3B cells.

FIG. 23 depicts genome editing at the HAO-1 locus by MG3-6/3-4 in wild type mice analyzed by next generation sequencing.

FIG. 24 depicts glycolate oxidase protein levels in the liver of mice treated with MG3-6/3-4 mRNA and guide RNA targeting the HAO-1 gene.

FIG. 25 depicts genome editing at the HAO-1 locus in wild type mice treated with MG3-6/3-4 mRNA and guide RNA 7 (G7) targeting HAO-1 with 4 different chemical modifications.

FIG. 26 depicts Western blot analysis of glycolate oxidase (GO) protein levels in the liver of mice at 11 days after treatment with LNP encapsulating MG3-6/3-4 mRNA and sgRNA 7 (G7) with 4 different chemical modifications.

### BRIEF DESCRIPTION OF THE SEQUENCE LISTING

The Sequence Listing filed herewith provides example polynucleotide and polypeptide sequences for use in methods, compositions, and systems according to the disclosure. Below are example descriptions of sequences therein.

### MG3-6 Chimeras

SEQ ID NOs: 1-27 show the full-length peptide sequences of MG3-6 chimeric nucleases.

SEQ ID NO: 108 shows the nucleotide sequence of an MG3-6/3-4 nuclease containing 5′ UTR, NLS, CDS, NLS, 3′ UTR, and polyA tail.

SEQ ID NOs: 28-45 and 605-610 show the nucleotide sequences of sgRNAs engineered to function with an MG3-6 chimeric nuclease.

SEQ ID NOs: 46-59 show the natural PAM specificities of various effectors.

SEQ ID NOs: 60-66 show the PAM specificities of chimeric nucleases described herein.

SEQ ID NO: 603 shows the DNA coding sequence for MG3-6/3-4.

SEQ ID NO: 604 shows the protein sequence of the MG3-6/3-4 cassette coding sequence.

### MG29-1 Chimeras

SEQ ID NOs: 109-110 show the full-length peptide sequences of MG29-1 chimeric nucleases.

SEQ ID NOs: 111-113 show the nucleotide sequences of sgRNAs engineered to function with an MG29-1 chimeric nuclease.

SEQ ID NOs: 114-116 show the natural PAM specificities of various effectors.

SEQ ID NO: 117 shows the PAM specificity of a chimeric nuclease described herein.

### TRAC Targeting

SEQ ID NOs: 119-138 show the nucleotide sequences of sgRNAs engineered to function with an MG3-6/3-4 nuclease in order to target TRAC.

SEQ ID NOs: 139-158 show the DNA sequences of TRAC target sites.

### B2M Targeting

SEQ ID NOs: 159-184 show the nucleotide sequences of sgRNAs engineered to function with an MG3-6/3-4 nuclease in order to target B2M.

SEQ ID NOs: 185-210 show the DNA sequences of B2M target sites.

### TRBC1 Targeting

SEQ ID NOs: 211-251 show the nucleotide sequences of sgRNAs engineered to function with an MG3-6/3-4 nuclease in order to target TRBC1.

SEQ ID NOs: 252-292 show the DNA sequences of TRBC1 target sites.

### TRBC2 Targeting

SEQ ID NOs: 293-337 show the nucleotide sequences of sgRNAs engineered to function with an MG3-6/3-4 nuclease in order to target TRBC2.

SEQ ID NOs: 338-382 show the DNA sequences of TRBC2 target sites.

### ANGPTL3 Targeting

SEQ ID NOs: 383-477 show the nucleotide sequences of sgRNAs engineered to function with an MG3-6/3-4 nuclease in order to target ANGPTL3.

SEQ ID NOs: 478-572 show the DNA sequences of ANGPTL3 target sites.

### PCSK9 Targeting

SEQ ID NOs: 573-587 show the nucleotide sequences of sgRNAs engineered to function with an MG3-6/3-4 nuclease in order to target PCSK9.

SEQ ID NOs: 588-602 show the DNA sequences of PCSK9 target sites.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional, or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions, or deletions. A non-native sequence may exhibit or encode for an activity (e.g., enzymatic activity, methyltransferase activity, acetyltransferase activity, kinase activity, ubiquitinating activity, etc.) that may also be exhibited by the nucleic acid or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters comprise, in some instances, a TATA-box or a CAAT box.

The term “expression”, as used herein, generally refers to the process by which a nucleic acid sequence or a polynucleotide is transcribed from a DNA template (such as into mRNA or other RNA transcript) or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity or sequence similarity to a wild type example tracrRNA sequence (e.g., a tracrRNA from S. pyogenes S. aureus, etc. or SEQ ID NOs: *_*). tracrRNA can refer to a nucleic acid with at most about 5%, 10%, 20%, 30%, 40%, 50%, 60%, 70%, 80%, 90%, or 100% sequence identity or sequence similarity to a wild type example tracrRNA sequence (e.g., a tracrRNA from S. pyogenes S. aureus, etc). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type example tracrRNA (e.g., a tracrRNA from S. pyogenes S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type example tracrRNA (e.g., a tracrRNA from S. pyogenes S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

As used herein, the term “RuvC_III domain” generally refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMIs) built based on documented domain sequences (e.g., Pfam HMM PF18541 for RuvC_III).

As used herein, the term “Wedge” (WED) domain generally refers to a domain (e.g. present in a Cas protein) interacting primarily with repeat:anti-repeat duplex of the sgRNA and PAM duplex. A WED domain can generally be identified by alignment to documented domain sequences, structural alignment to proteins with annotated domains, or by comparison to Hidden Markov Models (HMMs) built based on documented domain sequences.

As used herein, the term “PAM interacting domain” or “PI domain” generally refers to a domain interacting with the protospacer-adjacent motif (PAM) external to the seed sequence in a region targeted by a Cas protein. Examples of PAM-interacting domains include, but are not limited to, Topoisomerase-homology (TOPO) domains and C-terminal domains (CTD) present in Cas proteins. A PAM interacting domain or segments thereof can generally be identified by alignment to documented domain sequences, structural alignment to proteins with annotated domains, or by comparison to Hidden Markov Models (HMMs) built based on documented domain sequences.

As used herein, the term “REC domain” generally refers to a domain (e.g. present in a Cas protein) comprising at least one of two segments (REC1 or REC2) that are alpha helical domains thought to contact the guide RNA. A REC domain or segments thereof can generally be identified by alignment to documented domain sequences, structural alignment to proteins with annotated domains, or by comparison to Hidden Markov Models (HMIs) built based on documented domain sequences (e.g., Pfam PF19501 for domain REC1).

As used herein, the term “BH domain” generally refers to a domain (e.g. present in a Cas protein) that is a bridge helix between NUC and REC lobes of a Type II Cas enzyme. A BH domain or segments thereof can generally be identified by alignment to documented domain sequences, structural alignment to proteins with annotated domains, or by comparison to Hidden Markov Models (HMMs) built based on documented domain sequences (e.g., Pfam PF16593 for domain BH).

As used herein, the term “HNH domain” generally refers to an endonuclease domain having characteristic histidine and asparagine residues. An HNH domain can generally be identified by alignment to documented domain sequences, structural alignment to proteins with annotated domains, or by comparison to Hidden Markov Models (HMIs) built based on documented domain sequences (e.g., Pfam HMM PF01844 for domain HNH).

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g. non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein. In some embodiments, such conservatively substituted variants are functional variants. Such functional variants can encompass sequences with substitutions such that the activity of critical active site residues of the endonuclease are not disrupted. In some embodiments, a functional variant of any of the systems described herein lack substitution of at least one of the conserved or functional residues described herein. In some embodiments, a functional variant of any of the systems described herein lacks substitution of all of the conserved or functional residues described herein.

Conservative substitution tables providing functionally similar amino acids are available from a variety of references (see, for example, Creighton, Proteins: Structures and Molecular Properties (W H Freeman & Co.; 2nd Edition (December 1993))). The following eight groups each contain amino acids that are conservative substitutions for one another:
a. Alanine (A), Glycine (G);
b. Aspartic acid (D), Glutamic acid (E);
c. Asparagine (N), Glutamine (Q);
d. Arginine (R), Lysine (K);
e. Isoleucine (I), Leucine (L), Methionine (M), Valine (V);
f. Phenylalanine (F), Tyrosine (Y), Tryptophan (W);
g. Serine (S), Threonine (T); and
h. Cysteine (C), Methionine (M).

Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity.

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cash) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cash-like enzyme. Unlike type I and II systems, type III systems appear to target and cleave DNA-RNA duplexes (such as DNA strands being used as templates for an RNA polymerase).

Type IV CRISPR-Cas systems possess an effector complex that consists of a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are documented as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again documented as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRISPR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems also appear to, in some embodiments, not require a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class II CRISPR-Cas have been most widely adopted for engineering and development as designer nuclease/genome editing applications.

One of the early adaptations of such a system for in vitro use can be found in Jinek et al. (Science. 2012 Aug. 17; 337(6096):816-21, which is entirely incorporated herein by reference). The Jinek study first described a system that involved (i) recombinantly-expressed, purified full-length Cas9 (e.g., a Class II, Type II Cas enzyme) isolated from S. pyogenes SF370, (ii) purified mature ˜42 nt crRNA bearing a ˜20 nt 5′ sequence complementary to the target DNA sequence to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg2+. Jinek later described an improved, engineered system wherein the crRNA of (ii) is joined to the 5′ end of (iii) by a linker (e.g., GAAA) to form a single fused synthetic guide RNA (sgRNA) capable of directing Cas9 to a target by itself.

Mali et al. (Science. 2013 Feb. 15; 339(6121): 823-826), which is entirely incorporated herein by reference, later adapted this system for use in mammalian cells by providing DNA vectors encoding (i) an ORF encoding codon-optimized Cas9 (e.g., a Class II, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Engineered Nucleases

In some aspects, the present disclosure relates to the engineering of novel nucleic acid-guided nucleases and systems. In some embodiments, the engineered nucleases are functional in prokaryotic or eukaryotic cells for in vitro, in vivo or ex vivo applications. In some embodiments, the present disclosure relates to the engineering and optimization of systems, methods and compositions used for genome engineering involving sequence targeting, such as genome perturbation or gene-editing, that relate to nucleic acid-guided nuclease systems and components thereof.

In some aspects, the present disclosure provides engineered nucleases which may include nucleic acid guided nucleases, chimeric nucleases, and nuclease fusions.

Chimeric or Fusion Engineered Nucleases

Chimeric engineered nucleases as described herein may comprise one or more fragments or domains, and the fragments or domains may be of a nuclease, such as nucleic acid-guided nuclease, orthologs of organisms of genus, species, or other phylogenetic groups described herein. The fragments may be from nuclease orthologs of different species. A chimeric engineered nuclease may be comprised of fragments or domains from at least two different nucleases. A chimeric engineered nuclease may be comprised of fragments or domains from nucleases from at least two different species. A chimeric engineered nuclease may be comprised of fragments or domains from at least 2, 3, 4, 5, 6, 7, 8, 9, 10, or more different nucleases or nucleases from different species. In some embodiments, a chimeric engineered nuclease comprises more than one fragment or domain from one nuclease, wherein the more than one fragment or domain are separated by fragments or domains from a second nuclease. In some examples, a chimeric engineered nuclease comprises 2 fragments, each from a different protein or nuclease. In some examples, a chimeric engineered nuclease comprises 3 fragments, each from a different protein or nuclease. In some examples, a chimeric engineered nuclease comprises 4 fragments, each from a different protein or nuclease. In some examples, a chimeric engineered nuclease comprises 5 fragments, each from a different protein or nuclease. In some examples, a chimeric engineered nuclease comprises 3 fragments, wherein at least one fragment is from a different protein or nuclease. In some examples, a chimeric engineered nuclease comprises 4 fragments, wherein at least one fragment is from a different protein or nuclease. In some examples, a chimeric engineered nuclease comprises 5 fragments, wherein at least one fragment is from a different protein or nuclease.

Junctions between fragments or domains from different nucleases or species can occur in stretches of unstructured regions. Unstructured regions may include regions which are exposed within a protein structure or are not conserved within various nuclease orthologs.

MG Chimeric Enzymes

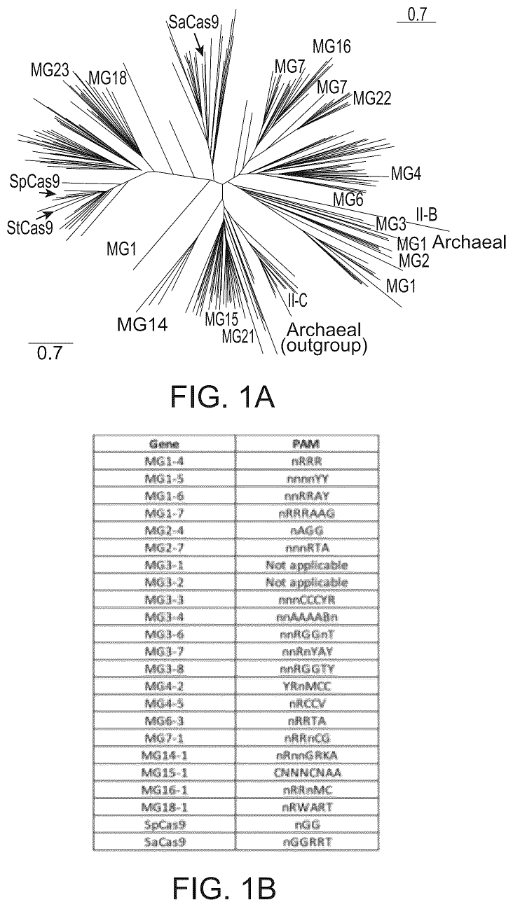

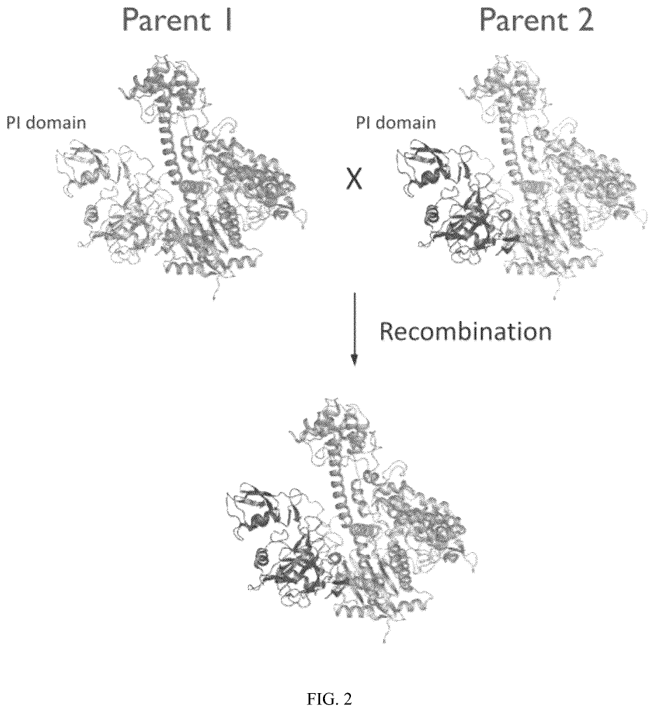

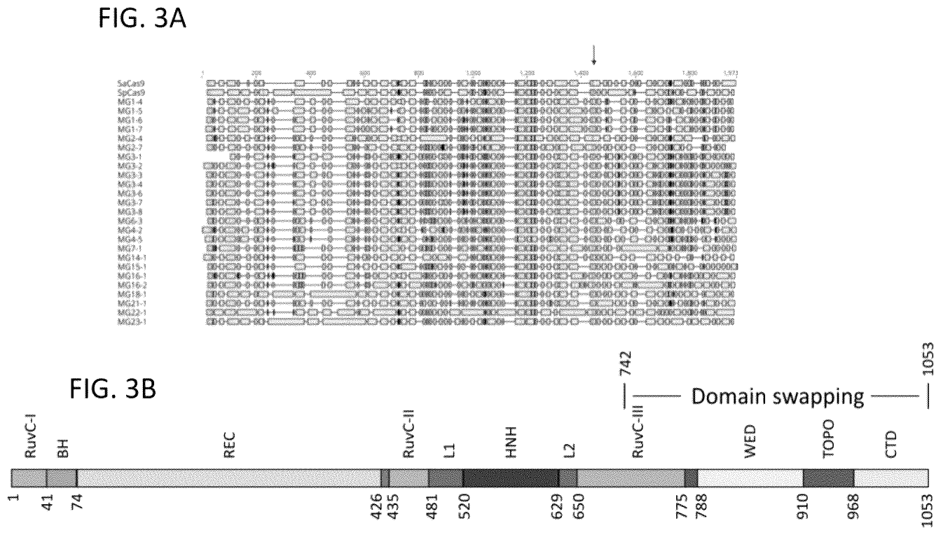

The CRISPR effectors described herein have natural PAM specificities (see FIG. 1). In one aspect, the present disclosure provides for the enablement of novel PAM specificity by protein engineering. This enablement of novel PAM specificity may be achieved by the domain swapping of RNA guided CRISPR-associated nucleases (see FIG. 2). There may be an optimal breakpoint in the process of domain swapping and recombination. The optimal breakpoint may be guided by the alignment of multiple sequences described herein (see FIG. 3).

In some aspects, the present disclosure provides for a fusion endonuclease comprising: (a) an N-terminal sequence comprising RuvC, REC, or HNH domains of a Cas endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696 or a variant thereof; and (b) a C-terminal sequence comprising WED, TOPO, or CTD domains of a Cas endonuclease having at least 55% at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 697-721 or variants thereof. In some embodiments the fusion endonuclease comprises RuvC, REC, and HNH domains in (a). In some embodiments, the fusion endonuclease comprises RuvC and HNH domains in (a). In some embodiments, the fusion endonuclease comprises WED, TOPO, and CTD domains in (b). In some embodiments, the N-terminal sequence and the C-terminal sequence do not naturally occur together in a same reading frame. In some embodiments, the N-terminal sequence and the C-terminal sequence are derived from different organisms. In some embodiments, the N-terminal sequence further comprises RuvC-I, BH, and RuvC-II domains. In some embodiments, the C-terminal sequence further comprises a PAM-interacting domain. In some embodiments, the fusion Cas endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity sequence identity to any one of SEQ ID NOs: 1-27 or 108. In some embodiments, the fusion endonuclease is configured to bind to a PAM that is not nnRGGnT (SEQ ID NO: 53). In some embodiments, the fusion endonuclease is configured to bind to a PAM that comprises any one of SEQ ID NOs:46-52 or 54-66.

In some aspects, the present disclosure provides an endonuclease comprising an engineered nucleic acid sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-27, 108, or 109-110. In one aspect, the present disclosure provides an endonuclease comprising an engineered nucleic acid sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 8-12, 26-27, or 108. In one aspect, the present disclosure provides an engineered nuclease system, comprising: the endonuclease described herein; and an engineered guide ribonucleic structure configured to form a complex with the endonuclease comprising: a guide ribonucleic acid sequence configured to hybridize to a target deoxyribonucleic acid sequence and configured to bind to the endonuclease. In some embodiments, and the engineered guide ribonucleic acid sequence further comprises a tracr ribonucleic acid sequence. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 86% identity to a SpyCas9 endonuclease. In some embodiments, the system further comprises a source of Mg′.

In some aspects, the present disclosure provides for an engineered nuclease system comprising: (a) any of the endonucleases described herein (e.g. a fusion endonuclease comprising: (a) an N-terminal sequence comprising RuvC, REC, or HNH domains of a Cas endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 696 or a variant thereof; and (b) a C-terminal sequence comprising WED, TOPO, or CTD domains of a Cas endonuclease having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 697-721 or variants thereof; and (b) an engineered guide ribonucleic structure configured to form a complex with the endonuclease comprising: a guide ribonucleic acid configured to hybridize to a target deoxyribonucleic acid sequence; wherein the guide ribonucleic acid sequence is configured to bind to the endonuclease. In some embodiments, the guide ribonucleic acid further comprises a tracr ribonucleic acid sequence. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 86% identity to a SpyCas9 endonuclease. In some embodiments, the system further comprises a source of Mg 2+. In some embodiments, the endonuclease comprises a sequence having at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 8-12, 26-27, or 108. In some embodiments, the guide ribonucleic acid sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to non-degenerate nucleotides of any one of SEQ ID NOs: 33, 34, 44, 78, 84, or 87.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

TABLE A
Selected Sequences Disclosed Herein

| | SEQ | | | | Other | |
|---|---|---|---|---|---|---|
| | ID | Descrip- | | | Inform- | |
| Category | NO: | tion | Type | Organism | ation | Sequence |
| MG3 | 696 | MG3-6 N- | protein | artificial | | MSTDMKNYRIGVDVGDRSVGLAAIEFDDDGL |
| chimeric | | terminal | | sequence | | PIQKLALVTFRHDGGLDPTKNKTPMSRKETR |
| effectors | | fragment | | | | GIARRTMRMNRERKRRLRNLDNVLENLGYSV |
| | | (1-742) | | | | PEGPEPETYEAWTSRALLASIKLASADELNE |
| | | | | | | HLVRAVRHMARHRGWANPWWSLDQLEKASQE |
| | | | | | | PSETFEIILARARELFGEKVPANPTLGMLGA |
| | | | | | | LAANNEVLLRPRDEKKRKTGYVRGTPLMFAQ |
| | | | | | | VRQGDQLAELRRICEVQGIEDQYEALRLGVF |
| | | | | | | DHKHPYVPKERVGKDPLNPSTNRTIRASLEF |
| | | | | | | QEFRILDSVANLRVRIGSRAKRELTEAEYDA |
| | | | | | | AVEFLMDYADKEQPSWADVAEKIGVPGNRLV |
| | | | | | | APVLEDVQQKTAPYDRSSAAFEKAMGKKTEA |
| | | | | | | ROWWESTDDDQLRSLLIAFLVDATNDTEEAA |
| | | | | | | AEAGLSELYKSWPAEEREALSNIDFEKGRVA |
| | | | | | | YSQETLSKLSEYMHEYRVGLHEARKAVFGVD |
| | | | | | | DTWRPPLDKLEEPTGQPAVDRVLTILRRFVL |
| | | | | | | DCERQWGRPRAITVEHTRTGLMGPTQRQKIL |
| | | | | | | NEQKKNRADNERIRDELRESGVDNPSRAEVR |
| | | | | | | RHLIVQEQECQCLYCGTMITTTTSELDHIVP |
| | | | | | | RAGGGSSRRENLAAVCRACNAKKKRELFYAW |
| | | | | | | AGPVKSQETIERVRQLKAFKDSKKAKMFKNQ |
| | | | | | | IRRLNQTEADEPIDERSLASTSYAAVAVRER |
| | | | | | | LEQHFNEGLALDDKSRVVLDVYAGAVTRESR |
| | | | | | | RAGGIDERILLRGERDKNRFDVRHHAVDA |
| MG1 | 697 | MG1-4 C- | protein | artificial | | ICISFSRDFKYDKEIKKDIIKGFNPEIVKNA |
| chimeric | | terminal | | sequence | | IDKIMPYPYANDKPFKGNTKPLETIYGLRTY |
| effector | | fragment | | | | GDKSYITQRVELNSIDKKATKIKSIIDETIK |
| | | | | | | NDLLNKLKENPTEQEWKLMLQNYIHPKKQTK |
| | | | | | | VKKVMISVSEGEITKDSNNRERMGEFVDFGT |
| | | | | | | KGTQHQFKHSKRHKGQILYFNEKGVVEVMPV |
| | | | | | | YSNIKTTDVKDKLQNMGCKLYNKGQMFYSGC |
| | | | | | | LVDIPKPFKAGSKEYPAGRYQIKTIRSDKVA |
| | | | | | | ELEDACGNKISTNVKYLVPAEFKKVESK |
| MG1 | 698 | MG1-5 C- | protein | artificial | | MCICFAPTSNAKKALSRKNILPEEIAKNPES |
| chimeric | | terminal | | sequence | | DDARNFFAKYLAEVVPTKVAIKKPELEQTIY |
| effector | | fragment | | | | SKRVIGGRQTIVKKCNVRDLAYKGQNPKYDF |
| | | | | | | DTLTKRIKDIINPVSKRVIEDFAKTEPTEAE |
| | | | | | | WEDWCKYEAAIPSKNGSPTRLLRVLCKTKDD |
| | | | | | | AERFKDLSKDGCGAYRKSKSHKGQFIWKDNK |
| | | | | | | GNYLVAPVYIYSSKQKVYAELKNNPKCMGIC |
| | | | | | | DFFKTGCLVKISNEVVDEKKNRLWLKAGFYN |
| | | | | | | LNSIAKEKRVYLTDVNGQEHKKIPLQHLMNA |
| | | | | | | GMKRVETNTI |
| MG1 | 699 | MG1-6 C- | protein | artificial | | MCLCFAPTGVDSRRAKLGEILPEKLRSEKAA |
| chimeric | | terminal | | sequence | | REFFKSYLDKIMPVDVAPKKPRLEDGIYSKR |
| effector | | fragment | | | | IIGGKACMVKRNNLVDLAYKSGLKPVFDIPT |
| | | | | | | LIKLVDKKEKGIINPQIRKMIGEFAATNPDE |
| | | | | | | SAWRKWCEEVRLPSKSGLGARVLRVLVYYGE |
| | | | | | | ADEYKDLSKDGCGAYRKGDGHKGQVVWESVD |
| | | | | | | GKYYVEPVYVHASKAGVMAALNANPKKKRIC |
| | | | | | | GMFNSHCTVDVGDVYNDRGDFILPAGRYMVN |
| | | | | | | TILTTGRCVLTNADGEKRNPININYLMRAGM |
| | | | | | | RRVELSEL |
| MG1 | 700 | MG1-7 C- | protein | artificial | | MCLCFAPTGVNSKRARVDMLLPPKIRSEKEA |
| chimeric | | terminal | | sequence | | ELFFRKYLDKLIPVDVAPKKPKLEDGIYSMR |
| effector | | fragment | | | | TVGGKKIMARRVNLVDLAYKSGLKPVYDVSV |
| | | | | | | LIKLLDKKERGIINPQIRKLVADFARTNPSE |
| | | | | | | DEWKKWCGECRLPSKNGLGTRVIRVLLNYGE |
| | | | | | | PAEYKDLSKDGRGAFRRGDGHKGQIVWESTD |
| | | | | | | GKYCVLPIYVHASKAKLLAELCANPKKKRIC |
| | | | | | | GIFTSHCMVKVGNTYNNKGELLLPEGVYMLN |
| | | | | | | TIRTDGWIQLTSANGDKSKPININYLMKAGM |
| | | | | | | KKVPVKDL |
| MG2 | 701 | MG2-4 C- | protein | artificial | | LTLGLATALVPGIERKELRRALSLRQAKGDD |
| chimeric | | terminal | | sequence | | ATLLRSDPKLGEALRWRTEDRFEAAPLSGKL |
| effector | | fragment | | | | ESAVRRALAEGRVVQHVPAKRQGMKVDSNFF |
| | | | | | | GFVEFDETGRLRVRQKMRSPTTRRREIKTTV |
| | | | | | | KNGKNLHTLSHLSLDPKSWLGAPDHPLRRKQ |
| | | | | | | LEHGLRTENDLANPKLGNIRGMLPIRENWGI |
| | | | | | | ALITKDGSPRLDVIPYINVHQWLEVLALENG |
| | | | | | | GGSPVVLRKGHLVGFDAEKCPEEYCGAWMLL |
| | | | | | | GVKDGRSGTTLELIRPWMVAPRKGGTKESSA |
| | | | | | | KQAIKPASGYSEKEGKASGVFLQRSADVFLK |
| | | | | | | LGLRPLDHDLTGIAAF |
| MG2 | 702 | MG2-7 C- | protein | artificial | | VTQGLALLLFAPEDWPLLVKRNLPDSEQRHL |
| chimeric | | terminal | | sequence | | KARYPFLDFSADKHISIQDLPEDTLHTISER |
| effector | | fragment | | | | LAECRVVRHIPAKMHGIIVDQTTWGTVAAGA |
| | | | | | | ITTLRQKTTEKNARCDENGKRFIKTTEKKRS |
| | | | | | | LLLGGPDAPDGKLAKIKGAILVTENWGCALD |
| | | | | | | PSPTVIPHFKVYPQLRALREKNGGRPIRILR |
| | | | | | | KGSLIQVKAGTYQGIWSVASIKDNADGICLD |
| | | | | | | INAADKVKLENRSDDSKINVRLDSLRKSGLK |
| | | | | | | ILKPKLTGACPTTSSP |
| MG3 | 703 | MG3-1 C- | protein | artificial | | AVLTLQSPAIYRVLLTRVNLKHEHEVTGEAP |
| chimeric | | terminal | | sequence | | EWRDYEGADQAEKVLYRRWQKNIATLAELMR |
| effector | | fragment | | | | QEIENNRVPVTRPIRLRKSRGAVHDATVMKA |
| | | | | | | LERDLWGEWDAQAIDRLVDPELHLALRKLFT |
| | | | | | | STKSKKIDVDATSQGLPERYLANQTVQLFDA |
| | | | | | | DAPSVMSPRGILRIGAGTHHARLLTWDDPKK |
| | | | | | | GPQLGIQRVFAAEFGEILKDASSNDLFEAPI |
| | | | | | | PFHTMSHRDLQPKVRAAVEQGLTRQIGWITQ |
| | | | | | | GDELEIDPADFVGEANAFGNFLREFPERSWS |
| | | | | | | IAGLKKSNTIVIRPLLLSQEGVTAAISPHAA |
| | | | | | | KIVENGIELSNSTLFTAPGTGIIRRTGLGRP |
| | | | | | | RWDSGPAHLPESFNVHARMTQQSARD |
| MG3 | 704 | MG3-2 C- | protein | artificial | | AVLTLLDPSVAKTLAMRLDLKREQQDSGRDT |
| chimeric | | terminal | | sequence | | RWKEFKGLTPASQERFIKWCQASECLADMLR |
| effector | | fragment | | | | QQIEADRVPVVVPLRISPSNGAVHDDSVRPL |
| | | | | | | TRQKIDSTWDRKSINRIVDPEIHVAMRRLLN |
| | | | | | | NGTSLPEDKNRVLDLPDGNELGPHDEVELFS |
| | | | | | | TSAASIKLRRGGSAEIGGSIHHARVYAWMGA |
| | | | | | | KGQLEYGMMRVFGAEFPTLTKLSGSKDILRM |
| | | | | | | PIHAGSMSYRDMQDRVRKPIESDIAVELGWI |
| | | | | | | TQGDELEILPEAHLETAGGLGDFLKSFPETQ |
| | | | | | | WTIDGFNDPSRLRVRPRLMSLEGRDTIDAMG |
| | | | | | | HLSDTEKLKIKQALSKGLMVSASELLSHGAK |
| | | | | | | IIRRDHLGRPRWRGNARPVSIELEQVANQLV |
| | | | | | | NHRSVDGQ |
| MG3 | 705 | MG3-3 C- | protein | artificial | | AVMTLLNPSVAVTLEQRRMLKQENDYSSPRG |
| chimeric | | terminal | | sequence | | QHDNGWRDFIGRGEASQSKFLHWKKTAVVLA |
| effector | | fragment | | | | DLISEAIEQDTIPVVNPLRLRPQNGSVHKDT |
| | | | | | | VEAVLERTVGDSWTDKQVSRIVDPNTYIAFL |
| | | | | | | SLLGRKKELDADHQRLVSVSAGVKLLADERV |
| | | | | | | QIFPEEAASILTPRGVVKIGDSIHHARLYGW |
| | | | | | | KNQRGDIQVGMLRVFGAEFPWFMRESGVKDI |
| | | | | | | LRVPIPQGSQSYRDLAATTRKFIENGQATEF |
| | | | | | | GWITQNDEIEISAEEYLATDKGDILSDFLGI |
| | | | | | | LPEIRWKVTGIEDNRRIRLRPLLLSSEAIPN |
| | | | | | | MLNGRLLTQEEHDLIALVINKGVRVVVSTFL |
| | | | | | | ALPSTKIIRRNNLGIPRWRGNGHLPTSLDIQ |
| | | | | | | RAATQALEGRD |
| MG3 | 706 | MG3-4 C- | protein | artificial | | AVMTLLNRSVALTLEQRSQLRRAFYELELDK |
| chimeric | | terminal | | sequence | | LDRDQLKPGEDWRNFTGLYEASQNKFSEWKK |
| effector | | fragment | | | | AATVLGDLLAEAIEDDAIAVVSPLRLRPQNG |
| | | | | | | SVHDDTINAVKKLTLGSAWPADAVKRIVDPE |
| | | | | | | IYLAMKDVLGKLKELPEDSARSLELSDGRYI |
| | | | | | | EADDEVLFFPKKAASILTPRGAAEIGNSIHH |
| | | | | | | ARLYSWLTKKGELKFGMLRVYGAEFPWLMRE |
| | | | | | | SGSRDVLHMPIHPGSQSFRGMQDGVRKAVES |
| | | | | | | GEAVEFGWITQDDELEFDPEDYIAHGGDDEL |
| | | | | | | NRLLRVMPERRWRVDGFYNAGTLRIRPALLS |
| | | | | | | AEQLPSELQKKVADKTLSDVELILLRAVQRG |
| | | | | | | LFVAISSFLPLESLKVIRRNNLGFPRWRGNG |
| | | | | | | NLPTSFEVRSSALRALGVEG |
| MG3 | 707 | MG3-7 C- | protein | artificial | | AVLTLLNRSVAVTLEQRRLIKQQREYSLEKS |
| chimeric | | terminal | | sequence | | RRERDNVWRDFMGLGPAAQEKFAKWKKTAYV |
| effector | | fragment | | | | LADIIKEAISNDAIPVVSPLRLRPQNGSVHL |
| | | | | | | DTVDAVLERTIGDAWTVDQVHRIVNPQIYLA |
| | | | | | | FAGYLGNQKALDPDSSRVLALNDGRKLTAED |
| | | | | | | VIYVFPEKAASILTPRGVVKIGESVHHVRLY |
| | | | | | | AWKNRKGKAEVGMLRVFGAEFPWLMRESGVK |
| | | | | | | DVLRVPIHTGSQSYRDLSFTVRKNIEKGEAA |
| | | | | | | EIGWLTQNEELEFNPESYLQEGGKDKLAKFL |
| | | | | | | AFLPETRWRVDGFPMPDKLRIRPALLSREEI |
| | | | | | | PEGVFRTEEQSLLEEALTKGLIIATKGLLSL |
| | | | | | | PDVKVLRRNNLGIPRWRGGSYRPVSLDIQRA |
| | | | | | | ALAALDEQE |
| MG3 | 708 | MG3-8 C- | protein | artificial | | AVMTLLNRSVALTLEQRSQLRRAFYEQGLDK |
| chimeric | | terminal | | sequence | | LDRDQLKPEEDWRNFIGLSLASQEKFLEWKK |
| effector | | fragment | | | | VTTVLGDLLAEAIEDDSIAVVSPLRLRPQNG |
| | | | | | | RVHKDTIAAVKKQTLGSAWSADAVKRIVDPE |
| | | | | | | IYLAMKDALGKSKVLPEDSARTLELSDGRYL |
| | | | | | | EADDEVLFFPKNAASILTPRGVAEIGGSIHH |
| | | | | | | ARLYSWLTKKGELKIGMLRVYGAEFPWLMRE |
| | | | | | | SGSHDVLRMPIHPGSQSFRDMQDTTRKAVES |
| | | | | | | SEAVEFAWITQNDELEFEPEDYIAHGGKDEL |
| | | | | | | RQFLEFMPECRWRVDGFKKNYQIRIRPAMLS |
| | | | | | | REQLPSDIQRRLESKTLTENESLLLKALDTG |
| | | | | | | LVVAIGGLLPLGTLKVIRRNNLGFPRWRGNG |
| | | | | | | NLPTSFEVRSSALRALGVEG |
| MG4 | 709 | MG4-2 C- | protein | artificial | | VAIALTDPAALKSISQAASDERRGGRVSFGA |
| chimeric | | terminal | | sequence | | VALPWVDFIGDVQAAIEAINVSHRPSRKVNG |
| effector | | fragment | | | | ALHEETFYGPRGMDGDGRPTGYVQRKPVERL |
| | | | | | | SAKEIPNIPDPAVREAVQAKLDEVGGTPAQA |
| | | | | | | FKDPANHPVRKRGIPVHKVRLRLNINPVQVG |
| | | | | | | SGATERHVLTGSNHHMEIIEVRDAKGGKKWT |
| | | | | | | GRLVHRLEAKRRALGRETIVDRAVQAGRQFQ |
| | | | | | | FSLSPGDMIELTGEDGERKLHVVRSISEGRI |
| | | | | | | EYVDARDARKKADIRASGDWRKPAVGSLLRL |
| | | | | | | HCRKVVVTPFGEIRYAND |
| MG4 | 710 | MG4-5 C- | protein | artificial | | VVIALTGPGTVQALTRAALRAKELGRRLFVP |
| chimeric | | terminal | | sequence | | LDPPWADRDSFLRDVRASVEAITVSYRVDRK |
| effector | | fragment | | | | VSGQLHEESNYSKPHMTVDNKGNLVEHRHIR |
| | | | | | | KPLKDMSVEEVEAIVDDRVRKLVQEKLRQLG |
| | | | | | | QEPKKAFADEANHPYFTTADGRLVPIHKARI |
| | | | | | | RKTVATITVGPPQCPRHVAPGLNHHIEILAV |
| | | | | | | RDPAGAVTHWEGELVSLFEAARRVKAGEPVV |
| | | | | | | RRNHGPNKDFLFSLAKGEYVEMELQPGKRQL |
| | | | | | | FRVTVISAKQIEFRLHHDARPTMLLRKTPGA |
| | | | | | | RVIRSPGSLFKAKARKVAVDPLGNVFPAND |
| MG6 | 711 | MG6-3 C- | protein | artificial | | IVVAFTNRSTLKRLSDENKRIGTAEWMDADE |
| chimeric | | terminal | | sequence | | SGRATNDEIKRRLGGRIDLSEPWPTFRNDVE |
| effector | | fragment | | | | VSINNITVSHRVNRKVSGALHEETYYGPTDE |
| | | | | | | PAPKNKEMMVLRKSVHQLSKKDLGLIRDETI |
| | | | | | | RQIVNDEVQKRMDNGESQANAIASLEADPPF |
| | | | | | | IISPKAKVPIRKVRLLMKKDPQIMHYFENKN |
| | | | | | | GEEDRAALYGNNHHIAIYETSDKNGVKKQIG |
| | | | | | | IVIPMMEAARRVKDGDPIVMKDYRPDHTFLY |
| | | | | | | SLAKNDMIFNHEDEQIYRVQKINSDGTIMFR |
| | | | | | | QNNVAMKGQSDPGVYFKSGSRLGASKIKISP |
| | | | | | | IGEIFPAND |
| MG14 | 712 | MG14-1 C- | protein | artificial | | CVIAACSPSLVIKTARINQETHWSITRGMNE |
| chimeric | | terminal | | sequence | | TQRRDAIMKALESVMPWETFANEVRAAHDFV |
| effector | | fragment | | | | VPTRFVPRKGKGELFEQTVYRYAGVNAQGKD |
| | | | | | | IARKASSDKDIVMGNAVVSADEKSVIKVSEM |
| | | | | | | LCLRLWHDPEAKKGQGAWYADPVYKADIPAL |
| | | | | | | KDGTYVPRIAKAHTGRKAWKPVPESAMAKPP |
| | | | | | | LEIYFGDLVQIGDFIGRFSGYNINNANWSFT |
| | | | | | | DRLTRLNLSCPTVGQLNNDLSPVVIRESPIK |
| MG15 | 713 | MG15-1 C- | protein | artificial | | VIIACATQGIVNKVSRYSKSRELWDYEVDME |
| chimeric | | terminal | | sequence | | TGEVLQKKNKNTKDVFPEPWLNFRYELEQKV |
| effector | | fragment | | | | RVRPLDIPETADITEMEEPFVSHMPNRKIHG |
| | | | | | | PAHKETIRSGRLKEEGYTISKTALIDLKLTE |
| | | | | | | DKEEIKGYYNKESDRLLYEALKKQLQRYGGK |
| | | | | | | AKEAFKEPFHKPKADGTPGPIVNKVKIMEKS |
| | | | | | | TMLIPVNGGKGLASNGNMVRIDVFRAEEKGK |
| | | | | | | KKYYFIPVYVADTVKEELPNRAVLAHKPYEA |
| | | | | | | WKIMKEENFIFSLYPNDLIFVDAGKEIPFKA |
| | | | | | | ALKGSTLDPEKKASRFLMYYKGADIATGSIS |
| | | | | | | GVNHDETYKARGVGIQSLREIKKCCIDVLGN |
| | | | | | | ISFASKEKRQTFR |
| MG16 | 714 | MG16-1 C- | protein | artificial | | LTVALTRQSYIQRLNTLEASHEHMEKLVKEA |
| chimeric | | terminal | | sequence | | NTPYKEKKSLLEKWVALQPHFSVEEVTTQVD |
| effector | | fragment | | | | GILVSFRAGKRVTTPARRAVYHGGKRTIVQR |
| | | | | | | GIQVPRGALTEDTIYGKLGDKFVVKYALDHP |
| | | | | | | SMKPENIVDPTIRLLVENRITALGKKDAFKT |
| | | | | | | PLYSAEGMEIKSVRCYTSLSEKGVVPIKYNE |
| | | | | | | KGNAIGFAKKGNNHHVAIYKDQSGQYQEMVV |
| | | | | | | SFWDAVERKLYGVPTVITNPKTVWDELLEKE |
| | | | | | | LPQDFLEKLPKDNWQYVLSMQENEMFVLGME |
| | | | | | | EDEFNDAIDTQDYNTLNKHLYRVQKLSHADY |
| | | | | | | TFRFHTETKVDDKYDGVENGRNTSMSLKALV |
| | | | | | | RIRSFNGLFTQFPHKVKIDIMGRITKA |
| MG16 | 715 | MG16-2 C- | protein | artificial | | LVVACTKQSYIQRLNNLNTERDAMYQDIEAQ |
| chimeric | | terminal | | sequence | | SVEWKEKHSLLEKWIKLQPHPTVSEVTDKVD |
| effector | | fragment | | | | EILVSFKAGKRVATLGKRSVYKNGKKTVVQN |
| | | | | | | NIIVPRGALCEESVYGQINLIEKNKPIKYLF |
| | | | | | | ENPSLIFKPYIKALVEERLKEYNGDTSKAIS |
| | | | | | | SLKNNPIYLRKDKSVVLEYGTCYKKEYVKKY |
| | | | | | | SLNSIKAKDVDSIIDKHIREVVRQRLEDNNN |
| | | | | | | NEKAAFASPLYADKQKQIPIKSVRCTTGINI |
| | | | | | | AAPVNYNESNDPISFVKPGNNHHIAIYKDKD |
| | | | | | | GKRQEHIVTFWHAVERKKYGMPVVITNPKEI |
| | | | | | | WDLIIEKSLDLPESFLNCLPNSDWNYEISMQ |
| | | | | | | QNEMFVMGMSEDEFQDAIRNNDYKTLNKYLY |
| | | | | | | RVQSVSESDYWLRLHIETMNDKTPEGNIIKK |
| | | | | | | YYRIKSINTFFNFNPHKVKITLLGEIQSS |
| MG18 | 716 | MG18-1 C- | protein | artificial | | YLNAVVGNVYHEKFTKNPLRFVRSGQEYSLN |
| chimeric | | terminal | | sequence | | LSALFQNWNIYKGGRVIWQKGEDGSLETVRA |
| effector | | fragment | | | | RMAKNDPMVTRYCTEGRGALYDLQPMKKSKG |
| | | | | | | QLPLKSSDERLQHIDRYGGYNKLAGAYFTLA |
| | | | | | | AYYKKGKRVKSIESVPLYLAAKLQRDPAALQ |
| | | | | | | QYLADQLGTDRVEILVPEIKLGTLFKWNGYP |
| | | | | | | MTLSGRTGPQLLFRNAAELRTNAEQEQYIKK |
| | | | | | | MSRYLEKCKGRKEPLPIRPAYDKLTPEENLQ |
| | | | | | | LYDAFTQWLTSGIYAKRLSLQGKFLLEKRDA |
| | | | | | | FAALSPEAQVRQLMEILHLFQCNPVAANLSE |
| | | | | | | LGGAAHAGILLASKNIDGKVPVSIVHQSVTG |
| | | | | | | YFTQEVCLNDL |
| MG21 | 717 | MG21-1 C- | protein | artificial | | AVIACITPGMIQKITKYAQNHERFYATAKGY |
| chimeric | | terminal | | sequence | | VDIETGEVLTRSEYEAMDDIRFPEPWPGFRS |
| effector | | fragment | | | | ELEARVSEHPQEAIARLKLPHYENSEEIRPI |
| | | | | | | FVSRMPNHKVTGAAHLETIRSKKGGAGSTVT |
| | | | | | | KTALPDLKLDKNGEIAGYYRKEDDPLLYEAL |
| | | | | | | KARLKAFGGDGKKAFAEPFHKPKHNGEPGPI |
| | | | | | | VKKVKIQESATLTVPVNHGIAANGSMVRLDV |
| | | | | | | FHVDGDGYYFVPIYTSDTVKPELPNRAVVAG |
| | | | | | | RRVQEWKVMDDSYFKFSLYPKDLIRIRSKKG |
| | | | | | | IKLKAVNRNADLQEYSTNDCLCYFVKFNIST |
| | | | | | | GALSVENHDRKFEQPGLGGKTLLSIEKYQVD |
| | | | | | | VLGNYSPVALPEKRMKFR |
| MG22 | 718 | MG22-1 C- | protein | artificial | | IAIACINRSIVNYLNNAAANQTEREDLRRAV |
| chimeric | | terminal | | sequence | | CIPERNGQTKRQLRSPWHCFARDAENALRQI |
| effector | | fragment | | | | VVSFKQNLRVATKATNSYECFDTASGKKIRK |
| | | | | | | HQSNREHYAIRKPLHKDSVYGEVILTSIASV |
| | | | | | | NLKKALLKAERILDKRLKEKIFELRKLYNYS |
| | | | | | | NKQIEEHLTKVCINCPEWKNYDFKKIAVRIL |
| | | | | | | SNDADATHIVAIRKPLDESFDEVKINTITDT |
| | | | | | | GIQKILLNHLSRYADDPKKAFSPEGIEDMNA |
| | | | | | | NIASLNGGKQHLPIYKVRVSEKDNGGYFPIG |
| | | | | | | QKGNRPKKYVTTAKDTNLFFAVYADSKGKRS |
| | | | | | | YKTIDLRTAIECRKQGLSVAPSINEKGDKLL |
| | | | | | | FTLSPNDLVYMPSEGEEANGFAIDNNLNKDQ |
| | | | | | | IYKMVSANNKQCFFIPHTVADFISRGEEYNS |
| | | | | | | HNKIELTEDRRSIKEHCVPLKVNRLGK |
| MG23 | 719 | MG23-1 C- | protein | artificial | | YLNIVVGNTYSTKFTNNPLNFIKAGAKRPQD |
| chimeric | | terminal | | sequence | | NQFKYNMDKIFDYNVISRGERAWIAGSDGSI |
| effector | | fragment | | | | CTVKKFMSRNTVLITRKAKEVHGALSNKATI |
| | | | | | | WGKNVAKPGAYLPVKSTDLKAQDVTKYGGIT |
| | | | | | | SIANSGYTLAEYKVNGKTTRSLEALPVYLGR |
| | | | | | | AEQLTEKTVVDYLSSSLQESSKKKIEDIQVR |
| | | | | | | KLFIPQGSKVKIDGFCYYLGGKTGDSIYLNN |
| | | | | | | AVPLYLSSTSEEYLRKLLKAVENNNYNERDK |
| | | | | | | NGQIILTAPKNVQLLSSIFDKLRSKPFSNNK |
| | | | | | | WNIYFSIVNGKETKVEQLFSKLSIDKQAEVI |
| | | | | | | SQIVIWINSSRQNVNLSLIGGSAHSGTQALS |
| | | | | | | KTVSRLNECMLISQSITGIYEHSVDLLTI |
| SaCas | 720 | SaCas9 C- | protein | artificial | | LIIANADFIFKEWKKLDKAKKVMENQMFEEK |
| chimeric | | terminal | | sequence | | QAESMPEIETEQEYKEIFITPHQIKHIKDFK |
| effector | | fragment | | | | DYKYSHRVDKKPNRELINDTLYSTRKDDKGN |
| | | | | | | TLIVNNLNGLYDKDNDKLKKLINKSPEKLLM |
| | | | | | | YHHDPQTYQKLKLIMEQYGDEKNPLYKYYEE |
| | | | | | | TGNYLTKYSKKDNGPVIKKIKYYGNKLNAHL |
| | | | | | | DITDDYPNSRNKVVKLSLKPYRFDVYLDNGV |
| | | | | | | YKFVTVKNLDVIKKENYYEVNSKCYEEAKKL |
| | | | | | | KKISNQAEFIASFYNNDLIKINGELYRVIGV |
| | | | | | | NNDLLNRIEVNMIDITYREYLENMNDKRPPR |
| | | | | | | IIKTIASKTQSIKKYSTDILGNLYEVKSKKH |
| | | | | | | PQIIKKG |
| SpCas | 721 | SpCas9 C- | protein | artificial | | YLNAVVGTALIKKYPKLESEFVYGDYKVYDV |
| chimeric | | terminal | | sequence | | RKMIAKSEQEIGKATAKYFFYSNIMNFFKTE |
| effector | | fragment | | | | ITLANGEIRKRPLIETNGETGEIVWDKGRDF |
| | | | | | | ATVRKVLSMPQVNIVKKTEVQTGGFSKESIL |
| | | | | | | PKRNSDKLIARKKDWDPKKYGGFDSPTVAYS |
| | | | | | | VLVVAKVEKGKSKKLKSVKELLGITIMERSS |
| | | | | | | FEKNPIDFLEAKGYKEVKKDLIIKLPKYSLF |
| | | | | | | ELENGRKRMLASAGELQKGNELALPSKYVNF |
| | | | | | | LYLASHYEKLKGSPEDNEQKQLFVEQHKHYL |
| | | | | | | DEIIEQISEFSKRVILADANLDKVLSAYNKH |
| | | | | | | RDKPIREQAENIIHLFTLTNLGAPAAFKYFD |
| | | | | | | TTIDRKRYTSTKEVLDATLIHQSITGLYETR |
| | | | | | | IDLSQLGGD |
| MG3-6_3-4 | 722 | MG3-6_3-4 | Nucleo- | | | NNNNNNNNNNNNNNNNNNNNNNGTTGAGAAT |
| guide | | guide | tide | | | CGAAAGATTCTTAATAAGGCATCCTTCCGAT |
| sgRNA | | sequence | (RNA) | | | GCTGACTTCTCACCGTCCGTTTTCCAATAGG |
| scaffold | | scaffold | | | | AGCGGGCGGTATGTTTT |

### EXAMPLES

### Example 1—Plasmids

Chimera sequences were codon optimized for E. coli expression via Integrated DNA Technologies (IDT) website, and synthesized and cloned into pET21 vector at Twist Bioscience unless otherwise specified. To construct pET21-MG3-6+MG15-1 (WP) and pET21-MG3-6+MG15-1 (P), gene fragments were amplified from pMGX3-6 and pMGX15-1 using primers P441-P446. The resulting PCR products were purified by Zymo Gel DNA Recovery Kit and assembled into pAL3 (digested by ClaI and XhoI) via NEBUilder HiFi DNA assembly. DNA sequences of cloned chimeric genes were confirmed by Sanger sequencing service offered by Elim Biopharm.

### Example 2—Bioinformatic Analysis

CRISPR Type II endonucleases utilized herein were predicted to have nuclease activity based on the presence of putative HNH and RuvC catalytic residues. In addition, structural predictions suggested residues involved in guide, target, and recognition of and interaction with a PAM. Based on the location of important residues, the predicted domain architecture of Type II CRISPR endonucleases comprised three RuvC domains, an HNH endonuclease domain, a recognition domain and PAM interacting domain, among others. For genomic sequences encoding a full-length Type II endonuclease next to a CRISPR array, we predicted tracrRNA sequences, which were engineered to be used by the nuclease as single guide RNAs.

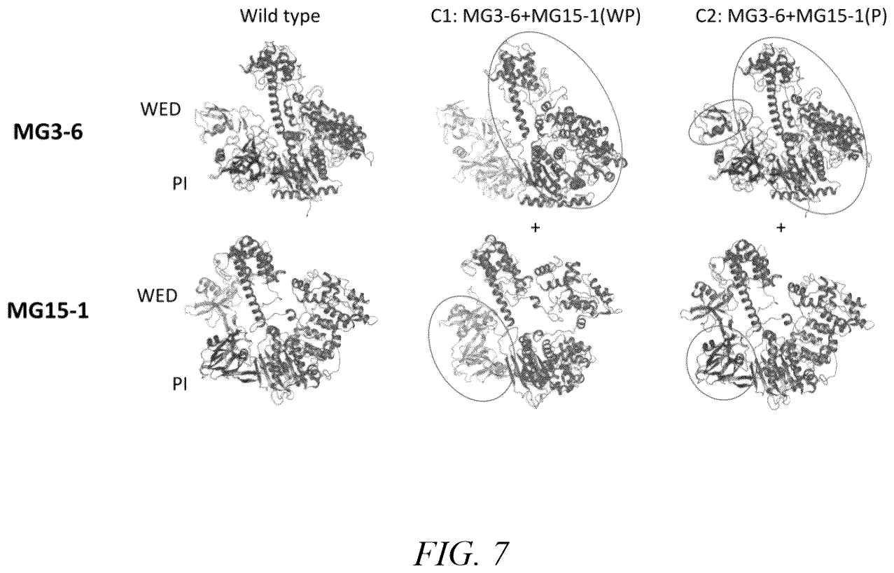

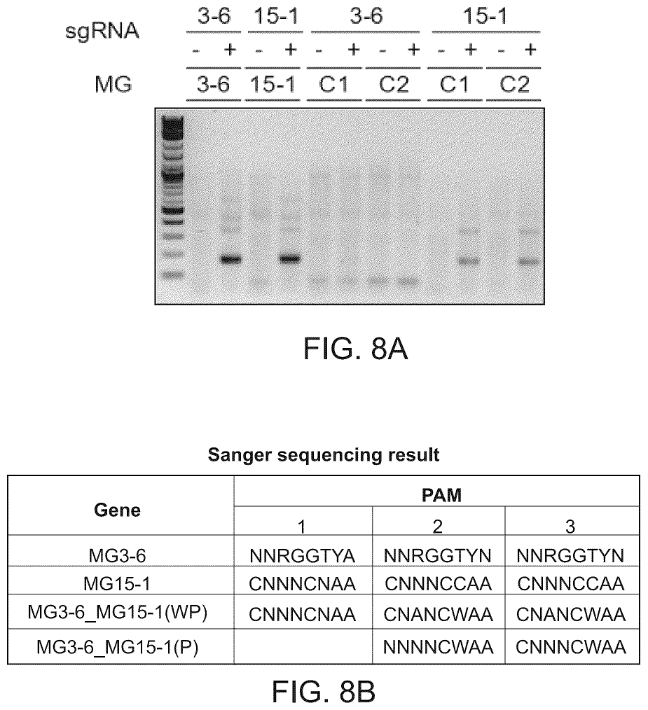

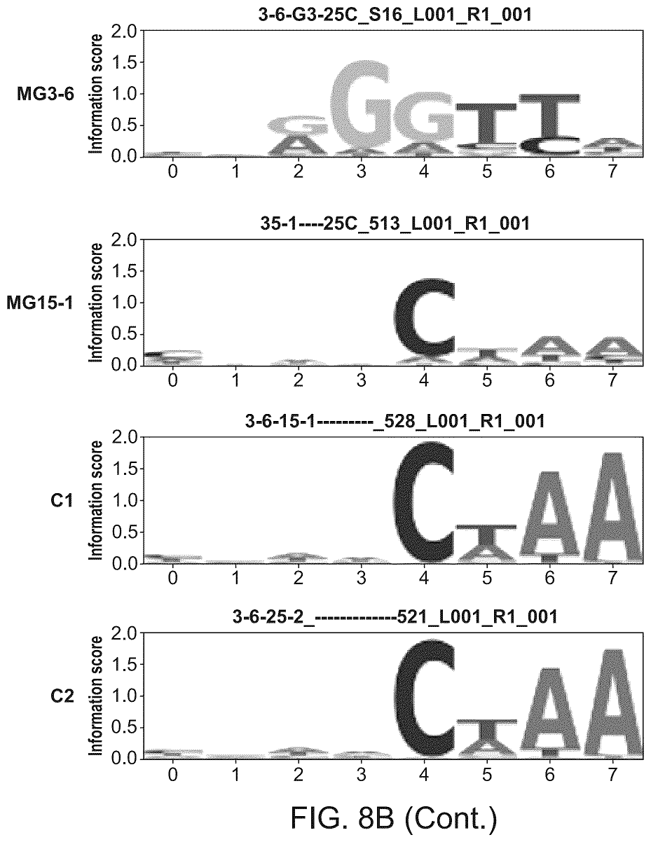

A multiple sequence alignment of selected RNA guided CRISPR Type II endonuclease sequences were performed using the built-in MUSCLE aligner on Geneious Primer Software (available at https://www.geneious.com/prime) (see FIG. 3). Protein structures of MG3-6 and MG15-1 were predicted with DNASTAR NovaFold and displayed via Protean 3D. Details of chimeric compositions are shown in Table 1. Guided by predicted structural model information along with guide RNA optimization (see FIG. 7), we engineered protein variants recognizing non-canonical PAMs by concatenating domains from closely, as well as distantly related Type II CRISPR endonucleases.

TABLE 1
Chimeric Compositions

| | | | Example Sequence |
|---|---|---|---|
| Chimera | N-terminus | C-terminus | (SEQ ID NO:) |
| MG3-6 + MG1-4 | MG3-6 (1-742) | MG1-4 (750-1025) | 1 |
| MG3-6 + MG1-5 | MG3-6 (1-742) | MG1-5 (789-1077) | 2 |
| MG3-6 + MG1-6 | MG3-6 (1-742) | MG1-6 (773-1059) | 3 |
| MG3-6 + MG1-7 | MG3-6 (1-742) | MG1-7 (775-1061) | 4 |
| MG3-6 + MG2-4 | MG3-6 (1-742) | MG2-4 (876-1201) | 5 |
| MG3-6 + MG2-7 | MG3-6 (1-742) | MG2-7 (817-1080) | 6 |
| MG3-6 + MG3-1 | MG3-6 (1-742) | MG3-1 (684-1050) | 7 |
| MG3-6 + MG3-2 | MG3-6 (1-742) | MG3-2 (755-1134) | 8 |
| MG3-6 + MG3-3 | MG3-6 (1-742) | MG3-3 (750-1132) | 9 |
| MG3-6 + MG3-4 | MG3-6 (1-742) | MG3-4 (743-1134) | 10 |
| MG3-6 + MG3-7 | MG3-6 (1-742) | MG3-7 (751-1131) | 11 |
| MG3-6 + MG3-8 | MG3-6 (1-742) | MG3-8 (741-1132) | 12[TB1] |
| MG3-6 + MG4-2 | MG3-6 (1-742) | MG4-2 (747-1043) | 13 |
| MG3-6 + MG4-5 | MG3-6 (1-742) | MG4-5 (747-1055) | 14 |
| MG3-6 + MG6-3 | MG3-6 (1-742) | MG6-3 (709-1027) | 15 |
| MG3-6 + MG14-1 | MG3-6 (1-742) | MG14-1 (756-1003) | 16 |
| MG3-6 + MG15-1 | MG3-6 (1-742) | MG15-1 (729-1082) | 17 |
| MG3-6 + MG16-1 | MG3-6 (1-742) | MG16-1 (787-1154) | 18 |
| MG3-6 + MG16-2 | MG3-6 (1-742) | MG16-2 (796-1227) | 19 |
| MG3-6 + MG18-1 | MG3-6 (1-742) | MG18-1 (997-1348) | 20 |
| MG3-6 + MG21-1 | MG3-6 (1-742) | MG21-1 (740-1098) | 21 |
| MG3-6 + MG22-1 | MG3-6 (1-742) | MG22-1 (1092-1521) | 22 |
| MG3-6 + MG23-1 | MG3-6 (1-742) | MG23-1 (1008-1377) | 23 |
| MG3-6 + SaCas9 | MG3-6 (1-742) | SaCas9 (706-1053) | 24 |
| MG3-6 + SpCas9 | MG3-6 (1-742) | SpCas9 (988-1368) | 25 |
| MG29-1 + MG29-5 (WP) | MG29-1 (1-560) | MG29-5 (556-856) | 109 |
| MG3-6 + MG15-1(WP) | MG3-6 (1-840) | MG15-1 (818-1082) | 26 |
| MG3-6 + MG15-1(P) | MG3-6 (1-922) | MG15-1 (931-1082) | 27 |
| MG29-1 + MG57-1 (WP) | MG29-1 (1-560) | MG57-1 (633-945) | 110 |

### Example 3—In Vitro PAM Enrichment Assay

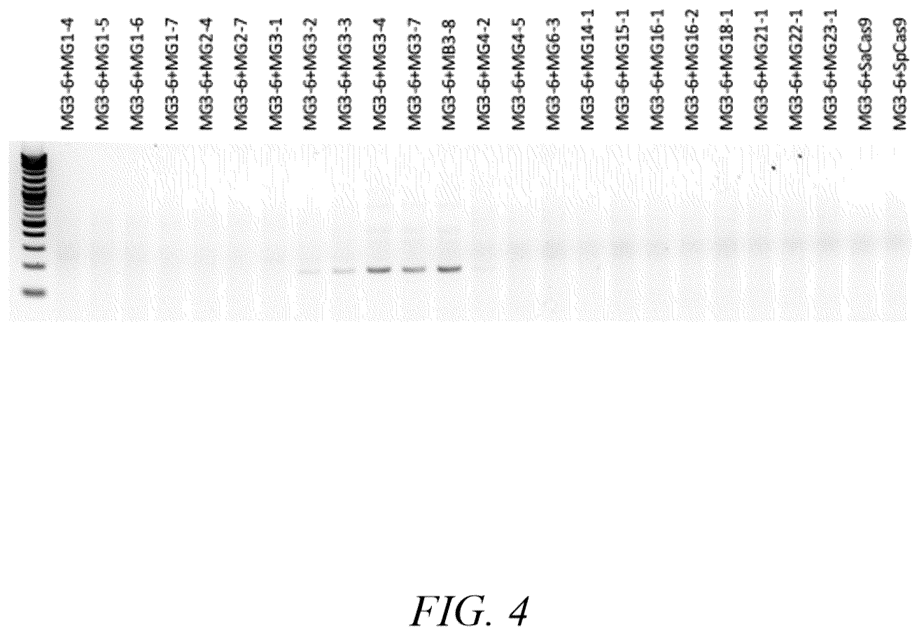

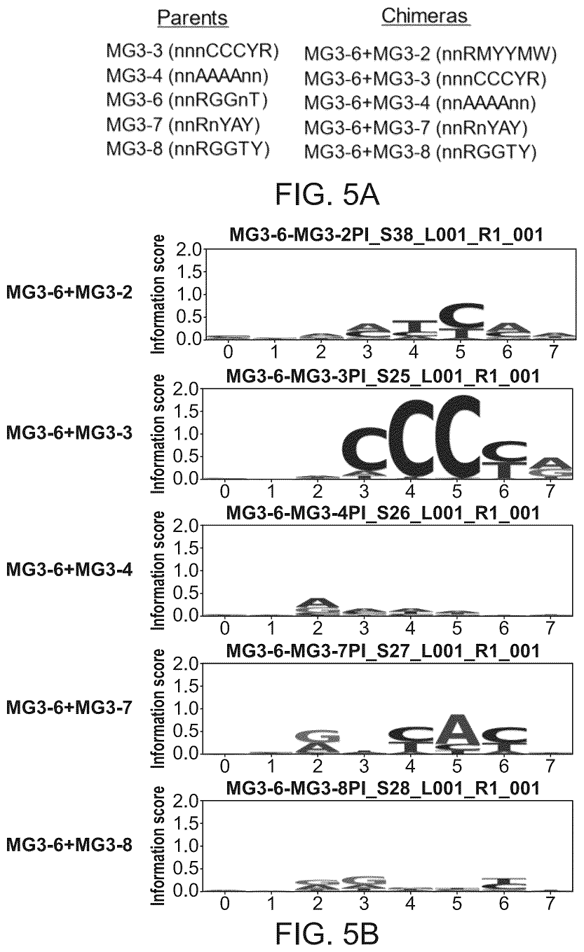

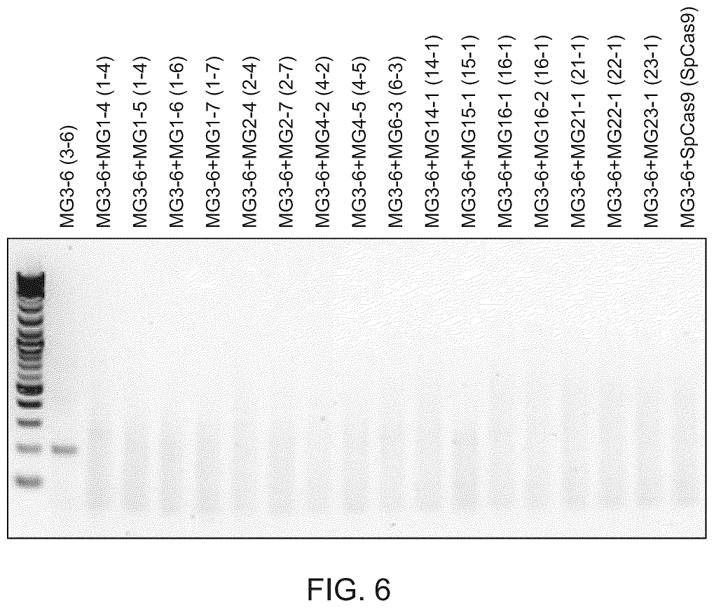

The PAM sequences of nucleases utilized herein were determined via expression in either an E. coli lysate-based expression system or reconstituted in vitro translation (myTXTL, Arbor Biosciences or PURExpress, New England Biolabs). The E. coli codon optimized protein sequence was transcribed and translated from a PCR fragment under control of a T7 promoter. This mixture was diluted into a reaction buffer (10 mM Tris pH 7.5, 100 mM NaCl, 10 mM MgCl₂) with protein-specific sgRNA and a PAM plasmid library (PAM library U67/U40). The library of plasmids contained a spacer sequence matching that in the single guide followed by 8N mixed bases, a subset of which were presumed to have the correct PAM. After 1-3 h, the reaction was stopped and the DNA was recovered via a DNA clean-up kit, e.g. Zymo DCC, AMPure XP beads, QiaQuick etc. The DNA was subjected to a blunt-end ligation reaction which added adapter sequences to cleaved library plasmids while leaving intact circular plasmids unchanged. A PCR was performed with primers (LA065 and LA125) specific to the library and the adapter sequence and resolved on a gel to identify active protein complexes (see FIG. 4 and FIG. 6). The resulting PCR products were further amplified by PCR using high throughput sequencing primers (TrueSeq) and KAPA HiFi HotStart with a cycling parameter of 8. Samples subjected to NGS analysis were quantified by 4200 TapeStation (Agilent Technologies) and pooled together. The NGS library was purified via AMPure XP beads and quantified with KAPA Library Quant Kit (Illumina) kit using AriaMx Real-Time PCR System (Agilent Technologies). Sequencing this library, which was a subset of the starting 8N library, revealed the sequences which contain the correct PAM (see FIG. 5).

### Example 4—Single Guide Design for In Vivo Targeting

The single guide (sgRNA) structures used herein comprised a structure of: 5′ 22nt protospacer-repeat—tracr—3′. 20 single guides targeting mouse albumin intron 1 were designed using Geneious Prime Software (https://www.geneious.com/prime/). In some instances, guides were chemically synthesized by IDT and included a chemical modification of the guide that had been optimized by IDT to improve the performance of Cas9 guides (“Alt-R” modifications).

### Example 5—In Vitro Transcription of mRNA

The coding sequences (CDS) encoding the chimeras (e.g. MG3-6+MG3-4 (SEQ ID NO: 10)) were codon-optimized for mouse and chemically synthesized at Twist biosciences. The CDS were cloned into mRNA production vector pMG010. The architecture of pMG010 comprised the sequence of elements: T7 promotor—5′UTR—start codon—nuclear localization signal 1—CDS—nuclear localization signal 2—stop codon—3′ UTR—107 nucleotide polyA tail (SEQ ID NO: 108). A plasmid pMG010 containing the MG3-6+MG 3-4 CDS was purified from a 200 ml bacterial culture using an EndoFree Plasmid Kit (Qiagen). The vector was digested with SapI overnight in order to linearize the plasmid downstream of the polyA tail. The linearized vector was purified using phenol/chloroform DNA extraction. In vitro transcription was carried out using HiT7 T7 RNA polymerase (New England Biolabs) at 50° C. for 1 h. In vitro transcribed mRNA was treated with DNase for 10 min at 37° C., and the mRNA was purified using the MEGAclear Transcription Clean-up kit (Thermo Fisher). mRNA was quantified by absorbance at 260 nm and its size and purity was assessed by automated electrophoresis (TapeStation, Agilent) and demonstrated to be of the expected size.

### Example 6—Transfection of Hepa1-6 Cells and Albumin Targeting

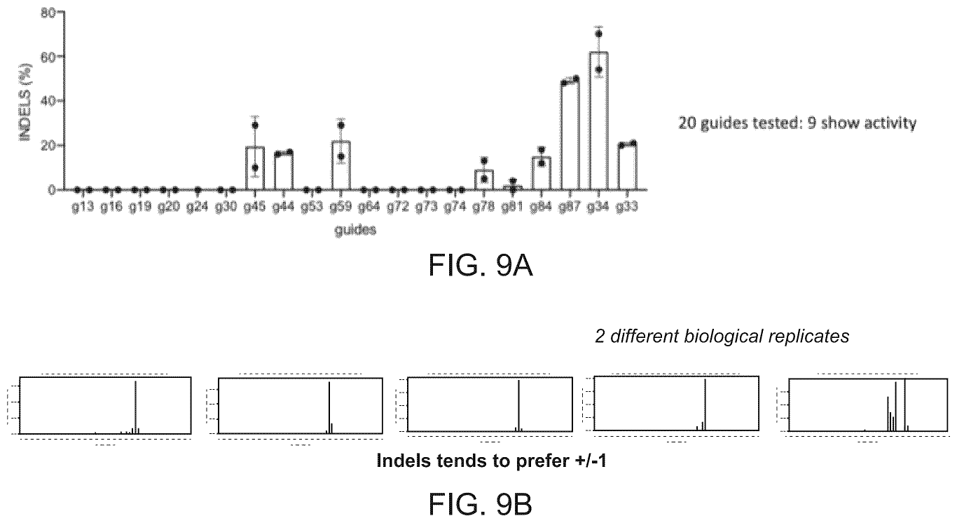

300 ng of mRNA and 350 ng of each single guide RNA (sgRNA) of SEQ ID NOs: 67-86 were co-transfected into Hepa1-6 cells as follows. One Day before transfection Hepa1-6 cells were seeded into 24 wells at a density to achieve 70% confluency 24 h later. The following day 25 μl of OptiMEM media and 1.25 μl of Lipofectamine Messenger Max Solution (Thermo Fisher) were mixed and vortexed for 5 s to make solution A. In a separate tube 300 ng of the MG3-6+MG3-4 chimera mRNA and 350 ng of a single guide were mixed together with 25 μl of OptiMEM to make Solution B. Solution A and B were mixed and incubated for 10 min at room temperature then added directly to the Hepa1-6 cells. Two days post transfection the media was aspirated, and genomic DNA was purified following the instructions from Purelink Genomic DNA mini kit (Thermo Fisher) (see FIG. 9). The results indicate that the best performing sgRNAs were those designated g87 (SEQ ID NO:72) and g34 (SEQ ID NO: 70), with appreciable editing occurring also for gRNAs g45 (SEQ ID NO: 67), g44 (SEQ ID NO: 71), g59 (SEQ ID NO: 76), g78 (SEQ ID NO: 68), g84 (SEQ ID NO: 79), and g33 (SEQ ID NO: 80).

### Example 7—Sanger Sequencing of Genome Edited Samples

Primers flanking the regions of the genome targeted by the single guide RNAs (e.g. the albumin gene) were designed. PCR amplification using primers 57F (SEQ ID NO: 97) and 1072R (SEQ ID NO: 98) was performed using Phusion Flash High-Fidelity PCR Master Mix (Thermo Fisher) resulting in a PCR product of 1016 bp. PCR products were purified and concentrated using DNA clean & concentrator 5 (Zymo Research) and 100 ng of PCR product subjected to Sanger sequencing (ELIM Biosciences) using 8 pmoles of individual sequencing primers (132F, 282F, 446R, and 460F, SEQ ID NOs: 99-102). Sanger sequencing results were analyzed by using an algorithm called Inference of CRISPR edits (available at https://github.com/synthego-open/ice) and data was plotted using GradPrism (see FIG. 9B).

### Example 8—MG3-6/3-4 Nuclease Guide Screen for Mouse HAO-1 Gene Using mRNA Transfection

Guide RNA for the MG3-6/3-4 nuclease targeting exons 1 to 4 of the mouse HAO-1 gene (encodes glycolate oxidase) were identified in silico by searching for the PAM sequence 3′ NNAAA(A/T)N 5′. A total of 23 guides with the fewest predicted off-target sites in the mouse genome were chemically synthesized as single guide RNAs. 300 ng mRNA and 120 ng single guide RNA were transfected into Hepa1-6 cells as follows. One day prior to transfection, Hepa1-6 cells that have been cultured for less than 10 days in DMEM, 10% FBS, 1×NEAA media, without Pen/Strep, were seeded into a TC-treated 24 well plate. Cells were counted, and the equivalent volume to 60,000 viable cells were added to each well. Additional pre-equilibrated media was added to each well to bring the total volume to 500 μL. On the day of transfection, 254, of OptiMEM media and 1.25 ul of Lipofectamine Messenger Max Solution (Thermo Fisher) were mixed in a mastermix solution, vortexed, and allowed to sit for at least 5 minutes at room temperature. In separate tubes, 300 ng of the MG3-6-MG-3-4-encoding mRNA (SEQ ID NO: 108) and 120 ng of the sgRNA (scaffold sequence SEQ ID NO:34) were mixed together with 254, of OptiMEM media, and vortexed briefly. The appropriate volume of MessengerMax solution was added to each RNA solution, mixed by flicking the tube, and briefly spun down at a low speed. The complete editing reagent solutions were allowed to incubate for 10 minutes at room temperature, then added directly to the Hepa1-6 cells. Two days post transfection, the media was aspirated off of each well of Hepa1-6 cells and genomic DNA was purified by automated magnetic bead purification, via the KingFisher Flex with the MagMAX™ DNA Multi-Sample Ultra 2.0 Kit. The activity of the guides is summarized in Tables 2 and 3, while the primers used are summarized in Table 4.

TABLE 2
Average Activity of MG3-6/3-4 guides at mouse HAO1
delivered by mRNA Transfection

| | | | | Editing |
|---|---|---|---|---|
| | | SEQ | | Activity |
| Guide | | ID | | (Average |
| Name | PAM | No. | Spacer Sequence | % INDELs) |
| mH364-1 | GCAAATG | 611 | GTATGACTATTACAGGTCTGGG | 0 |
| mH364-2 | GAAAATG | 612 | AAATAGCAAAGTTTCTTACCTA | 0 |
| mH364-3 | AGAAAAT | 613 | TAAATAGCAAAGTTTCTTACCT | 0 |
| mH364-6 | CTAAAAC | 614 | ATTGGCATGCTGACTCTCTGTC | 0 |
| mH364-7 | AGAAAAG | 615 | GAGCTGGCCACTGTGCGAGGTA | 45.7 |
| mH364-9 | ACAAATA | 616 | CAGGTAAGGGGTGTCCACAGTC | 0 |
| mH364-10 | TGAAAAA | 617 | ATTCTATGTATCTATTCTAGGA | 0 |
| mH364-11 | GAAAAAC | 618 | TTCTATGTATCTATTCTAGGAT | 31 |
| mH364-15 | CCAAATC | 619 | AAATTTCCCTTAGGAGAAAATG | 0 |
| mH364-16 | GAAAATG | 620 | GTCTCCAAAATTTCCCTTAGGA | 10.7 |
| mH364-17 | AGAAAAT | 621 | TGTCTCCAAAATTTCCCTTAGG | 0 |
| mH364-18 | GGAAATT | 622 | TGATTTGGCATTTTCTCCTAAG | 0 |
| mH364-19 | CAAAATT | 623 | TCAGCAAGTCCACTGTTGTCTC | 0 |
| mH364-20 | CCAAAAT | 624 | TTCAGCAAGTCCACTGTTGTCT | 25.9 |
| mH364-22 | CAAAATG | 625 | AGTAGAGAAATGACAAACCTCT | 0 |
| mH364-23 | TCAAAAT | 626 | AAGTAGAGAAATGACAAACCTC | 20.7 |

TABLE 3
Results of testing MG3-6/3-4 guides with a more permissive PAM design, at mouse
HAO1 delivered by mRNA Transfection

| | | | | Editing | |
|---|---|---|---|---|---|
| Guide | | SEQ | | Activity | |
| Name | PAM | ID No. | Spacer Sequence | (% INDELs) | R² |
| mH364-4 | AGAAACT | 627 | ACATCCAAGCATTTTCTAGGTA | 0 | 1 |
| mH364-5 | TAAAACA | 628 | TTGGCATGCTGACTCTCTGTCC | 0 | 1 |
| mH364-8 | ACAAAGA | 629 | CGCTGGATGCAACTGTACATCT | 0 | 0.99 |
| mH364-12 | AAAAACT | 630 | TCTATGTATCTATTCTAGGATG | 0 | 0.99 |
| mH364-13 | TGAAACC | 631 | TCTATTCTAGGATGAAAAACTT | 0 | 0.99 |
| mH364-14 | TCAAAGT | 632 | AGAAAATGCCAAATCATTGGTT | 0 | 0.99 |
| mH364-21 | GTAAAGG | 633 | ATTGACATCACTGCCTATTGTT | 0 | 1 |

TABLE 4
Primers designed for the mouse HAO1 gene, used for PCR at each of the first four
exons, and for sanger sequencing.

| Target | | | SEQ | |
|---|---|---|---|---|
| Exon | Use | Primer Name | ID No. | Primer Sequence |
| Mouse | Fwd PCR | PCR_mHE1_F_+233 | 634 | GTGACCAACCCTACCCGTTT |
| HAO1 | Rev PCR | PCR_mHE1_R_−553 | 635 | GCAAGCACCTACTGTCTCGT |
| Exon 1 | Sequencing | Seq_mHE1_F_+139 | 636 | GTCTAGGCATACAATGTTTGCTCA |
| Mouse | Fwd PCR | HAO1_E2_F5721 | 637 | CAACGAAGGTTCCCTCCAGG |
| HAO1 | Rev PCR | HAO1_E2_R6271 | 638 | GGAAGGGTGTTCGAGAAGGA |
| Exon 2 | Sequencing | 5938F Seq_HAO1_E2 | 639 | CTATGCAAGGAAAAGATTTGGCC |
| Mouse | Fwd PCR | HAO1_E3_F23198 | 640 | TGCCCTAGACAAGCTGACAC |
| HAO1 | Rev PCR | HAO1_E3_R23879 | 641 | CAGATTCTGGAAGTGGCCCA |
| Exon 3 | Sequencing | HAO1_E3_F23198 | 642 | Same as Fwd PCR Primer |
| Mouse | Fwd PCR | PCR_mHE4_F_+300 | 643 | GGCTGGCTGAAAATAGCATCC |
| HAO1 | Rev PCR | HAO1_E4_R31650 | 644 | AGGTTTGGTTCCCCTCACCT |
| Exon 4 | Sequencing | PCR_mHE4_R_−149 | 645 | TCTGCCATGAAGGCATATGGAC |

### Example 9—Guide Chemistry Optimization for the MG3-6/3-4 and MG3-6 Type II Nuclease

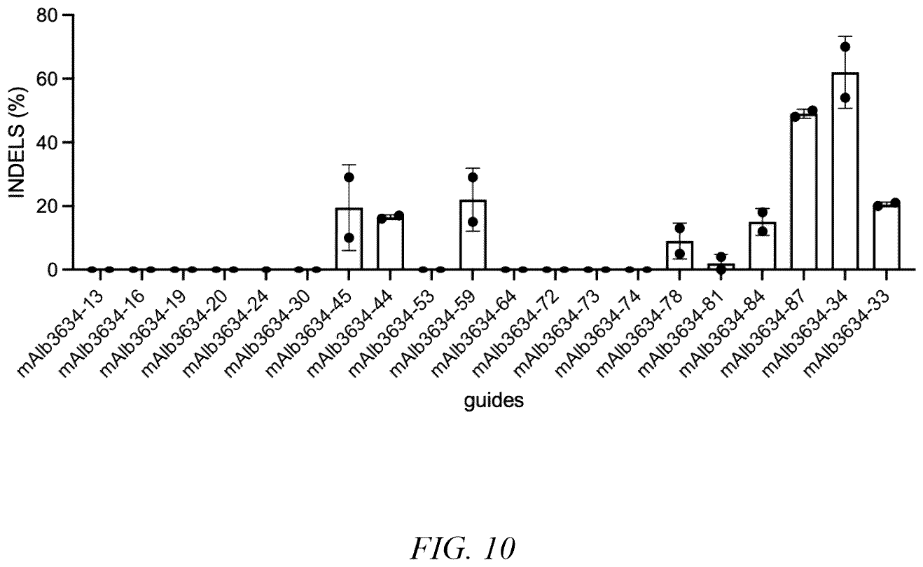

We designed 40 different chemically modified guides (named mAlb3634-34-0 to mAlb3634-34-44) and tested the activity of 39 of these guides. One guide, mH3634-34-32, failed RNA synthesis, thus it was not tested. The guide spacer sequence we chose as a model to insert various chemical modifications was mAlb3634-34 (targeting albumin intron 1) as it proved to be the most active guide in a guide screen in the mouse hepatocyte cell line Hepa1-6 cells (Table 5 and FIG. 10).

TABLE 5
Activity of chemically modified guides in Hepa1-6 cells

| | Editing Activity |
|---|---|
| Guide | (% INDELs) |
| mAlb3634-13 | 0 |
| mAlb3634-16 | 0 |
| mAlb3634-19 | 0 |
| mAlb3634-20 | 0 |
| mAlb3634-24 | 0 |
| mAlb3634-30 | 0 |
| mAlb3634-45 | 19.5 |
| mAlb3634-44 | 16.5 |
| mAlb3634-53 | 0 |
| mAlb3634-59 | 22 |
| mAlb3634-64 | 0 |
| mAlb3634-72 | 0 |
| mAlb3634-73 | 0 |
| mAlb3634-74 | 0 |
| mAlb3634-78 | 9 |
| mAlb3634-81 | 2 |
| mAlb3634-84 | 15 |
| mAlb3634-87 | 49 |
| mAlb3634-34 | 62 |
| mAlb3634-33 | 20.5 |

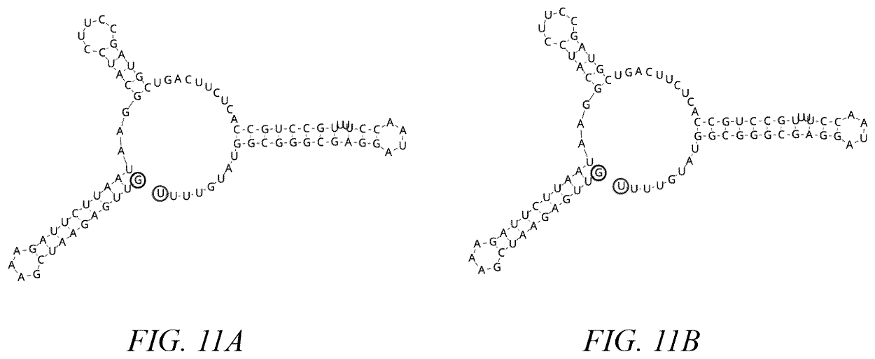

The sgRNA of MG3-6/3-4 comprises a spacer located at the 5′ end followed by the CRISPR repeat and the trans-activating CRISPR RNA (tracr). The CRISPR repeat and the tracr are identical to that of the MG3-6 nuclease (FIG. 11a, 11b). The CRISPR repeat and tracr form a structured RNA comprising 3 stem loops (FIG. 11a). We modified different areas of the stem loops by replacing the 2′ hydroxyl of the ribose with methyl groups or replacing the phosphodiester backbone by a phosphorothioate (PS). Moreover, the spacer at the 5′ of the guide was modified with a mixture of 2′-O-methyl or 2′-fluorine bases and PS bonds. The different combinations of chemical modifications designed are called mAlb3634-34-0 to mAlb3634-34-44 and the sequences are shown in Table 6.

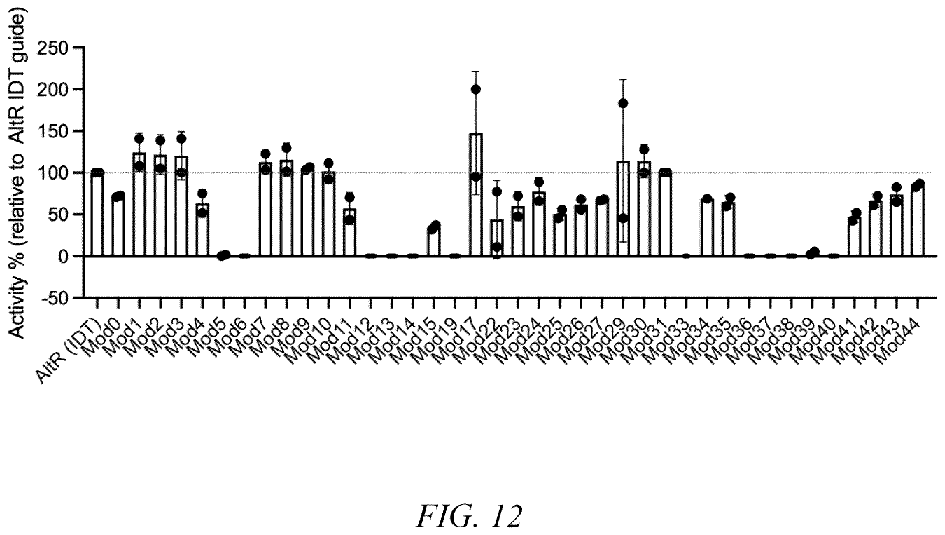

The editing activity of 39 single guides with the exact same base sequence but different chemical modifications was evaluated in Hepa1-6 cells by co-transfection of mRNA encoding MG3-6/3-4 and the guide; the results are shown in Table 6 and FIG. 12.

TABLE 6
Sequences of chemically modified MG3-6/3-4 guides and their activity in Hepa1-6
cells when co-transfected with MG3-6/3-4 mRNA

| | SEQ | | |
|---|---|---|---|
| Guide | ID No. | Sequence | Activity |
| mAlb3634-34-0 | 646 | rCrUrUrArGrGrUrCrArGrUrGrArArGrArGrArArGrArArGrUrUrG | 71.8 |
| | | rArGrArArUrCrGrArArArGrArUrUrCrUrUrArArUrArArGrGrC | |
| | | rArUrCrCrUrUrCrCrGrArUrGrCrUrGrArCrUrUrCrUrCrArCrC | |
| | | rGrUrCrCrGrUrUrUrUrCrCrArArUrArGrGrArGrCrGrGrGrCrGrG | |
| | | rUrArUrGrUrUrU | |
| mAlb3634-34-1 | 647 | mC*mU*mU*rArGrGrUrCrArGrUrGrArArGrArGrArArGrArArG | 124.5 |
| | | rUrUrGrArGrArArUrCrGrArArArGrArUrUrCrUrUrArArUrArA | |
| | | rGrGrCrArUrCrCrUrUrCrCrGrArUrGrCrUrGrArCrUrUrCrUrC | |
| | | rArCrCrGrUrCrCrGrUrUrUrUrCrCrArArUrArGrGrArGrCrGrGrG | |
| | | rCrGrGrUrArUrGrU*mU*mU*mU | |
| mAlb3634-34-2 | 648 | mC*mU*mU*rArGrGrUrCrArGrUrGrArArGrArGrArArGrArArG | 121.7 |
| | | rUrUrGrArGrArArUrCrGrArArArGrArUrUrCrUrUrArArUrArA | |
| | | rGrGrCrArUrCrCrUrUrCrCrGrArUrGrCrUrGrArCrUrUrCrUrC | |
| | | rArCrCrGrUrCrCrGrUrUrUrUrCrCrArArUrArGrGrArGrCrGrGrG | |
| | | rCrGrGrUrA*mU*mG*mU*mU*mU*mU | |
| mAlb3634-34-3 | 649 | mC*mU*mU*rArGrGrUrCrArGrUrGrArArGrArGrArArGrArArG | 120.5 |
| | | rUrUrGrArGmAmAmUmCmGmAmAmAmGmAmUmUrCrUrU | |
| | | rArArUrArArGrGrCrArUrCrCrUrUrCrCrGrArUrGrCrUrGrArC | |
| | | rUrUrCrUrCrArCrCrGrUrCrCrGrUrUrUrUrCrCrArArUrArGrGrA | |
| | | rGrCrGrGrGrCrGrGrUrArUrGrU*mU*mU*mU | |
| mAlb3634-34-4 | 650 | mC*mU*mU*rArGrGrUrCrArGrUrGrArArGrArGrArArGrArArG | 63.3 |
| | | rUrUrGrArG*mA*mA*mU*mC*mG*mA*mA*mA*mG*mA* | |
| | | mU*mUrCrUrUrArArUrArArGrGrCrArUrCrCrUrUrCrCrGrArUrG | |
| | | rCrUrGrArCrUrUrCrUrCrArCrCrGrUrCrCrGrUrUrUrUrCrCrA | |
| | | rArUrArGrGrArGrCrGrGrGrCrGrGrUrArUrGrU*mU*mU*mU | |
| mAlb3634-34-5 | 651 | mC*mU*mU*rArGrGrUrCrArGrUrGrArArGrArGrArArGrArAm | 0.8 |
| | | GmUmUmGmAmGmAmAmUmCmGmAmAmAmGmAmUmU | |
| | | mCmUmUmAmArUrArArGrGrCrArUrCrCrUrUrCrCrGrArUrGrC | |
| | | rUrGrArCrUrUrCrUrCrArCrCrGrUrCrCrGrUrUrUrUrCrCrArA | |
| | | rUrArGrGrArGrCrGrGrGrCrGrGrUrArUrGrU*mU*mU*mU | |
| mAlb3634-34-6 | 652 | mC*mU*mU*rArGrGrUrCrArGrUrGrArArGrArGrArArGrArAm | 0.0 |
| | | GmUmUmGmAmGmAmAmUmCrG*rA*rA*rA*mGmAmUmU | |
| | | mCmUmUmAmArUrArArGrGrCrArUrCrCrUrUrCrCrGrArUrGrC | |
| | | rUrGrArCrUrUrCrUrCrArCrCrGrUrCrCrGrUrUrUrUrCrCrArA | |
| | | rUrArGrGrArGrCrGrGrGrCrGrGrUrArUrGrU*mU*mU*mU | |
| mAlb3634-34-7 | 653 | mC*mU*mU*rArGrGrUrCrArGrUrGrArArGrArGrArArGrArArG | 113.0 |
| | | rUrUrGrArGrArArUrCrGrArArArGrArUrUrCrUrUrArArUrArA | |
| | | mGmGmCmAmUmCrCrUrUrCrCrGrArUrGrCrUrGrArCrUrUrC | |
| | | rUrCrArCrCrGrUrCrCrGrUrUrUrUrCrCrArArUrArGrGrArGrC | |
| | | rGrGrGrCrGrGrUrArUrGrU*mU*mU*mU | |
| mAlb3634-34-8 | 654 | mC*mU*mU*rArGrGrUrCrArGrUrGrArArGrArGrArArGrArArG | 115.6 |
| | | rUrUrGrArGrArArUrCrGrArArArGrArUrUrCrUrUrArArUrArA | |
| | | mGmGmCmAmUmCrC*rU*rU*rC*rC*rGrArUrGrCrUrGrArC | |
| | | rUrUrCrUrCrArCrCrGrUrCrCrGrUrUrUrUrCrCrArArUrArGrGrA | |
| | | rGrCrGrGrGrCrGrGrUrArUrGrU*mU*mU*mU | |
| mAlb3634-34-9 | 655 | mC*mU*mU*rArGrGrUrCrArGrUrGrArArGrArGrArArGrArArG | 105.0 |
| | | rUrUrGrArGrArArUrCmGmAmAmArGrArUrUrCrUrUrArArU | |
| | | rArArGrGrCrArUrCmCmUmUmCmCrGrArUrGrCrUrGrArCrUrU | |
| | | rCrUrCrArCrCrGrUrCrCrGrUrUrUrUrCrCmAmAmUmArGrGrA | |
| | | rGrCrGrGrGrCrGrGrUrArUrGrU*mU*mU*mU | |
| mAlb3634-34-10 | 656 | mC*mU*mU*rArGrGrUrCrArGrUrGrArArGrArGrArArGrArArG | 101.6 |
| | | rUrUrGrArGrArArUrCrG*rA*rA*rA*rGrArUrUrCrUrUrArArU | |
| | | rArArGrGrCrArUrCrC*rU*rU*rC*rC*rGrArUrGrCrUrGrArCrU | |
| | | rUrCrUrCrArCrCrGrUrCrCrGrUrUrUrUrCrCrA*rA*rU*rA*rG | |
| | | rGrArGrCrGrGrGrCrGrGrUrArUrGrU*mU*mU*mU | |
| mAlb3634-34-11 | 657 | mC*mU*mU*rArGrGrUrCrArGrUrGrArArGrArGrArArGrArArG | 57.0 |
| | | rUrUrGrArGrArArUrCrGrArArArGrArUrUrCrUrUrArArUrArA | |
| | | rGrGrCrArUrCrCrUrUrCrCrGrArUrGrCrUrGrArCrUrUrCrUrC* | |
| | | mA*mC*mC*mG*mU*mC*mC*mG*mU*mU*mU*mU*mC* | |
| | | mC*mA*mA*mU*mArGrGrArGrCrGrGrGrCrGrGrUrA*mU*mG* | |
| | | mU*mU*mU*mU | |
| mAlb3634-34-12 | 658 | mC*mU*mU*rArGrGrUrCrArGrUrGrArArGrArGrArArGrArAm | 0.0 |
| | | GmUmUmGmAmGmAmAmUmCrG*rA*rA*rA*rGrArUrUrCrU | |
| | | rUrArArUrArArGrGrCrArUrCrCrUrUrCrCrGrArUrGrCrUrGrArC | |
| | | rUrUrCrUrC*mA*mC*mC*mG*mU*mC*mC*mG*mU*mU* | |
| | | mU*mU*mC*mC*mA*mA*mU*mA*mG*mG*mA*mG*mC*mG* | |
| | | mG*mG*mC*mG*mG*mU*mA*mU*mG*mU*mU*mU*mU | |
| mAlb3634-34-13 | 659 | mC*mU*mU*rArGrGrUrCrArGrUrGrArArGrArGrArArGrArAm | 0.0 |
| | | GmUmUmGmAmGmAmAmUmCmGmAmAmAmGmAmUmU | |
| | | mCmUmUmAmArUrArArGrGrCrArUrCrCrUrUrCrCrGrArUrGrC | |
| | | rUrGrArCrUrUrCrUrCrArCrCrGrUrCrCrGrUrUrUrUrCrCrArA | |
| | | rUrArGrGrArGrCrGrGrGrCrGrGrUrArUrGrU*mU*mU*mU | |
| mAlb3634-34-14 | 670 | mC*mU*mU*rArGrGrUrCrArGrUrGrArArGrArGrArArGrArArG | 0.0 |
| | | rUrUrGrArGmAmAmUmCmGmAmAmAmGmAmUmUrCrUrU | |
| | | rArArUrArArGrGrCrArUrCrCrUrUrCrCrGrArUrGrCrUrGrArC | |
| | | rUrUrCrUrC*mA*mC*mC*mG*mU*mC*mC*mG*mU*mU*mU* | |
| | | mU*mC*mC*mA*mA*mU*mA*mG*mG*mA*mG*mC*mG* | |
| | | mG*mG*mC*mG*mG*mU*mA*mU*mG*mU*mU*mU*mU | |
| mAlb3634-34-15 | 671 | mC*mU*mU*rArGrGrUrCrArGrUrGrArArGrArGrArArGrArArG | 34.5 |
| | | rUrUrGrArGmAmAmUmCmGmAmAmAmGmAmUmUrCrUrU | |
| | | rArArUrArAmGmGmCmAmUmCrCrUrUrCrCrGrArUrGrCrUr | |
| | | GrArCrUrUrCmUmCmAmCmCmGmUmCmCmGmUmUmUmU | |
| | | mCmCmAmAmUmAmGmGmAmGmCmGmGmGmCmGmGmU | |
| | | mAmUmGmU*mU*mU*mU | |
| mAlb3634-34-19 | 672 | mC*mU*mU*rArGrGrUrCrArGrUrGrArArGrArGrArArGrArArG | 0.0 |
| | | rUrUrGrArGrArArUrC*mG*mA*mA*mArGrArUrUrCrUrUrA | |
| | | rA*mU*mA*mArGrGrCrArUrC*mC*mU*mU*mC*mCrGrArUrG | |
| | | rCrU*mG*mA*mC*mU*mU*mC*mU*mCrArCrCrGrUrCrCrG | |
| | | rUrUrUrUrCrC*mA*mA*mU*mArGrGrArGrCrGrGrGrCrGrGrU | |
| | | rA*mU*mG*mU*mU*mU*mU | |
| mAlb3634-34-17 | 673 | mC*mU*mU*i2FAi2FGi2FGi2FUi2FCi2FAi2FGi2FUi2FGi2FAi2 | 147.7 |
| | | FAi2FGi2FAi2FGrArArGrArArGrUrUrGrArGrArArUrCrGrArA | |
| | | rArGrArUrUrCrUrUrArArUrArArGrGrCrArUrCrCrUrUrCrCrGrA | |
| | | rUrGrCrUrGrArCrUrUrCrUrCrArCrCrGrUrCrCrGrUrUrUrUrC | |
| | | rCrArArUrArGrGrArGrCrGrGrGrCrGrGrUrArUrGrU*mU*mU* | |
| | | mU | |
| mAlb3634-34-22 | 674 | mC*mU*mU*rArGrGrUrCrArGrUrGrArArGrArGrArArGrArArG | 44.2 |
| | | rUrUrGrArGrArArUrCrG*rA*rA*rA*rGrArUrUrCrUrUrArArU | |
| | | rArArGrGrCrArUrCrC*rU*rU*rC*rC*rGrArUrGrCrUrGrArCrU | |
| | | rUrCrUrCmAmCmCmGmUmCmCmGmUmUmUmUmCmCrA | |
| | | *rA*rU*rA*mGmGmAmGmCmGmGmGmCmGmGmU*mA*mU* | |
| | | mG*mU*mU*mU*mU | |
| mAlb3634-34-23 | 675 | mC*mU*mU*rArGrGrUrCrArGrUrGrArArGrArGrArArGrArArG | 60.0 |
| | | rUrUrGrArGrArArUrCrG*A*rA*rA*rGrArUrUrCrUrUrArArU | |
| | | rArArGrGrCrArUrCrC*rU*rU*rC*rC*rGrArUrGrCrUrG*rA* | |
| | | rC*rU*rU*rC*rU*rC*mAmCmCmGmUmCmCmGmUmUmUmU | |
| | | mCmCrA*rA*rU*rA*mGmGmAmGmCmGmGmGmCmGmGm | |
| | | UmA*mU*mG*mU*mU*mU*mU | |
| mAlb3634-34-24 | 676 | mC*mU*mU*rArGrGrUrCrArGrUrGrArArGrArGrArArGrArArG | 77.4 |
| | | rUrUrGrArGrArArUrCrG*rA*rA*rA*rGrArUrUrCrUrUrArArU | |
| | | rArAmGmGmCmAmUmCrC*rU*rU*rC*rC*rGrArUrGrCrUrG | |
| | | rArCrUrUrCrUrCmAmCmCmGmUmCmCmGmUmUmUmUmC | |
| | | mCrA*rA*rU*rA*mGmGmAmGmCmGmGmGmCmGmGmU* | |
| | | mA*mU*mG*mU*mU*mU*mU | |
| mAlb3634-34-25 | 677 | mC*mU*mU*rArGrGrUrCrArGrUrGrArArGrArGrArArGrArArG | 50.5 |
| | | rUrUrGrArGrArArUrCrG*rA*rA*rA*rGrArUrUrCrUrUrArArU | |
| | | rArAmGmGmCmAmUmCrC*rU*rU*rC*rC*rGrArUrGrCrUrG* | |
| | | rA*rC*rU*rU*rC*rU*rC*mAmCmCmGmUmCmCmGmUmUm | |
| | | UmUmCmCrA*rA*rU*rA*mGmGmAmGmCmGmGmGmCmG | |
| | | mGmU*mA*mU*mG*mU*mU*mU*mU | |
| mAlb3634-34-26 | 678 | mC*mU*mU*mA*rGrGrUrCrArGrUrGrArArGrArGrArArGrArA | 61.9 |
| | | rGrUrUrGrArGrArArUrCrGrArArArGrArUrUrCrUrUrArArUrA | |
| | | rArGrGrCrArUrCrC*rU*rU*rC*rC*rGrArUrGrCrUrGrArCrUrU | |
| | | rCrUrCrArCrCrGrUrCrCrGrUrUrUrUrCrCrArArUrArGrGrArG | |
| | | rCrGrGrGrCrGrGrUrA*mU*mG*mU*mU*mU*mU | |
| mAlb3634-34-27 | 679 | mC*mU*mU*mA*rGrGrUrCrArGrUrGrArArGrArGrArArGrArA | 67.4 |
| | | rGrUrUrGrArGrArArUrCrG*rA*rA*rA*rGrArUrUrCrUrUrArA | |
| | | rUrArArGrGrCrArUrCrCrUrUrCrCrGrArUrGrCrUrGrArCrUrU | |
| | | rCrUrCmAmCmCmGmUmCmCmGmUmUmUmUmCmCrA*rA* | |
| | | rU*rA*rGrGrArGrCrGrGrGrCrGrGrUrA*mU*mG*mU*mU*mU* | |
| | | mU | |
| mAlb3634-34-29 | 680 | mC*i2FU*i2FU*i2FA*rGrGrUrCrArGrUrGrArArGrArGrArArGrA | 114.4 |
| | | rAmGmUmUmGmAmGmAmAmUmCrGrArArArGrArUrUrCrU | |
| | | rUrArArUrArArGrGrCrArUrCrCrUrUrCrCrGrArUrGrCrUrGrA | |
| | | rCrUrUrCrUrCrArCrCrGrUrCrCrGrUrUrUrUrCrCrArArUrArGrG | |
| | | rArGrCrGrGrGrCrGrGrUrA*mU*mG*mU*mU*mU*mU | |
| mAlb3634-34-30 | 681 | mC*i2FU*i2FU*i2FA*rGrGrUrCrArGrUrGrArArGrArGrArArGrA | 113.9 |
| | | rArGrUrUrGrArGrArArUrCrGrArArArGrArUrUrCrUrUrArArU | |
| | | rArAmGmGmCmAmUmCrCrUrUrCrCrGrArUrGrCrUrGrArCrU | |
| | | rUrCrUrCrArCrCrGrUrCrCrGrUrUrUrUrCrCrArArUrArGrGrA | |
| | | rGrCrGrGrGrCrGrGrUrA*mU*mG*mU*mU*mU*mU | |
| mAlb3634-34-31 | 682 | mC*i2FU*i2FU*i2FA*rGrGrUrCrArGrUrGrArArGrArGrArArGrA | 100.0 |
| | | rArGrUrUrGrArGrArArUrCrGrArArArGrArUrUrCrUrUrArArU | |
| | | rArArGrGrCrArUrCrCrUrUrCrCrGrArUrGrCrUrGrArCrUrUrC | |
| | | rUrCmAmCmCmGmUmCmCmGmUmUmUmUmCmCrArArUr | |
| | | ArGrGrArGrCrGrGrGrCrGrGrUrA*mU*mG*mU*mU*mU*mU | |
| mAlb3634-34-32 | 683 | mC*mU*mU*i2FA*i2FGi2FGi2FUi2FCi2FAi2FGi2FUi2FGi2FA | NT |
| | | i2FAi2FGi2FAi2FGrArArGrArAmGmUmUmGmAmGmAmA | |
| | | mUmCrG*rA*rA*rA*mGmAmUmUrCrUrUrArArUrArAmGmG | |
| | | mCmAmUmCrC*rU*rU*rC*rC*rGrArUrGrCrUrGrArCrUrUrCrU | |
| | | rCmAmCmCmGmUmCmCmGmUmUmUmUmCmCrA*rA*rU* | |
| | | rA*mGmGmAmGmCmGmGmGmCmGmGmUmA*mU*mG*mU* | |
| | | mU*mU*mU | |
| mAlb3634-34-33 | 684 | mC*mU*mU*i2FA*i2FGi2FGi2FUi2FCi2FAi2FGi2FUi2FGi2FA | 0.0 |
| | | i2FAi2FGi2FAi2FGrArArGrArAmGmUmUmGmAmGmAmAm | |
| | | UmCrG*rA*rA*A*mGmAmUmUrCrUrUrArArUrArAmGmGm | |
| | | CmAmUmCrC*rU*rU*rC*rC*rGrArUrGrCrUrGrArCrUrUrCrU | |
| | | rCrArCrCrGrUrCrCrGrUrUrUrUrCrCrA*rA*U*rA*rGrGrArGrC | |
| | | rGrGrGrCrGrGrUrA*mU*mG*mU*mU*mU*mU | |
| mAlb3634-34-34 | 685 | mC*mU*mU*mA*i2FGi2FGi2FUi2FCi2FAi2FGi2FUi2FGi2FA | 68.9 |
| | | i2FAi2FGi2FAi2FGrArArGrArArGrUrUrGrArGrArArUrCrG*rA* | |
| | | rA*rA*rGrArUrUrCrUrUrArArUrArArGrGrCrArUrCrC*rU*rU* | |
| | | rC*rC*rGrArUrGrCrUrGrArCrUrUrCrUrCrArCrCrGrUrCrCrGrU | |
| | | rUrUrUrCrCrA*rA*U*rA*rGrGrArGrCrGrGrGrCrGrGrUrA* | |
| | | mU*mG*mU*mU*mU*mU | |
| mAlb3634-34-35 | 686 | mC*mU*mU*mA*rGrGrUrCrArGrUrGrArArGrArGrArArGrArA | 65.0 |
| | | rGrUrUrGrArGrArArUrCmG*mA*mA*mA*rGrArUrUrCrUrUrA | |
| | | rArUrArArGrGrCrArUrCmC*mU*mU*mC*mC*rGrArUrGrCrU | |
| | | rGrArCrUrUrCrUrCrArCrCrGrUrCrCrGrUrUrUrUrCrCmA* | |
| | | mA*mU*mA*rGrGrArGrCrGrGrGrCrGrGrUrA*mU*mG*mU* | |
| | | mU*mU*mU | |
| mAlb3634-34-36 | 687 | mC*mU*mU*mA*rGrGrUrCrArGrUrGrArArGrArGrArArGrArA | 0.0 |
| | | mGmUmUmGmAmGmAmAmUmCrG*rA*rA*rA*rGrArUrUrC | |
| | | rUrUrArArUrArAmGmGmCmAmUmCrC*rU*rU*rC*rC*rGrArU | |
| | | rGrCrUrGrArCrUrUrCrUrCrArCrCrGrUrCrCrGrUrUrUrUrCrC | |
| | | rA*rA*rU*rA*rGrGrArGrCrGrGrGrCrGrGrUrA*mU*mG*mU* | |
| | | mU*mU*mU | |
| mAlb3634-34-37 | 688 | mC*mU*mU*mA*rGrGrUrCrArGrUrGrArArGrArGrArArGrArA | 0.0 |
| | | mGmUmUmGmAmGmAmAmUmCrG*rA*rA*rA*rGrArUrUrU | |
| | | rUrUrArArUrArAmGmGmCmAmUmCrC*rU*rU*rC*rC*rGrArU | |
| | | rGrCrUrGrArCrUrUrCrUrCmAmCmCmGmUmCmCmGmUm | |
| | | UmUmUmCmCrA*rA*rU*rA*rGrGrArGrCrGrGrGrCrGrGrUrA* | |
| | | mU*mG*mU*mU*mU*mU | |
| mAlb3634-34-38 | 689 | mC*mU*mU*mA*rGrGrUrCrArGrUrGrArArGrArGrArArGrArA | 0.0 |
| | | mGmUmUmGmAmGmAmAmUmCrG*rA*rA*rA*rGrArUrUrC | |
| | | rUrUrArArUrArAmGmGmCmAmUmCrC*rU*rU*rC*rC*rGrArU | |
| | | rGrCrUrGrArCrUrUrCrUrCrArCrCmGmUmCmCmGmUmUm | |
| | | UmUmCmCrA*rA*rU*rA*mGmGmAmGmCmGmGmGmCmGr | |
| | | GrUrA*mU*mG*mU*mU*mU*mU | |
| mAlb3634-34-39 | 690 | mC*mU*mU*mA*rGrGrUrCrArGrUrGrArArGrArGrArArGrArA | 3.7 |
| | | rGrUrUrGrArGmAmAmUmCrG*rA*rA*rA*rGrArUrUrCrUrUrA | |
| | | rArUrArArGrGrCrArUrCrC*rU*rU*rC*C*rGrArUrGrCrUrG* | |
| | | rA*rC*rU*rU*rC*rU*rC*rArCrCrGrUrCrCrGrUrUrUrUrCrCrA* | |
| | | rA*rU*rA*rGrGrArGrCrGrGrGrCrGrGrUrA*mU*mG*mU*mU* | |
| | | mU*mU | |
| mAlb3634-34-40 | 691 | mC*mU*mU*mA*rGrGrUrCrArGrUrGrArArGrArGrArArGrArA | 0.0 |
| | | rGrUrUrGrArGmAmAmUmCrG*rA*rA*rA*mGmAmUmUrCrU | |
| | | rUrArArUmAmAmGmGmCmAmUmCrC*rU*rU*rC*rC*mGm | |
| | | AmUmGmCrU*rG*rA*mCmUmUrCrUrCrArCrCrGrUrCrCrGrUrU | |
| | | rUrUrCrCrA*rA*rU*rA*rGrGrArGrCrGrGrGrCrGrGrUrA* | |
| | | mU*mG*mU*mU*mU*mU | |
| mAlb3634-34-41 | 692 | mC*mU*mU*mA*rGrGrUrCrArGrUrGrArArGrArGrArArGrArA | 47.1 |
| | | rGrUrUrGrArGmAmAmUmCrG*rA*rA*rA*rGrArUrUrCrUrUrA | |
| | | rArUrArAmGmGmCmAmUmCrC*rU*rU*C*rC*rGrArUrGrC | |
| | | rUrGrArCrUrUrCrUrCmAmCmCmGmUmCmCmGmUmUmUm | |
| | | UmCmCrA*rA*rU*rA*rGrGrArGrCrGrGrGrCrGrGrUrA*mU* | |
| | | mG*mU*mU*mU*mU | |
| mAlb3634-34-42 | 693 | mC*mU*mU*mA*i2FGi2FGi2FUi2FCi2FAi2FGi2FUi2FGi2FA | 66.7 |
| | | i2FAi2FGi2FAi2FGi2FAi2FAi2FGi2FArArGrUrUrGrArGrArArU | |
| | | rCrG*rA*rA*rA*rGrArUrUrCrUrUrArArUrArArGrGrCrArUrCrC | |
| | | *rU*rU*rC*rC*rGrArUrGrCrUrGrArCrUrUrCrUrCrArCrCrGrU | |
| | | rCrCrGrUrUrUrUrCrCrA*rA*rU*rA*rGrGrArGrCrGrGrGrCrGrG | |
| | | rUrA*mU*mG*mU*mU*mU*mU | |
| mAlb3634-34-43 | 694 | mC*mU*mU*rArGrGrUrCrArGrUrGrArArGrArGrArArGrArArG | 73.8 |
| | | rUrUrGrArGrArArUrCrGrArArArGrArUrUrCrUrUrArArUrArA | |
| | | rGrGrCrArUrCrCrUrUrCrCrGrArUrGrCrUrGrArCrUrUrCrUrCrA | |
| | | rCrCrGrUrCrCrGrUrUrUrUrCrCrArArUrAmGmGmAmGmCm | |
| | | GmGmGmCmGmGmUmA*mU*mG*mU*mU*mU*mU | |
| mAlb3634-34-44 | 695 | mC*mU*mU*rArGrGrUrCrArGrUrGrArArGrArGrArArGrArArG | 84.9 |
| | | rUrUrGrArGrArArUrCrGrArArArGrArUrUrCrUrUrArArUrArA | |
| | | rGrGrCrArUrCrCrUrUrCrCrGrArUrGrCrUrGrArCrUrUrCrUrCm | |
| | | AmCmCmGmUmCmCmGmUmUmUmUmCmCrArArUrArGrG | |
| | | rArGrCrGrGrGrCrGrGrUrA*mU*mG*mU*mU*mU*mU | |

(r = native ribose base, m = 2′-O methyl modified base, F = 2′ Fluro modified base, * = phosphorothioate bond)

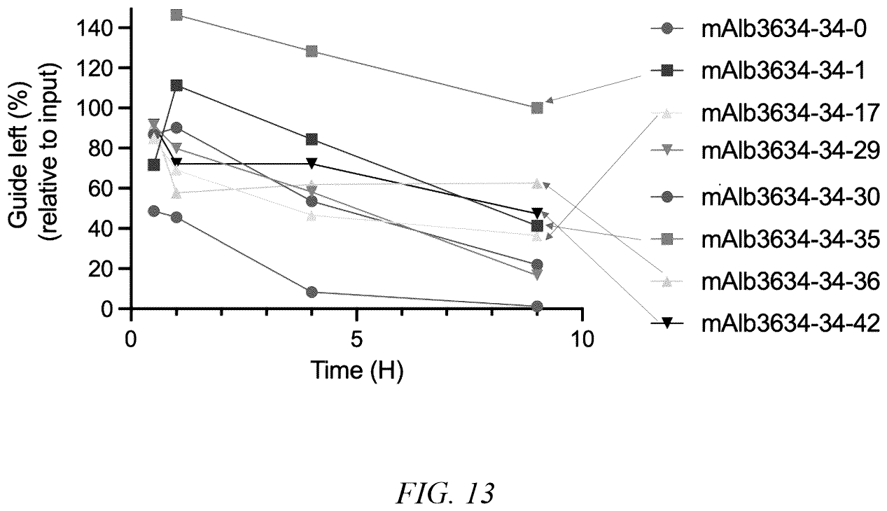

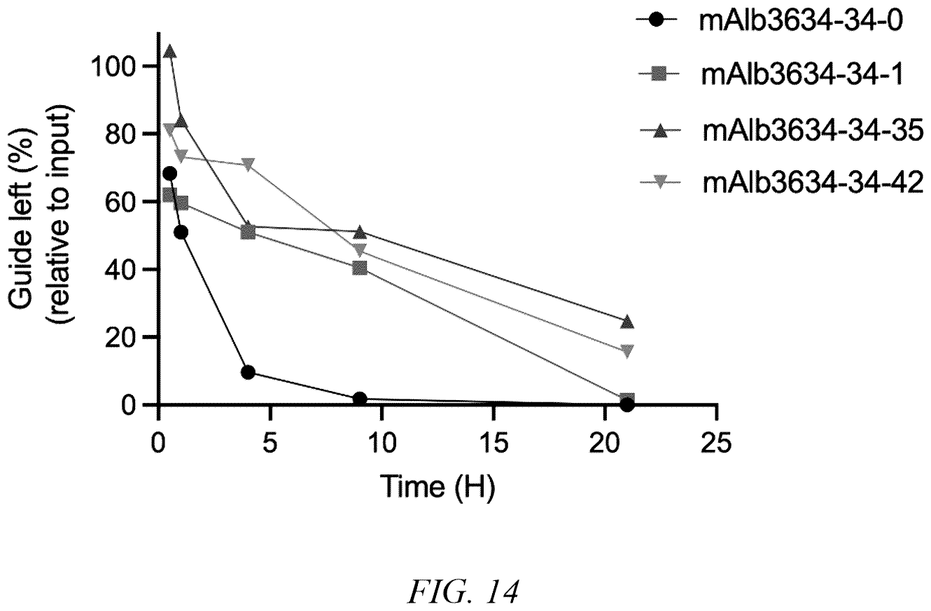

A guide with the same base sequence and a commercially available chemical modification called AltR1/AltR2 was used as a control. The spacer sequence in these guides targets a 22-nucleotide region in albumin intron1 of the mouse genome. Guide mAlb3634-34-0 (no chemical modifications) showed 72% activity relative to the AltR1/AltR2 guide. Guide mAlb3634-34-1 showed 124% activity relative to the AltR1/AltR2 guide, showing the importance of stability of guides for editing: mAlb3634-34-1 is more stable than mAlb3634-34-0 (FIG. 13 and FIG. 14). Importantly, mAlb3634-34-17 retained 147% of the activity relative to AltR1/AltR2. The incorporation of 2′-O-fluorines in the spacer greatly increased the stability of mAlb3634-34-35, and the guide retained 65% activity. mAlb3634-34-35 contains 2′-O-methyl and PS bonds in the loops of the three stem loops of the MG3-6/3-4 guide. Importantly, mAlb3634-34-42 retained 66% of activity and this guide contains as many fluorines in the spacer as mAlb3634-34-17, but it also contains PS bonds in all the loops present in the gRNA. mAlb3634-34-27 retained 67% activity and mAlb3634-34-29 retained 114% activity. Among the modifications these guides contain are PS bonds in the loop of the first stem loop and 2′-O-methyl groups in the first strand of the first stem loop for mAlb3634-34-27 and mAlb3634-34-29, respectively. When these 2 modifications were combined (2′-O-methyl in the first strand of the first stem loop and PS bonds in the loop of the first stem loop), the guides lost their activity (mAlb3634-34-33, mAlb3634-34-36, mAlb3634-34-38), showing the complexity of the gRNA/protein interaction and demonstrating that the results of simple extrapolations are difficult to predict.

In order to test the stability of these chemically modified guides compared to the guide with no chemical modification (native RNA), a stability assay using crude cell extracts was used. Crude cell extracts from mammalian cells were selected because they contain the mixture of nucleases that a guide RNA will be exposed to when delivered to mammalian cells in vitro or in vivo. Hepa1-6 cells were collected by adding 3 ml of cold PBS per 15 cm dish of confluent cells and releasing the cells from the surface of the dish using a cell scraper. The cells were pelleted at 200 g for 10 min and frozen at −80° C. for future use. For the stability assays, cells were resuspended in 4 volumes of cold PBS (e.g. for a 100 mg pellet, cells were resuspended in 400 ul of cold PBS). Triton X-100 was added to a concentration of 0.2% (v/v), cells were vortexed for 10 seconds, put on ice for 10 minutes, and vortexed again for 10 seconds. Triton X-100 is a mild non-ionic detergent that disrupts cell membranes but does not inactivate or denature proteins at the concentration used. Stability reactions were set up on ice and comprised 2011.1 of cell crude extract with 2 pmoles of each guide (1 ul of a 2 uM stock). Six reactions were set up per guide comprising: input, 0.5 hour, 1 hour, 4 hours, 9 hours, and in some cases 21 hours (The time in hours referring to the length of time each sample was incubated). Samples were incubated at 37° C. from 0.5 hours up to 21 hours while the input control was left on ice for 5 minutes. After each incubation period, the reaction was stopped by adding 300 ul of a mixture of phenol and guanidine thiocyanate (Tri reagent, Zymo Research), which immediately denatures all proteins and efficiently inhibits ribonucleases and facilitates the subsequent recovery of RNA. After adding Tri Reagent, the samples were vortexed for 15 seconds and stored at −20° C. RNA was extracted from the samples using Direct-zol RNA miniprep kit (Zymo Research) and eluted in 100 ul of nuclease-free water. Detection of the modified guide was performed using Taqman RT—qPCR using the Taqman miRNA Assay technology (Thermo Fisher), and primers and probes were designed to specifically detect the sequence in the mAlb3634-34 sgRNA, which is the same for all of the guides. Data was plotted as a function of percentage of sgRNA remaining in relation to the input sample (Tables 7 and 8; FIG. 13 and FIG. 14).

TABLE 7
Stability of MG3-6/3-4 chemically modified guides over 9 hours at 37° C.

| | Percentage guide left | | | |
|---|---|---|---|---|
| Time (H) | mAlb3634-34-0 | mAlb3634-34-1 | mAlb3634-34-17 | mAlb3634-34-29 |
| 0.5 | 48.6327474 | 71.6977624 | 84.9684999 | 91.383145 |
| 1 | 45.5334917 | 111.342162 | 69.2554734 | 79.8298386 |
| 4 | 8.33311673 | 84.3815796 | 46.6516496 | 58.2366793 |
| 9 | 1.23016871 | 41.3225159 | 36.6021424 | 16.5511114 |
| Time (H) | mAlb3634-34-30 | mAlb3634-34-35 | mAlb3634-34-36 | mAlb3634-34-42 |
| 0.5 | 86.7538687 | | 91.7004043 | 91.7004043 |
| 1 | 90.1250463 | 146.40857 | 57.8344092 | 72.1964598 |
| 4 | 53.5886731 | 128.34259 | 61.985385 | 72.1964598 |
| 9 | 21.9912269 | 100 | 62.6332219 | 47.3028823 |

TABLE 8
Stability of MG3-6/3-4 chemically modified guides over 21 hours at 37° C.

| | Percentage guide left | | | |
|---|---|---|---|---|
| Time (H) | mAlb3634-34-0 | mAlb3634-34-1 | mAlb3634-34-35 | mAlb3634-34-42 |
| 0.5 | 68.3020128 | 61.98539 | 104.6085 | 80.94422 |
| 1 | 51.0506063 | 59.66679 | 84.08964 | 73.20428 |
| 4 | 9.67228121 | 51.05061 | 52.66805 | 70.71068 |
| 9 | 1.75790388 | 40.47211 | 51.22784 | 45.37596 |
| 21 | 0.03405136 | 1.447794 | 24.82731 | 15.60413 |

The stability assays showed that introducing three 2′-O-methyls and three PS bonds in the 5′ and 3′ end of the guides significantly improved stability (FIG. 13 and FIG. 14). Adding extra 2′-fluors to the 5′ and 3′ modifications, as in mAlb3634-17 and mAlb3634-42, did not show an apparent advantage at early time points (up to 9 hr) as shown in FIG. 13, but a slight improvement in stability was apparent when the stability assays were run for 21 hr (FIG. 14). Including 2-O-methyl and PS bonds in all the loops of the stem loops (mAlb3634-35) gave an apparent larger increment in stability compared to the guide with chemical modifications on the 5′ and 3′ ends (mAlb3634-1), as seen in FIG. 13. However, when these results were repeated and at longer time points, this increment became less apparent at earlier time points and was became apparent at longer time points up to 21 hr, as seen in FIG. 14. Including 2′-O-methyl in the first strand of distinct stem loops did not provide an advantage in stability for up to 9 hr, as shown by comparing mAlb3634-0 and mAlb3634-29 and mAlb3634-30. mAlb3634-36, which has a combination of 2′-O-methyl in the first strand of all stem loops and PS bonds in the loops of all stem loops, showed an apparent increased stability at 9 hr when compared to end modified guide (mAlb3634-0). However, this guide was not active when tested via mRNA transfection in Hepa1-6 cells. In general, adding extra modifications (e.g. 2′-O-methyl, 2′-O-fluor or PS bonds) to the end modified guide did not confer a large advantage in stability at earlier time points up to 9 hr (FIG. 13), and a small increase in stability was apparent at longer time points (FIG. 14). The large size (110nt) and highly structured nature of this gRNA may make it inherently more stable than shorter or less structured guide RNA and thereby limit the benefit of chemical modifications on stability. Modifying the 5′ and 3′ ends of the guide appears to provide a good level of protection against nucleases. However adding the extra modifications in the guides might provide more benefit in vivo, as these types of modifications may reduce immunogenicity.

### Example 10—Protein Recombination of Type V-A Nucleases

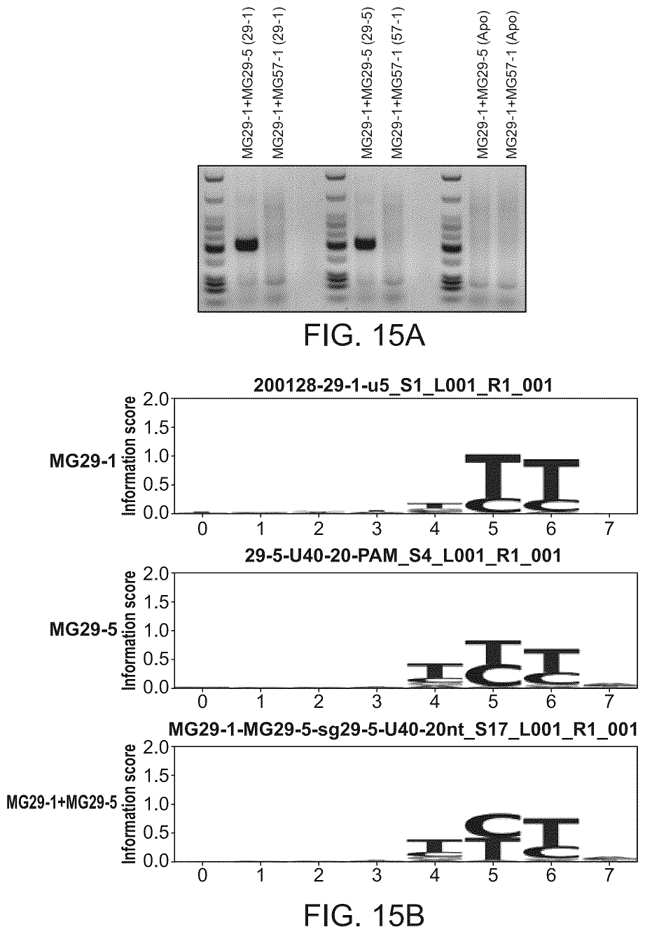

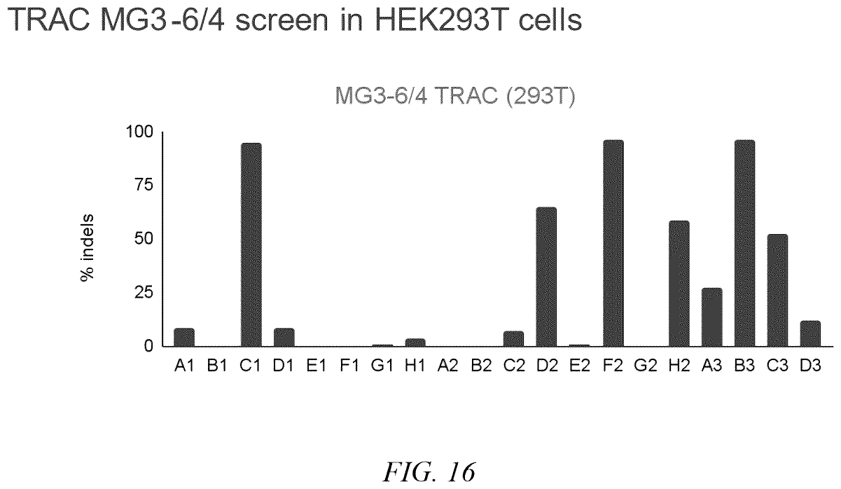

To expand the capability of rapid PAM exchange beyond type II nucleases, three type V-A nucleases were chosen for protein recombination. The breakpoint was chosen based on the predicted structural information (Table 1). Similar to type II enzyme recombinants, the type V chimera showed activity when proteins were recombined from a closely related family. In vitro PAM enrichment and NGS analysis revealed a consistent result that the PAM of a chimera is inherited from C-terminal parent. It may be possible to avoid potential structural disruptions of protein recombination from distantly related families by utilizing breakpoint optimization (FIG. 15).

### Example 11—Analysis of Gene-Editing Outcomes at the DNA Level for TRAC in HEK293T Cells

Nucleofection of MG3-6/4 RNPs (104 pmol protein/300 pmol guide) comprising sgRNAs described below in Table 7A and SEQ ID NOs: 119-158 was performed into HEK293T cells (200,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 16). Results indicated that sgRNAs C1, F2, and B3 were most effective at inducing indels, with appreciable editing also occurring for sgRNAs D2, H2, A3, and C3.

TABLE 7A
gRNAs and Targeting Sequences Used in Example 11

| | SEQ | | |
|---|---|---|---|
| | ID | | |
| Category | NO: | Name | Sequence |
| MG3-6/3- | 119 | MG3- | mG*mC*mC*rGrUrGrUrArCrCrArGrCrUrGrArGrArGrArCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | A1 | rUrCrCrArArUrArGrGrArGrCrGrGrGrCrGrGrUrArUrGrU*mU*mU*mU |
| MG3-6/3- | 120 | MG3- | mA*mU*mU*rCrArCrCrGrArUrUrUrUrGrArUrUrCrUrCrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | B1 | rUrCrCrArArUrArGrGrArGrCrGrGrGrCrGrGrUrArUrGrU*mU*mU*mU |
| MG3-6/3- | 121 | MG3- | mG*mA*mU*rUrCrUrGrArUrGrUrGrUrArUrArUrCrArCrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | C1 | rUrCrCrArArUrArGrGrArGrCrGrGrGrCrGrGrUrArUrGrU*mU*mU*mU |
| MG3-6/3- | 122 | MG3- | mA*mA*mC*rArGrUrGrCrUrGrUrGrGrCrCrUrGrGrArGrCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | D1 | rUrCrCrArArUrArGrGrArGrCrGrGrGrCrGrGrUrArUrGrU*mU*mU*mU |
| MG3-6/3- | 123 | MG3- | mG*mG*mC*rUrGrGrGrGrArArGrArArGrGrUrGrUrCrUrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | E1 | rUrCrCrArArUrArGrGrArGrCrGrGrGrCrGrGrUrArUrGrU*mU*mU*mU |
| MG3-6/3- | 124 | MG3- | mG*mU*mU*rUrUrGrUrCrUrGrUrGrArUrArUrArCrArCrArUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | F1 | rUrCrCrArArUrArGrGrArGrCrGrGrGrCrGrGrUrArUrGrU*mU*mU*mU |
| MG3-6/3- | 125 | MG3- | mU*mU*mA*rCrUrUrUrGrUrGrArCrArCrArUrUrUrGrUrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | G1 | rUrCrCrArArUrArGrGrArGrCrGrGrGrCrGrGrUrArUrGrU*mU*mU*mU |
| MG3-6/3- | 126 | MG3- | mU*mU*mG*rUrGrArCrArCrArUrUrUrGrUrUrUrGrArGrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | H1 | rUrCrCrArArUrArGrGrArGrCrGrGrGrCrGrGrUrArUrGrU*mU*mU*mU |
| MG3-6/3- | 127 | MG3- | mU*mG*mU*rGrArCrArCrArUrUrUrGrUrUrUrGrArGrArArUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | A2 | rUrCrCrArArUrArGrGrArGrCrGrGrGrCrGrGrUrArUrGrU*mU*mU*mU |
| MG3-6/3- | 128 | MG3- | mA*mU*mU*rUrGrUrUrUrGrArGrArArUrCrArArArArUrCrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | B2 | rUrCrCrArArUrArGrGrArGrCrGrGrGrCrGrGrUrArUrGrU*mU*mU*mU |
| MG3-6/3- | 129 | MG3- | mU*mU*mC*rCrUrGrUrGrArUrGrUrCrArArGrCrUrGrGrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | C2 | rUrCrCrArArUrArGrGrArGrCrGrGrGrCrGrGrUrArUrGrU*mU*mU*mU |
| MG3-6/3- | 130 | MG3- | mU*mC*mC*rUrGrUrGrArUrGrUrCrArArGrCrUrGrGrUrCrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | D2 | rUrCrCrArArUrArGrGrArGrCrGrGrGrCrGrGrUrArUrGrU*mU*mU*mU |
| MG3-6/3- | 131 | MG3- | mG*mU*mC*rArArGrCrUrGrGrUrCrGrArGrArArArArGrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | E2 | rUrCrCrArArUrArGrGrArGrCrGrGrGrCrGrGrUrArUrGrU*mU*mU*mU |
| MG3-6/3- | 132 | MG3- | mA*mG*mC*rUrUrGrArCrArUrCrArCrArGrGrArArCrUrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | F2 | rUrCrCrArArUrArGrGrArGrCrGrGrGrCrGrGrUrArUrGrU*mU*mU*mU |
| MG3-6/3- | 133 | MG3- | mG*mA*mC*rArUrCrArCrArGrGrArArCrUrUrUrCrUrArArArGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | G2 | rUrCrCrArArUrArGrGrArGrCrGrGrGrCrGrGrUrArUrGrU*mU*mU*mU |
| MG3-6/3- | 134 | MG3- | mU*mU*mA*rCrArGrArUrArCrGrArArCrCrUrArArArCrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | H2 | rUrCrCrArArUrArGrGrArGrCrGrGrGrCrGrGrUrArUrGrU*mU*mU*mU |
| MG3-6/3- | 135 | MG3- | mA*mA*mA*rArCrCrUrGrUrCrArGrUrGrArUrUrGrGrGrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | A3 | rUrCrCrArArUrArGrGrArGrCrGrGrGrCrGrGrUrArUrGrU*mU*mU*mU |
| MG3-6/3- | 136 | MG3- | mG*mA*mU*rUrGrGrGrUrUrCrCrGrArArUrCrCrUrCrCrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | B3 | rUrCrCrArArUrArGrGrArGrCrGrGrGrCrGrGrUrArUrGrU*mU*mU*mU |
| MG3-6/3- | 137 | MG3- | mG*mG*mA*rArCrCrCrArArUrCrArCrUrGrArCrArGrGrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | C3 | rUrCrCrArArUrArGrGrArGrCrGrGrGrCrGrGrUrArUrGrU*mU*mU*mU |
| MG3-6/3- | 138 | MG3- | mU*mU*mG*rArArArGrUrUrUrArGrGrUrUrCrGrUrArUrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRAC | rUrCrCrGrArUrGrCrUrGrArCrUrUrCrUrCrArCrCrGrUrCrCrGrUrUrU |
| TRAC | | D3 | rUrCrCrArArUrArGrGrArGrCrGrGrGrCrGrGrUrArUrGrU*mU*mU*mU |
| DNA | 139 | MG3- | GCCGTGTACCAGCTGAGAGACT |
| sequence | | 6/3-4 | |
| of TRAC | | TRAC | |
| target site | | A1 | |
| DNA | 140 | MG3- | ATTCACCGATTTTGATTCTCAA |
| sequence | | 6/3-4 | |
| of TRAC | | TRAC | |
| target site | | B1 | |
| DNA | 141 | MG3- | GATTCTGATGTGTATATCACAG |
| sequence | | 6/3-4 | |
| of TRAC | | TRAC | |
| target site | | C1 | |
| DNA | 142 | MG3- | AACAGTGCTGTGGCCTGGAGCA |
| sequence | | 6/3-4 | |
| of TRAC | | TRAC | |
| target site | | D1 | |
| DNA | 143 | MG3- | GGCTGGGGAAGAAGGTGTCTTC |
| sequence | | 6/3-4 | |
| of TRAC | | TRAC | |
| target site | | E1 | |
| DNA | 144 | MG3- | GTTTTGTCTGTGATATACACAT |
| sequence | | 6/3-4 | |
| of TRAC | | TRAC | |
| target site | | F1 | |
| DNA | 145 | MG3- | TTACTTTGTGACACATTTGTTT |
| sequence | | 6/3-4 | |
| of TRAC | | TRAC | |
| target site | | G1 | |
| DNA | 146 | MG3- | TTGTGACACATTTGTTTGAGAA |
| sequence | | 6/3-4 | |
| of TRAC | | TRAC | |
| target site | | H1 | |
| DNA | 147 | MG3- | TGTGACACATTTGTTTGAGAAT |
| sequence | | 6/3-4 | |
| of TRAC | | TRAC | |
| target site | | A2 | |
| DNA | 148 | MG3- | ATTTGTTTGAGAATCAAAATCG |
| sequence | | 6/3-4 | |
| of TRAC | | TRAC | |
| target site | | B2 | |
| DNA | 149 | MG3- | TTCCTGTGATGTCAAGCTGGTC |
| sequence | | 6/3-4 | |
| of TRAC | | TRAC | |
| target site | | C2 | |
| DNA | 150 | MG3- | TCCTGTGATGTCAAGCTGGTCG |
| sequence | | 6/3-4 | |
| of TRAC | | TRAC | |
| target site | | D2 | |
| DNA | 151 | MG3- | GTCAAGCTGGTCGAGAAAAGCT |
| sequence | | 6/3-4 | |
| of TRAC | | TRAC | |
| target site | | E2 | |
| DNA | 152 | MG3- | AGCTTGACATCACAGGAACTTT |
| sequence | | 6/3-4 | |
| of TRAC | | TRAC | |
| target site | | F2 | |
| DNA | 153 | MG3- | GACATCACAGGAACTTTCTAAA |
| sequence | | 6/3-4 | |
| of TRAC | | TRAC | |
| target site | | G2 | |
| DNA | 154 | MG3- | TTACAGATACGAACCTAAACTT |
| sequence | | 6/3-4 | |
| of TRAC | | TRAC | |
| target site | | H2 | |
| DNA | 155 | MG3- | AAAACCTGTCAGTGATTGGGTT |
| sequence | | 6/3-4 | |
| of TRAC | | TRAC | |
| target site | | A3 | |
| DNA | 156 | MG3- | GATTGGGTTCCGAATCCTCCTC |
| sequence | | 6/3-4 | |
| of TRAC | | TRAC | |
| target site | | B3 | |
| DNA | 157 | MG3- | GGAACCCAATCACTGACAGGTT |
| sequence | | 6/3-4 | |
| of TRAC | | TRAC | |
| target site | | C3 | |
| DNA | 158 | MG3- | TTGAAAGTTTAGGTTCGTATCT |
| sequence | | 6/3-4 | |
| of TRAC | | TRAC | |
| target site | | D3 | |

(r = native ribose base, m = 2′-O methyl modified base, F = 2′ Fluro modified base, * = phosphorothioate bond)

### Example 12—Analysis of Gene-Editing Outcomes at the DNA Level for B2M in HEK293T Cells

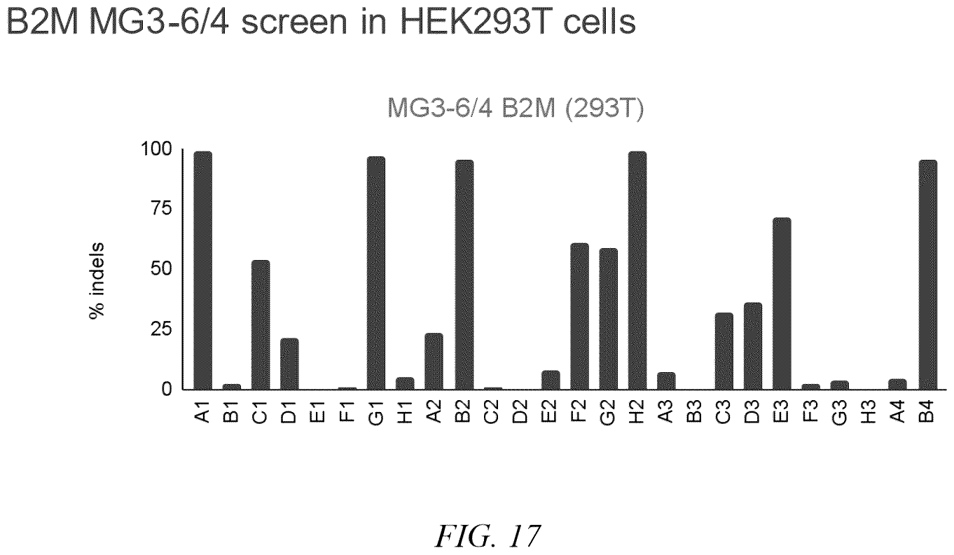

Nucleofection of MG3-6/4 RNPs (104 pmol protein/300 pmol guide) comprising sgRNAs described below in Table 7B and SEQ ID NOs: 159-210 was performed into HEK293T cells (200,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 17). Results indicated that sgRNAs A1, G1, B2, H2, and B4 were the most effective for inducing editing, with appreciable editing also being detected for sgRNAs C1, D1, A2, H1, E2, F2, G2, A3, C3, and D3.

TABLE 7B
gRNAs and Targeting Sequences Used in Example 12

| | SEQ | | |
|---|---|---|---|
| | ID | | |
| Category | NO: | Name | Sequence |
| MG3-6/3- | 159 | MG3- | mU*mC*mA*rCrGrCrUrGrGrArUrArGrCrCrUrCrCrArGrGrCrGrUrUrGrA |
| 4 sgRNA | | 6/3-4 | rGrArArUrCrGrArArArGrArUrUrCrUrUrArArUrArArGrGrCrArUrCrCrU |
| targeting | | B2M A1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 160 | MG3- | mG*mG*mU*rUrUrArCrUrCrArCrGrUrCrArUrCrCrArGrCrArGrUrUrGrA |
| 4 sgRNA | | 6/3-4 | rGrArArUrCrGrArArArGrArUrUrCrUrUrArArUrArArGrGrCrArUrCrCrU |
| targeting | | B2M B1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 161 | MG3- | mA*mC*mU*rCrArCrGrUrCrArUrCrCrArGrCrArGrArGrArArGrUrUrGrA |
| 4 sgRNA | | 6/3-4 | rGrArArUrCrGrArArArGrArUrUrCrUrUrArArUrArArGrGrCrArUrCrCrU |
| targeting | | B2M C1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 162 | MG3- | mU*mC*mA*rUrCrCrArGrCrArGrArGrArArUrGrGrArArArGrGrUrUrGrA |
| 4 sgRNA | | 6/3-4 | rGrArArUrCrGrArArArGrArUrUrCrUrUrArArUrArArGrGrCrArUrCrCrU |
| targeting | | B2M D1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 163 | MG3- | mA*mG*mA*rGrArArUrGrGrArArArGrUrCrArArArUrUrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | B2M E1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 164 | MG3- | mC*mG*mA*rCrArUrUrGrArArGrUrUrGrArCrUrUrArCrUrGrGrUrUrGrA |
| 4 sgRNA | | 6/3-4 | rGrArArUrCrGrArArArGrArUrUrCrUrUrArArUrArArGrGrCrArUrCrCrU |
| targeting | | B2M F1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 165 | MG3- | mU*mU*mG*rArCrUrUrArCrUrGrArArGrArArUrGrGrArGrArGrUrUrGrA |
| 4 sgRNA | | 6/3-4 | rGrArArUrCrGrArArArGrArUrUrCrUrUrArArUrArArGrGrCrArUrCrCrU |
| targeting | | B2M G1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 166 | MG3- | mU*mU*mA*rCrUrGrArArGrArArUrGrGrArGrArGrArGrArArGrUrUrGrA |
| 4 sgRNA | | 6/3-4 | rGrArArUrCrGrArArArGrArUrUrCrUrUrArArUrArArGrGrCrArUrCrCrU |
| targeting | | B2M H1 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 167 | MG3- | mU*mA*mC*rUrGrArArGrArArUrGrGrArGrArGrArGrArArUrGrUrUrGrA |
| 4 sgRNA | | 6/3-4 | rGrArArUrCrGrArArArGrArUrUrCrUrUrArArUrArArGrGrCrArUrCrCrU |
| targeting | | B2M A2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 168 | MG3- | mA*mC*mU*rGrArArGrArArUrGrGrArGrArGrArGrArArUrUrGrUrUrGrA |
| 4 sgRNA | | 6/3-4 | rGrArArUrCrGrArArArGrArUrUrCrUrUrArArUrArArGrGrCrArUrCrCrU |
| targeting | | B2M B2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 169 | MG3- | mU*mC*mU*rUrUrCrUrArUrCrUrCrUrUrGrUrArCrUrArCrArGrUrUrGrA |
| 4 sgRNA | | 6/3-4 | rGrArArUrCrGrArArArGrArUrUrCrUrUrArArUrArArGrGrCrArUrCrCrU |
| targeting | | B2M C2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 170 | MG3- | mU*mA*mC*rUrArCrArCrUrGrArArUrUrCrArCrCrCrCrCrArGrUrUrGrA |
| 4 sgRNA | | 6/3-4 | rGrArArUrCrGrArArArGrArUrUrCrUrUrArArUrArArGrGrCrArUrCrCrU |
| targeting | | B2M D2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 171 | MG3- | mA*mC*mU*rArCrArCrUrGrArArUrUrCrArCrCrCrCrCrArCrGrUrUrGrA |
| 4 sgRNA | | 6/3-4 | rGrArArUrCrGrArArArGrArUrUrCrUrUrArArUrArArGrGrCrArUrCrCrU |
| targeting | | B2ME2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 172 | MG3- | mC*mU*mA*rCrArCrUrGrArArUrUrCrArCrCrCrCrCrArCrUrGrUrUrGrA |
| 4 sgRNA | | 6/3-4 | rGrArArUrCrGrArArArGrArUrUrCrUrUrArArUrArArGrGrCrArUrCrCrU |
| targeting | | B2MF2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 173 | MG3- | mA*mU*mA*rCrUrCrArUrCrUrUrUrUrUrCrArGrUrGrGrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | B2M G2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 174 | MG3- | mG*mA*mA*rUrUrCrArGrUrGrUrArGrUrArCrArArGrArGrArGrUrUrGrA |
| 4 sgRNA | | 6/3-4 | rGrArArUrCrGrArArArGrArUrUrCrUrUrArArUrArArGrGrCrArUrCrCrU |
| targeting | | B2M H2 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 175 | MG3- | mG*mA*mG*rArUrArGrArArArGrArCrCrArGrUrCrCrUrUrGrGrUrUrGrA |
| 4 sgRNA | | 6/3-4 | rGrArArUrCrGrArArArGrArUrUrCrUrUrArArUrArArGrGrCrArUrCrCrU |
| targeting | | B2M A3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 176 | MG3- | mC*mA*mG*rUrCrCrUrUrGrCrUrGrArArArGrArCrArArGrUrGrUrUrGrA |
| 4 sgRNA | | 6/3-4 | rGrArArUrCrGrArArArGrArUrUrCrUrUrArArUrArArGrGrCrArUrCrCrU |
| targeting | | B2M B3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 177 | MG3- | mA*mG*mU*rCrArArCrUrUrCrArArUrGrUrCrGrGrArUrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | B2M C3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 178 | MG3- | mA*mA*mA*rCrCrCrArGrArCrArCrArUrArGrCrArArUrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | B2M D3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 179 | MG3- | mA*mA*mC*rCrCrArGrArCrArCrArUrArGrCrArArUrUrCrArGrUrUrGrA |
| 4 sgRNA | | 6/3-4 | rGrArArUrCrGrArArArGrArUrUrCrUrUrArArUrArArGrGrCrArUrCrCrU |
| targeting | | B2ME3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 180 | MG3- | mC*mU*mG*rCrUrGrGrArUrGrArCrGrUrGrArGrUrArArArCrGrUrUrGrA |
| 4 sgRNA | | 6/3-4 | rGrArArUrCrGrArArArGrArUrUrCrUrUrArArUrArArGrGrCrArUrCrCrU |
| targeting | | B2M F3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 181 | MG3- | mA*mC*mC*rUrGrArArUrCrUrUrUrGrGrArGrUrArCrCrUrGrGrUrUrGrA |
| 4 sgRNA | | 6/3-4 | rGrArArUrCrGrArArArGrArUrUrCrUrUrArArUrArArGrGrCrArUrCrCrU |
| targeting | | B2M G3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 182 | MG3- | mU*mG*mC*rUrGrCrUrUrArCrArUrGrUrCrUrCrGrArUrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | B2M H3 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 183 | MG3- | mG*mC*mU*rGrCrUrUrArCrArUrGrUrCrUrCrGrArUrCrUrArGrUrUrGrA |
| 4 sgRNA | | 6/3-4 | rGrArArUrCrGrArArArGrArUrUrCrUrUrArArUrArArGrGrCrArUrCrCrU |
| targeting | | B2M A4 | rUrCrCrGrArUrGrCrUrGrArCrUrUrCrUrCrArCrCrGrUrCrCrGrUrUrU |
| B2M | | | rUrCrCrArArUrArGrGrArGrCrGrGrGrCrGrGrUrArUrGrU*mU*mU*mU |
| MG3-6/3- | 184 | MG3- | mC*mU*mG*rCrUrUrArCrArUrGrUrCrUrCrGrArUrCrUrArUrGrUrUrGrA |
| 4 sgRNA | | 6/3-4 | rGrArArUrCrGrArArArGrArUrUrCrUrUrArArUrArArGrGrCrArUrCrCrU |
| targeting | | B2M | rUrCrCrGrArUrGrCrUrGrArCrUrUrCrUrCrArCrCrGrUrCrCrGrUrUrU |
| B2M | | B4 | rUrCrCrArArUrArGrGrArGrCrGrGrGrCrGrGrUrArUrGrU*mU*mU*mU |
| DNA | 185 | MG3- | TCACGCTGGATAGCCTCCAGGC |
| sequence | | 6/3-4 | |
| of B2M | | B2M A1 | |
| target site | | | |
| DNA | 186 | MG3- | GGTTTACTCACGTCATCCAGCA |
| sequence | | 6/3-4 | |
| of B2M | | B2MB1 | |
| target site | | | |
| DNA | 187 | MG3- | ACTCACGTCATCCAGCAGAGAA |
| sequence | | 6/3-4 | |
| of B2M | | B2M C1 | |
| target site | | | |
| DNA | 188 | MG3- | TCATCCAGCAGAGAATGGAAAG |
| sequence | | 6/3-4 | |
| of B2M | | B2M D1 | |
| target site | | | |
| DNA | 189 | MG3- | AGAGAATGGAAAGTCAAATTTC |
| sequence | | 6/3-4 | |
| of B2M | | B2M E1 | |
| target site | | | |
| DNA | 190 | MG3- | CGACATTGAAGTTGACTTACTG |
| sequence | | 6/3-4 | |
| of B2M | | B2M F1 | |
| target site | | | |
| DNA | 191 | MG3- | TTGACTTACTGAAGAATGGAGA |
| sequence | | 6/3-4 | |
| of B2M | | B2M G1 | |
| target site | | | |
| DNA | 192 | MG3- | TTACTGAAGAATGGAGAGAGAA |
| sequence | | 6/3-4 | |
| of B2M | | B2M H1 | |
| target site | | | |
| DNA | 193 | MG3- | TACTGAAGAATGGAGAGAGAAT |
| sequence | | 6/3-4 | |
| of B2M | | B2M A2 | |
| target site | | | |
| DNA | 194 | MG3- | ACTGAAGAATGGAGAGAGAATT |
| sequence | | 6/3-4 | |
| of B2M | | B2M B2 | |
| target site | | | |
| DNA | 195 | MG3- | TCTTTCTATCTCTTGTACTACA |
| sequence | | 6/3-4 | |
| of B2M | | B2M C2 | |
| target site | | | |
| DNA | 196 | MG3- | TACTACACTGAATTCACCCCCA |
| sequence | | 6/3-4 | |
| of B2M | | B2M D2 | |
| target site | | | |
| DNA | 197 | MG3- | ACTACACTGAATTCACCCCCAC |
| sequence | | 6/3-4 | |
| of B2M | | B2M E2 | |
| target site | | | |
| DNA | 198 | MG3- | CTACACTGAATTCACCCCCACT |
| sequence | | 6/3-4 | |
| of B2M | | B2M F2 | |
| target site | | | |
| DNA | 199 | MG3- | ATACTCATCTTTTTCAGTGGGG |
| sequence | | 6/3-4 | |
| of B2M | | B2M G2 | |
| target site | | | |
| DNA | 200 | MG3- | GAATTCAGTGTAGTACAAGAGA |
| sequence | | 6/3-4 | |
| of B2M | | B2M H2 | |
| target site | | | |
| DNA | 201 | MG3- | GAGATAGAAAGACCAGTCCTTG |
| sequence | | 6/3-4 | |
| of B2M | | B2M A3 | |
| target site | | | |
| DNA | 202 | MG3- | CAGTCCTTGCTGAAAGACAAGT |
| sequence | | 6/3-4 | |
| of B2M | | B2M B3 | |
| target site | | | |
| DNA | 203 | MG3- | AGTCAACTTCAATGTCGGATGG |
| sequence | | 6/3-4 | |
| of B2M | | B2M C3 | |
| target site | | | |
| DNA | 204 | MG3- | AAACCCAGACACATAGCAATTC |
| sequence | | 6/3-4 | |
| of B2M | | B2M D3 | |
| target site | | | |
| DNA | 205 | MG3- | AACCCAGACACATAGCAATTCA |
| sequence | | 6/3-4 | |
| of B2M | | B2ME3 | |
| target site | | | |
| DNA | 206 | MG3- | CTGCTGGATGACGTGAGTAAAC |
| sequence | | 6/3-4 | |
| of B2M | | B2M F3 | |
| target site | | | |
| DNA | 207 | MG3- | ACCTGAATCTTTGGAGTACCTG |
| sequence | | 6/3-4 | |
| of B2M | | B2M G3 | |
| target site | | | |
| DNA | 208 | MG3- | TGCTGCTTACATGTCTCGATCT |
| sequence | | 6/3-4 | |
| of B2M | | B2M H3 | |
| target site | | | |
| DNA | 209 | MG3- | GCTGCTTACATGTCTCGATCTA |
| sequence | | 6/3-4 | |
| of B2M | | B2M A4 | |
| target site | | | |
| DNA | 210 | MG3- | CTGCTTACATGTCTCGATCTAT |
| sequence | | 6/3-4 | |
| of B2M | | B2M B4 | |
| target site | | | |

(r = native ribose base, m = 2′-O methyl modified base, F = 2′ Fluro modified base,* = phosphorothioate bond)

### Example 13—Analysis of Gene-Editing Outcomes at the DNA and Phenotypic Levels for TRAC in T Cells

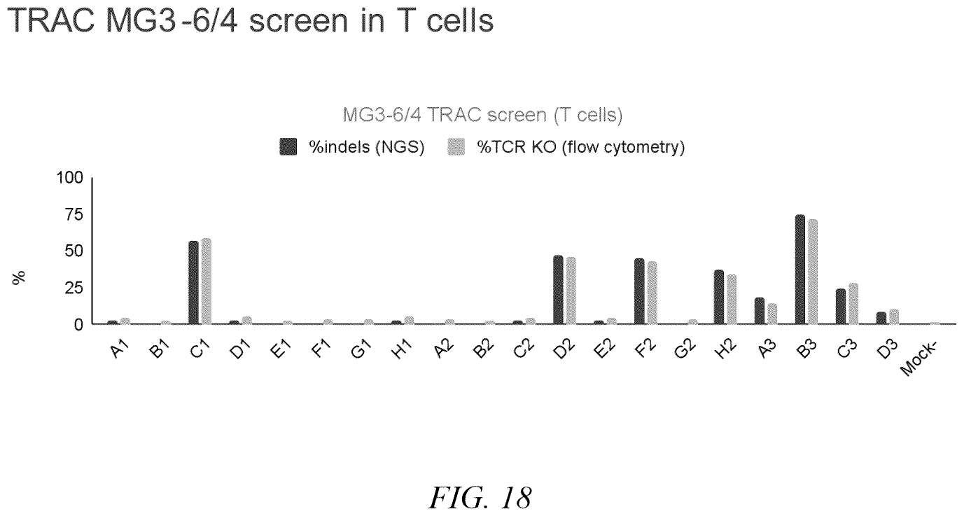

Primary T cells were purified from PMBCs using a negative selection kit (Miltenyi) according to the manufacturer's recommendations. Nucleofection of MG3-6/4 RNPs (104 pmol protein/120 pmol guide) comprising sgRNAs described in Table 7A and SEQ ID NOs: 119-158 was performed into T cells (200,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing. For analysis by flow cytometry, 3 days post-nucleofection, 100,000 T cells were stained with anti-CD3 antibody for 30 minutes at 4C and analyzed on an Attune Nxt flow cytometer (FIG. 18). Results indicated that sgRNAs C1, D2, F2, H2, A3, B3, C3, and D3 showed appreciable editing, with the most editing performed by sgRNAs C1 and B3.

### Example 14—Analysis of Gene-Editing Outcomes at the DNA Level for B2M in T Cells

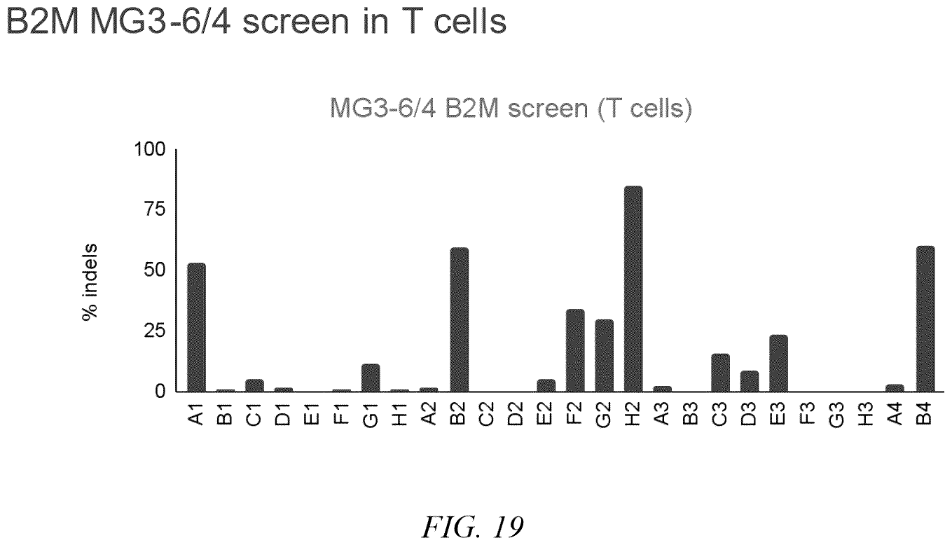

Primary T cells were purified from PMBCs using a negative selection kit (Miltenyi) according to the manufacturer's recommendations. Nucleofection of MG3-6/4 RNPs (104 pmol protein/120 pmol guide) comprising sgRNAs described in Table 7B and SEQ ID NOs: 159-210 was performed into T cells (200,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 19).

### Example 15—Analysis of Gene-Editing Outcomes at the Phenotypic Level for TRBC1 and TRBC2 in T Cells

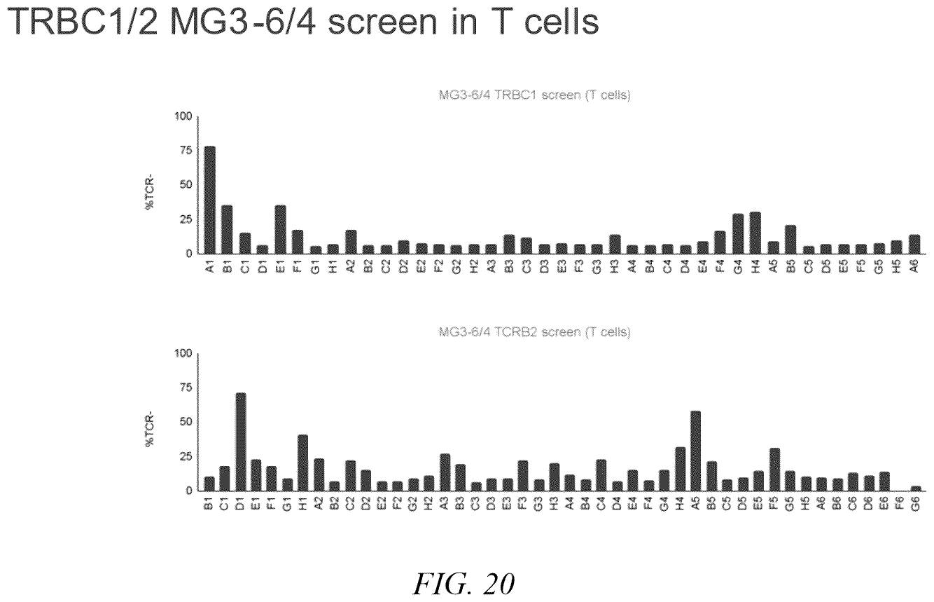

Primary T cells were purified from PBMCs using a negative selection kit (Miltenyi) according to the manufacturer's recommendations. Nucleofection of MG3-6/4 RNPs (104 pmol protein/120 pmol guide) comprising sgRNAs described below in Table 7C below and SEQ ID NOs: 211-382 was performed into T cells (200,000) using the Lonza 4D electroporator. For analysis by flow cytometry, 3 days post-nucleofection, 100,000 T cells were stained with anti-CD3 antibody for 30 minutes at 4C and analyzed on an Attune Nxt flow cytometer (FIG. 20). As can be seen from the results in FIG. 20, the highest-performing sgRNAs for TRBC1 were A1, B1, E1, G4, H4, and B5. Similarly, the highest performing sgRNAs for TRBC2 were D1, H1, and A5.

TABLE 7C
gRNAs and Targeting Sequences Used in Example 15

| | SEQ | | |
|---|---|---|---|
| | ID | | |
| Category | NO: | Name | Sequence |
| MG3-6/3- | 211 | MG3- | mC*mA*mG*rArArGrCrArGrArGrArUrCrUrCrCrCrArCrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | A1 | rUrCrCrArArUrArGrGrArGrCrGrGrGrCrGrGrUrArUrGrU*mU*mU*mU |
| MG3-6/3- | 212 | MG3- | mC*mC*mA*rCrGrUrGrGrArGrCrUrGrArGrCrUrGrGrUrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | B1 | rUrCrCrArArUrArGrGrArGrCrGrGrGrCrGrGrUrArUrGrU*mU*mU*mU |
| MG3-6/3- | 213 | MG3- | mA*mG*mU*rCrCrArGrUrUrCrUrArCrGrGrGrCrUrCrUrCrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | C1 | rUrCrCrArArUrArGrGrArGrCrGrGrGrCrGrGrUrArUrGrU*mU*mU*mU |
| MG3-6/3- | 214 | MG3- | mG*mA*mU*rUrArGrGrUrGrArGrArCrCrArGrCrUrArCrCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | D1 | rUrCrCrArArUrArGrGrArGrCrGrGrGrCrGrGrUrArUrGrU*mU*mU*mU |
| MG3-6/3- | 215 | MG3- | mA*mU*mU*rArGrGrUrGrArGrArCrCrArGrCrUrArCrCrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | E1 | rUrCrCrArArUrArGrGrArGrCrGrGrGrCrGrGrUrArUrGrU*mU*mU*mU |
| MG3-6/3- | 216 | MG3- | mU*mU*mA*rGrGrUrGrArGrArCrCrArGrCrUrArCrCrArGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBCI | rUrCrCrGrArUrGrCrUrGrArCrUrUrCrUrCrArCrCrGrUrCrCrGrUrUrU |
| TRBC1 | | F1 | rUrCrCrArArUrArGrGrArGrCrGrGrGrCrGrGrUrArUrGrU*mU*mU*mU |
| MG3-6/3- | 217 | MG3- | mU*mG*mA*rGrArCrCrArGrCrUrArCrCrArGrGrGrArArArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | G1 | rUrCrCrArArUrArGrGrArGrCrGrGrGrCrGrGrUrArUrGrU*mU*mU*mU |
| MG3-6/3- | 218 | MG3- | mC*mA*mG*rGrUrArGrCrArGrArCrArArGrArCrUrArGrArUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | H1 | rUrCrCrArArUrArGrGrArGrCrGrGrGrCrGrGrUrArUrGrU*mU*mU*mU |
| MG3-6/3- | 219 | MG3- | mA*mG*mG*rUrArGrCrArGrArCrArArGrArCrUrArGrArUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | A2 | rUrCrCrArArUrArGrGrArGrCrGrGrGrCrGrGrUrArUrGrU*mU*mU*mU |
| MG3-6/3- | 220 | MG3- | mA*mG*mC*rArGrArCrArArGrArCrUrArGrArUrCrCrArArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | B2 | rUrCrCrArArUrArGrGrArGrCrGrGrGrCrGrGrUrArUrGrU*mU*mU*mU |
| MG3-6/3- | 221 | MG3- | mG*mG*mA*rArCrCrArGrCrGrCrArCrArCrCrArUrGrArArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | C2 | rUrCrCrArArUrArGrGrArGrCrGrGrGrCrGrGrUrArUrGrU*mU*mU*mU |
| MG3-6/3- | 222 | MG3- | mG*mU*mG*rGrCrUrGrArCrArUrCrUrGrCrArUrGrGrCrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | D2 | rUrCrCrArArUrArGrGrArGrCrGrGrGrCrGrGrUrArUrGrU*mU*mU*mU |
| MG3-6/3- | 223 | MG3- | mG*mG*mC*rCrUrGrGrGrArGrUrCrUrGrUrGrCrCrArArCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | E2 | rUrCrCrArArUrArGrGrArGrCrGrGrGrCrGrGrUrArUrGrU*mU*mU*mU |
| MG3-6/3- | 224 | MG3- | mC*mU*mG*rArCrUrUrUrArCrUrUrUrUrArArUrUrGrCrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | F2 | rUrCrCrArArUrArGrGrArGrCrGrGrGrCrGrGrUrArUrGrU*mU*mU*mU |
| MG3-6/3- | 225 | MG3- | mU*mG*mA*rCrUrUrUrArCrUrUrUrUrArArUrUrGrCrCrUrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | G2 | rUrCrCrArArUrArGrGrArGrCrGrGrGrCrGrGrUrArUrGrU*mU*mU*mU |
| MG3-6/3- | 226 | MG3- | mG*mA*mC*rUrUrUrArCrUrUrUrUrArArUrUrGrCrCrUrArUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | H2 | rUrCrCrArArUrArGrGrArGrCrGrGrGrCrGrGrUrArUrGrU*mU*mU*mU |
| MG3-6/3- | 227 | MG3- | mG*mG*mG*rArArGrGrArGrArArGrCrUrGrGrArGrUrCrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | A3 | rUrCrCrArArUrArGrGrArGrCrGrGrGrCrGrGrUrArUrGrU*mU*mU*mU |
| MG3-6/3- | 228 | MG3- | mG*mG*mA*rArGrGrArGrArArGrCrUrGrGrArGrUrCrArCrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | B3 | rUrCrCrArArUrArGrGrArGrCrGrGrGrCrGrGrUrArUrGrU*mU*mU*mU |
| MG3-6/3- | 229 | MG3- | mA*mA*mC*rUrCrCrUrGrGrCrUrCrUrUrArArUrArArCrCrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | C3 | rUrCrCrArArUrArGrGrArGrCrGrGrGrCrGrGrUrArUrGrU*mU*mU*mU |
| MG3-6/3- | 230 | MG3- | mA*mA*mC*rUrUrUrCrUrCrUrUrCrUrGrCrArGrGrUrCrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | D3 | rUrCrCrArArUrArGrGrArGrCrGrGrGrCrGrGrUrArUrGrU*mU*mU*mU |
| MG3-6/3- | 231 | MG3- | mA*mC*mU*rCrCrArCrUrUrCrCrArGrGrGrCrUrGrCrCrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | E3 | rUrCrCrArArUrArGrGrArGrCrGrGrGrCrGrGrUrArUrGrU*mU*mU*mU |
| MG3-6/3- | 232 | MG3- | mC*mU*mC*rCrArCrUrUrCrCrArGrGrGrCrUrGrCrCrUrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | F3 | rUrCrCrArArUrArGrGrArGrCrGrGrGrCrGrGrUrArUrGrU*mU*mU*mU |
| MG3-6/3- | 233 | MG3- | mU*mC*mC*rUrUrUrCrUrCrUrUrGrArCrCrUrGrCrArGrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | G3 | rUrCrCrArArUrArGrGrArGrCrGrGrGrCrGrGrUrArUrGrU*mU*mU*mU |
| MG3-6/3- | 234 | MG3- | mA*mG*mC*rCrArGrGrArGrUrUrGrUrGrArGrGrArUrUrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | H3 | rUrCrCrArArUrArGrGrArGrCrGrGrGrCrGrGrUrArUrGrU*mU*mU*mU |
| MG3-6/3- | 235 | MG3- | mA*mG*mU*rArGrUrArGrGrGrCrCrCrArUrUrGrArCrCrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | A4 | rUrCrCrArArUrArGrGrArGrCrGrGrGrCrGrGrUrArUrGrU*mU*mU*mU |
| MG3-6/3- | 236 | MG3- | mU*mG*mC*rArArGrUrUrArUrCrUrUrCrUrGrArGrGrCrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | B4 | rUrCrCrArArUrArGrGrArGrCrGrGrGrCrGrGrUrArUrGrU*mU*mU*mU |
| MG3-6/3- | 237 | MG3- | mA*mG*mU*rUrArUrCrUrUrCrUrGrArGrGrCrArCrCrUrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | C4 | rUrCrCrArArUrArGrGrArGrCrGrGrGrCrGrGrUrArUrGrU*mU*mU*mU |
| MG3-6/3- | 238 | MG3- | mG*mU*mU*rArUrCrUrUrCrUrGrArGrGrCrArCrCrUrGrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | D4 | rUrCrCrArArUrArGrGrArGrCrGrGrGrCrGrGrUrArUrGrU*mU*mU*mU |
| MG3-6/3- | 239 | MG3- | mU*mC*mA*rArGrArArCrCrArUrGrArGrArGrArGrGrGrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | E4 | rUrCrCrArArUrArGrGrArGrCrGrGrGrCrGrGrUrArUrGrU*mU*mU*mU |
| MG3-6/3- | 240 | MG3- | mC*mA*mA*rGrArArCrCrArUrGrArGrArGrArGrGrGrArGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | F4 | rUrCrCrArArUrArGrGrArGrCrGrGrGrCrGrGrUrArUrGrU*mU*mU*mU |
| MG3-6/3- | 241 | MG3- | mU*mU*mA*rCrCrCrGrArGrGrUrArArArGrCrCrArCrArGrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | G4 | rUrCrCrArArUrArGrGrArGrCrGrGrGrCrGrGrUrArUrGrU*mU*mU*mU |
| MG3-6/3- | 242 | MG3- | mC*mC*mG*rArGrGrUrArArArGrCrCrArCrArGrUrCrUrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | H4 | rUrCrCrArArUrArGrGrArGrCrGrGrGrCrGrGrUrArUrGrU*mU*mU*mU |
| MG3-6/3- | 243 | MG3- | mC*mA*mG*rUrCrUrGrArArArGrArArArGrCrArGrGrGrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | A5 | rUrCrCrArArUrArGrGrArGrCrGrGrGrCrGrGrUrArUrGrU*mU*mU*mU |
| MG3-6/3- | 244 | MG3- | mA*mG*mU*rCrUrGrArArArGrArArArGrCrArGrGrGrArGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | B5 | rUrCrCrArArUrArGrGrArGrCrGrGrGrCrGrGrUrArUrGrU*mU*mU*mU |
| MG3-6/3- | 245 | MG3- | mG*mU*mC*rUrGrArArArGrArArArGrCrArGrGrGrArGrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | C5 | rUrCrCrArArUrArGrGrArGrCrGrGrGrCrGrGrUrArUrGrU*mU*mU*mU |
| MG3-6/3- | 246 | MG3- | mG*mA*mA*rArGrArArArGrCrArGrGrGrArGrArGrGrArArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | D5 | rUrCrCrArArUrArGrGrArGrCrGrGrGrCrGrGrUrArUrGrU*mU*mU*mU |
| MG3-6/3- | 247 | MG3- | mG*mA*mG*rArCrCrUrUrArUrUrUrUrCrArUrArGrGrCrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | E5 | rUrCrCrArArUrArGrGrArGrCrGrGrGrCrGrGrUrArUrGrU*mU*mU*mU |
| MG3-6/3- | 248 | MG3- | mG*mA*mU*rGrArGrArGrUrUrArCrArCrArGrGrCrCrArCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | F5 | rUrCrCrArArUrArGrGrArGrCrGrGrGrCrGrGrUrArUrGrU*mU*mU*mU |
| MG3-6/3- | 249 | MG3- | mA*mG*mC*rUrGrCrUrUrGrGrCrUrCrUrGrUrUrGrGrGrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | G5 | rUrCrCrArArUrArGrGrArGrCrGrGrGrCrGrGrUrArUrGrU*mU*mU*mU |
| MG3-6/3- | 250 | MG3- | mU*mG*mU*rUrGrGrGrCrUrGrArGrArArUrCrUrGrGrGrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | H5 | rUrCrCrArArUrArGrGrArGrCrGrGrGrCrGrGrUrArUrGrU*mU*mU*mU |
| MG3-6/3- | 251 | MG3- | mG*mG*mA*rArCrArCrCrUrUrGrUrUrCrArGrGrUrCrCrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC1 | rUrCrCrGrArUrGrCrUrGrArCrUrUrCrUrCrArCrCrGrUrCrCrGrUrUrU |
| TRBC1 | | A6 | rUrCrCrArArUrArGrGrArGrCrGrGrGrCrGrGrUrArUrGrU*mU*mU*mU |
| DNA | 252 | MG3- | CAGAAGCAGAGATCTCCCACAC |
| sequence | | 6/3-4 | |
| of TRBC1 | | TRBC1 | |
| target site | | A1 | |
| DNA | 253 | MG3- | CCACGTGGAGCTGAGCTGGTGG |
| sequence | | 6/3-4 | |
| of TRBC1 | | TRBC1 | |
| target site | | B1 | |
| DNA | 254 | MG3- | AGTCCAGTTCTACGGGCTCTCG |
| sequence | | 6/3-4 | |
| of TRBC1 | | TRBC1 | |
| target site | | C1 | |
| DNA | 255 | MG3- | GATTAGGTGAGACCAGCTACCA |
| sequence | | 6/3-4 | |
| of TRBC1 | | TRBC1 | |
| target site | | D1 | |
| DNA | 256 | MG3- | ATTAGGTGAGACCAGCTACCAG |
| sequence | | 6/3-4 | |
| of TRBC1 | | TRBC1 | |
| target site | | E1 | |
| DNA | 257 | MG3- | TTAGGTGAGACCAGCTACCAGG |
| sequence | | 6/3-4 | |
| of TRBC1 | | TRBC1 | |
| target site | | F1 | |
| DNA | 258 | MG3- | TGAGACCAGCTACCAGGGAAAA |
| sequence | | 6/3-4 | |
| of TRBC1 | | TRBC1 | |
| target site | | G1 | |
| DNA | 259 | MG3- | CAGGTAGCAGACAAGACTAGAT |
| sequence | | 6/3-4 | |
| of TRBC1 | | TRBC1 | |
| target site | | H1 | |
| DNA | 260 | MG3- | AGGTAGCAGACAAGACTAGATC |
| sequence | | 6/3-4 | |
| of TRBC1 | | TRBC1 | |
| target site | | A2 | |
| DNA | 261 | MG3- | AGCAGACAAGACTAGATCCAAA |
| sequence | | 6/3-4 | |
| of TRBC1 | | TRBC1 | |
| target site | | B2 | |
| DNA | 262 | MG3- | GGAACCAGCGCACACCATGAAG |
| sequence | | 6/3-4 | |
| of TRBC1 | | TRBC1 | |
| target site | | C2 | |
| DNA | 263 | MG3- | GTGGCTGACATCTGCATGGCAG |
| sequence | | 6/3-4 | |
| of TRBC1 | | TRBC1 | |
| target site | | D2 | |
| DNA | 264 | MG3- | GGCCTGGGAGTCTGTGCCAACT |
| sequence | | 6/3-4 | |
| of TRBC1 | | TRBC1 | |
| target site | | E2 | |
| DNA | 265 | MG3- | CTGACTTTACTTTTAATTGCCT |
| sequence | | 6/3-4 | |
| of TRBC1 | | TRBC1 | |
| target site | | F2 | |
| DNA | 266 | MG3- | TGACTTTACTTTTAATTGCCTA |
| sequence | | 6/3-4 | |
| of TRBC1 | | TRBC1 | |
| target site | | G2 | |
| DNA | 267 | MG3- | GACTTTACTTTTAATTGCCTAT |
| sequence | | 6/3-4 | |
| of TRBC1 | | TRBC1 | |
| target site | | H2 | |
| DNA | 268 | MG3- | GGGAAGGAGAAGCTGGAGTCAC |
| sequence | | 6/3-4 | |
| of TRBC1 | | TRBC1 | |
| target site | | A3 | |
| DNA | 269 | MG3- | GGAAGGAGAAGCTGGAGTCACC |
| sequence | | 6/3-4 | |
| of TRBC1 | | TRBC1 | |
| target site | | B3 | |
| DNA | 270 | MG3- | AACTCCTGGCTCTTAATAACCC |
| sequence | | 6/3-4 | |
| of TRBC1 | | TRBC1 | |
| target site | | C3 | |
| DNA | 271 | MG3- | AACTTTCTCTTCTGCAGGTCAA |
| sequence | | 6/3-4 | |
| of TRBC1 | | TRBC1 | |
| target site | | D3 | |
| DNA | 272 | MG3- | ACTCCACTTCCAGGGCTGCCTT |
| sequence | | 6/3-4 | |
| of TRBC1 | | TRBC1 | |
| target site | | E3 | |
| DNA | 273 | MG3- | CTCCACTTCCAGGGCTGCCTTC |
| sequence | | 6/3-4 | |
| of TRBC1 | | TRBC1 | |
| target site | | F3 | |
| DNA | 274 | MG3- | TCCTTTCTCTTGACCTGCAGAA |
| sequence | | 6/3-4 | |
| of TRBC1 | | TRBC1 | |
| target site | | G3 | |
| DNA | 275 | MG3- | AGCCAGGAGTTGTGAGGATTGA |
| sequence | | 6/3-4 | |
| of TRBC1 | | TRBC1 | |
| target site | | H3 | |
| DNA | 276 | MG3- | AGTAGTAGGGCCCATTGACCAC |
| sequence | | 6/3-4 | |
| of TRBC1 | | TRBC1 | |
| target site | | A4 | |
| DNA | 277 | MG3- | TGCAAGTTATCTTCTGAGGCAC |
| sequence | | 6/3-4 | |
| of TRBC1 | | TRBC1 | |
| target site | | B4 | |
| DNA | 278 | MG3- | AGTTATCTTCTGAGGCACCTGA |
| sequence | | 6/3-4 | |
| of TRBC1 | | TRBC1 | |
| target site | | C4 | |
| DNA | 279 | MG3- | GTTATCTTCTGAGGCACCTGAA |
| sequence | | 6/3-4 | |
| of TRBC1 | | TRBC1 | |
| target site | | D4 | |
| DNA | 280 | MG3- | TCAAGAACCATGAGAGAGGGAG |
| sequence | | 6/3-4 | |
| of TRBC1 | | TRBC1 | |
| target site | | E4 | |
| DNA | 281 | MG3- | CAAGAACCATGAGAGAGGGAGA |
| sequence | | 6/3-4 | |
| of TRBC1 | | TRBC1 | |
| target site | | F4 | |
| DNA | 282 | MG3- | TTACCCGAGGTAAAGCCACAGT |
| sequence | | 6/3-4 | |
| of TRBC1 | | TRBC1 | |
| target site | | G4 | |
| DNA | 283 | MG3- | CCGAGGTAAAGCCACAGTCTGA |
| sequence | | 6/3-4 | |
| of TRBC1 | | TRBC1 | |
| target site | | H4 | |
| DNA | 284 | MG3- | CAGTCTGAAAGAAAGCAGGGAG |
| sequence | | 6/3-4 | |
| of TRBC1 | | TRBC1 | |
| target site | | A5 | |
| DNA | 285 | MG3- | AGTCTGAAAGAAAGCAGGGAGA |
| sequence | | 6/3-4 | |
| of TRBC1 | | TRBC1 | |
| target site | | B5 | |
| DNA | 286 | MG3- | GTCTGAAAGAAAGCAGGGAGAG |
| sequence | | 6/3-4 | |
| of TRBC1 | | TRBC1 | |
| target site | | C5 | |
| DNA | 287 | MG3- | GAAAGAAAGCAGGGAGAGGAAA |
| sequence | | 6/3-4 | |
| of TRBC1 | | TRBC1 | |
| target site | | D5 | |
| DNA | 288 | MG3- | GAGACCTTATTTTCATAGGCAA |
| sequence | | 6/3-4 | |
| of TRBC1 | | TRBC1 | |
| target site | | E5 | |
| DNA | 289 | MG3- | GATGAGAGTTACACAGGCCACA |
| sequence | | 6/3-4 | |
| of TRBC1 | | TRBC1 | |
| target site | | F5 | |
| DNA | 290 | MG3- | AGCTGCTTGGCTCTGTTGGGCT |
| sequence | | 6/3-4 | |
| of TRBC1 | | TRBC1 | |
| target site | | G5 | |
| DNA | 291 | MG3- | TGTTGGGCTGAGAATCTGGGAG |
| sequence | | 6/3-4 | |
| of TRBC1 | | TRBC1 | |
| target site | | H5 | |
| DNA | 292 | MG3- | GGAACACCTTGTTCAGGTCCTC |
| sequence | | 6/3-4 | |
| of TRBC1 | | TRBC1 | |
| target site | | A6 | |
| MG3-6/3- | 293 | MG3- | mA*mC*mC*rUrCrUrUrCrCrCrUrUrUrCrCrArGrArGrGrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | A1 | rUrCrCrArArUrArGrGrArGrCrGrGrGrCrGrGrUrArUrGrU*mU*mU*mU |
| MG3-6/3- | 294 | MG3- | mC*mC*mU*rCrUrUrCrCrCrUrUrUrCrCrArGrArGrGrArCrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | B1 | rUrCrCrArArUrArGrGrArGrCrGrGrGrCrGrGrUrArUrGrU*mU*mU*mU |
| MG3-6/3- | 295 | MG3- | mC*mU*mC*rUrUrCrCrCrUrUrUrCrCrArGrArGrGrArCrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | C1 | rUrCrCrArArUrArGrGrArGrCrGrGrGrCrGrGrUrArUrGrU*mU*mU*mU |
| MG3-6/3- | 296 | MG3- | mC*mA*mG*rArArGrCrArGrArGrArUrCrUrCrCrCrArCrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | D1 | rUrCrCrArArUrArGrGrArGrCrGrGrGrCrGrGrUrArUrGrU*mU*mU*mU |
| MG3-6/3- | 297 | MG3- | mC*mC*mA*rCrGrUrGrGrArGrCrUrGrArGrCrUrGrGrUrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | E1 | rUrCrCrArArUrArGrGrArGrCrGrGrGrCrGrGrUrArUrGrU*mU*mU*mU |
| MG3-6/3- | 298 | MG3- | mA*mG*mU*rCrCrArGrUrUrCrUrArCrGrGrGrCrUrCrUrCrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | F1 | rUrCrCrArArUrArGrGrArGrCrGrGrGrCrGrGrUrArUrGrU*mU*mU*mU |
| MG3-6/3- | 299 | MG3- | mG*mA*mU*rUrArGrGrUrGrArGrArCrCrArGrCrUrArCrCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | G1 | rUrCrCrArArUrArGrGrArGrCrGrGrGrCrGrGrUrArUrGrU*mU*mU*mU |
| MG3-6/3- | 300 | MG3- | mA*mU*mU*rArGrGrUrGrArGrArCrCrArGrCrUrArCrCrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | H1 | rUrCrCrArArUrArGrGrArGrCrGrGrGrCrGrGrUrArUrGrU*mU*mU*mU |
| MG3-6/3- | 301 | MG3- | mU*mU*mA*rGrGrUrGrArGrArCrCrArGrCrUrArCrCrArGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | A2 | rUrCrCrArArUrArGrGrArGrCrGrGrGrCrGrGrUrArUrGrU*mU*mU*mU |
| MG3-6/3- | 302 | MG3- | mU*mG*mA*rGrArCrCrArGrCrUrArCrCrArGrGrGrArArArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | B2 | rUrCrCrArArUrArGrGrArGrCrGrGrGrCrGrGrUrArUrGrU*mU*mU*mU |
| MG3-6/3- | 303 | MG3- | mU*mA*mG*rCrGrGrArCrArArGrArCrUrArGrArUrCrCrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | C2 | rUrCrCrArArUrArGrGrArGrCrGrGrGrCrGrGrUrArUrGrU*mU*mU*mU |
| MG3-6/3- | 304 | MG3- | mC*mC*mC*rCrCrArCrCrArArGrArArGrCrArUrArGrArGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | D2 | rUrCrCrArArUrArGrGrArGrCrGrGrGrCrGrGrUrArUrGrU*mU*mU*mU |
| MG3-6/3- | 305 | MG3- | mU*mC*mU*rGrCrUrCrUrCrGrArArCrCrArGrGrGrCrArUrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | E2 | rUrCrCrArArUrArGrGrArGrCrGrGrGrCrGrGrUrArUrGrU*mU*mU*mU |
| MG3-6/3- | 306 | MG3- | mG*mG*mA*rArCrArUrCrArCrArCrArUrGrGrGrCrArUrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | F2 | rUrCrCrArArUrArGrGrArGrCrGrGrGrCrGrGrUrArUrGrU*mU*mU*mU |
| MG3-6/3- | 307 | MG3- | mC*mC*mU*rArArUrArUrArUrCrCrUrArUrCrArCrCrUrCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | G2 | rUrCrCrArArUrArGrGrArGrCrGrGrGrCrGrGrUrArUrGrU*mU*mU*mU |
| MG3-6/3- | 308 | MG3- | mA*mC*mC*rArUrArArUrGrArArGrCrCrArGrArCrUrGrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | H2 | rUrCrCrArArUrArGrGrArGrCrGrGrGrCrGrGrUrArUrGrU*mU*mU*mU |
| MG3-6/3- | 309 | MG3- | mC*mC*mA*rUrArArUrGrArArGrCrCrArGrArCrUrGrGrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | A3 | rUrCrCrArArUrArGrGrArGrCrGrGrGrCrGrGrUrArUrGrU*mU*mU*mU |
| MG3-6/3- | 310 | MG3- | mC*mA*mU*rArArUrGrArArGrCrCrArGrArCrUrGrGrGrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | B3 | rUrCrCrArArUrArGrGrArGrCrGrGrGrCrGrGrUrArUrGrU*mU*mU*mU |
| MG3-6/3- | 311 | MG3- | mG*mC*mC*rArGrArCrUrGrGrGrGrArGrArArArArUrGrCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | C3 | rUrCrCrArArUrArGrGrArGrCrGrGrGrCrGrGrUrArUrGrU*mU*mU*mU |
| MG3-6/3- | 312 | MG3- | mG*mG*mA*rGrArArArArUrGrCrArGrGrGrArArUrArUrCrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | D3 | rUrCrCrArArUrArGrGrArGrCrGrGrGrCrGrGrUrArUrGrU*mU*mU*mU |
| MG3-6/3- | 313 | MG3- | mG*mG*mA*rGrArCrArArCrCrArGrCrGrArGrCrCrCrUrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | E3 | rUrCrCrArArUrArGrGrArGrCrGrGrGrCrGrGrUrArUrGrU*mU*mU*mU |
| MG3-6/3- | 314 | MG3- | mU*mA*mC*rUrCrCrUrGrCrUrGrUrGrCrCrArUrArGrCrCrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | F3 | rUrCrCrArArUrArGrGrArGrCrGrGrGrCrGrGrUrArUrGrU*mU*mU*mU |
| MG3-6/3- | 315 | MG3- | mC*mU*mG*rUrGrCrCrArUrArGrCrCrCrCrUrGrArArArCrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | G3 | rUrCrCrArArUrArGrGrArGrCrGrGrGrCrGrGrUrArUrGrU*mU*mU*mU |
| MG3-6/3- | 316 | MG3- | mU*mG*mU*rGrCrCrArUrArGrCrCrCrCrUrGrArArArCrCrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | H3 | rUrCrCrArArUrArGrGrArGrCrGrGrGrCrGrGrUrArUrGrU*mU*mU*mU |
| MG3-6/3- | 317 | MG3- | mG*mU*mG*rCrCrArUrArGrCrCrCrCrUrGrArArArCrCrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | A4 | rUrCrCrArArUrArGrGrArGrCrGrGrGrCrGrGrUrArUrGrU*mU*mU*mU |
| MG3-6/3- | 318 | MG3- | mU*mG*mU*rUrCrUrCrUrCrUrUrCrCrArCrArGrGrUrCrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | B4 | rUrCrCrArArUrArGrGrArGrCrGrGrGrCrGrGrUrArUrGrU*mU*mU*mU |
| MG3-6/3- | 319 | MG3- | mG*mA*mA*rArGrGrArUrUrCrCrArGrArGrGrCrUrArGrCrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | C4 | rUrCrCrArArUrArGrGrArGrCrGrGrGrCrGrGrUrArUrGrU*mU*mU*mU |
| MG3-6/3- | 320 | MG3- | mG*mG*mA*rUrGrGrUrUrUrUrGrGrArGrCrUrArGrCrCrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | D4 | rUrCrCrArArUrArGrGrArGrCrGrGrGrCrGrGrUrArUrGrU*mU*mU*mU |
| MG3-6/3- | 321 | MG3- | mC*mC*mC*rUrGrGrUrUrCrGrArGrArGrCrArGrArGrArCrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | E4 | rUrCrCrArArUrArGrGrArGrCrGrGrGrCrGrGrUrArUrGrU*mU*mU*mU |
| MG3-6/3- | 322 | MG3- | mA*mG*mC*rArGrArGrArCrGrGrCrGrArArArGrArUrArGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | F4 | rUrCrCrArArUrArGrGrArGrCrGrGrGrCrGrGrUrArUrGrU*mU*mU*mU |
| MG3-6/3- | 323 | MG3- | mG*mC*mA*rGrArGrArCrGrGrCrGrArArArGrArUrArGrArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | G4 | rUrCrCrArArUrArGrGrArGrCrGrGrGrCrGrGrUrArUrGrU*mU*mU*mU |
| MG3-6/3- | 324 | MG3- | mC*mA*mG*rArGrArCrGrGrCrGrArArArGrArUrArGrArGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | H4 | rUrCrCrArArUrArGrGrArGrCrGrGrGrCrGrGrUrArUrGrU*mU*mU*mU |
| MG3-6/3- | 325 | MG3- | mU*mU*mA*rCrCrGrGrArGrGrUrGrArArGrCrCrArCrArGrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | A5 | rUrCrCrArArUrArGrGrArGrCrGrGrGrCrGrGrUrArUrGrU*mU*mU*mU |
| MG3-6/3- | 326 | MG3- | mC*mG*mG*rArGrGrUrGrArArGrCrCrArCrArGrUrCrUrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | B5 | rUrCrCrArArUrArGrGrArGrCrGrGrGrCrGrGrUrArUrGrU*mU*mU*mU |
| MG3-6/3- | 327 | MG3- | mG*mG*mA*rGrGrUrGrArArGrCrCrArCrArGrUrCrUrGrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | C5 | rUrCrCrArArUrArGrGrArGrCrGrGrGrCrGrGrUrArUrGrU*mU*mU*mU |
| MG3-6/3- | 328 | MG3- | mA*mC*mA*rGrUrCrUrGrArArArGrArArArArCrArGrGrGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | D5 | rUrCrCrArArUrArGrGrArGrCrGrGrGrCrGrGrUrArUrGrU*mU*mU*mU |
| MG3-6/3- | 329 | MG3- | mC*mA*mG*rUrCrUrGrArArArGrArArArArCrArGrGrGrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | E5 | rUrCrCrArArUrArGrGrArGrCrGrGrGrCrGrGrUrArUrGrU*mU*mU*mU |
| MG3-6/3- | 330 | MG3- | mA*mG*mU*rCrUrGrArArArGrArArArArCrArGrGrGrGrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | F5 | rUrCrCrArArUrArGrGrArGrCrGrGrGrCrGrGrUrArUrGrU*mU*mU*mU |
| MG3-6/3- | 331 | MG3- | mG*mU*mC*rUrGrArArArGrArArArArCrArGrGrGrGrArArGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | G5 | rUrCrCrArArUrArGrGrArGrCrGrGrGrCrGrGrUrArUrGrU*mU*mU*mU |
| MG3-6/3- | 332 | MG3- | mA*mC*mA*rGrGrGrGrArArGrArArArArArUrGrGrArUrGrArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | H5 | rUrCrCrArArUrArGrGrArGrCrGrGrGrCrGrGrUrArUrGrU*mU*mU*mU |
| MG3-6/3- | 333 | MG3- | mG*mC*mG*rArArGrUrGrGrUrCrArCrUrArUrGrArUrCrUrUrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | A6 | rUrCrCrArArUrArGrGrArGrCrGrGrGrCrGrGrUrArUrGrU*mU*mU*mU |
| MG3-6/3- | 334 | MG3- | mU*mU*mA*rGrGrArArArCrCrArGrGrArCrCrCrCrArGrArArGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | B6 | rUrCrCrArArUrArGrGrArGrCrGrGrGrCrGrGrUrArUrGrU*mU*mU*mU |
| MG3-6/3- | 335 | MG3- | mU*mA*mU*rGrGrCrUrGrGrUrCrCrUrCrArGrGrGrArGrArCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | C6 | rUrCrCrArArUrArGrGrArGrCrGrGrGrCrGrGrUrArUrGrU*mU*mU*mU |
| MG3-6/3- | 336 | MG3- | mC*mU*mA*rArGrGrUrGrUrCrArGrGrArUrCrUrGrArArGrGrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | D6 | rUrCrCrArArUrArGrGrArGrCrGrGrGrCrGrGrUrArUrGrU*mU*mU*mU |
| MG3-6/3- | 337 | MG3- | mG*mG*mA*rArCrArCrGrUrUrUrUrUrCrArGrGrUrCrCrUrCrGrUrUrGrA |
| 4 sgRNA | | 6/3-4 | rGrArArUrCrGrArArArGrArUrUrCrUrUrArArUrArArGrGrCrArUrCrCrU |
| targeting | | TRBC2 | rUrCrCrGrArUrGrCrUrGrArCrUrUrCrUrCrArCrCrGrUrCrCrGrUrUrU |
| TRBC2 | | E6 | rUrCrCrArArUrArGrGrArGrCrGrGrGrCrGrGrUrArUrGrU*mU*mU*mU |
| DNA | 338 | MG3- | ACCTCTTCCCTTTCCAGAGGAC |
| sequence | | 6/3-4 | |
| of TRBC2 | | TRBC2 | |
| target site | | A1 | |
| DNA | 339 | MG3- | CCTCTTCCCTTTCCAGAGGACC |
| sequence | | 6/3-4 | |
| of TRBC2 | | TRBC2 | |
| target site | | B1 | |
| DNA | 340 | MG3- | CTCTTCCCTTTCCAGAGGACCT |
| sequence | | 6/3-4 | |
| of TRBC2 | | TRBC2 | |
| target site | | C1 | |
| DNA | 341 | MG3- | CAGAAGCAGAGATCTCCCACAC |
| sequence | | 6/3-4 | |
| of TRBC2 | | TRBC2 | |
| target site | | D1 | |
| DNA | 342 | MG3- | CCACGTGGAGCTGAGCTGGTGG |
| sequence | | 6/3-4 | |
| of TRBC2 | | TRBC2 | |
| target site | | E1 | |
| DNA | 343 | MG3- | AGTCCAGTTCTACGGGCTCTCG |
| sequence | | 6/3-4 | |
| of TRBC2 | | TRBC2 | |
| target site | | F1 | |
| DNA | 344 | MG3- | GATTAGGTGAGACCAGCTACCA |
| sequence | | 6/3-4 | |
| of TRBC2 | | TRBC2 | |
| target site | | G1 | |
| DNA | 345 | MG3- | ATTAGGTGAGACCAGCTACCAG |
| sequence | | 6/3-4 | |
| of TRBC2 | | TRBC2 | |
| target site | | H1 | |
| DNA | 346 | MG3- | TTAGGTGAGACCAGCTACCAGG |
| sequence | | 6/3-4 | |
| of TRBC2 | | TRBC2 | |
| target site | | A2 | |
| DNA | 347 | MG3- | TGAGACCAGCTACCAGGGAAAA |
| sequence | | 6/3-4 | |
| of TRBC2 | | TRBC2 | |
| target site | | B2 | |
| DNA | 348 | MG3- | TAGCGGACAAGACTAGATCCAG |
| sequence | | 6/3-4 | |
| of TRBC2 | | TRBC2 | |
| target site | | C2 | |
| DNA | 349 | MG3- | CCCCCACCAAGAAGCATAGAGG |
| sequence | | 6/3-4 | |
| of TRBC2 | | TRBC2 | |
| target site | | D2 | |
| DNA | 350 | MG3- | TCTGCTCTCGAACCAGGGCATG |
| sequence | | 6/3-4 | |
| of TRBC2 | | TRBC2 | |
| target site | | E2 | |
| DNA | 351 | MG3- | GGAACATCACACATGGGCATAA |
| sequence | | 6/3-4 | |
| of TRBC2 | | TRBC2 | |
| target site | | F2 | |
| DNA | 352 | MG3- | CCTAATATATCCTATCACCTCA |
| sequence | | 6/3-4 | |
| of TRBC2 | | TRBC2 | |
| target site | | G2 | |
| DNA | 353 | MG3- | ACCATAATGAAGCCAGACTGGG |
| sequence | | 6/3-4 | |
| of TRBC2 | | TRBC2 | |
| target site | | H2 | |
| DNA | 354 | MG3- | CCATAATGAAGCCAGACTGGGG |
| sequence | | 6/3-4 | |
| of TRBC2 | | TRBC2 | |
| target site | | A3 | |
| DNA | 355 | MG3- | CATAATGAAGCCAGACTGGGGA |
| sequence | | 6/3-4 | |
| of TRBC2 | | TRBC2 | |
| target site | | B3 | |
| DNA | 356 | MG3- | GCCAGACTGGGGAGAAAATGCA |
| sequence | | 6/3-4 | |
| of TRBC2 | | TRBC2 | |
| target site | | C3 | |
| DNA | 357 | MG3- | GGAGAAAATGCAGGGAATATCA |
| sequence | | 6/3-4 | |
| of TRBC2 | | TRBC2 | |
| target site | | D3 | |
| DNA | 358 | MG3- | GGAGACAACCAGCGAGCCCTAC |
| sequence | | 6/3-4 | |
| of TRBC2 | | TRBC2 | |
| target site | | E3 | |
| DNA | 359 | MG3- | TACTCCTGCTGTGCCATAGCCC |
| sequence | | 6/3-4 | |
| of TRBC2 | | TRBC2 | |
| target site | | F3 | |
| DNA | 360 | MG3- | CTGTGCCATAGCCCCTGAAACC |
| sequence | | 6/3-4 | |
| of TRBC2 | | TRBC2 | |
| target site | | G3 | |
| DNA | 361 | MG3- | TGTGCCATAGCCCCTGAAACCC |
| sequence | | 6/3-4 | |
| of TRBC2 | | TRBC2 | |
| target site | | H3 | |
| DNA | 362 | MG3- | GTGCCATAGCCCCTGAAACCCT |
| sequence | | 6/3-4 | |
| of TRBC2 | | TRBC2 | |
| target site | | A4 | |
| DNA | 363 | MG3- | TGTTCTCTCTTCCACAGGTCAA |
| sequence | | 6/3-4 | |
| of TRBC2 | | TRBC2 | |
| target site | | B4 | |
| DNA | 364 | MG3- | GAAAGGATTCCAGAGGCTAGCT |
| sequence | | 6/3-4 | |
| of TRBC2 | | TRBC2 | |
| target site | | C4 | |
| DNA | 365 | MG3- | GGATGGTTTTGGAGCTAGCCTC |
| sequence | | 6/3-4 | |
| of TRBC2 | | TRBC2 | |
| target site | | D4 | |
| DNA | 366 | MG3- | CCCTGGTTCGAGAGCAGAGACG |
| sequence | | 6/3-4 | |
| of TRBC2 | | TRBC2 | |
| target site | | E4 | |
| DNA | 367 | MG3- | AGCAGAGACGGCGAAAGATAGA |
| sequence | | 6/3-4 | |
| of TRBC2 | | TRBC2 | |
| target site | | F4 | |
| DNA | 368 | MG3- | GCAGAGACGGCGAAAGATAGAG |
| sequence | | 6/3-4 | |
| of TRBC2 | | TRBC2 | |
| target site | | G4 | |
| DNA | 369 | MG3- | CAGAGACGGCGAAAGATAGAGA |
| sequence | | 6/3-4 | |
| of TRBC2 | | TRBC2 | |
| target site | | H4 | |
| DNA | 370 | MG3- | TTACCGGAGGTGAAGCCACAGT |
| sequence | | 6/3-4 | |
| of TRBC2 | | TRBC2 | |
| target site | | A5 | |
| DNA | 371 | MG3- | CGGAGGTGAAGCCACAGTCTGA |
| sequence | | 6/3-4 | |
| of TRBC2 | | TRBC2 | |
| target site | | B5 | |
| DNA | 372 | MG3- | GGAGGTGAAGCCACAGTCTGAA |
| sequence | | 6/3-4 | |
| of TRBC2 | | TRBC2 | |
| target site | | C5 | |
| DNA | 373 | MG3- | ACAGTCTGAAAGAAAACAGGGG |
| sequence | | 6/3-4 | |
| of TRBC2 | | TRBC2 | |
| target site | | D5 | |
| DNA | 374 | MG3- | CAGTCTGAAAGAAAACAGGGGA |
| sequence | | 6/3-4 | |
| of TRBC2 | | TRBC2 | |
| target site | | E5 | |
| DNA | 375 | MG3- | AGTCTGAAAGAAAACAGGGGAA |
| sequence | | 6/3-4 | |
| of TRBC2 | | TRBC2 | |
| target site | | F5 | |
| DNA | 376 | MG3- | GTCTGAAAGAAAACAGGGGAAG |
| sequence | | 6/3-4 | |
| of TRBC2 | | TRBC2 | |
| target site | | G5 | |
| DNA | 377 | MG3- | ACAGGGGAAGAAAAATGGATGA |
| sequence | | 6/3-4 | |
| of TRBC2 | | TRBC2 | |
| target site | | H5 | |
| DNA | 378 | MG3- | GCGAAGTGGTCACTATGATCTT |
| sequence | | 6/3-4 | |
| of TRBC2 | | TRBC2 | |
| target site | | A6 | |
| DNA | 379 | MG3- | TTAGGAAACCAGGACCCCAGAA |
| sequence | | 6/3-4 | |
| of TRBC2 | | TRBC2 | |
| target site | | B6 | |
| DNA | 380 | MG3- | TATGGCTGGTCCTCAGGGAGAC |
| sequence | | 6/3-4 | |
| of TRBC2 | | TRBC2 | |
| target site | | C6 | |
| DNA | 381 | MG3- | CTAAGGTGTCAGGATCTGAAGG |
| sequence | | 6/3-4 | |
| of TRBC2 | | TRBC2 | |
| target site | | D6 | |
| DNA | 382 | MG3- | GGAACACGTTTTTCAGGTCCTC |
| sequence | | 6/3-4 | |
| of TRBC2 | | TRBC2 | |
| target site | | E6 | |

(r =native ribose base, m = 2′-O methyl modified base, F = 2′ Fluro modified base, * = phosphorothioate bond)

### Example 16—Analysis of Gene-Editing Outcomes at the DNA Level for ANGPTL3 in Hep3B Cells

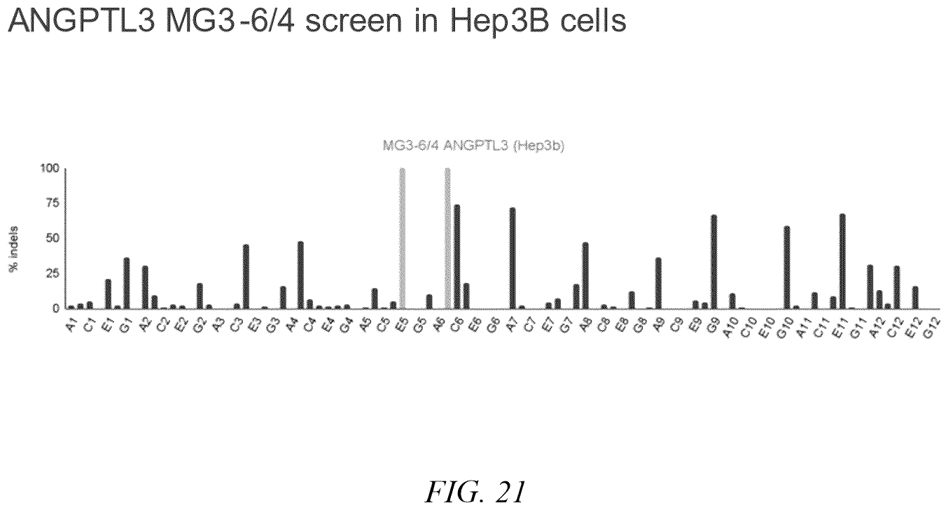

Nucleofection of MG3-6/4 RNPs (104 pmol protein/120 pmol guide) comprising sgRNAs described below in Table 7D below and SEQ ID NOs: 383-572 was performed into Hep3B cells (100,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 21). The results indicate that sgRNA E5, C6, A7, A8, A9, G9, G10, E11, A12, and C12 are the highest performing sgRNAs in this assay.

TABLE 7D
gRNAs and Targeting Sequences Used in Example 16

| | SEQ | | |
|---|---|---|---|
| | ID | | |
| Category | NO: | Name | Sequence |
| MG3-6/3- | 383 | MG3- | mU*mU*mG*rUrUrCrCrUrCrUrArGrUrUrArUrUrUrCrCrUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A1 | rUrCrCrArArUrArGrGrArGrCrGrGrGrCrGrGrUrArUrGrU*mU*mU*mU |
| MG3-6/3- | 384 | MG3- | mA*mU*mU*rUrGrArUrUrCrUrCrUrArUrCrUrCrCrArGrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B1 | rUrCrCrArArUrArGrGrArGrCrGrGrGrCrGrGrUrArUrGrU*mU*mU*mU |
| MG3-6/3- | 385 | MG3- | mU*mU*mU*rGrArUrUrCrUrCrUrArUrCrUrCrCrArGrArGrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C1 | rUrCrCrArArUrArGrGrArGrCrGrGrGrCrGrGrUrArUrGrU*mU*mU*mU |
| MG3-6/3- | 386 | MG3- | mA*mA*mG*rArUrUrUrGrCrUrArUrGrUrUrArGrArCrGrArUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D1 | rUrCrCrArArUrArGrGrArGrCrGrGrGrCrGrGrUrArUrGrU*mU*mU*mU |
| MG3-6/3- | 387 | MG3- | mA*mG*mA*rUrUrUrGrCrUrArUrGrUrUrArGrArCrGrArUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E1 | rUrCrCrArArUrArGrGrArGrCrGrGrGrCrGrGrUrArUrGrU*mU*mU*mU |
| MG3-6/3- | 388 | MG3- | mG*mA*mU*rUrUrGrCrUrArUrGrUrUrArGrArCrGrArUrGrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F1 | rUrCrCrArArUrArGrGrArGrCrGrGrGrCrGrGrUrArUrGrU*mU*mU*mU |
| MG3-6/3- | 389 | MG3- | mA*mC*mU*rUrUrGrUrCrCrArUrArArGrArCrGrArArGrGrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G1 | rUrCrCrArArUrArGrGrArGrCrGrGrGrCrGrGrUrArUrGrU*mU*mU*mU |
| MG3-6/3- | 390 | MG3- | mA*mG*mG*rGrCrCrArArArUrUrArArUrGrArCrArUrArUrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H1 | rUrCrCrArArUrArGrGrArGrCrGrGrGrCrGrGrUrArUrGrU*mU*mU*mU |
| MG3-6/3- | 391 | MG3- | mG*mG*mG*rCrCrArArArUrUrArArUrGrArCrArUrArUrUrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A2 | rUrCrCrArArUrArGrGrArGrCrGrGrGrCrGrGrUrArUrGrU*mU*mU*mU |
| MG3-6/3- | 392 | MG3- | mU*mA*mU*rGrArUrCrUrArUrCrGrCrUrGrCrArArArCrCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B2 | rUrCrCrArArUrArGrGrArGrCrGrGrGrCrGrGrUrArUrGrU*mU*mU*mU |
| MG3-6/3- | 393 | MG3- | mA*mU*mG*rArUrCrUrArUrCrGrCrUrGrCrArArArCrCrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C2 | rUrCrCrArArUrArGrGrArGrCrGrGrGrCrGrGrUrArUrGrU*mU*mU*mU |
| MG3-6/3- | 394 | MG3- | mC*mA*mA*rArCrCrArGrUrGrArArArUrCrArArArGrArArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D2 | rUrCrCrArArUrArGrGrArGrCrGrGrGrCrGrGrUrArUrGrU*mU*mU*mU |
| MG3-6/3- | 395 | MG3- | mA*mA*mA*rCrCrArGrUrGrArArArUrCrArArArGrArArGrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E2 | rUrCrCrArArUrArGrGrArGrCrGrGrGrCrGrGrUrArUrGrU*mU*mU*mU |
| MG3-6/3- | 396 | MG3- | mA*mC*mA*rArGrUrCrArArArArArUrGrArArGrArGrGrUrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F2 | rUrCrCrArArUrArGrGrArGrCrGrGrGrCrGrGrUrArUrGrU*mU*mU*mU |
| MG3-6/3- | 397 | MG3- | mG*mA*mA*rUrArUrGrUrCrArCrUrUrGrArArCrUrCrArArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G2 | rUrCrCrArArUrArGrGrArGrCrGrGrGrCrGrGrUrArUrGrU*mU*mU*mU |
| MG3-6/3- | 398 | MG3- | mU*mC*mA*rCrUrUrGrArArCrUrCrArArCrUrCrArArArArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H2 | rUrCrCrArArUrArGrGrArGrCrGrGrGrCrGrGrUrArUrGrU*mU*mU*mU |
| MG3-6/3- | 399 | MG3- | mU*mC*mA*rArArArCrUrUrGrArArArGrCrCrUrCrCrUrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A3 | rUrCrCrArArUrArGrGrArGrCrGrGrGrCrGrGrUrArUrGrU*mU*mU*mU |
| MG3-6/3- | 400 | MG3- | mC*mA*mA*rArArCrUrUrGrArArArGrCrCrUrCrCrUrArGrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B3 | rUrCrCrArArUrArGrGrArGrCrGrGrGrCrGrGrUrArUrGrU*mU*mU*mU |
| MG3-6/3- | 401 | MG3- | mA*mA*mA*rArCrUrUrGrArArArGrCrCrUrCrCrUrArGrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C3 | rUrCrCrArArUrArGrGrArGrCrGrGrGrCrGrGrUrArUrGrU*mU*mU*mU |
| MG3-6/3- | 402 | MG3- | mA*mA*mA*rCrUrUrGrArArArGrCrCrUrCrCrUrArGrArArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D3 | rUrCrCrArArUrArGrGrArGrCrGrGrGrCrGrGrUrArUrGrU*mU*mU*mU |
| MG3-6/3- | 403 | MG3- | mA*mA*mC*rUrUrGrArArArGrCrCrUrCrCrUrArGrArArGrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E3 | rUrCrCrArArUrArGrGrArGrCrGrGrGrCrGrGrUrArUrGrU*mU*mU*mU |
| MG3-6/3- | 404 | MG3- | mG*mU*mU*rCrUrGrGrArGrUrUrUrCrArGrGrUrUrGrArUrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F3 | rUrCrCrArArUrArGrGrArGrCrGrGrGrCrGrGrUrArUrGrU*mU*mU*mU |
| MG3-6/3- | 405 | MG3- | mC*mA*mC*rUrGrGrUrUrUrGrCrArGrCrGrArUrArGrArUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G3 | rUrCrCrArArUrArGrGrArGrCrGrGrGrCrGrGrUrArUrGrU*mU*mU*mU |
| MG3-6/3- | 406 | MG3- | mA*mC*mU*rGrGrUrUrUrGrCrArGrCrGrArUrArGrArUrCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H3 | rUrCrCrArArUrArGrGrArGrCrGrGrGrCrGrGrUrArUrGrU*mU*mU*mU |
| MG3-6/3- | 407 | MG3- | mC*mG*mA*rUrArGrArUrCrArUrArArArArArGrArCrUrGrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A4 | rUrCrCrArArUrArGrGrArGrCrGrGrGrCrGrGrUrArUrGrU*mU*mU*mU |
| MG3-6/3- | 408 | MG3- | mC*mC*mC*rArArCrUrGrArArGrGrArGrGrCrCrArUrUrGrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B4 | rUrCrCrArArUrArGrGrArGrCrGrGrGrCrGrGrUrArUrGrU*mU*mU*mU |
| MG3-6/3- | 409 | MG3- | mC*mC*mA*rArCrUrGrArArGrGrArGrGrCrCrArUrUrGrGrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C4 | rUrCrCrArArUrArGrGrArGrCrGrGrGrCrGrGrUrArUrGrU*mU*mU*mU |
| MG3-6/3- | 410 | MG3- | mC*mUmU*rGrArUrUrUrUrGrGrCrUrCrUrGrGrArGrArUrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D4 | rUrCrCrArArUrArGrGrArGrCrGrGrGrCrGrGrUrArUrGrU*mU*mU*mU |
| MG3-6/3- | 411 | MG3- | mU*mUmU*rUrGrGrCrUrCrUrGrGrArGrArUrArGrArGrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E4 | rUrCrCrArArUrArGrGrArGrCrGrGrGrCrGrGrUrArUrGrU*mU*mU*mU |
| MG3-6/3- | 412 | MG3- | mU*mC*mU*rGrGrArGrArUrArGrArGrArArUrCrArArArUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F4 | rUrCrCrArArUrArGrGrArGrCrGrGrGrCrGrGrUrArUrGrU*mU*mU*mU |
| MG3-6/3- | 413 | MG3- | mG*mA*mA*rUrUrGrUrCrUrUrGrArUrCrArArUrUrCrUrGrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G4 | rUrCrCrArArUrArGrGrArGrCrGrGrGrCrGrGrUrArUrGrU*mU*mU*mU |
| MG3-6/3- | 414 | MG3- | mA*mA*mU*rUrGrUrCrUrUrGrArUrCrArArUrUrCrUrGrGrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H4 | rUrCrCrArArUrArGrGrArGrCrGrGrGrCrGrGrUrArUrGrU*mU*mU*mU |
| MG3-6/3- | 415 | MG3- | mG*mG*mA*rGrGrArArArUrArArCrUrArGrArGrGrArArCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A5 | rUrCrCrArArUrArGrGrArGrCrGrGrGrCrGrGrUrArUrGrU*mU*mU*mU |
| MG3-6/3- | 416 | MG3- | mG*mA*mG*rGrArArArUrArArCrUrArGrArGrGrArArCrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B5 | rUrCrCrArArUrArGrGrArGrCrGrGrGrCrGrGrUrArUrGrU*mU*mU*mU |
| MG3-6/3- | 417 | MG3- | mA*mC*mU*rCrUrCrUrArUrArUrCrCrArGrArCrUrUrUrUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C5 | rUrCrCrArArUrArGrGrArGrCrGrGrGrCrGrGrUrArUrGrU*mU*mU*mU |
| MG3-6/3- | 418 | MG3- | mC*mU*mC*rUrCrUrArUrArUrCrCrArGrArCrUrUrUrUrGrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D5 | rUrCrCrArArUrArGrGrArGrCrGrGrGrCrGrGrUrArUrGrU*mU*mU*mU |
| MG3-6/3- | 419 | MG3- | mU*mC*mU*rCrUrArUrArUrCrCrArGrArCrUrUrUrUrGrUrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E5 | rUrCrCrArArUrArGrGrArGrCrGrGrGrCrGrGrUrArUrGrU*mU*mU*mU |
| MG3-6/3- | 420 | MG3- | mA*mA*mC*rArArUrUrArArArCrCrArArCrArGrCrArUrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F5 | rUrCrCrArArUrArGrGrArGrCrGrGrGrCrGrGrUrArUrGrU*mU*mU*mU |
| MG3-6/3- | 421 | MG3- | mA*mU*mU*rArArArCrCrArArCrArGrCrArUrArGrUrCrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G5 | rUrCrCrArArUrArGrGrArGrCrGrGrGrCrGrGrUrArUrGrU*mU*mU*mU |
| MG3-6/3- | 422 | MG3- | mA*mA*mC*rCrArArCrArGrCrArUrArGrUrCrArArArUrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H5 | rUrCrCrArArUrArGrGrArGrCrGrGrGrCrGrGrUrArUrGrU*mU*mU*mU |
| MG3-6/3- | 423 | MG3- | mA*mC*mC*rArArCrArGrCrArUrArGrUrCrArArArUrArArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A6 | rUrCrCrArArUrArGrGrArGrCrGrGrGrCrGrGrUrArUrGrU*mU*mU*mU |
| MG3-6/3- | 424 | MG3- | mG*mA*mU*rGrCrUrArUrUrArUrCrUrUrGrUrUrUrUrUrCrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B6 | rUrCrCrArArUrArGrGrArGrCrGrGrGrCrGrGrUrArUrGrU*mU*mU*mU |
| MG3-6/3- | 425 | MG3- | mA*mG*mG*rArCrUrArGrUrArUrUrCrArArGrArArCrCrCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C6 | rUrCrCrArArUrArGrGrArGrCrGrGrGrCrGrGrUrArUrGrU*mU*mU*mU |
| MG3-6/3- | 426 | MG3- | mG*mG*mA*rCrUrArGrUrArUrUrCrArArGrArArCrCrCrArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D6 | rUrCrCrArArUrArGrGrArGrCrGrGrGrCrGrGrUrArUrGrU*mU*mU*mU |
| MG3-6/3- | 427 | MG3- | mA*mA*mG*rArArCrUrArCrUrCrCrCrUrUrUrCrUrUrCrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E6 | rUrCrCrArArUrArGrGrArGrCrGrGrGrCrGrGrUrArUrGrU*mU*mU*mU |
| MG3-6/3- | 428 | MG3- | mA*mC*mU*rArCrUrCrCrCrUrUrUrCrUrUrCrArGrUrUrGrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F6 | rUrCrCrArArUrArGrGrArGrCrGrGrGrCrGrGrUrArUrGrU*mU*mU*mU |
| MG3-6/3- | 429 | MG3- | mC*mU*mA*rCrUrCrCrCrUrUrUrCrUrUrCrArGrUrUrGrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G6 | rUrCrCrArArUrArGrGrArGrCrGrGrGrCrGrGrUrArUrGrU*mU*mU*mU |
| MG3-6/3- | 430 | MG3- | mC*mC*mU*rUrUrCrUrUrCrArGrUrUrGrArArUrGrArArArUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H6 | rUrCrCrArArUrArGrGrArGrCrGrGrGrCrGrGrUrArUrGrU*mU*mU*mU |
| MG3-6/3- | 431 | MG3- | mG*mG*mU*rGrCrUrCrUrUrGrGrCrUrUrGrGrArArGrArUrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A7 | rUrCrCrArArUrArGrGrArGrCrGrGrGrCrGrGrUrArUrGrU*mU*mU*mU |
| MG3-6/3- | 432 | MG3- | mG*mU*mG*rCrUrCrUrUrGrGrCrUrUrGrGrArArGrArUrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B7 | rUrCrCrArArUrArGrGrArGrCrGrGrGrCrGrGrUrArUrGrU*mU*mU*mU |
| MG3-6/3- | 433 | MG3- | mA*mU*mA*rGrArGrArArArUrUrUrCrUrGrUrGrGrGrUrUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C7 | rUrCrCrArArUrArGrGrArGrCrGrGrGrCrGrGrUrArUrGrU*mU*mU*mU |
| MG3-6/3- | 434 | MG3- | mG*mA*mA*rUrArCrUrArGrUrCrCrUrUrCrUrGrArGrCrUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D7 | rUrCrCrArArUrArGrGrArGrCrGrGrGrCrGrGrUrArUrGrU*mU*mU*mU |
| MG3-6/3- | 435 | MG3- | mU*mU*mA*rUrUrGrArUrUrCrUrArGrGrCrArUrUrCrCrUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E7 | rUrCrCrArArUrArGrGrArGrCrGrGrGrCrGrGrUrArUrGrU*mU*mU*mU |
| MG3-6/3- | 436 | MG3- | mG*mU*mC*rUrArCrUrGrUrGrArUrGrUrUrArUrArUrCrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F7 | rUrCrCrArArUrArGrGrArGrCrGrGrGrCrGrGrUrArUrGrU*mU*mU*mU |
| MG3-6/3- | 437 | MG3- | mC*mU*mG*rArUrArUrArArCrArUrCrArCrArGrUrArGrArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G7 | rUrCrCrArArUrArGrGrArGrCrGrGrGrCrGrGrUrArUrGrU*mU*mU*mU |
| MG3-6/3- | 438 | MG3- | mU*mG*mA*rUrArUrArArCrArUrCrArCrArGrUrArGrArCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H7 | rUrCrCrArArUrArGrGrArGrCrGrGrGrCrGrGrUrArUrGrU*mU*mU*mU |
| MG3-6/3- | 439 | MG3- | mG*mA*mU*rArUrArArCrArUrCrArCrArGrUrArGrArCrArUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A8 | rUrCrCrArArUrArGrGrArGrCrGrGrGrCrGrGrUrArUrGrU*mU*mU*mU |
| MG3-6/3- | 440 | MG3- | mC*mA*mC*rUrUrGrUrArUrGrUrUrCrArCrCrUrCrUrGrUrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B8 | rUrCrCrArArUrArGrGrArGrCrGrGrGrCrGrGrUrArUrGrU*mU*mU*mU |
| MG3-6/3- | 441 | MG3- | mU*mA*mU*rArArArUrGrGrUrGrGrUrArCrArUrUrCrArGrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C8 | rUrCrCrArArUrArGrGrArGrCrGrGrGrCrGrGrUrArUrGrU*mU*mU*mU |
| MG3-6/3- | 442 | MG3- | mU*mG*mG*rUrArCrArUrUrCrArGrCrArGrGrArArUrGrCrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D8 | rUrCrCrArArUrArGrGrArGrCrGrGrGrCrGrGrUrArUrGrU*mU*mU*mU |
| MG3-6/3- | 443 | MG3- | mG*mU*mC*rCrArUrGrGrArCrArUrUrArArUrUrCrArArCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E8 | rUrCrCrArArUrArGrGrArGrCrGrGrGrCrGrGrUrArUrGrU*mU*mU*mU |
| MG3-6/3- | 444 | MG3- | mU*mU*mC*rArArCrArUrCrGrArArUrArGrArUrGrGrArUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F8 | rUrCrCrArArUrArGrGrArGrCrGrGrGrCrGrGrUrArUrGrU*mU*mU*mU |
| MG3-6/3- | 445 | MG3- | mA*mU*mA*rGrArUrGrGrArUrCrArCrArArArArCrUrUrCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G8 | rUrCrCrArArUrArGrGrArGrCrGrGrGrCrGrGrUrArUrGrU*mU*mU*mU |
| MG3-6/3- | 446 | MG3- | mU*mU*mC*rArArUrGrArArArCrGrUrGrGrGrArGrArArCrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H8 | rUrCrCrArArUrArGrGrArGrCrGrGrGrCrGrGrUrArUrGrU*mU*mU*mU |
| MG3-6/3- | 447 | MG3- | mA*mG*mU*rCrCrCrCrUrUrArCrCrArUrCrArArGrCrCrUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A9 | rUrCrCrArArUrArGrGrArGrCrGrGrGrCrGrGrUrArUrGrU*mU*mU*mU |
| MG3-6/3- | 448 | MG3- | mU*mU*mU*rGrUrGrArUrCrCrArUrCrUrArUrUrCrGrArUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B9 | rUrCrCrArArUrArGrGrArGrCrGrGrGrCrGrGrUrArUrGrU*mU*mU*mU |
| MG3-6/3- | 449 | MG3- | mU*mG*mA*rArUrUrArArUrGrUrCrCrArUrGrGrArCrUrArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C9 | rUrCrCrArArUrArGrGrArGrCrGrGrGrCrGrGrUrArUrGrU*mU*mU*mU |
| MG3-6/3- | 450 | MG3- | mU*mU*mU*rArCrGrArArUrUrGrArGrUrUrGrGrArArGrArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D9 | rUrCrCrArArUrArGrGrArGrCrGrGrGrCrGrGrUrArUrGrU*mU*mU*mU |
| MG3-6/3- | 451 | MG3- | mG*mG*mC*rArArUrGrUrCrCrCrCrArArUrGrCrArArUrCrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E9 | rUrCrCrArArUrArGrGrArGrCrGrGrGrCrGrGrUrArUrGrU*mU*mU*mU |
| MG3-6/3- | 452 | MG3- | mG*mC*mA*rArUrGrUrCrCrCrCrArArUrGrCrArArUrCrCrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F9 | rUrCrCrArArUrArGrGrArGrCrGrGrGrCrGrGrUrArUrGrU*mU*mU*mU |
| MG3-6/3- | 453 | MG3- | mG*mU*mU*rUrUrCrUrArCrUrUrGrGrGrArUrCrArCrArArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G9 | rUrCrCrArArUrArGrGrArGrCrGrGrGrCrGrGrUrArUrGrU*mU*mU*mU |
| MG3-6/3- | 454 | MG3- | mC*mC*mU*rUrUrUrGrCrUrUrUrGrUrGrArUrCrCrCrArArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H9 | rUrCrCrArArUrArGrGrArGrCrGrGrGrCrGrGrUrArUrGrU*mU*mU*mU |
| MG3-6/3- | 455 | MG3- | mC*mU*mU*rUrUrGrCrUrUrUrGrUrGrArUrCrCrCrArArGrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A10 | rUrCrCrArArUrArGrGrArGrCrGrGrGrCrGrGrUrArUrGrU*mU*mU*mU |
| MG3-6/3- | 456 | MG3- | mU*mU*mG*rUrGrArUrCrCrCrArArGrUrArGrArArArArCrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B10 | rUrCrCrArArUrArGrGrArGrCrGrGrGrCrGrGrUrArUrGrU*mU*mU*mU |
| MG3-6/3- | 457 | MG3- | mA*mG*mU*rUrGrGrUrUrUrCrGrUrGrArUrUrUrCrCrCrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C10 | rUrCrCrArArUrArGrGrArGrCrGrGrGrCrGrGrUrArUrGrU*mU*mU*mU |
| MG3-6/3- | 458 | MG3- | mG*mU*mU*rGrGrUrUrUrCrGrUrGrArUrUrUrCrCrCrArArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D10 | rUrCrCrArArUrArGrGrArGrCrGrGrGrCrGrGrUrArUrGrU*mU*mU*mU |
| MG3-6/3- | 459 | MG3- | mG*mU*mU*rUrCrGrUrGrArUrUrUrCrCrCrArArGrUrArArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E10 | rUrCrCrArArUrArGrGrArGrCrGrGrGrCrGrGrUrArUrGrU*mU*mU*mU |
| MG3-6/3- | 460 | MG3- | mU*mU*mC*rCrArGrUrCrUrUrCrCrArArCrUrCrArArUrUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F10 | rUrCrCrArArUrArGrGrArGrCrGrGrGrCrGrGrUrArUrGrU*mU*mU*mU |
| MG3-6/3- | 461 | MG3- | mA*mG*mU*rArUrArUrCrUrUrCrUrCrUrArGrGrCrCrCrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G10 | rUrCrCrArArUrArGrGrArGrCrGrGrGrCrGrGrUrArUrGrU*mU*mU*mU |
| MG3-6/3- | 462 | MG3- | mG*mU*mA*rUrArUrCrUrUrCrUrCrUrArGrGrCrCrCrArArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H10 | rUrCrCrArArUrArGrGrArGrCrGrGrGrCrGrGrUrArUrGrU*mU*mU*mU |
| MG3-6/3- | 463 | MG3- | mU*mC*mU*rArGrGrCrCrCrArArCrCrArArArArUrUrCrUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A11 | rUrCrCrArArUrArGrGrArGrCrGrGrGrCrGrGrUrArUrGrU*mU*mU*mU |
| MG3-6/3- | 464 | MG3- | mC*mU*mA*rGrGrCrCrCrArArCrCrArArArArUrUrCrUrCrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B11 | rUrCrCrArArUrArGrGrArGrCrGrGrGrCrGrGrUrArUrGrU*mU*mU*mU |
| MG3-6/3- | 465 | MG3- | mG*mC*mC*rCrArArCrCrArArArArUrUrCrUrCrCrUrGrArArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C11 | rUrCrCrArArUrArGrGrArGrCrGrGrGrCrGrGrUrArUrGrU*mU*mU*mU |
| MG3-6/3- | 466 | MG3- | mU*mG*mG*rUrGrGrUrGrGrCrArUrGrArUrGrArGrUrGrUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D11 | rUrCrCrArArUrArGrGrArGrCrGrGrGrCrGrGrUrArUrGrU*mU*mU*mU |
| MG3-6/3- | 467 | MG3- | mG*mG*mU*rGrGrUrGrGrCrArUrGrArUrGrArGrUrGrUrGrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E11 | rUrCrCrArArUrArGrGrArGrCrGrGrGrCrGrGrUrArUrGrU*mU*mU*mU |
| MG3-6/3- | 468 | MG3- | mU*mG*mA*rUrGrArGrUrGrUrGrGrArGrArArArArCrArArCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F11 | rUrCrCrArArUrArGrGrArGrCrGrGrGrCrGrGrUrArUrGrU*mU*mU*mU |
| MG3-6/3- | 469 | MG3- | mU*mG*mU*rGrGrArGrArArArArCrArArCrCrUrArArArUrGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G11 | rUrCrCrArArUrArGrGrArGrCrGrGrGrCrGrGrUrArUrGrU*mU*mU*mU |
| MG3-6/3- | 470 | MG3- | mG*mG*mU*rArArArUrArUrArArCrArArArCrCrArArGrArGrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 H11 | rUrCrCrArArUrArGrGrArGrCrGrGrGrCrGrGrUrArUrGrU*mU*mU*mU |
| MG3-6/3- | 471 | MG3- | mG*mA*mA*rGrArGrGrArUrUrArUrCrUrUrGrGrArArGrUrCrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 A12 | rUrCrCrArArUrArGrGrArGrCrGrGrGrCrGrGrUrArUrGrU*mU*mU*mU |
| MG3-6/3- | 472 | MG3- | mA*mA*mG*rArGrGrArUrUrArUrCrUrUrGrGrArArGrUrCrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 B12 | rUrCrCrArArUrArGrGrArGrCrGrGrGrCrGrGrUrArUrGrU*mU*mU*mU |
| MG3-6/3- | 473 | MG3- | mU*mC*mA*rArArArUrGrGrArArGrGrUrUrArUrArCrUrCrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 C12 | rUrCrCrArArUrArGrGrArGrCrGrGrGrCrGrGrUrArUrGrU*mU*mU*mU |
| MG3-6/3- | 474 | MG3- | mC*mA*mA*rArArUrGrGrArArGrGrUrUrArUrArCrUrCrUrArGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 D12 | rUrCrCrArArUrArGrGrArGrCrGrGrGrCrGrGrUrArUrGrU*mU*mU*mU |
| MG3-6/3- | 475 | MG3- | mA*mU*mG*rUrUrGrArUrCrCrArUrCrCrArArCrArGrArUrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 E12 | rUrCrCrArArUrArGrGrArGrCrGrGrGrCrGrGrUrArUrGrU*mU*mU*mU |
| MG3-6/3- | 476 | MG3- | mC*mA*mU*rCrCrArArCrArGrArUrUrCrArGrArArArGrCrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 F12 | rUrCrCrArArUrArGrGrArGrCrGrGrGrCrGrGrUrArUrGrU*mU*mU*mU |
| MG3-6/3- | 477 | MG3- | mG*mC*mC*rUrCrArGrUrUrCrArUrUrCrArArArGrCrUrUrUrGrUrUrGrA |
| 4 sgRNA | | 6/3-4 | rGrArArUrCrGrArArArGrArUrUrCrUrUrArArUrArArGrGrCrArUrCrCr |
| targeting | | ANGPT | UrUrCrCrGrArUrGrCrUrGrArCrUrUrCrUrCrArCrCrGrUrCrCrGrUrUrU |
| ANGPTL3 | | L3 G12 | rUrCrCrArArUrArGrGrArGrCrGrGrGrCrGrGrUrArUrGrU*mU*mU*mU |
| DNA | 478 | MG3- | TTGTTCCTCTAGTTATTTCCTC |
| sequence | | 6/3-4 | |
| of | | ANGPT | |
| ANGPTL3 | | L3 A1 | |
| target site | | | |
| DNA | 479 | MG3- | ATTTGATTCTCTATCTCCAGAG |
| sequence | | 6/3-4 | |
| of | | ANGPT | |
| ANGPTL3 | | L3 B1 | |
| target site | | | |
| DNA | 480 | MG3- | TTTGATTCTCTATCTCCAGAGC |
| sequence | | 6/3-4 | |
| of | | ANGPT | |
| ANGPTL3 | | L3 C1 | |
| target site | | | |
| DNA | 481 | MG3- | AAGATTTGCTATGTTAGACGAT |
| sequence | | 6/3-4 | |
| of | | ANGPT | |
| ANGPTL3 | | L3 D1 | |
| target site | | | |
| DNA | 482 | MG3- | AGATTTGCTATGTTAGACGATG |
| sequence | | 6/3-4 | |
| of | | ANGPT | |
| ANGPTL3 | | L3 E1 | |
| target site | | | |
| DNA | 483 | MG3- | GATTTGCTATGTTAGACGATGT |
| sequence | | 6/3-4 | |
| of | | ANGPT | |
| ANGPTL3 | | L3 F1 | |
| target site | | | |
| DNA | 484 | MG3- | ACTTTGTCCATAAGACGAAGGG |
| sequence | | 6/3-4 | |
| of | | ANGPT | |
| ANGPTL3 | | L3 G1 | |
| target site | | | |
| DNA | 485 | MG3- | AGGGCCAAATTAATGACATATT |
| sequence | | 6/3-4 | |
| of | | ANGPT | |
| ANGPTL3 | | L3 H1 | |
| target site | | | |
| DNA | 486 | MG3- | GGGCCAAATTAATGACATATTT |
| sequence | | 6/3-4 | |
| of | | ANGPT | |
| ANGPTL3 | | L3 A2 | |
| target site | | | |
| DNA | 487 | MG3- | TATGATCTATCGCTGCAAACCA |
| sequence | | 6/3-4 | |
| of | | ANGPT | |
| ANGPTL3 | | L3 B2 | |
| target site | | | |
| DNA | 488 | MG3- | ATGATCTATCGCTGCAAACCAG |
| sequence | | 6/3-4 | |
| of | | | |
| ANGPTL3 | | ANGPT | |
| target site | | L3 C2 | |
| DNA | 489 | MG3- | CAAACCAGTGAAATCAAAGAAG |
| sequence | | 6/3-4 | |
| of | | ANGPT | |
| ANGPTL3 | | L3 D2 | |
| target site | | | |
| DNA | 490 | MG3- | AAACCAGTGAAATCAAAGAAGA |
| sequence | | 6/3-4 | |
| of | | ANGPT | |
| ANGPTL3 | | L3 E2 | |
| target site | | | |
| DNA | 491 | MG3- | ACAAGTCAAAAATGAAGAGGTA |
| sequence | | 6/3-4 | |
| of | | ANGPT | |
| ANGPTL3 | | L3 F2 | |
| target site | | | |
| DNA | 492 | MG3- | GAATATGTCACTTGAACTCAAC |
| sequence | | 6/3-4 | |
| of | | ANGPT | |
| ANGPTL3 | | L3 G2 | |
| target site | | | |
| DNA | 493 | MG3- | TCACTTGAACTCAACTCAAAAC |
| sequence | | 6/3-4 | |
| of | | ANGPT | |
| ANGPTL3 | | L3 H2 | |
| target site | | | |
| DNA | 494 | MG3- | TCAAAACTTGAAAGCCTCCTAG |
| sequence | | 6/3-4 | |
| of | | ANGPT | |
| ANGPTL3 | | L3 A3 | |
| target site | | | |
| DNA | 495 | MG3- | CAAAACTTGAAAGCCTCCTAGA |
| sequence | | 6/3-4 | |
| of | | ANGPT | |
| ANGPTL3 | | L3 B3 | |
| target site | | | |
| DNA | 496 | MG3- | AAAACTTGAAAGCCTCCTAGAA |
| sequence | | 6/3-4 | |
| of | | ANGPT | |
| ANGPTL3 | | L3 C3 | |
| target site | | | |
| DNA | 497 | MG3- | AAACTTGAAAGCCTCCTAGAAG |
| sequence | | 6/3-4 | |
| of | | ANGPT | |
| ANGPTL3 | | L3 D3 | |
| target site | | | |
| DNA | 498 | MG3- | AACTTGAAAGCCTCCTAGAAGA |
| sequence | | 6/3-4 | |
| of | | ANGPT | |
| ANGPTL3 | | L3 E3 | |
| target site | | | |
| DNA | 499 | MG3- | GTTCTGGAGTTTCAGGTTGATT |
| sequence | | 6/3-4 | |
| of | | | |
| ANGPTL3 | | ANGPT | |
| target site | | L3 F3 | |
| DNA | 500 | MG3- | CACTGGTTTGCAGCGATAGATC |
| sequence | | 6/3-4 | |
| of | | ANGPT | |
| ANGPTL3 | | L3 G3 | |
| target site | | | |
| DNA | 501 | MG3- | ACTGGTTTGCAGCGATAGATCA |
| sequence | | 6/3-4 | |
| of | | ANGPT | |
| ANGPTL3 | | L3 H3 | |
| target site | | | |
| DNA | 502 | MG3- | CGATAGATCATAAAAAGACTGA |
| sequence | | 6/3-4 | |
| of | | ANGPT | |
| ANGPTL3 | | L3 A4 | |
| target site | | | |
| DNA | 503 | MG3- | CCCAACTGAAGGAGGCCATTGG |
| sequence | | 6/3-4 | |
| of | | ANGPT | |
| ANGPTL3 | | L3 B4 | |
| target site | | | |
| DNA | 504 | MG3- | CCAACTGAAGGAGGCCATTGGC |
| sequence | | 6/3-4 | |
| of | | ANGPT | |
| ANGPTL3 | | L3 C4 | |
| target site | | | |
| DNA | 505 | MG3- | CTTGATTTTGGCTCTGGAGATA |
| sequence | | 6/3-4 | |
| of | | ANGPT | |
| ANGPTL3 | | L3 D4 | |
| target site | | | |
| DNA | 506 | MG3- | TTTTGGCTCTGGAGATAGAGAA |
| sequence | | 6/3-4 | |
| of | | ANGPT | |
| ANGPTL3 | | L3 E4 | |
| target site | | | |
| DNA | 507 | MG3- | TCTGGAGATAGAGAATCAAATG |
| sequence | | 6/3-4 | |
| of | | ANGPT | |
| ANGPTL3 | | L3 F4 | |
| target site | | | |
| DNA | 508 | MG3- | GAATTGTCTTGATCAATTCTGG |
| sequence | | 6/3-4 | |
| of | | ANGPT | |
| ANGPTL3 | | L3 G4 | |
| target site | | | |
| DNA | 509 | MG3- | AATTGTCTTGATCAATTCTGGA |
| sequence | | 6/3-4 | |
| of | | ANGPT | |
| ANGPTL3 | | L3 H4 | |
| target site | | | |
| DNA | 510 | MG3- | GGAGGAAATAACTAGAGGAACA |
| sequence | | 6/3-4 | |
| of | | | |
| ANGPTL3 | | ANGPT | |
| target site | | L3 A5 | |
| DNA | 511 | MG3- | GAGGAAATAACTAGAGGAACAA |
| sequence | | 6/3-4 | |
| of | | ANGPT | |
| ANGPTL3 | | L3 B5 | |
| target site | | | |
| DNA | 512 | MG3- | ACTCTCTATATCCAGACTTTTG |
| sequence | | 6/3-4 | |
| of | | ANGPT | |
| ANGPTL3 | | L3 C5 | |
| target site | | | |
| DNA | 513 | MG3- | CTCTCTATATCCAGACTTTTGT |
| sequence | | 6/3-4 | |
| of | | ANGPT | |
| ANGPTL3 | | L3 D5 | |
| target site | | | |
| DNA | 514 | MG3- | TCTCTATATCCAGACTTTTGTA |
| sequence | | 6/3-4 | |
| of | | ANGPT | |
| ANGPTL3 | | L3 E5 | |
| target site | | | |
| DNA | 515 | MG3- | AACAATTAAACCAACAGCATAG |
| sequence | | 6/3-4 | |
| of | | ANGPT | |
| ANGPTL3 | | L3 F5 | |
| target site | | | |
| DNA | 516 | MG3- | ATTAAACCAACAGCATAGTCAA |
| sequence | | 6/3-4 | |
| of | | ANGPT | |
| ANGPTL3 | | L3 G5 | |
| target site | | | |
| DNA | 517 | MG3- | AACCAACAGCATAGTCAAATAA |
| sequence | | 6/3-4 | |
| of | | ANGPT | |
| ANGPTL3 | | L3 H5 | |
| target site | | | |
| DNA | 518 | MG3- | ACCAACAGCATAGTCAAATAAA |
| sequence | | 6/3-4 | |
| of | | ANGPT | |
| ANGPTL3 | | L3 A6 | |
| target site | | | |
| DNA | 519 | MG3- | GATGCTATTATCTTGTTTTTCT |
| sequence | | 6/3-4 | |
| of | | ANGPT | |
| ANGPTL3 | | L3 B6 | |
| target site | | | |
| DNA | 520 | MG3- | AGGACTAGTATTCAAGAACCCA |
| sequence | | 6/3-4 | |
| of | | ANGPT | |
| ANGPTL3 | | L3 C6 | |
| target site | | | |
| DNA | 521 | MG3- | GGACTAGTATTCAAGAACCCAC |
| sequence | | 6/3-4 | |
| of | | | |
| ANGPTL3 | | ANGPT | |
| target site | | L3 D6 | |
| DNA | 522 | MG3- | AAGAACTACTCCCTTTCTTCAG |
| sequence | | 6/3-4 | |
| of | | ANGPT | |
| ANGPTL3 | | L3 E6 | |
| target site | | | |
| DNA | 523 | MG3- | ACTACTCCCTTTCTTCAGTTGA |
| sequence | | 6/3-4 | |
| of | | ANGPT | |
| ANGPTL3 | | L3 F6 | |
| target site | | | |
| DNA | 524 | MG3- | CTACTCCCTTTCTTCAGTTGAA |
| sequence | | 6/3-4 | |
| of | | ANGPT | |
| ANGPTL3 | | L3 G6 | |
| target site | | | |
| DNA | 525 | MG3- | CCTTTCTTCAGTTGAATGAAAT |
| sequence | | 6/3-4 | |
| of | | ANGPT | |
| ANGPTL3 | | L3 H6 | |
| target site | | | |
| DNA | 526 | MG3- | GGTGCTCTTGGCTTGGAAGATA |
| sequence | | 6/3-4 | |
| of | | ANGPT | |
| ANGPTL3 | | L3 A7 | |
| target site | | | |
| DNA | 527 | MG3- | GTGCTCTTGGCTTGGAAGATAG |
| sequence | | 6/3-4 | |
| of | | ANGPT | |
| ANGPTL3 | | L3 B7 | |
| target site | | | |
| DNA | 528 | MG3- | ATAGAGAAATTTCTGTGGGTTC |
| sequence | | 6/3-4 | |
| of | | ANGPT | |
| ANGPTL3 | | L3 C7 | |
| target site | | | |
| DNA | 529 | MG3- | GAATACTAGTCCTTCTGAGCTG |
| sequence | | 6/3-4 | |
| of | | ANGPT | |
| ANGPTL3 | | L3 D7 | |
| target site | | | |
| DNA | 530 | MG3- | TTATTGATTCTAGGCATTCCTG |
| sequence | | 6/3-4 | |
| of | | ANGPT | |
| ANGPTL3 | | L3 E7 | |
| target site | | | |
| DNA | 531 | MG3- | GTCTACTGTGATGTTATATCAG |
| sequence | | 6/3-4 | |
| of | | ANGPT | |
| ANGPTL3 | | L3 F7 | |
| target site | | | |
| DNA | 532 | MG3- | CTGATATAACATCACAGTAGAC |
| sequence | | 6/3-4 | |
| of | | | |
| ANGPTL3 | | ANGPT | |
| target site | | L3 G7 | |
| DNA | 533 | MG3- | TGATATAACATCACAGTAGACA |
| sequence | | 6/3-4 | |
| of | | ANGPT | |
| ANGPTL3 | | L3 H7 | |
| target site | | | |
| DNA | 534 | MG3- | GATATAACATCACAGTAGACAT |
| sequence | | 6/3-4 | |
| of | | ANGPT | |
| ANGPTL3 | | L3 A8 | |
| target site | | | |
| DNA | 535 | MG3- | CACTTGTATGTTCACCTCTGTT |
| sequence | | 6/3-4 | |
| of | | ANGPT | |
| ANGPTL3 | | L3 B8 | |
| target site | | | |
| DNA | 536 | MG3- | TATAAATGGTGGTACATTCAGC |
| sequence | | 6/3-4 | |
| of | | ANGPT | |
| ANGPTL3 | | L3 C8 | |
| target site | | | |
| DNA | 537 | MG3- | TGGTACATTCAGCAGGAATGCC |
| sequence | | 6/3-4 | |
| of | | ANGPT | |
| ANGPTL3 | | L3 D8 | |
| target site | | | |
| DNA | 538 | MG3- | GTCCATGGACATTAATTCAACA |
| sequence | | 6/3-4 | |
| of | | ANGPT | |
| ANGPTL3 | | L3 E8 | |
| target site | | | |
| DNA | 539 | MG3- | TTCAACATCGAATAGATGGATC |
| sequence | | 6/3-4 | |
| of | | ANGPT | |
| ANGPTL3 | | L3 F8 | |
| target site | | | |
| DNA | 540 | MG3- | ATAGATGGATCACAAAACTTCA |
| sequence | | 6/3-4 | |
| of | | ANGPT | |
| ANGPTL3 | | L3 G8 | |
| target site | | | |
| DNA | 541 | MG3- | TTCAATGAAACGTGGGAGAACT |
| sequence | | 6/3-4 | |
| of | | ANGPT | |
| ANGPTL3 | | L3 H8 | |
| target site | | | |
| DNA | 542 | MG3- | AGTCCCCTTACCATCAAGCCTC |
| sequence | | 6/3-4 | |
| of | | ANGPT | |
| ANGPTL3 | | L3 A9 | |
| target site | | | |
| DNA | 543 | MG3- | TTTGTGATCCATCTATTCGATG |
| sequence | | 6/3-4 | |
| of | | | |
| ANGPTL3 | | ANGPT | |
| target site | | L3 B9 | |
| DNA | 544 | MG3- | TGAATTAATGTCCATGGACTAC |
| sequence | | 6/3-4 | |
| of | | ANGPT | |
| ANGPTL3 | | L3 C9 | |
| target site | | | |
| DNA | 545 | MG3- | TTTACGAATTGAGTTGGAAGAC |
| sequence | | 6/3-4 | |
| of | | ANGPT | |
| ANGPTL3 | | L3 D9 | |
| target site | | | |
| DNA | 546 | MG3- | GGCAATGTCCCCAATGCAATCC |
| sequence | | 6/3-4 | |
| of | | ANGPT | |
| ANGPTL3 | | L3 E9 | |
| target site | | | |
| DNA | 547 | MG3- | GCAATGTCCCCAATGCAATCCC |
| sequence | | 6/3-4 | |
| of | | ANGPT | |
| ANGPTL3 | | L3 F9 | |
| target site | | | |
| DNA | 548 | MG3- | GTTTTCTACTTGGGATCACAAA |
| sequence | | 6/3-4 | |
| of | | ANGPT | |
| ANGPTL3 | | L3 G9 | |
| target site | | | |
| DNA | 549 | MG3- | CCTTTTGCTTTGTGATCCCAAG |
| sequence | | 6/3-4 | |
| of | | ANGPT | |
| ANGPTL3 | | L3 H9 | |
| target site | | | |
| DNA | 550 | MG3- | CTTTTGCTTTGTGATCCCAAGT |
| sequence | | 6/3-4 | |
| of | | ANGPT | |
| ANGPTL3 | | L3 A10 | |
| target site | | | |
| DNA | 551 | MG3- | TTGTGATCCCAAGTAGAAAACA |
| sequence | | 6/3-4 | |
| of | | ANGPT | |
| ANGPTL3 | | L3 B10 | |
| target site | | | |
| DNA | 552 | MG3- | AGTTGGTTTCGTGATTTCCCAA |
| sequence | | 6/3-4 | |
| of | | ANGPT | |
| ANGPTL3 | | L3 C10 | |
| target site | | | |
| DNA | 553 | MG3- | GTTGGTTTCGTGATTTCCCAAG |
| sequence | | 6/3-4 | |
| of | | ANGPT | |
| ANGPTL3 | | L3 D10 | |
| target site | | | |
| DNA | 554 | MG3- | GTTTCGTGATTTCCCAAGTAAA |
| sequence | | 6/3-4 | |
| of | | | |
| ANGPTL3 | | ANGPT | |
| target site | | L3 E10 | |
| DNA | 555 | MG3- | TTCCAGTCTTCCAACTCAATTC |
| sequence | | 6/3-4 | |
| of | | ANGPT | |
| ANGPTL3 | | L3 F10 | |
| target site | | | |
| DNA | 556 | MG3- | AGTATATCTTCTCTAGGCCCAA |
| sequence | | 6/3-4 | |
| of | | ANGPT | |
| ANGPTL3 | | L3 G10 | |
| target site | | | |
| DNA | 557 | MG3- | GTATATCTTCTCTAGGCCCAAC |
| sequence | | 6/3-4 | |
| of | | ANGPT | |
| ANGPTL3 | | L3 H10 | |
| target site | | | |
| DNA | 558 | MG3- | TCTAGGCCCAACCAAAATTCTC |
| sequence | | 6/3-4 | |
| of | | ANGPT | |
| ANGPTL3 | | L3 A11 | |
| target site | | | |
| DNA | 559 | MG3- | CTAGGCCCAACCAAAATTCTCC |
| sequence | | 6/3-4 | |
| of | | ANGPT | |
| ANGPTL3 | | L3 B11 | |
| target site | | | |
| DNA | 560 | MG3- | GCCCAACCAAAATTCTCCTGAA |
| sequence | | 6/3-4 | |
| of | | ANGPT | |
| ANGPTL3 | | L3 C11 | |
| target site | | | |
| DNA | 561 | MG3- | TGGTGGTGGCATGATGAGTGTG |
| sequence | | 6/3-4 | |
| of | | ANGPT | |
| ANGPTL3 | | L3 D11 | |
| target site | | | |
| DNA | 562 | MG3- | GGTGGTGGCATGATGAGTGTGG |
| sequence | | 6/3-4 | |
| of | | ANGPT | |
| ANGPTL3 | | L3 E11 | |
| target site | | | |
| DNA | 563 | MG3- | TGATGAGTGTGGAGAAAACAAC |
| sequence | | 6/3-4 | |
| of | | ANGPT | |
| ANGPTL3 | | L3 F11 | |
| target site | | | |
| DNA | 564 | MG3- | TGTGGAGAAAACAACCTAAATG |
| sequence | | 6/3-4 | |
| of | | ANGPT | |
| ANGPTL3 | | L3 G11 | |
| target site | | | |
| DNA | 565 | MG3- | GGTAAATATAACAAACCAAGAG |
| sequence | | 6/3-4 | |
| of | | | |
| ANGPTL3 | | ANGPT | |
| target site | | L3 H11 | |
| DNA | 566 | MG3- | GAAGAGGATTATCTTGGAAGTC |
| sequence | | 6/3-4 | |
| of | | ANGPT | |
| ANGPTL3 | | L3 A12 | |
| target site | | | |
| DNA | 567 | MG3- | AAGAGGATTATCTTGGAAGTCT |
| sequence | | 6/3-4 | |
| of | | ANGPT | |
| ANGPTL3 | | L3 B12 | |
| target site | | | |
| DNA | 568 | MG3- | TCAAAATGGAAGGTTATACTCT |
| sequence | | 6/3-4 | |
| of | | ANGPT | |
| ANGPTL3 | | L3 C12 | |
| target site | | | |
| DNA | 569 | MG3- | CAAAATGGAAGGTTATACTCTA |
| sequence | | 6/3-4 | |
| of | | ANGPT | |
| ANGPTL3 | | L3 D12 | |
| target site | | | |
| DNA | 570 | MG3- | ATGTTGATCCATCCAACAGATT |
| sequence | | 6/3-4 | |
| of | | ANGPT | |
| ANGPTL3 | | L3 E12 | |
| target site | | | |
| DNA | 571 | MG3- | CATCCAACAGATTCAGAAAGCT |
| sequence | | 6/3-4 | |
| of | | ANGPT | |
| ANGPTL3 | | L3 F12 | |
| target site | | | |
| DNA | 572 | MG3- | GCCTCAGTTCATTCAAAGCTTT |
| sequence | | 6/3-4 | |
| of | | ANGPT | |
| ANGPTL3 | | L3 G12 | |
| target site | | | |

(r = native ribose base, m = 2′-O methyl modified base, F = 2′ Fluro modified base, *= phosphorothioate bond)

### Example 17—Analysis of Gene-Editing Outcomes at the DNA Level for PCSK9 in Hep3B Cells

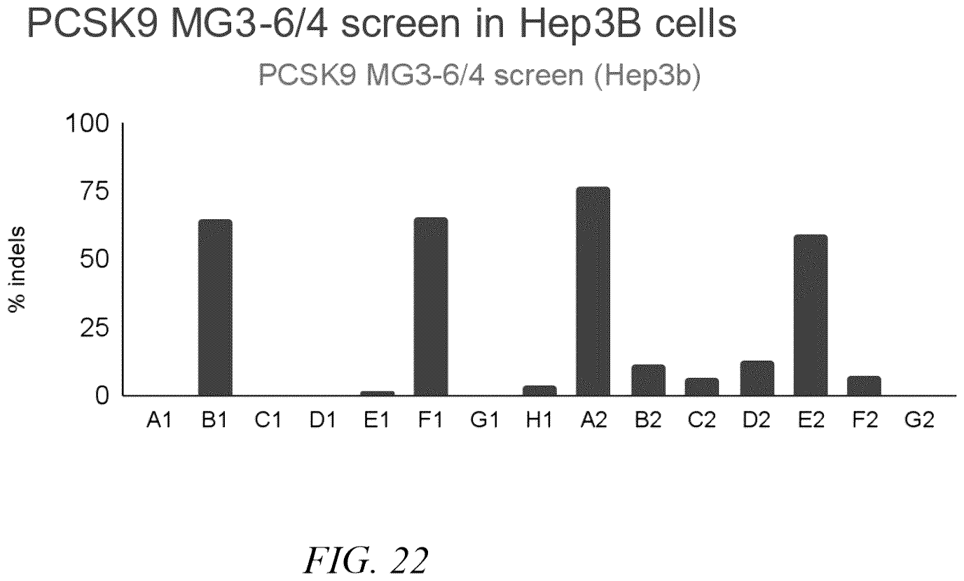

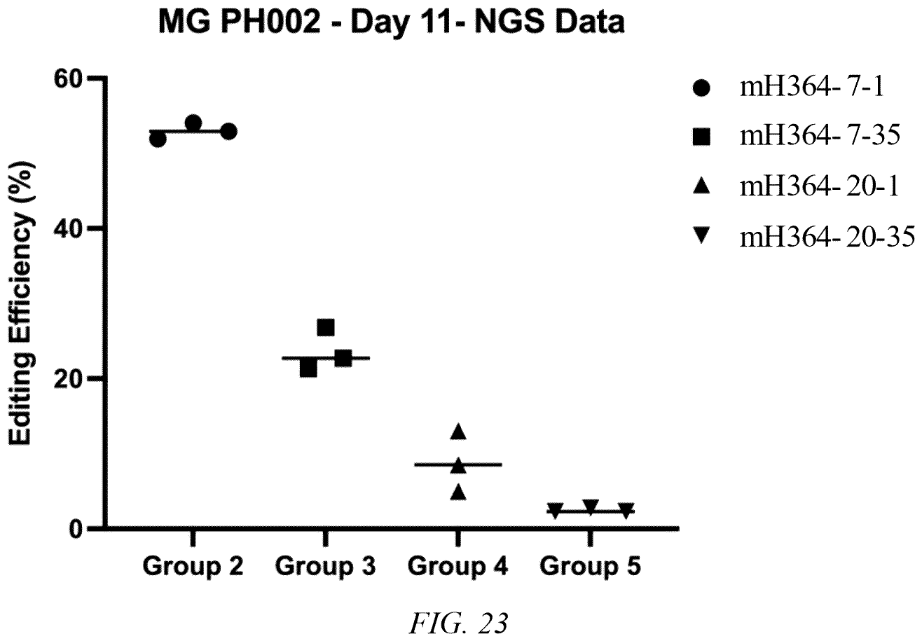

Nucleofection of MG3-6/4 RNPs (104 pmol protein/120 pmol guide) comprising sgRNAs described below in Table 7E below and SEQ ID NOs: 573-602 was performed into Hep3B cells (100,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 22). Results indicate that the highest editing performance was achieved with sgRNAs B1, F1, A2, and E2, with appreciable editing also occurring with D2, C2, B2, H1, and F2.

TABLE 7E
gRNAs and Targeting Sequences Used in Example 17

| | SEQ | | |
|---|---|---|---|
| | ID | | |
| Category | NO: | Name | Sequence |
| MG3-6/3- | 573 | MG3- | mA*mC*mC*rCrCrUrCrCrArCrGrGrUrArCrCrGrGrGrCrGrGrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | A1 | rUrCrCrArArUrArGrGrArGrCrGrGrGrCrGrGrUrArUrGrU*mU*mU*mU |
| MG3-6/3- | 574 | MG3- | mA*mC*mC*rArGrCrArUrArCrArGrArGrUrGrArCrCrArCrCrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | B1 | rUrCrCrArArUrArGrGrArGrCrGrGrGrCrGrGrUrArUrGrU*mU*mU*mU |
| MG3-6/3- | 575 | MG3- | mC*mC*mA*rGrCrArUrArCrArGrArGrUrGrArCrCrArCrCrGrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | C1 | rUrCrCrArArUrArGrGrArGrCrGrGrGrCrGrGrUrArUrGrU*mU*mU*mU |
| MG3-6/3- | 576 | MG3- | mC*mA*mG*rGrGrUrCrArUrGrGrUrCrArCrCrGrArCrUrUrCrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | D1 | rUrCrCrArArUrArGrGrArGrCrGrGrGrCrGrGrUrArUrGrU*mU*mU*mU |
| MG3-6/3- | 577 | MG3- | mC*mC*mU*rCrCrCrArGrGrCrCrUrGrGrArGrUrUrUrArUrUrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | E1 | rUrCrCrArArUrArGrGrArGrCrGrGrGrCrGrGrUrArUrGrU*mU*mU*mU |
| MG3-6/3- | 578 | MG3- | mC*mU*mC*rCrCrArGrGrCrCrUrGrGrArGrUrUrUrArUrUrCrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | F1 | rUrCrCrArArUrArGrGrArGrCrGrGrGrCrGrGrUrArUrGrU*mU*mU*mU |
| MG3-6/3- | 579 | MG3- | mC*mA*mG*rGrCrUrGrGrArCrCrArGrCrUrGrGrCrUrUrUrUrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | G1 | rUrCrCrArArUrArGrGrArGrCrGrGrGrCrGrGrUrArUrGrU*mU*mU*mU |
| MG3-6/3- | 580 | MG3- | mG*mG*mU*rGrGrCrCrCrCrArArCrUrGrUrGrArUrGrArCrCrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | H1 | rUrCrCrArArUrArGrGrArGrCrGrGrGrCrGrGrUrArUrGrU*mU*mU*mU |
| MG3-6/3- | 581 | MG3- | mG*mC*mC*rCrCrGrCrCrGrCrUrUrCrCrCrArCrUrCrCrUrGrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | A2 | rUrCrCrArArUrArGrGrArGrCrGrGrGrCrGrGrUrArUrGrU*mU*mU*mU |
| MG3-6/3- | 582 | MG3- | mA*mG*mU*rGrUrGrCrUrGrArCrCrArUrArCrArGrUrCrCrUrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | B2 | rUrCrCrArArUrArGrGrArGrCrGrGrGrCrGrGrUrArUrGrU*mU*mU*mU |
| MG3-6/3- | 583 | MG3- | mC*mC*mU*rGrCrArArArArCrArGrCrUrGrCrCrArArCrCrUrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | C2 | rUrCrCrArArUrArGrGrArGrCrGrGrGrCrGrGrUrArUrGrU*mU*mU*mU |
| MG3-6/3- | 584 | MG3- | mC*mU*mG*rCrArArArArCrArGrCrUrGrCrCrArArCrCrUrGrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | D2 | rUrCrCrArArUrArGrGrArGrCrGrGrGrCrGrGrUrArUrGrU*mU*mU*mU |
| MG3-6/3- | 585 | MG3- | mA*mA*mU*rGrGrCrGrUrArGrArCrArCrCrCrUrCrArCrCrCrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | E2 | rUrCrCrArArUrArGrGrArGrCrGrGrGrCrGrGrUrArUrGrU*mU*mU*mU |
| MG3-6/3- | 586 | MG3- | mU*mC*mC*rUrGrCrUrGrCrCrArUrGrCrCrCrCrArGrGrUrCrGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | F2 | rUrCrCrArArUrArGrGrArGrCrGrGrGrCrGrGrUrArUrGrU*mU*mU*mU |
| MG3-6/3- | 587 | MG3- | mU*mG*mG*rArArUrGrCrArArArGrUrCrArArGrGrArGrCrArGrUrUrGrA |
| 4 sgRNA | | 6/3-4 | rGrArArUrCrGrArArArGrArUrUrCrUrUrArArUrArArGrGrCrArUrCrCr |
| targeting | | PCSK9 | UrUrCrCrGrArUrGrCrUrGrArCrUrUrCrUrCrArCrCrGrUrCrCrGrUrUrU |
| PCSK9 | | G2 | rUrCrCrArArUrArGrGrArGrCrGrGrGrCrGrGrUrArUrGrU*mU*mU*mU |
| DNA | 588 | MG3- | ACCCCTCCACGGTACCGGGCGG |
| sequence | | 6/3-4 | |
| of PCSK9 | | PCSK9 | |
| target site | | A1 | |
| DNA | 589 | MG3- | ACCAGCATACAGAGTGACCACC |
| sequence | | 6/3-4 | |
| of PCSK9 | | PCSK9 | |
| target site | | B1 | |
| DNA | 590 | MG3- | CCAGCATACAGAGTGACCACCG |
| sequence | | 6/3-4 | |
| of PCSK9 | | PCSK9 | |
| target site | | C1 | |
| DNA | 591 | MG3- | CAGGGTCATGGTCACCGACTTC |
| sequence | | 6/3-4 | |
| of PCSK9 | | PCSK9 | |
| target site | | D1 | |
| DNA | 592 | MG3- | CCTCCCAGGCCTGGAGTTTATT |
| sequence | | 6/3-4 | |
| of PCSK9 | | PCSK9 | |
| target site | | E1 | |
| DNA | 593 | MG3- | CTCCCAGGCCTGGAGTTTATTC |
| sequence | | 6/3-4 | |
| of PCSK9 | | PCSK9 | |
| target site | | F1 | |
| DNA | 594 | MG3- | CAGGCTGGACCAGCTGGCTTTT |
| sequence | | 6/3-4 | |
| of PCSK9 | | PCSK9 | |
| target site | | G1 | |
| DNA | 595 | MG3- | GGTGGCCCCAACTGTGATGACC |
| sequence | | 6/3-4 | |
| of PCSK9 | | PCSK9 | |
| target site | | H1 | |
| DNA | 596 | MG3- | GCCCCGCCGCTTCCCACTCCTG |
| sequence | | 6/3-4 | |
| of PCSK9 | | PCSK9 | |
| target site | | A2 | |
| DNA | 597 | MG3- | AGTGTGCTGACCATACAGTCCT |
| sequence | | 6/3-4 | |
| of PCSK9 | | PCSK9 | |
| target site | | B2 | |
| DNA | 598 | MG3- | CCTGCAAAACAGCTGCCAACCT |
| sequence | | 6/3-4 | |
| of PCSK9 | | PCSK9 | |
| target site | | C2 | |
| DNA | 599 | MG3- | CTGCAAAACAGCTGCCAACCTG |
| sequence | | 6/3-4 | |
| of PCSK9 | | PCSK9 | |
| target site | | D2 | |
| DNA | 600 | MG3- | AATGGCGTAGACACCCTCACCC |
| sequence | | 6/3-4 | |
| of PCSK9 | | PCSK9 | |
| target site | | E2 | |
| DNA | 601 | MG3- | TCCTGCTGCCATGCCCCAGGTC |
| sequence | | 6/3-4 | |
| of PCSK9 | | PCSK9 | |
| target | | F2 | |
| site | | | |
| DNA | 602 | MG3- | TGGAATGCAAAGTCAAGGAGCA |
| sequence | | 6/3-4 | |
| of PCSK9 | | PCSK9 | |
| target site | | G2 | |

(r = native ribose base, m = 2′-O methyl modified base, F = 2′ Fluro modified base, *= phosphorothioate bond)

### Example 18—In Vivo Gene Editing in the Liver of Mice by the Chimeric Nuclease MG3-6/3-4 Delivered by Systemic Administration of a Lipid Nanoparticle

To evaluate the ability of the MG3-6/3-4 chimeric Type II nuclease to edit the genome in vivo in a living animal, a lipid nanoparticle was used to deliver an mRNA encoding the MG3-6/3-4 nuclease (e.g. RNA version of SEQ ID NO: 603) and single guide RNAs (sgRNA) that target different parts of the coding sequence of the mouse HAO-1 gene (e.g. described in the tables below). The HAO-1 gene encodes glycolate oxidase which is an enzyme involved in glycolate metabolism and is expressed primarily in hepatocytes in the liver. A screen of sgRNAs that target the HAO-1 coding sequence was performed in the mouse liver cell line Hepa1-6 to identify active guides. The sgRNAs mH364-7 and mH364-20, which exhibited 46% and 26% editing in Hepa1-6 cells when transfected with the mRNA encoding the MG3-6/3-4 nuclease, were selected for testing in mice. mH364-7 targets exon 2 and mH364-20 targets exon 4.

A number of chemical modifications of the native RNA structure were incorporated into these sgRNAs. These chemical modifications were selected based on their ability to improve the stability of the sgRNA in vitro when incubated in extracts from mammalian cells without negatively impacting editing activity. For initial testing in mice, sgRNAs mH364-7 and mH364-20 incorporating chemistry 1 and chemistry 35 were selected for testing and designated as mH364-7-1, mH364-20-1, mH364-7-35, mH364-20-35. The sequences of these guides including the chemical modifications are shown below in Table 9.

TABLE 9
Sequences and chemical modifications of
guide RNA tested in vivo in mice

| Guide | |
|---|---|
| name | Sequence |
| mH364- | mG*mA*mG*CUGGCCACUGUGCGAG |
| 7-1 | GUAGUUGAGAAUCGAAAGAUUCUUA |
| | AUAAGGCAUCCUUCCGAUGCUGACU |
| | UCUCACCGUCCGUUUUCCAAUAGGA |
| | GCGGGCGGUAUGU*mU*mU*mU |
| mH364- | mU*mU*mC*AGCAAGUCCACUGUUG |
| 20-1 | UCUGUUGAGAAUCGAAAGAUUCUUA |
| | AUAAGGCAUCCUUCCGAUGCUGACU |
| | UCUCACCGUCCGUUUUCCAAUAGGA |
| | GCGGGCGGUAUGU*mU*mU*mU |
| mH364- | mG*mA*mG*mC*UGGCCACUGUGCG |
| 7-35 | AGGUAGUUGAGAAUCmG*mA*mA*m |
| | A*GAUUCUUAAUAAGGCAUCmC*mU |
| | *mU*mC*mC*GAUGCUGACUUCUCA |
| | CCGUCCGUUUUCCmA*mA*mU*mA* |
| | GGAGCGGGCGGUA*mU*mG*mU*mU |
| | *mU*mU |
| mH364- | mU*mU*mC*mA*GCAAGUCCACUGU |
| 20-35 | UGUCUGUUGAGAAUCmG*mA*mA*m |
| | A*GAUUCUUAAUAAGGCAUCmC*mU |
| | *mU*mC*mC*GAUGCUGACUUCUCA |
| | CCGUCCGUUUUCCmA*mA*mU*mA* |
| | GGAGCGGGCGGUA*mU*mG*mU*mU |
| | *mU*mU |

m: 2′-O methyl modified base, *phosphorothioate backbone

The mRNA encoding the MG3-6/3-4 nuclease was generated by in vitro transcription of a linearized plasmid template using T7 RNA polymerase, nucleotides, and enzymes purchased from New England Biolabs or Trilink Biotechnologies.

The DNA sequence (SEQ ID No: 603) that was transcribed into RNA comprised the following elements in order from 5′ to 3′: the T7 RNA polymerase promoter, a 5′ untranslated region (5′ UTR), a nuclear localization signal, a short linker, the coding sequence for the MG3-6/3-4 nuclease, a short linker, a nuclear localization signal, and a 3′ untranslated region and an approximately 100 nucleotide polyA tail (not included in SEQ ID No: 603).

The protein sequence encoded in the synthetic mRNA encoded in this MG3-6/3-4 cassette comprises the following elements from 5′ to 3′: the nuclear localization signal from SV40, a five amino acid linker (GGGS), the protein coding sequence of the MG3-6/3-4 nuclease from which the initiating methionine codon was removed, a 3 amino acid linker (SGG) and the nuclear localization signal from nucleoplasmin. The DNA sequence of the protein coding region of this cassette was modified to reflect the codon usage in humans using a commercially available algorithm. An approximately 100-nucleotide polyA tail was encoded in the plasmid used for in vitro transcription and the mRNA was co-transcriptionally capped using the CleanCAP™ reagent purchased from Trilink Biotechnologies. Uridine in the mRNA was replaced with N1-methyl pseudouridine.

The lipid nanoparticle (LNP) formulation used to deliver the MG3-6/3-4 mRNA and the guide RNA is based on LNP formulations described in the literature including Kauffman et al (Nano Lett. 2015, 15, 11, 7300-7306 (https://doi.org/10.1021/acs.nanolett.5b024970). The four lipid components were dissolved in ethanol and mixed in an appropriate molar ratio to make the lipid working mix. The mRNA and the guide RNA were either mixed prior to formulation at a 1:1 mass ratio or formulated in separate LNP that were later co-injected into mice at a 1:1 mass ratio of the two RNA's. In either case, the RNA was diluted in 100 mM Sodium Acetate (pH 4.0) to make the RNA working stock. The lipid working stock and the RNA working stock were mixed in a microfluidics device (Ignite NanoAssembler, Precision Nanosystems) at a flow rate ratio of 1:3, respectively and a flow rate of 12 mLs/min. The LNP were dialyzed against phosphate buffered saline (PBS) for 2 to 16 hours and then concentrated using Amicon spin concentrators (Millipore) until the reduced volume was achieved. The concentration of RNA in the LNP formulation was measured using the Ribogreen reagent (Thermo Fisher). The diameter and polydispersity (PDI) of the LNP were determined by dynamic light scattering. Representative LNP had diameters ranged from 65 nm to 120 nm with PDI of 0.05 to 0.20. LNP were injected intravenously into 8- to 12-week-old C57B16 wild type mice via the tail vein (0.1 mL per mouse) at a total RNA dose of 1 mg RNA per kg body weight. Eleven days post-dosing, 3 of the 5 mice in each group were sacrificed and the liver was collected and homogenized using a bead beater (Omni International) in a digestion buffer supplied in the PureLink Genomic DNA Isolation Kit (Thermo Fisher Scientific). Genomic DNA was purified from the resulting homogenate using the PureLink Genomic DNA Isolation Kit (Thermo Fisher Scientific) and quantified by measuring the absorbance at 260 nm. Genomic DNA purified from mice injected with buffer alone was used as a control. At 28 days post-dosing, the remaining 2 mice in each group were sacrificed and the liver was collected and homogenized using a bead beater (Omni International) in a digestion buffer supplied in the PureLink Genomic DNA Isolation Kit (Thermo Fisher Scientific). Genomic DNA was purified from the resulting homogenate using the PureLink Genomic DNA Isolation Kit (Thermo Fisher Scientific) and quantified by measuring the absorbance at 260 nm. Genomic DNA purified from mice injected with buffer alone was used as a control.

The liver genomic DNA was then PCR amplified using a first set of primers flanking the region targeted by the two guides. The PCR primers used are shown below in Table 10.

TABLE 10
Sequences of PCR primers and Next
Generation Sequencing primers used
to analyze in vivo genome editing
in mice

| Primer | | Left | Right |
|---|---|---|---|
| Set | | Prime | Primer |
| Name | Purpose | Sequence | Sequence |
| mHAO1-NGS- | Amplify the | GTAAAGAAA | ATCTGTCAA |
| P4 | target site in | AACAAGGAA | CTTCTGTTT |
| | HAO1 exon 2 | TGTAAT | TAGGAC |
| | for guide | | |
| | mH364-7 | | |
| mHAO1-NGS- | Amplify the | GCAAAGTAG | ACCAAGTCA |
| P5 | target site in | AGAAATG | GATATAAAC |
| | HAOI exon 4 | ACAAACC | TGTCT |
| | for guide | | |
| | mH364-20 | | |

The 5′ end of these primers comprise conserved regions complementary to the PCR primers used in the second PCR, followed by 5 Ns in order to give sequence diversity and improve MiSeq sequencing quality, and end with sequences complementary to the target region in the mouse genome. PCR was performed using Q5® Hot Start High-Fidelity 2× Master Mix (New England Biolabs) on 100 ng of genomic DNA and an annealing temperature of 60° C. for a total of 30 cycles. This was followed by a 2nd round of 10 cycles of PCR using primers designed to add unique dual Illumina barcodes (IDT) for next generation sequencing on a MiSeq instrument. Each sample was sequenced to a depth of greater than 10,000 reads using 150 bp paired end reads. Reads were merged to generate a single 250 bp sequence from which Indel percentage and INDEL profile was calculated using a proprietary Python Script.

The results of the NGS analysis of INDELS from mice at day 11 post dosing are shown in Table 11 for individual mice and are summarized in FIG. 32.

TABLE 11
Genome editing at the HAO-1 locus by MG3-6/3-4 in
the whole liver of wild type mice at day 11 post LNP
dosing analyzed by next generation sequencing.

| | | | | | | Mean |
|---|---|---|---|---|---|---|
| Ani- | | Total | | % of | | total |
| mal | | NGS | Indel | Indels | Mean | OOF |
| # | Guide RNA | reads | % | OOF | INDELS | % |
| 1 | PBS control | 210962 | 0.09 | 100 | 0.2 | 0.2 |
| 2 | PBS control | 259982 | 0.29 | 99.87 | | |
| 3 | PBS control | 211193 | 0.08 | 100 | | |
| 6 | 364mHA-G7-1 | 164396 | 54.06 | 87.02 | 53.0 | 46.0 |
| 7 | 364mHA-G7-1 | 163409 | 51.93 | 85.9 | | |
| 8 | 364mHA-G7-1 | 183054 | 52.94 | 87.6 | | |
| 11 | 364mHA-G7-35 | 38835 | 22.71 | 91.57 | 23.6 | 21.1 |
| 12 | 364mHA-G7-35 | 269963 | 26.83 | 89.59 | | |
| 13 | 364mHA-G7-35 | 190007 | 21.32 | 87.11 | | |
| 16 | 364mHA-G20-1 | 227766 | 8.53 | 88.62 | 8.9 | 7.5 |
| 17 | 364mHA-G20-1 | 202915 | 5.01 | 90.36 | | |
| 18 | 364mHA-G20-1 | 236757 | 13.06 | 80.52 | | |
| 21 | 364mHA-G20-35 | 177059 | 2.78 | 80.98 | 2.5 | 2.0 |
| 22 | 364mHA-G20-35 | 163515 | 2.29 | 67.62 | | |
| 23 | 364mHA-G20-35 | 136634 | 2.31 | 89.32 | | |

Data for individual mice is shown.
All mice that received guide RNA LNP also received LNP encapsulating the MG3-6/3-4 mRNA.
% of indels OOF is the percentage of all the INDELS that resulted in a sequence where the HAO1 coding sequence is out of frame.
The mean total OOF % is the average percentage of all alleles in which the HAO1 coding sequence is out of frame.
The total number of NGS sequencing reads is given.

Group 2 mice received LNP encapsulating guide RNA mH364-7-1. Group 3 mice received LNP encapsulating guide RNAmH364-7-35. Group 4 mice received LNP encapsulating guide RNA mH364-20-1. Group 5 mice received LNP encapsulating guide RNAmH364-20-35. All mice in groups 2 to 5 also received LNP encapsulating the MG3-6/3-4 mRNA that was mixed with the guide RNA containing LNP at a 1:1 RNA mass ratio prior to injection. No INDELS were detected in the liver of mice injected with PBS buffer (see Table 11). Mice injected with LNPs encapsulating guide 364mHA-G7-1 and MG3-6/3-4 mRNA exhibited INDELS at the target site in HAO-1 at a mean frequency of 53.0%. Mice injected with LNPs encapsulating guide 364mHA-G7-35 and MG3-6/3-4 mRNA exhibited INDELS at the target site in HAO-1 at a mean frequency of 23.6%. Mice injected with LNPs encapsulating guide 364mHA-G20-1 and MG3-6/3-4 mRNA exhibited INDELS at the target site in HAO-1 at a mean frequency of 8.9%. Mice injected with LNPs encapsulating guide 364mHA-G20-35 and MG3-6/3-4 mRNA exhibited indels at the target site in HAO-1 at a mean frequency of 2.5%. These data demonstrate that the guides with spacer 7 (364mHA-G7-1 and 364mHA-G7-35) are significantly more potent in vivo than the guides with spacer 20 (364mHA-G20-1 and 364mHA-G20-35) when guides with the same chemical modifications are compared. This is consistent with the higher level of editing observed with these 2 guide sequences in Hepa1-6 cells by mRNA-based transfection (mH364-7 exhibited 46% INDELS and mH364-20 26% INDELS in Hepa1-6 cells). Guide chemistry #1 resulted in higher levels of editing than chemistry #35 for both guide 7 (2.2-fold higher editing with chemistry #1) and guide 20 (3.5-fold higher editing with chemistry #1). These data demonstrate that the MG3-6/3-4 nuclease can edit in vivo in mice at the target site specified by the sgRNA. Moreover, an sgRNA with a set of chemical modifications designated chemistry #1 was able to promote editing at 53% of the genomic DNA in whole liver when delivered using an LNP. The LNP used in these studies is taken up via binding of apolipoprotein E (apoE) to the LNP which is a ligand for binding to the low-density lipoprotein receptor (see e.g. Yan et al, Biochem Biophys Res Commun 2005 328(1):57-62.doi: 10.1016/j.bbrc.2004.12.137, Akinc et al Mol Ther 2010 (7):1357-64, doi: 10.1038/mt.2010.85).

The liver is composed of a number of different cell types. In the liver of mice, the hepatocytes make up about 52% of all cells (and 35% of hepatocytes contain two nuclei), with Kupffer cells (18%), Ito cells (8%), and endothelial cells (22%) making up the remaining cells (Histochem Cell Biol 131, 713-726 https://doi.org/10.1007/s00418-009-0577-1). By extrapolation, without wishing to be bound by theory, about 60% [((52+(0.35×52))/(48+(52+(0.35×52)))] of the total nuclei in the mouse liver are predicted to be derived from hepatocytes. Because the LDL receptor is expressed mainly on hepatocytes in the liver (see e.g. https://www.proteinatlas.org/ENSG00000130164-LDLR/tissue/liver#imid_2815831), the LNP used in the mouse studies described herein is expected to be taken up primarily by hepatocytes. Because hepatocyte nuclei make up about 60% of all nuclei in the whole liver of mice, it can be predicted that if all the hepatocyte nuclei were edited, the level of INDELS measured in the whole liver are predicted to be about 60%. The finding that LNP delivery of MG3-6/3-4 was able to achieve INDEL rates of 53% suggests that the majority of hepatocyte nuclei were edited.

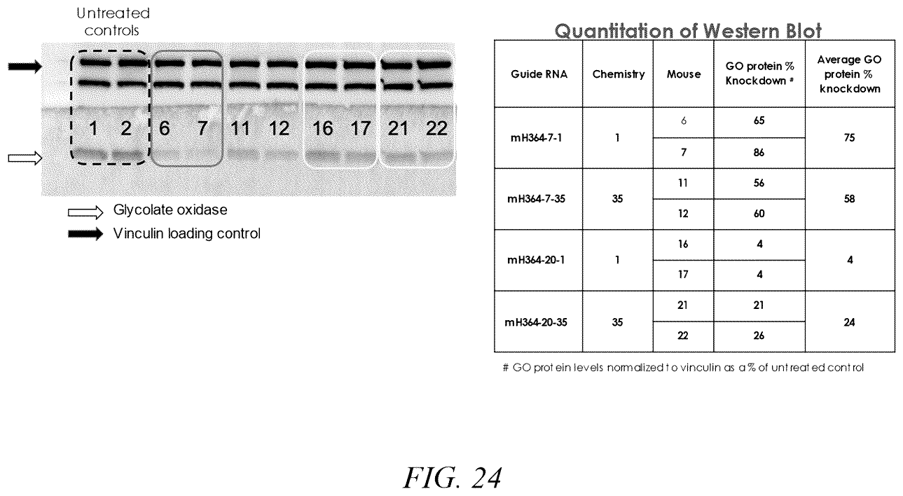

The HAO1 gene encodes the protein glycolate oxidase (GO), an intracellular enzyme involved in glycolate metabolism. To determine if the observed gene editing in the HAO1 gene resulted in a reduction in the expression of the GO protein in the liver, we extracted total protein from a separate lobe of the liver from mice in the same study. The GO protein was detected using a Western blot assay with commercially available antibodies against the mouse GO protein. The protein vinculin was used as a loading control on the Western blot, as Vinculin levels are predicted to not be impacted by gene editing of the HAO1 gene. As shown in FIG. 24, the level of GO protein was significantly reduced in the livers of mice treated with LNP encapsulating MG3-6/3-4 mRNA and sgRNA targeting HAO1. Quantification of the Western blot using image analysis software (Biorad) and normalization of GO to the level of vinculin demonstrated that GO levels were reduced by an average of 75%, 58%, 4%, and 24% in mice treated with sgRNA mH364-7-1, mH364-7-35, mH364-20-1, and mH364-20-35, respectively. The degree of GO protein reduction correlates with the INDEL frequency in these groups of mice (see Table 11). These data demonstrate that the MG3-6/3-4 nuclease combined with an appropriately designed sgRNA can be used to create indels in a gene of interest in vivo in a living mammal and reduce (knockdown) the production of the protein encoded by that gene. Reducing the expression of specific genes can be therapeutically beneficial in specific diseases. In the case of the HAO1 gene that encodes the GO protein, reduction of the levels of GO protein in the liver is expected to be beneficial in patients with the hereditary disease primary hyperoxaluria type I (Martin-Higueras, Mol. Ther. 24, 719-725). Thus, the MG3-6/3-4 nuclease, together with an appropriate sgRNA containing appropriate chemical modifications targeting the HAO1 gene, is a potential approach for the treatment of primary hyperoxaluria type I.

### Example 19— Comparison of MG3-6/3-4 Gene Editing Efficiency in Mice Using the Same Guide RNA Sequence with Four Different Chemical Modifications

The impact of chemical modifications to the sgRNA upon in vivo editing efficiency was further investigated by testing 4 different guide chemistries introduced into the same guide RNA sequence. Guide RNA 7 that targets the mouse HAO1 gene was synthesized with chemical modifications #1, #35, #42, or #45. The sequences of these guides are shown below in Table 12.

TABLE 12
Sequences of MG3-6/3-4 sgRNA guide
7 targeting mouse HA01

| Guide name | Sequence |
|---|---|
| mH364-7-1 | mG*mA*mG*CUGGCC |
| | ACUGUGCGAGGUAGU |
| | UGAGAAUCGAAAGAU |
| | UCUUAAUAAGGCAUC |
| | CUUCCGAUGCUGACU |
| | UCUCACCGUCCGUUU |
| | UCCAAUAGGAGCGGG |
| | CGGUAUGU*mU*mU* |
| | mU |
| mH364-7-35 | mG*mA*mG*mC*UGG |
| | CCACUGUGCGAGGUA |
| | GUUGAGAAUCmG*mA |
| | *mA*mA*GAUUCUUA |
| | AUAAGGCAUCmC*mU |
| | *mU*mC*mC*GAUGC |
| | UGACUUCUCACCGUC |
| | CGUUUUCCmA*mA*m |
| | U*mA*GGAGCGGGCG |
| | GUA*mU*mG*mU*mU |
| | *mU*mU |
| mH364-7-42 | mG*mA*mG*mC*fUf |
| | GfGfCfCfAfCfUfG |
| | fUfGfCfGfAfGfGf |
| | UAGUUGAGAAUCG*A |
| | *A*A*GAUUCUUAAU |
| | AAGGCAUCC*U*U*C |
| | *C*GAUGCUGACUUC |
| | UCACCGUCCGUUUUC |
| | CA*A*U*A*GGAGCG |
| | GGCGGUA*mU*mG*m |
| | U*mU*mU*mU |
| mH364-7-45 | mG*mA*mG*mC*fUf |
| | GfGfCfCfAfCfUfG |
| | fUfGfCfGfAfGfGf |
| | UAGUUGAGAAUCmG* |
| | mA*mA*mA*GAUUCU |
| | UAAUAAGGCAUCmC* |
| | mU*mU*mC*mC*GAU |
| | GCUGACUUCUCACCG |
| | UCCGUUUUCCmA*mA |
| | *mU*mA*GGAGCGGG |
| | CGGUA*mU*mG*mU* |
| | mU*mU*mU |

m: 2′-O methyl modified base, *phosphorothioate backbone

The mRNA encoding MG3-6/3-4 nuclease was generated by in vitro transcription of a linearized plasmid template using T7 RNA polymerase, nucleotides, and enzymes purchased from New England Biolabs or Trilink Biotechnologies. The DNA sequence that was transcribed into RNA comprised the following elements in order from 5′ to 3′: the T7 RNA polymerase promoter, a untranslated region (5′ UTR), a nuclear localization signal, a short linker, the coding sequence for the MG3-6/3-4 nuclease, a short linker, a nuclear localization signal, and a 3′ untranslated region (SEQ ID No: 603) and an approximately 100 nucleotide polyA tail (not included in SEQ ID No: 603)

The protein sequence encoded in the synthetic mRNA encoded in this MG3-6/3-4 cassette comprises the following elements from 5′ to 3′: the nuclear localization signal from SV40, a five amino acid linker (GGGS), the protein coding sequence of the MG3-6/3-4 nuclease from which the initiating methionine codon was removed, a 3 amino acid linker (SGG), and the nuclear localization signal from nucleoplasmin. The DNA sequence of the protein coding region of this cassette was modified to reflect the codon usage in humans using a commercially available algorithm. An approximately 100 nucleotide polyA tail was encoded in the plasmid used for in vitro transcription, and the mRNA was co-transcriptionally capped using the CleanCAP™ reagent purchased from Trilink Biotechnologies. Uridine in the mRNA was replaced with N1-methyl pseudouridine. The lipid nanoparticle (LNP) formulation used to deliver the MG3-6/3-4 mRNA and the guide RNA is based on LNP formulations described in the literature including Kauffman et al (Nano Lett. 2015, 15, 11, 7300-7306, https://doi.org/10.1021/acs.nanolett.5b024970). The four lipid components were dissolved in ethanol and mixed in an appropriate molar ratio to make the lipid working mix. The mRNA and the guide RNA were either mixed prior to formulation at a 1:1 mass ratio or formulated in separate LNP that were later co-injected into mice at a 1:1 mass ratio of the two RNA's. In either case, the RNA was diluted in 100 mM Sodium Acetate (pH 4.0) to make the RNA working stock. The lipid working stock and the RNA working stock were mixed in a microfluidics device (Ignite NanoAssembler, Precision Nanosystems) at a flow rate ratio of 1:3, respectively, and a flow rate of 12 mLs/min. The LNP were dialyzed against phosphate buffered saline (PBS) for 2 to 16 hours and then concentrated using Amicon spin concentrators (Milipore) until the reduced volume was achieved. The concentration of RNA in the LNP formulation was measured using the Ribogreen reagent (Thermo Fisher). The diameter and polydispersity (PDI) of the LNP were determined by dynamic light scattering. Representative LNP had diameters ranged from 65 nm to 120 nm with PDI of 0.05 to 0.20. LNP were injected intravenously into 8- to 12-week-old C57B16 wild type mice via the tail vein (0.1 mL per mouse) at a total RNA dose of 1 mg RNA per kg body weight. Ten days post-dosing, 3 of the 5 mice in each group were sacrificed and the liver was collected and homogenized using a bead beater (Omni International) in a digestion buffer supplied in the PureLink Genomic DNA Isolation Kit (Thermo Fisher Scientific). Genomic DNA was purified from the resulting homogenate using the PureLink Genomic DNA Isolation Kit (Thermo Fisher Scientific) and quantified by measuring the absorbance at 260 nm. Genomic DNA purified from mice injected with buffer alone was used as a control. At 28 days post-dosing, the remaining 2 mice in each group were sacrificed and the liver was collected and homogenized using a bead beater (Omni International) in a digestion buffer supplied in the PureLink Genomic DNA Isolation Kit (Thermo Fisher Scientific). Genomic DNA was purified from the resulting homogenate using the PureLink Genomic DNA Isolation Kit (Thermo Fisher Scientific) and quantified by measuring the absorbance at 260 nm. Genomic DNA purified from mice injected with buffer alone was used as a control.

The liver genomic DNA was then PCR amplified using a first set of primers flanking the region targeted by the two guides. The PCR primers used are shown in Table 10. The 5′ end of these primers comprise conserved regions complementary to the PCR primers used in the second PCR, followed by 5 Ns in order to give sequence diversity and improve MiSeq sequencing quality, and end with sequences complementary to the target region in the mouse genome. PCR was performed using Q5® Hot Start High-Fidelity 2× Master Mix (New England Biolabs) on 100 ng of genomic DNA and an annealing temperature of 60° C. for a total of 30 cycles. This was followed by a 2nd round of 10 cycles of PCR using primers designed to add unique dual Illumina barcodes (IDT) for next generation sequencing on a MiSeq instrument. Each sample was sequenced to a depth of greater than 10,000 reads using 150 bp paired end reads. Reads were merged to generate a single 250 bp sequence from which Indel percentage and INDEL profile was calculated using a proprietary Python Script.

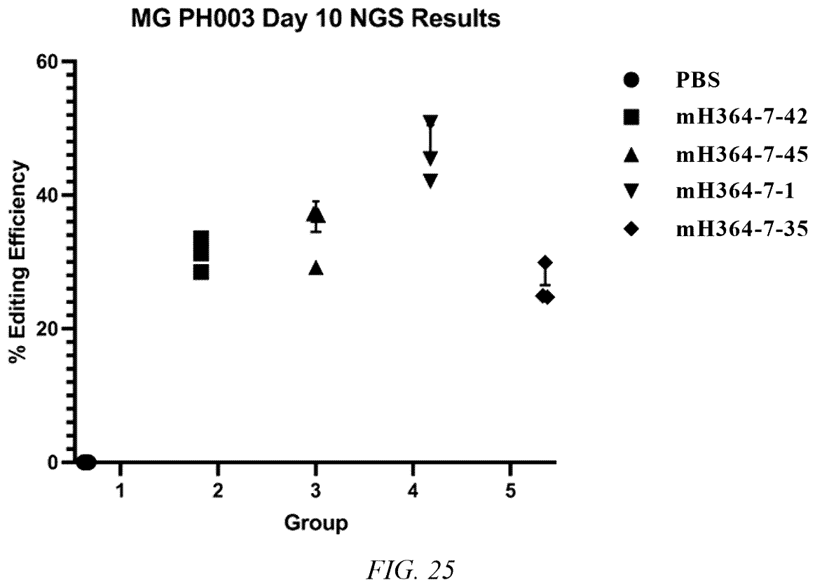

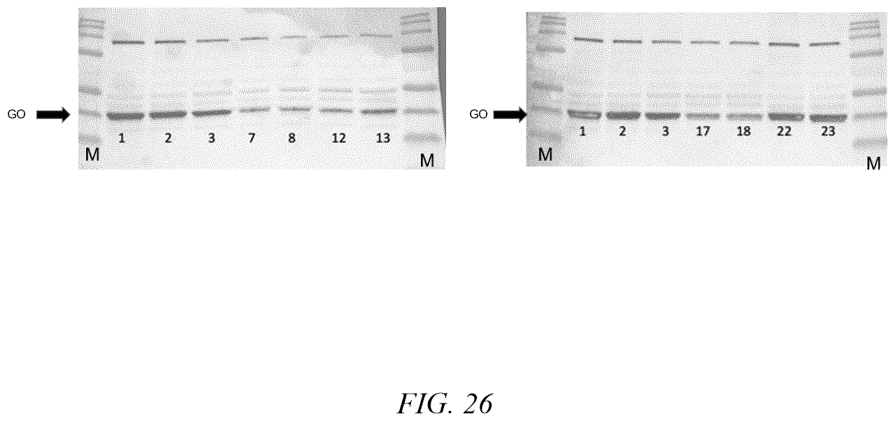

The editing results are summarized in FIG. 25 and tabulated in Table 13.

TABLE 13
Genome editing frequencies in the HAO1 gene in the whole
liver of individual mice treated with LNP encapsulating
MG3-6/3-4 mRNA and guide RNA 7 targeting the HAO-1
gene with chemical modifications 42 (mH364-7-42), 45
(mH364-7-45), 1 (mH364-7-1), and 35 (mH364-7-35)

| | mH364 Guide 7 | | | Mean Group | |
|---|---|---|---|---|---|
| DAY | chemistry | Mouse | INDEL % | INDELS | Stdev |
| 10 | PBS control | 1 | 0.01 | | |
| 10 | PBS control | 2 | 0.01 | | |
| 10 | PBS control | 3 | 0.01 | 0.0 | 0.0 |
| 28 | PBS control | 4 | 0.02 | | |
| 28 | PBS control | 5 | 0.02 | | |
| 10 | 42 | 6 | 33.54 | 32.4 | 2.5 |
| 10 | 42 | 7 | 28.48 | | |
| 10 | 42 | 8 | 31.3 | | |
| 28 | 42 | 9 | 34.43 | | |
| 28 | 42 | 10 | 34.19 | | |
| 10 | 45 | 11 | 29.22 | 32.1 | 5.8 |
| 10 | 45 | 12 | 37.04 | | |
| 10 | 45 | 13 | 37.24 | | |
| 28 | 45 | 14 | 33.57 | | |
| 28 | 45 | 15 | 23.63 | | |
| 10 | 1 | 16 | 42.04 | 46.1 | 3.1 |
| 10 | 1 | 17 | 45.38 | | |
| 10 | 1 | 18 | 50.8 | | |
| 28 | 1 | 19 | 46.31 | | |
| 28 | 1 | 20 | 45.98 | | |
| 10 | 35 | 21 | 24.95 | 26.6 | 2.3 |
| 10 | 35 | 22 | 29.93 | | |
| 10 | 35 | 23 | 24.75 | | |
| 28 | 35 | 24 | 28.14 | | |
| 28 | 35 | 25 | 25.22 | | |

Control mice injected with PBS buffer did not contain measurable INDELS at the target site for guide 7. The mean INDEL frequency in mice that received LNP containing guides mH364-7-1, mH364-7-35, mH364-7-42, and mH364-7-45 was 46.1%, 26.6%, 32.4%, and 32.1%, respectively, demonstrating that guide RNA chemistry #1 was the most potent followed by #42 and #45, with chemistry #35 being the least potent. These data suggest that chemical modifications to the bases and backbone at the 5′ and 3′ ends of the guide RNA provided the highest in vivo potency amongst the chemistries tested. Additional modifications of internal bases did not improve in vivo potency. These findings are in contrast with published data for the spCas9 sgRNA where modifications of bases or the backbone at both the ends of the sgRNA and at internal sequences was required for optimal in vivo editing (Yin et al, Nature Biotechnology, doi:10.1038/nbt.4005) and modifications of just the 5′ and 3′ ends of the sgRNA enabled low levels of editing (20% INDELS) in the liver using delivery in a similar LNP.

Total RNA was purified from a separate lobe of the liver from the same mice described in Table 13 and used to measure level of HAO-1 mRNA by digital droplet PCR (dd-PCR). The PBS injected mice were used as controls and the levels of HAO-1 mRNA in the livers of edited mice were compared to these controls. The dd-PCR assay was designed and optimized using standard techniques. ddPCR is a highly accurate method for determining the absolute copy number of a specific nucleic acid in a complex mixture (e.g. Taylor et al Sci Rep 7, 2409 (2017). doi:10.1038/s41598-017-02217-x). The total liver RNA was first converted to cDNA by reverse transcription then quantified in the dd-PCR assay using GAPDH as an internal control to normalize between samples. As shown in Table 14, the level of HAO1 mRNA in the individual mice treated with LNP encapsulating MG3-6/3-4 mRNA and sgRNA targeting the mouse HAO1 gene was decreased, and the magnitude of decrease was correlated with the INDEL frequency.

TABLE 14
HAO1 mRNA levels in the whole liver of individual mice treated
with LNP encapsulating MG3-6/3-4 mRNA and guide RNA 7 targeting
the HAO-1 gene with chemical modifications 42 (mH364-7-42),
45 (mH364-7-45), 1 (mH364-7-1), and 35 (mH364-7-35).

| | | | | Mean Group | |
|---|---|---|---|---|---|
| Harvest | mH364 Guide 7 | | % Decrease in | % decrease in | |
| Day | chemistry | Mouse | HAO mRNA | HAO mRNA | Stdev |
| 10 | 42 | 6 | 47.4 | 35.5 | 8.8 |
| 10 | 42 | 7 | 42.4 | | |
| 10 | 42 | 8 | 29.0 | | |
| 28 | 42 | 9 | 29.6 | | |
| 28 | 42 | 10 | 28.9 | | |
| 10 | 45 | 11 | 20.3 | 38.0 | 10.2 |
| 10 | 45 | 12 | 38.6 | | |
| 10 | 45 | 13 | 41.8 | | |
| 28 | 45 | 14 | 45.9 | | |
| 28 | 45 | 15 | 43.2 | | |
| 10 | 1 | 16 | 57.0 | 60.0 | 3.9 |
| 10 | 1 | 17 | 54.7 | | |
| 10 | 1 | 18 | 62.5 | | |
| 28 | 1 | 19 | 63.1 | | |
| 28 | 1 | 20 | 62.6 | | |
| 10 | 35 | 21 | 18.3 | 23.4 | 20.8 |
| 10 | 35 | 22 | −2.5 | | |
| 10 | 35 | 23 | 14.8 | | |
| 28 | 35 | 24 | 52.6 | | |
| 28 | 35 | 25 | 33.8 | | |

The same mice in Table 10 were analyzed

The largest reduction in HAO1 mRNA was seen in the group of mice treated with sgRNA mH364-7-1, while the smallest reduction of HAO-1 mRNA was observed in mice treated with sgRNA mH364-7-35. A reduction in HAO1 mRNA can occur when frameshift mutations are introduced into the coding sequence of a gene via a mechanism called nonsense mediated decay (Brogna et al, Nat Struct Mol Biol 16, 107-113 (2009), doi:10.1038/nsmb.1550). The observation of reduced HAO-1 mRNA in the liver of mice edited at the HAO-1 gene with MG3-6/3-4 is consistent with the presence of INDELS that result in a high rate of frame shifts as shown in Table 15.

TABLE 15
Analysis of the frequency of edits that result in frame shifts
in the liver of mice treated with LNP encapsulating MG3-6/3-4
mRNA and sgRNA number 7 (G7) that targets the HAO-1 gene

| | Mean | Stdev of | Mean OOF % | Stdev OFF % |
|---|---|---|---|---|
| Treatment | INDELS | INDELS | total | total |
| PBS control | 0.0 | 0.0 | 0.0 | 0.0 |
| mH364-7-42 | 31.1 | 2.1 | 28.6 | 1.7 |
| mH364-7-45 | 34.5 | 3.7 | 31.2 | 3.2 |
| mH364-7-1 | 46.1 | 3.6 | 41.9 | 3.4 |
| mH364-7-35 | 26.5 | 2.4 | 24.3 | 2.5 |

The out of frame percentage (OOF %) was calculated by analyzing the NGS data using a custom algorithm

In Table 15, the mean frequency of INDELS that result in a frame shift in the HAO1 coding sequence were determined from the NGS data. This analysis shows that the majority of the INDELS resulted in a frameshift for all four of the sgRNA tested.

The HAO1 gene encodes the protein glycolate oxidase (GO) that is an intracellular enzyme involved in glycolate metabolism. To determine if the observed gene editing in the HAO1 gene resulted in a reduction in the expression of the GO protein in the liver, we extracted total protein from a separate lobe of the liver from mice in the same study described in FIG. 25 and Tables 13 to 15. The GO protein was detected using a Western blot assay with commercially available antibodies against the mouse GO protein. Equal amounts of protein were loaded on the Western blot. As shown in FIG. 25, the level of GO protein was reduced in the livers of mice treated with LNP encapsulating MG3-6/3-4 mRNA and sgRNA targeting HAO1. Guides mH364-7-42 (mice 7,8), mH364-7-45 (mice 12, 13), and mH364-7-1 (mice 17,18) resulted in clear reductions in GO protein. Guide mH364-7-35 (mice 22,23) which had the lowest levels of INDELS among the 4 guides tested, did not appreciably reduce GO protein levels. These data demonstrate that the MG3-6/3-4 nuclease combined with an appropriately designed sgRNA can be used to create INDELS in a gene of interest in vivo in a living mammal and reduce (knockdown) the production of the protein encoded by that gene. Reducing the expression of specific genes can be therapeutically beneficial in specific diseases. In the case of the HAO1 gene that encodes the GO protein, reduction of the levels of GO protein in the liver is expected to be beneficial in patients with the hereditary disease primary hyperoxaluria type I (Martin-Higueras, Mol. Ther. 24, 719-725). Thus the MG3-6/3-4 nuclease, together with an appropriate sgRNA containing appropriate chemical modifications targeting the HAO1 gene, is a potential approach for the treatment of primary hyperoxaluria type I.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A fusion endonuclease comprising:
(a) an N-terminal sequence comprising at least part of a RuvC domain, a REC domain, or an HNH domain of an endonuclease having at least 80% sequence identity to SEQ ID NO: 696 or a variant thereof; and
(b) a C-terminal sequence comprising WED, TOPO, or CTD domains of an endonuclease having at least 80% sequence identity to SEQ ID NO: 706 or 708 or variants thereof, wherein said N-terminal sequence and said C-terminal sequence do not naturally occur together in a same reading frame.

2. The fusion endonuclease of claim 1, wherein said C-terminal sequence comprising WED, TOPO, or CTD domains of said fusion endonuclease has at least 80% sequence identity to SEQ ID NO: 706 or a variant thereof.

3. The fusion endonuclease of claim 1, wherein said N-terminal sequence and said C-terminal sequence are derived from different organisms.

4. The fusion endonuclease of claim 1, wherein said N-terminal sequence further comprises RuvC-I, BH, or RuvC-II domains of an endonuclease having at least 80% sequence identity to SEQ ID NO:696 or a variant thereof.

5. The fusion endonuclease of claim 1, wherein said C-terminal sequence further comprises a PAM-interacting domain.

6. The fusion endonuclease of claim 1, wherein said fusion endonuclease comprises a sequence having at least 80% sequence identity to SEQ ID NO: 10 or a variant thereof.

7. The fusion endonuclease claim 1, wherein said fusion endonuclease is configured to have selectivity for a PAM that is not nnRGGnT (SEQ ID NO: 53).

8. The fusion endonuclease of claim 7, wherein said fusion endonuclease is configured to have selectivity for a PAM that comprises any one of SEQ ID NOs: 62 or 64.

9. The fusion endonuclease of claim 8, wherein said fusion endonuclease is configured to have selectivity for a PAM that comprises SEQ ID NO: 62.

10. The fusion endonuclease of claim 1, wherein said fusion endonuclease is a class II, type II Cas endonuclease.

11. The fusion endonuclease of claim 10, wherein said class II, type II Cas endonuclease is derived from an uncultivated microorganism.

12. The fusion endonuclease of claim 1, wherein said fusion endonuclease has less than 86% identity to a SpyCas9 endonuclease.

13. A fusion endonuclease comprising an engineered amino acid sequence having at least 80% sequence identity to any one of SEQ ID NOs: 10 or 12, or a variant thereof.

14. The fusion endonuclease of claim 13, wherein said fusion endonuclease is configured to have selectivity for a PAM that comprises any one of SEQ ID NOs: 62 or 64.

15. The fusion endonuclease of claim 13, wherein said endonuclease has at least 55% sequence identity to SEQ ID NO: 10 or a variant thereof.

16. An engineered nuclease system, comprising:
(a) the fusion endonuclease of claim 1; and
(b) an engineered guide ribonucleic acid structure configured to form a complex with said fusion endonuclease comprising:
a guide ribonucleic acid sequence configured to hybridize to a target deoxyribonucleic acid sequence.

17. The engineered nuclease system of claim 16, wherein said engineered guide ribonucleic acid structure further comprises a tracr ribonucleic acid sequence configured to bind said fusion endonuclease.

18. The engineered nuclease system of claim 16, wherein said fusion endonuclease is derived from an uncultivated microorganism.

19. The engineered nuclease system of claim 16, wherein said fusion endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas13d endonuclease.

20. The engineered nuclease system of claim 16, wherein said fusion endonuclease has less than 86% identity to a SpyCas9 endonuclease.

21. The engineered nuclease system of claim 16, wherein said fusion endonuclease comprises a sequence having at least 80% sequence identity to any one of SEQ ID NOs: 10 or 12, or a variant thereof.

22. The engineered nuclease system of claim 16, wherein said engineered guide ribonucleic acid structure comprises a sequence having at least 80% identity to non-degenerate nucleotides of SEQ ID NO: 35.
